(19) United States
(12) Patent Application Publication
(10) Pub. No.: US 2022/0056444 A1
(43) Pub. Date: Feb. 24, 2022

(54) PROCESS TO INHIBIT OR ELIMINATE EOSINOPHILIC DISEASES OF THE AIRWAY AND RELATED CONDITIONS

(71) Applicant: EMPIRICO INC., San Diego, CA (US)

(72) Inventors: Omri GOTTESMAN, San Diego, CA (US); Shannon BRUSE, San Diego, CA (US); Brian CAJES, San Diego, CA (US); David LEWIS, Madison, WI (US); David ROZEMA, Cross Plains, WI (US)

(21) Appl. No.: 17/298,896

(22) PCT Filed: Dec. 3, 2019
(86) PCT No.: PCT/US2019/064286

§ 371 Date: Jun. 1, 2021

Related U.S. Application Data
(60) Provisional application No. 62/775,729, filed on Dec. 5, 2018.
(60) Provisional application No. 62/882,400, filed on Aug. 2, 2019.

(51) Int. Cl. C12N 15/113; A61K 31/713

(52) U.S. Cl. CPC C12N 15/113; A61K 31/713; C12N 2310/14; C12N 2310/315; C12N 2320/30; C12N 2310/321; C12N 2310/351; C12N 2310/11; C12N 2310/322

(57) ABSTRACT

Molecules for inhibiting arachidonate 15-lipoxygenase (ALOX-15) gene products including dsRNA (dsRNA) agents such as small interfering RNAs (siRNAs) for therapeutic use, additionally, methods to inhibit the expression of a target gene by administering these agents for the treatment of diseases involving ALOX-15 gene products.

### PRIORITY

This application claims the benefit of U.S. Provisional Application No. 62/775,729 filed Dec. 5, 2018, and U.S. Provisional Application No. 62/882,400 filed Aug. 2, 2019, the entirety of both are incorporated herein by reference.

### SEQUENCE LISTING

This application contains a Sequence Listing which has been submitted electronically in ASCII format and is hereby incorporated by reference in its entirety. Said ASCII copy, created on Nov. 22, 2019, is named 54462-708_601_SL.txt and is 1,451,425 bytes in size.

### FIELD

Provided herein are molecules for inhibition of arachidonate 15-lipoxygenase (ALOX-15) gene products, including dsRNA (dsRNA) agents such as small interfering RNAs (siRNAs) for therapeutic use. Additionally, methods of inhibiting the expression of a target gene by administering these agents, e.g., for the treatment of various diseases involving ALOX-15 gene products, are described herein.

### BACKGROUND

The newest generation of therapeutic modalities is designed to directly augment or interfere with the cellular processes that cause disease. These approaches represent a paradigm shift in our ability to directly target or mimic molecular lesions that are observed to be causal for a disease or that lead to an observable trait, either beneficial or deleterious. A preferred example of a therapeutic modality is RNA interference, or “RNAi,” in which double-stranded RNA (dsRNA, also known as interfering RNA, small interfering RNA, or siRNA) can block gene expression. Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

### SUMMARY

Stabilization of synthetic dsRNA, or siRNA, against rapid nuclease degradation is generally regarded as a prerequisite for in vivo and therapeutic applications. This can be achieved using a variety of stabilization chemistries, such as chemical modifications to the native 2′—OH group in the ribose sugar backbone, such as 2′-O-methyl (2′OMe) and 2′-Fluoro (2′F) substitutions that can be readily introduced into siRNA as 2′-modified nucleotides during RNA synthesis. Although a number of reports have demonstrated that chemically stabilized siRNA containing 2′OMe, 2′F, 2′-deoxy, or “locked nucleic acid” (LNA) modifications can be designed that retain functional RNAi activity, in some cases, such modifications may be specific to certain positional or sequence-related contexts. In some cases, the introduction of chemical modifications to native siRNA duplexes can have a negative impact on RNAi activity as noted below. As a result, the design of chemically modified siRNA has required a stochastic screening approach to identify duplexes that retain potent gene silencing activity.

An initial step in RNAi is the activation of the RISC, which requires degradation of the sense strand of the dsRNA duplex. The sense strand acts as a RISC substrate that is cleaved by the endonuclease Argonaute 2 (Ago2) in the middle of the duplex region. Immediately after the cleaved 5′-end and 3′-end fragments of the sense strand are removed from Ago2, the RISC becomes activated by the antisense strand. It has been shown that incorporation of a 2′-O-Me ribose to the Ago2 cleavage site in the sense strand can inhibit RNAi in HeLa cells. A similar effect was observed with phosphorothioate modifications, showing that cleavage of the sense strand was required for efficient RNAi also in mammals. An siRNA duplex containing modifications by 2′-OMe or various combinations of 2′-F, 2′-OMe and phosphorothioate modifications to stabilize siRNA in serum can be active. However, it may be desirable to avoid modification of the cleavage site with 2′-OMe in order to increase the stability of the siRNA.

In some aspects, provided herein is a pharmaceutical composition comprising an siRNA molecule comprising a sense strand and an antisense strand, which targets SEQ ID NO: 5357 and when administered to a patient in an effective amount reduces eosinophil count, wherein (i) the siRNA comprises a modification comprising a modified nucleoside and/or a modified internucleoside linkage, and/or (ii) the pharmaceutical composition comprises a pharmaceutically acceptable carrier; and wherein the patient comprises nasal polyposis, chronic sinusitis, allergic rhinitis, or NSAID-exacerbated respiratory disease, or a combination thereof. In some embodiments, the systemic or local eosinophil count is reduced by about 10% or more as compared to the eosinophil count prior to administration. In some aspects, provided herein is a pharmaceutical composition comprising an siRNA molecule which targets SEQ ID NO: 5357 and when administered in an effective amount to a patient comprising nasal polyps, the nasal polyps are reduced in number and/or size the patient, wherein (i) the siRNA comprises a modification comprising a modified nucleoside and/or a modified internucleoside linkage, and/or (ii) the pharmaceutical composition comprises a pharmaceutically acceptable carrier. In some embodiments, the nasal polyps are reduced in number and/or size by about 10% or more by CT scan or endoscopic assessment, as compared to the number and/or size prior to administration. In some aspects, provided herein is a pharmaceutical composition comprising an siRNA molecule which targets SEQ ID NO: 5357 and when administered to a patient in an effective amount increases nasal inspiratory peak flow in the patient, wherein (i) the siRNA comprises a modification comprising a modified nucleoside and/or a modified internucleoside linkage, and/or (ii) the pharmaceutical composition comprises a pharmaceutically acceptable carrier. In some embodiments, the nasal inspiratory peak flow is increased by about 10% or more, as compared to prior to administration. In some aspects, provided herein is a pharmaceutical composition comprising an siRNA molecule which targets SEQ ID NO: 5357 and when administered to a patient in an effective amount reduces airway symptoms in the patient, wherein the patient comprises nasal polyposis, chronic sinusitis, allergic rhinitis, or NSAID-exacerbated respiratory disease, or a combination thereof, and wherein (i) the siRNA comprises a modification comprising a modified nucleoside and/or a modified internucleoside linkage, and/or (ii) the pharmaceutical composition comprises a pharmaceutically acceptable carrier. In some embodiments, the airway inflammation symptoms are reduced by about 10% or more on a patient-reported outcome measure, as compared to prior to administration. In some aspects, provided herein is a pharmaceutical composition comprising an siRNA molecule which targets SEQ ID NO: 5357 and when administered to a patient in an effective amount improves sense of smell in the patient, wherein the patient comprises nasal polyposis, chronic sinusitis, allergic rhinitis, or NSAID-exacerbated respiratory disease, or a combination thereof, and wherein (i) the siRNA comprises a modification comprising a modified nucleoside and/or a modified internucleoside linkage, and/or (ii) the pharmaceutical composition comprises a pharmaceutically acceptable carrier. In some embodiments, the sense of smell is improved by about 10% or more on a patient-reported outcome measure, as compared to prior to administration. In some aspects, provided herein is a pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6000 (C AGAGCTGCAG ACCTGGTGTC GAGAGATCAC TGAAATCGGG CTGCAAGGGG CCCAGGACCG AGGGTTTCCT GTCTCTTTAC AGGCTCGGGA CCAGGTTTGC CACTTTGTCA CCATGTGTAT CTTCACCTGC ACCGGCCAAC ACGCCTCTGT); wherein (i) the siRNA comprises a modification comprising a modified nucleoside and/or a modified internucleoside linkage, and/or (ii) the pharmaceutical composition comprises a pharmaceutically acceptable carrier. In some embodiments, the sense strand comprises about 14-30 contiguous nucleosides of SEQ ID NO: 6001 (C AGAGCTGCAG ACCTGGTGTC GAGAGATCAC TGAAATCGGG CTGCAAGGGG) or SEQ ID NO: 6002 (CTTTAC AGGCTCGGGA CCAGGTTTGC CACTTTGTCA CCATGTGTAT CTTCA). In some aspects, provided herein is a pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6003 (A CCCTCTTCCC ATGTCCCACC CTCCCTAGAG GGGCACCTTT TCATGGTCTC TGCACCCAGT GAACACATTT TACTCTAGAG GCATCACCTG GGACCTTACT); wherein (i) the siRNA comprises a modification comprising a modified nucleoside and/or a modified internucleoside linkage, and/or (ii) the pharmaceutical composition comprises a pharmaceutically acceptable carrier. In some embodiments, the sense strand comprises about 14-30 contiguous nucleosides of SEQ ID NO: 6004 (TAGAG GGGCACCTTT TCATGGTCTC TGCACCCAGT GAACACATTT TACTC). In some aspects, provided herein is a pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6005 (G ACATGGGAAT TTTCGACCAG ATAATGAGCA CTGGTGGGGG AGGCCACGTG CAGCTGCTCA AGCAAGCTGG AGCCTTCCTA ACCTACAGCT CCTTCTGTCC CCCTGATGAC TTGGCCGACC GGGGGCTCCT GGGAGTGAAG TCTTCCTTCT); wherein (i) the siRNA comprises a modification comprising a modified nucleoside and/or a modified internucleoside linkage, and/or (ii) the pharmaceutical composition comprises a pharmaceutically acceptable carrier. In some embodiments, the sense strand comprises about 14-30 contiguous nucleosides of SEQ ID NO: 6006 (G CAGCTGCTCA AGCAAGCTGG AGCCTTCCTA ACCTACAGCT CCTTCTGTCC).

In some embodiments, the pharmaceutical composition comprises the modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the pharmaceutical composition comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the siRNA comprises a ribose. In some embodiments, the pharmaceutical composition further comprises a lipid attached at either 3′ or 5′ terminus of the sense strand and/or antisense strand of the siRNA. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the sense strand and the antisense strand form a double-stranded RNA duplex. In some embodiments, the double-stranded RNA duplex comprises from about 14 to about 30 nucleosides. In some embodiments, the first base pair of the double-stranded RNA duplex is an AU base pair.

In some embodiments, the sense strand comprises pattern 1S: 5′ fN s mN s fN-mN-fN-mN-fN-fN-fN-mN-fN-mN-fN-mN-fN-mN-fN-mN-fN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the sense strand comprises pattern 2S: 5′ mN s mN s mN-mN-fN-mN-fN-fN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the sense strand comprises pattern 3S: 5′ mN s mN s mN-mN-fN-mN-fN-mN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the sense strand comprises pattern 4S: 5′ fN s mN s fN-mN-fN-mN-fN-fN-fN-mN-fN-mN-fN-mN-fN-mN-fN-mN-fN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides. In some embodiments, the sense strand comprises pattern 5S: 5′ mN s mN s mN-mN-fN-mN-fN-fN-fN-mN-mN-mN-mN-mN-mN-mN-mN-mN-mN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides. In some embodiments, the antisense strand comprises pattern 1AS: 5′ mN s fN s mN-fN-mN-fN-mN-fN-mN-fN-mN-mN-mN-fN-mN-fN-mN-fN-mN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the antisense strand comprises pattern 2AS: 5′ mN s fN s mN-mN-mN-fN-mN-fN-fN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the antisense strand comprises pattern 3AS: 5′ mN s fN s mN-mN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the antisense strand comprises pattern 4AS: 5′ mN s fN s mN-fN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. In some embodiments, the sense strand comprises pattern 1S and the antisense strand comprises pattern 1AS, 2AS, 3AS, or 4AS. In some embodiments, the sense strand comprises pattern 2S and the antisense strand comprises pattern 1AS, 2AS, 3AS, or 4AS. In some embodiments, the sense strand comprises pattern 3S and the antisense strand comprises pattern 1AS, 2AS, 3AS, or 4AS. In some embodiments, the sense strand comprises pattern 4S and the antisense strand comprises pattern 1AS, 2AS, 3AS, or 4AS.

In some embodiments, (i) the sense strand comprises SEQ ID NO: 3027 or a nucleic acid sequence thereof having about 1 or 2 nucleoside substitutions, additions, or deletions, and the antisense strand comprises SEQ ID NO: 3028 or a nucleic acid sequence thereof having about 1 or 2 nucleoside substitutions, additions, or deletions; (ii) the sense strand comprises SEQ ID NO: 5664 or a nucleic acid sequence thereof having about 1 or 2 nucleoside substitutions, additions, or deletions, and the antisense strand comprises SEQ ID NO: 5765 or a nucleic acid sequence thereof having about 1 or 2 nucleoside substitutions, additions, or deletions; (iii) the sense strand comprises SEQ ID NO: 5866 or a nucleic acid sequence thereof having about 1 or 2 nucleoside substitutions, additions, or deletions, and the antisense strand comprises SEQ ID NO: 5967 or a nucleic acid sequence thereof having about 1 or 2 nucleoside substitutions, additions, or deletions; (iv) the sense strand comprises SEQ ID NO: 5558 or a nucleic acid sequence thereof having about 1 or 2 nucleoside substitutions, additions, or deletions, and the antisense strand comprises SEQ ID NO: 5559 or a nucleic acid sequence thereof having about 1 or 2 nucleoside substitutions, additions, or deletions; or (v) the sense strand comprises SEQ ID NO: 5456 or a nucleic acid sequence thereof having about 1 or 2 nucleoside substitutions, additions, or deletions, and the antisense strand comprises SEQ ID NO: 5457 or a nucleic acid sequence thereof having about 1 or 2 nucleoside substitutions, additions, or deletions.

In some embodiments, (i) the sense strand comprises SEQ ID NO: 3037 or a nucleic acid sequence thereof having about 1 or 2 nucleoside substitutions, additions, or deletions, and the antisense strand comprises SEQ ID NO: 3038 or a nucleic acid sequence thereof having about 1 or 2 nucleoside substitutions, additions, or deletions; (ii) the sense strand comprises SEQ ID NO: 5665 or a nucleic acid sequence thereof having about 1 or 2 nucleoside substitutions, additions, or deletions, and the antisense strand comprises SEQ ID NO: 5766 or a nucleic acid sequence thereof having about 1 or 2 nucleoside substitutions, additions, or deletions; (iii) the sense strand comprises SEQ ID NO: 5867 or a nucleic acid sequence thereof having about 1 or 2 nucleoside substitutions, additions, or deletions, and the antisense strand comprises SEQ ID NO: 5968 or a nucleic acid sequence thereof having about 1 or 2 nucleoside substitutions, additions, or deletions; (iv) the sense strand comprises SEQ ID NO: 5560 or a nucleic acid sequence thereof having about 1 or 2 nucleoside substitutions, additions, or deletions, and the antisense strand comprises SEQ ID NO: 5561 or a nucleic acid sequence thereof having about 1 or 2 nucleoside substitutions, additions, or deletions; or (v) the sense strand comprises SEQ ID NO: 5460 or a nucleic acid sequence thereof having about 1 or 2 nucleoside substitutions, additions, or deletions, and the antisense strand comprises SEQ ID NO: 5461 or a nucleic acid sequence thereof having about 1 or 2 nucleoside substitutions, additions, or deletions.

In some embodiments, (i) the sense strand comprises SEQ ID NO: 3183 or a nucleic acid sequence thereof having about 1 or 2 nucleoside substitutions, additions, or deletions, and the antisense strand comprises SEQ ID NO: 3184 or a nucleic acid sequence thereof having about 1 or 2 nucleoside substitutions, additions, or deletions; (ii) the sense strand comprises SEQ ID NO: 5666 or a nucleic acid sequence thereof having about 1 or 2 nucleoside substitutions, additions, or deletions, and the antisense strand comprises SEQ ID NO: 5767 or a nucleic acid sequence thereof having about 1 or 2 nucleoside substitutions, additions, or deletions; (iii) the sense strand comprises SEQ ID NO: 5868 or a nucleic acid sequence thereof having about 1 or 2 nucleoside substitutions, additions, or deletions, and the antisense strand comprises SEQ ID NO: 5969 or a nucleic acid sequence thereof having about 1 or 2 nucleoside substitutions, additions, or deletions; (iv) or the sense strand comprises SEQ ID NO: 5562 or a nucleic acid sequence thereof having about 1 or 2 nucleoside substitutions, additions, or deletions, and the antisense strand comprises SEQ ID NO: 5563 or a nucleic acid sequence thereof having about 1 or 2 nucleoside substitutions, additions, or deletions; or (v) the sense strand comprises SEQ ID NO: 5476 or a nucleic acid sequence thereof having about 1 or 2 nucleoside substitutions, additions, or deletions, and the antisense strand comprises SEQ ID NO: 5477 or a nucleic acid sequence thereof having about 1 or 2 nucleoside substitutions, additions, or deletions.

In some embodiments, (i) the sense strand comprises SEQ ID NO: 4265 or a nucleic acid sequence thereof having about 1 or 2 nucleoside substitutions, additions, or deletions, and the antisense strand comprises SEQ ID NO: 4266 or a nucleic acid sequence thereof having about 1 or 2 nucleoside substitutions, additions, or deletions; (ii) the sense strand comprises SEQ ID NO: 5667 or a nucleic acid sequence thereof having about 1 or 2 nucleoside substitutions, additions, or deletions, and the antisense strand comprises SEQ ID NO: 5768 or a nucleic acid sequence thereof having about 1 or 2 nucleoside substitutions, additions, or deletions; (iii) the sense strand comprises SEQ ID NO: 5869 or a nucleic acid sequence thereof having about 1 or 2 nucleoside substitutions, additions, or deletions, and the antisense strand comprises SEQ ID NO: 5970 or a nucleic acid sequence thereof having about 1 or 2 nucleoside substitutions, additions, or deletions; (iv) the sense strand comprises SEQ ID NO: 5564 or a nucleic acid sequence thereof having about 1 or 2 nucleoside substitutions, additions, or deletions, and the antisense strand comprises SEQ ID NO: 5565 or a nucleic acid sequence thereof having about 1 or 2 nucleoside substitutions, additions, or deletions; or (v) the sense strand comprises SEQ ID NO: 5510 or a nucleic acid sequence thereof having about 1 or 2 nucleoside substitutions, additions, or deletions, and the antisense strand comprises SEQ ID NO: 5511 or a nucleic acid sequence thereof having about 1 or 2 nucleoside substitutions, additions, or deletions.

In some embodiments, (i) the sense strand comprises SEQ ID NO: 2629 or a nucleic acid sequence thereof having about 1 or 2 nucleoside substitutions, additions, or deletions, and the antisense strand comprises SEQ ID NO: 2630 or a nucleic acid sequence thereof having about 1 or 2 nucleoside substitutions, additions, or deletions; (ii) the sense strand comprises SEQ ID NO: 5668 or a nucleic acid sequence thereof having about 1 or 2 nucleoside substitutions, additions, or deletions, and the antisense strand comprises SEQ ID NO: 5769 or a nucleic acid sequence thereof having about 1 or 2 nucleoside substitutions, additions, or deletions; (iii) the sense strand comprises SEQ ID NO: 5870 or a nucleic acid sequence thereof having about 1 or 2 nucleoside substitutions, additions, or deletions, and the antisense strand comprises SEQ ID NO: 5971 or a nucleic acid sequence thereof having about 1 or 2 nucleoside substitutions, additions, or deletions; (iv) the sense strand comprises SEQ ID NO: 5566 or a nucleic acid sequence thereof having about 1 or 2 nucleoside substitutions, additions, or deletions, and the antisense strand comprises SEQ ID NO: 5567 or a nucleic acid sequence thereof having about 1 or 2 nucleoside substitutions, additions, or deletions; or (v) the sense strand comprises SEQ ID NO: 5524 or a nucleic acid sequence thereof having about 1 or 2 nucleoside substitutions, additions, or deletions, and the antisense strand comprises SEQ ID NO: 5525 or a nucleic acid sequence thereof having about 1 or 2 nucleoside substitutions, additions, or deletions.

In some embodiments, the sense strand comprises

(SEQ ID NO: 5558)
mUsmGsmGmUfGmUfCfGfAmGmAmGmAmUmCmAmCmUmAsmUsmU,
(SEQ ID NO: 5560)
mUsmCsmGmAfGmAfGfAfUmCmAmCmUmGmAmAmAmUmAsmUsmU,
(SEQ ID NO: 5562)
mCsmAsmGmGfUmUfUfGfCmCmAmCmUmUmUmGmUmCmAsmUsmU,
(SEQ ID NO: 5564)
mGsmCsmAmCfCmUfUfUfUmCmAmUmGmGmUmCmUmCmAsmUsmU,
(SEQ ID NO: 5566)
mAsmGsmCmUfGmGfAfGfCmCmUmUmCmCmUmAmAmCmAsmUsmU,
(SEQ ID NO: 5456)
fUsmGsfGmUfGmUfCfGfAmGfAmGfAmUfCmAfCmUfAsmUsmU,
(SEQ ID NO: 5460)
fUsmCsfGmAfGmAfGfAfUmCfAmCfUmGfAmAfAmUfAsmUsmU,
(SEQ ID NO: 5476)
fCsmAsfGmGfUmUfUfGfCmCfAmCfUmUfUmGfUmCfAsmUsmU,
(SEQ ID NO: 5510)
fGsmCsfAmCfCmUfUfUfUmCfAmUfGmGfUmCfUmCfAsmUsmU,
(SEQ ID NO: 5524)
fAsmGsfCmUfGmGfAfGfCmCfUmUfCmCfUmAfAmCfAsmUsmU,
(SEQ ID NO: 6050)
mUsmGsmGmUfGmUfCmGfAmGmAmGmAmUmCmAmCmUmAsmUsmU,
(SEQ ID NO: 6051)
mUsmCsmGmAfGmAfGmAfUmCmAmCmUmGmAmAmAmUmAsmUsmU,
(SEQ ID NO: 6052)
mCsmAsmGmGfUmUfUmGfCmCmAmCmUmUmUmGmUmCmAsmUsmU,
(SEQ ID NO: 6053)
mGsmCsmAmCfCmUfUmUfUmCmAmUmGmGmUmCmUmCmAsmUsmU,
(SEQ ID NO: 6054)
mAsmGsmCmUfGmGfAmGfCmCmUmUmCmCmUmAmAmCmAsmUsmU,
(SEQ ID NO: 6055)
fUsmGsfGmUfGmUfCfGfAmGfAmGfAmUfCmAfCmUfAsmUsmUNNN-
lipid,
(SEQ ID NO: 6056)
fUsmCsfGmAfGmAfGfAfUmCfAmCfUmGfAmAfAmUfAsmUsmUNNN-
lipid,
(SEQ ID NO: 6057)
fCsmAsfGmGfUmUfUfGfCmCfAmCfUmUfUmGfUmCfAsmUsmUNNN-
lipid,
(SEQ ID NO: 6058)
fGsmCsfAmCfCmUfUfUfUmCfAmUfGmGfUmCfUmCfAsmUsmUNNN-
lipid,
(SEQ ID NO: 6059)
fAsmGsfCmUfGmGfAfGfCmCfUmUfCmCfUmAfAmCfAsmUsmUNNN-
lipid,
(SEQ ID NO: 6060)
mUsmGsmGmUfGmUfCfGfAmGmAmGmAmUmCmAmCmUmAsmUsmUNNN-
lipid,
(SEQ ID NO: 6061)
mUsmCsmGmAfGmAfGfAfUmCmAmCmUmGmAmAmAmUmAsmUsmUNNN-
lipid,
(SEQ ID NO: 6062)
mCsmAsmGmGfUmUfUfGfCmCmAmCmUmUmUmGmUmCmAsmUsmUNNN-
lipid,
(SEQ ID NO: 6063)
mGsmCsmAmCfCmUfUfUfUmCmAmUmGmGmUmCmUmCmAsmUsmUNNN-
lipid,
or
(SEQ ID NO: 6064)
mAsmGsmCmUfGmGfAfGfCmCmUmUmCmCmUmAmAmCmAsmUsmUNNN-
lipid,

or any combination thereof.

In some embodiments, the antisense strand comprises

(SEQ ID NO: 5559)
mUsfAsmGmUmGfAmUmCmUmCmUmCmGfAmCfAmCmCmAsmUsmU,
(SEQ ID NO: 5561)
mUsfAsmUmUmUfCmAmGmUmGmAmUmCfUmCfUmCmGmAsmUsmU,
(SEQ ID NO: 5563)
mUsfGsmAmCmAfAmAmGmUmGmGmCmAfAmAfCmCmUmGsmUsmU,
(SEQ ID NO: 5565)
mUsfGsmAmGmAfCmCmAmUmGmAmAmAfAmGfGmUmGmCsmUsmU,
(SEQ ID NO: 5567)
mUsfGsmUmUmAfGmGmAmAmGmGmCmUfCmCfAmGmCmUsmUsmU,
(SEQ ID NO: 5457)
mUsfAsmGfUmGfAmUfCmUfCmUmCmGfAmCfAmCfCmAsmUsmU,
(SEQ ID NO: 5461)
mUsfAsmUfUmUfCmAfGmUfGmAmUmCfUmCfUmCfGmAsmUsmU,
(SEQ ID NO: 5477)
mUsfGsmAfCmAfAmAfGmUfGmGmCmAfAmAfCmCfUmGsmUsmU,
(SEQ ID NO: 5511)
mUsfGsmAfGmAfCmCfAmUfGmAmAmAfAmGfGmUfGmCsmUsmU,
(SEQ ID NO: 5525)
mUsfGsmUfUmAfGmGfAmAfGmGmCmUfCmCfAmGfCmUsmUsmU,
(SEQ ID NO: 6065)
mUsfAsmGmUmGfAmUfCfUmCmUmCmGfAmCfAmCmCmAsmUsmU,
(SEQ ID NO: 6066)
mUsfAsmUmUmUfCmAfGfUmGmAmUmCfUmCfUmCmGmAsmUsmU,
(SEQ ID NO: 6067)
mUsfGsmAmCmAfAmAfGfUmGmGmCmAfAmAfCmCmUmGsmUsmU,
(SEQ ID NO: 6068)
mUsfGsmAmGmAfCmCfAfUmGmAmAmAfAmGfGmUmGmCsmUsmU,
(SEQ ID NO: 6069)
mUsfGsmUmUmAfGmGfAfAmGmGmCmUfCmCfAmGmCmUsmUsmU,
(SEQ ID NO: 6070)
mUsfAsmGfUmGfAmUmCmUmCmUmCmGfAmCfAmCmCmAsmUsmU,
(SEQ ID NO: 6071)
mUsfAsmUfUmUfCmAmGmUmGmAmUmCfUmCfUmCmGmAsmUsmU,
(SEQ ID NO: 6072)
mUsfGsmAfCmAfAmAmGmUmGmGmCmAfAmAfCmCmUmGsmUsmU,
(SEQ ID NO: 6073)
mUsfGsmAfGmAfCmCmAmUmGmAmAmAfAmGfGmUmGmCsmUsmU,
or
(SEQ ID NO: 6074)
mUsfGsmUfUmAfGmGmAmAmGmGmCmUfCmCfAmGmCmUsmUsmU,

or any combination thereof.

In some embodiments, the sense strand or antisense strand comprises one or more sequences selected from SEQ ID NOS: 1-5349, 5366-5971, 6000-6038, 6050-6074, or a nucleic acid sequence thereof having about 1 or 2 nucleoside substitutions, additions, or deletions. In some embodiments, the sense strand or antisense strand comprises one or more sequences comprising the first 19 nucleobases of a sequence selected from SEQ ID NOS: 1-5349, 5366-5971, 6000-6038, 6050-6074, or a nucleic acid sequence thereof having about 1 or 2 nucleoside substitutions, additions, or deletions.

In some aspects, provided herein is a pharmaceutical composition comprising a nucleic acid sequence comprising Formula IA: 5′ Z1-U-Z2-G-Z3-U-C-Z4-A-U-U-Z5-L 3′ wherein Z1 is 0, 3, 5, 11, or 13 nucleosides; Z2 is 1 nucleoside; Z3 is 0, 2, 5, 8, or 10 nucleosides; Z4 is 0, 2, 3, 5, 8, or 11 nucleosides; Z5 is 0 or 3 nucleosides; each “-” is independently a phosphodiester or modified internucleoside linkage; and L is an optional lipid; wherein the nucleic acid comprises a modified nucleoside and/or modified internucleoside linkage. In some embodiments, Z1 comprises 0 nucleosides or UGGUG, CAGGU, CAGGUUUGCCACU (SEQ ID NO: 6007), GCACCUUUUCA (SEQ ID NO: 6008), or AGC; Z2 comprises C, G, or U; Z3 comprises 0 nucleosides or UG, UGUCGAGAGA (SEQ ID NO: 6009), AGAGA, AGAGA, CCACUUUG, or AGCCU; Z4 comprises 0 nucleosides or GAGAGAUCACU (SEQ ID NO: 6010), ACU, ACUGAAAU, UC, or CUAAC; and Z5 comprises 0 nucleosides or UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some aspects, provided herein is a pharmaceutical composition comprising a nucleic acid sequence comprising Formula IB: 5′ U-Z6-G-A-Z7-C-Z8-A-Z9-U-U 3′ wherein Z6 represents 0, 2, 3, 5, 8, or 11 nucleosides; Z7 represents 0, 2, 5, 8, or 10 nucleosides; Z8 represents 1 nucleoside; Z9 represents 0, 3, 5, 11, or 13 nucleosides; and each “-” is independently a phosphodiester or modified internucleoside linkage. In some embodiments, the nucleic acid sequence comprising Formula IA is a sense strand, and the pharmaceutical composition further comprises an anti-sense strand comprising Formula IB: 5′ U-Z6-G-A-Z7-C-Z8-A-Z9-U-U 3′ wherein Z6 represents 0, 2, 3, 5, 8, or 11 nucleosides; Z7 represents 0, 2, 5, 8, or 10 nucleosides; Z8 represents 1 nucleoside; Z9 represents 0, 3, 5, 11, or 13 nucleosides; and each “-” is independently a phosphodiester or modified internucleoside linkage. In some embodiments, Z6 is 0 nucleosides or AGU, AGUGAUCUCUC (SEQ ID NO: 6016), AUUUCAGU, GA, or GUUAG; Z7 is 0 nucleosides or UCUCU, CA, UCUCU, CAAAGUGG, GA, or AGGCU; Z8 is C, G, or A; and Z9 is CACCA, AGUGGCAAACCUG (SEQ ID NO: 6017), ACCUG, UGAAAAGGUGC (SESQ ID NO: 6018), or GCU.

In some aspects, provided herein is a pharmaceutical composition comprising a sense strand and an antisense strand, wherein (i) the sense strand comprises SEQ ID NO: 3027 or a nucleic acid sequence thereof having about 1 or 2 nucleoside substitutions, additions, or deletions, and the antisense strand comprises SEQ ID NO: 3028 or a nucleic acid sequence thereof having about 1 or 2 nucleoside substitutions, additions, or deletions; (ii) the sense strand comprises SEQ ID NO: 5664 or a nucleic acid sequence thereof having about 1 or 2 nucleoside substitutions, additions, or deletions, and the antisense strand comprises SEQ ID NO: 5765 or a nucleic acid sequence thereof having about 1 or 2 nucleoside substitutions, additions, or deletions; (iii) the sense strand comprises SEQ ID NO: 5866 or a nucleic acid sequence thereof having about 1 or 2 nucleoside substitutions, additions, or deletions, and the antisense strand comprises SEQ ID NO: 5967 or a nucleic acid sequence thereof having about 1 or 2 nucleoside substitutions, additions, or deletions; (iv) the sense strand comprises SEQ ID NO: 5558 or a nucleic acid sequence thereof having about 1 or 2 nucleoside substitutions, additions, or deletions, and the antisense strand comprises SEQ ID NO: 5559 or a nucleic acid sequence thereof having about 1 or 2 nucleoside substitutions, additions, or deletions; or (v) the sense strand comprises SEQ ID NO: 5456 or a nucleic acid sequence thereof having about 1 or 2 nucleoside substitutions, additions, or deletions, and the antisense strand comprises SEQ ID NO: 5457 or a nucleic acid sequence thereof having about 1 or 2 nucleoside substitutions, additions, or deletions.

In some aspects, provided herein is a pharmaceutical composition comprising a sense strand and an antisense strand, wherein (i) the sense strand comprises SEQ ID NO: 3037 or a nucleic acid sequence thereof having about 1 or 2 nucleoside substitutions, additions, or deletions, and the antisense strand comprises SEQ ID NO: 3038 or a nucleic acid sequence thereof having about 1 or 2 nucleoside substitutions, additions, or deletions; (ii) the sense strand comprises SEQ ID NO: 5665 or a nucleic acid sequence thereof having about 1 or 2 nucleoside substitutions, additions, or deletions, and the antisense strand comprises SEQ ID NO: 5766 or a nucleic acid sequence thereof having about 1 or 2 nucleoside substitutions, additions, or deletions; (iii) the sense strand comprises SEQ ID NO: 5867 or a nucleic acid sequence thereof having about 1 or 2 nucleoside substitutions, additions, or deletions, and the antisense strand comprises SEQ ID NO: 5968 or a nucleic acid sequence thereof having about 1 or 2 nucleoside substitutions, additions, or deletions; (iv) the sense strand comprises SEQ ID NO: 5560 or a nucleic acid sequence thereof having about 1 or 2 nucleoside substitutions, additions, or deletions, and the antisense strand comprises SEQ ID NO: 5561 or a nucleic acid sequence thereof having about 1 or 2 nucleoside substitutions, additions, or deletions; or (v) the sense strand comprises SEQ ID NO: 5460 or a nucleic acid sequence thereof having about 1 or 2 nucleoside substitutions, additions, or deletions, and the antisense strand comprises SEQ ID NO: 5461 or a nucleic acid sequence thereof having about 1 or 2 nucleoside substitutions, additions, or deletions.

In some aspects, provided herein is a pharmaceutical composition comprising a sense strand and an antisense strand, wherein (i) the sense strand comprises SEQ ID NO: 3183 or a nucleic acid sequence thereof having about 1 or 2 nucleoside substitutions, additions, or deletions, and the antisense strand comprises SEQ ID NO: 3184 or a nucleic acid sequence thereof having about 1 or 2 nucleoside substitutions, additions, or deletions; (ii) the sense strand comprises SEQ ID NO: 5666 or a nucleic acid sequence thereof having about 1 or 2 nucleoside substitutions, additions, or deletions, and the antisense strand comprises SEQ ID NO: 5767 or a nucleic acid sequence thereof having about 1 or 2 nucleoside substitutions, additions, or deletions; (iii) the sense strand comprises SEQ ID NO: 5868 or a nucleic acid sequence thereof having about 1 or 2 nucleoside substitutions, additions, or deletions, and the antisense strand comprises SEQ ID NO: 5969 or a nucleic acid sequence thereof having about 1 or 2 nucleoside substitutions, additions, or deletions; (iv) or the sense strand comprises SEQ ID NO: 5562 or a nucleic acid sequence thereof having about 1 or 2 nucleoside substitutions, additions, or deletions, and the antisense strand comprises SEQ ID NO: 5563 or a nucleic acid sequence thereof having about 1 or 2 nucleoside substitutions, additions, or deletions; or (v) the sense strand comprises SEQ ID NO: 5476 or a nucleic acid sequence thereof having about 1 or 2 nucleoside substitutions, additions, or deletions, and the antisense strand comprises SEQ ID NO: 5477 or a nucleic acid sequence thereof having about 1 or 2 nucleoside substitutions, additions, or deletions.

In some aspects, provided herein is a pharmaceutical composition comprising a sense strand and an antisense strand, wherein (i) the sense strand comprises SEQ ID NO: 4265 or a nucleic acid sequence thereof having about 1 or 2 nucleoside substitutions, additions, or deletions, and the antisense strand comprises SEQ ID NO: 4266 or a nucleic acid sequence thereof having about 1 or 2 nucleoside substitutions, additions, or deletions; (ii) the sense strand comprises SEQ ID NO: 5667 or a nucleic acid sequence thereof having about 1 or 2 nucleoside substitutions, additions, or deletions, and the antisense strand comprises SEQ ID NO: 5768 or a nucleic acid sequence thereof having about 1 or 2 nucleoside substitutions, additions, or deletions; (iii) the sense strand comprises SEQ ID NO: 5869 or a nucleic acid sequence thereof having about 1 or 2 nucleoside substitutions, additions, or deletions, and the antisense strand comprises SEQ ID NO: 5970 or a nucleic acid sequence thereof having about 1 or 2 nucleoside substitutions, additions, or deletions; (iv) the sense strand comprises SEQ ID NO: 5564 or a nucleic acid sequence thereof having about 1 or 2 nucleoside substitutions, additions, or deletions, and the antisense strand comprises SEQ ID NO: 5565 or a nucleic acid sequence thereof having about 1 or 2 nucleoside substitutions, additions, or deletions; or (v) the sense strand comprises SEQ ID NO: 5510 or a nucleic acid sequence thereof having about 1 or 2 nucleoside substitutions, additions, or deletions, and the antisense strand comprises SEQ ID NO: 5511 or a nucleic acid sequence thereof having about 1 or 2 nucleoside substitutions, additions, or deletions.

In some aspects, provided herein is a pharmaceutical composition comprising a sense strand and an antisense strand, wherein (i) the sense strand comprises SEQ ID NO: 2629 or a nucleic acid sequence thereof having about 1 or 2 nucleoside substitutions, additions, or deletions, and the antisense strand comprises SEQ ID NO: 2630 or a nucleic acid sequence thereof having about 1 or 2 nucleoside substitutions, additions, or deletions; (ii) the sense strand comprises SEQ ID NO: 5668 or a nucleic acid sequence thereof having about 1 or 2 nucleoside substitutions, additions, or deletions, and the antisense strand comprises SEQ ID NO: 5769 or a nucleic acid sequence thereof having about 1 or 2 nucleoside substitutions, additions, or deletions; (iii) the sense strand comprises SEQ ID NO: 5870 or a nucleic acid sequence thereof having about 1 or 2 nucleoside substitutions, additions, or deletions, and the antisense strand comprises SEQ ID NO: 5971 or a nucleic acid sequence thereof having about 1 or 2 nucleoside substitutions, additions, or deletions; (iv) the sense strand comprises SEQ ID NO: 5566 or a nucleic acid sequence thereof having about 1 or 2 nucleoside substitutions, additions, or deletions, and the antisense strand comprises SEQ ID NO: 5567 or a nucleic acid sequence thereof having about 1 or 2 nucleoside substitutions, additions, or deletions; or (v) the sense strand comprises SEQ ID NO: 5524 or a nucleic acid sequence thereof having about 1 or 2 nucleoside substitutions, additions, or deletions, and the antisense strand comprises SEQ ID NO: 5525 or a nucleic acid sequence thereof having about 1 or 2 nucleoside substitutions, additions, or deletions.

In some aspects, provided herein is a pharmaceutical composition comprising a sense strand and an antisense strand, wherein the sense strand or antisense strand comprises one or more sequences selected from SEQ ID NOS: 1-5349, 5366-5971, 6000-6038, 6050-6074; or a nucleic acid sequence thereof having about 1 or 2 nucleoside substitutions, additions, or deletions. In some embodiments, the sense strand comprises SEQ ID NO: 5866. In some embodiments, the sense strand comprises SEQ ID NO: 5867. In some embodiments, the sense strand comprises SEQ ID NO: 5868. In some embodiments, the sense strand comprises SEQ ID NO: 5869. In some embodiments, the sense strand comprises SEQ ID NO: 5870. In some embodiments, the antisense strand comprises SEQ ID NO: 5967. In some embodiments, the antisense strand comprises SEQ ID NO: 5968. In some embodiments, the antisense strand comprises SEQ ID NO: 5969. In some embodiments, the antisense strand comprises SEQ ID NO: 5970. In some embodiments, the antisense strand comprises SEQ ID NO: 5971. In some embodiments, the sense strand comprises pattern 1S. In some embodiments, the sense strand comprises pattern 2S. In some embodiments, the sense strand comprises pattern 3S. In some embodiments, the sense strand comprises pattern 4S. In some embodiments, the sense strand comprises pattern 5S. In some embodiments, the antisense strand comprises pattern 1AS. In some embodiments, the antisense strand comprises pattern 2AS. In some embodiments, the antisense strand comprises pattern 3AS. In some embodiments, the antisense strand comprises pattern 4AS. In some aspects, provided herein is a pharmaceutical composition comprising a sense strand and an antisense strand, wherein the sense strand or antisense strand comprises one or more sequences comprising the first 19 nucleobases of a sequence selected from SEQ ID NOS: 1-5349, 5366-5971, 6000-6038, 6050-6074; or a nucleic acid sequence thereof having about 1 or 2 nucleoside substitutions, additions, or deletions.

In some embodiments, the pharmaceutical composition comprises a pharmaceutically acceptable carrier. In some embodiments, the pharmaceutical composition comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the pharmaceutical composition comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the pharmaceutical composition comprises a ribose. In some embodiments, the pharmaceutical composition comprises a lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the sense strand and the antisense strand form a double-stranded RNA duplex. In some embodiments, the first base pair of the double-stranded RNA duplex is an AU base pair.

In some aspects, provided herein is a method of treating one or more disorders of the upper and lower airway in a patient in need thereof, the method comprising administering to the patient a pharmaceutical composition provided herein. In some embodiments, the one or more disorders of the upper and lower airways comprises nasal polyposis, chronic sinusitis, allergic rhinitis, or NSAID-exacerbated respiratory disease, or a combination thereof. In some embodiments, the siRNA is administered in an effective amount to reduce eosinophil count in the patient. In some embodiments, the siRNA is administered in an effective amount to reduce number and/or size of nasal polyps in the patient. In some embodiments, the siRNA is administered in an effective amount to increase nasal inspiratory peak in the patient. In some embodiments, the siRNA is administered in an effective amount to reduce airway symptoms in the patient.

In some embodiments, provided herein is an RNA interference (RNAi) agent capable of inhibiting the expression of a target gene, wherein the RNAi agent comprises a double-stranded RNA (dsRNA) comprising a sense strand and an antisense strand, each strand having 14 to 30 nucleotides. In some instances, the RNAi comprises a double-stranded region of 17-30 nucleotide pairs in length. In some instances, the sense strand and antisense strand each have 17-30 nucleotides. In some instances, the sequence of the sense strand consists of the sequence set forth as SEQ ID NO: 5360 and the sequence of the antisense strand consists of the sequence set forth as SEQ ID NO: 5361. In some instances, the RNAi comprises one or more nucleotide modifications selected from the group consisting of LNA, HNA, CeNA, 2′-methoxyethyl, 2′-O-alkyl, 2′-O-allyl, 2′-C-allyl, 2′-fluoro, 2′-deoxy. In some instances, the nucleotides are modified with either 2′-OCH3 or 2′-F. In some instances, the RNAi further comprises at least one ligand. In some instances, the RNAi comprises one or more nucleotide modifications selected from the group consisting of 2′-O-methyl nucleotide, 2′-deoxyfluoro nucleotide, 2′-O—N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, 2′-ara-F. In some instances, the RNAi comprises at least one phosphorothioate or methylphosphonate internucleotide linkage. In some instances, the antisense strand nucleotide at the 1 position of the 5′-end of the double-stranded region is selected from the group consisting of A, dA, dU, U, and dT. In some instances, the base pair at the 1 position of the 5′-end of the double-stranded region is an AU base pair.

In some embodiments, provided herein is an RNA interference (RNAi) agent capable of inhibiting the expression of a target gene, wherein the RNAi agent comprises a double-stranded RNA (dsRNA) comprising a sense strand and an antisense strand, each of the strands having 14 to 30 nucleotides, wherein the sense strand contains at least two motifs of three identical modifications on three consecutive nucleotides, a first of said sense strand motifs occurring at a cleavage site in the sense strand and a second of said sense strand motifs occurring at a different region of the sense strand that is separated from the first sense strand motif by at least one nucleotide; and wherein the antisense strand contains at least two motifs of three identical modifications on three consecutive nucleotides, a first of said antisense strand motifs occurring at or near the cleavage site in the antisense strand and a second of said antisense strand motifs occurring at a different region of the antisense strand that is separated from the first antisense strand motif by at least one nucleotide; wherein the modification in the first antisense strand motif is different than the modification in the second antisense strand motif. In some instances, at least one of the nucleotides occurring in the first sense strand motif forms a base pair with one of the nucleotides in the first antisense strand motif. In some instances, the duplex has 17-30 nucleotides. In some instances, the duplex has 17-19 nucleotides. In some instances, each strand has 17-23 nucleotides. In some instances, the modifications on the nucleotides are selected from the group consisting of LNA, HNA, CeNA, 2′-methoxyethyl, 2-O-alkyl, 2-O-allyl, 2′-C-allyl, 2′-fluoro, 2′-deoxy, and combinations thereof. In some instances, the modifications on the nucleotide are 2′-OCH3 or 2′-F. In some instances, the RNAi comprises a ligand attached to the 3′ end of the sense strand.

In some embodiments, provided herein is an RNA interference (RNAi) agent capable of inhibiting the expression of a target gene, wherein the RNAi agent comprises a double-stranded RNA (dsRNA) comprising a sense strand and an antisense strand, each of the strands having 14 to 30 nucleotides, wherein the sense strand contains at least one motif of three 2′-F modifications on three consecutive nucleotides, one of said motifs occurring at or near the cleavage site in the strand; and wherein the antisense strand contains at least one motif of three 2-O-methyl modifications on three consecutive nucleotides, one of said motifs occurring at or near the cleavage site.

In some aspects, it is desirable to overcome at least some problems of drug solubility, deposition and delivery of RNAi using the technologies and strategies that have been employed to deliver active pharmaceutical ingredients to the nasal cavity. In some embodiments, an oligonucleotide described herein conjugated with cholesterol or lipid containing >18 carbon atoms administered to nasal epithelium increases residence time with tissue and increases intracellular delivery resulting in mRNA reduction, as compared to an unconjugated oligonucleotide. In some embodiments, an oligonucleotide is administered to nasal epithelium in a solution that is hypotonic.

In some embodiments, a small molecule inhibitor of lipoxygenase 15 is formulated with a cyclodextrin and administered to nasal epithelial tissue. In some embodiments, a small molecule inhibitor of lipoxygenase 15 is formulated with a surfactant and administered to nasal epithelial tissue.

In some embodiments, an agent for decreasing lipoxygenase expression or activity is administered using an aerosol spray device. In some embodiments, an agent for decreasing lipoxygenase expression or activity is formulated as powder and administered using a using a powder delivery device.

### BRIEF DESCRIPTION OF THE DRAWINGS

The novel features described herein are set forth with particularity in the appended claims. A better understanding of the features and advantages of the present invention will be obtained by reference to the following detailed description that sets forth illustrative embodiments and the accompanying drawings of which:

FIG. 1 is a pathway diagram showing the metabolism of arachidonic acid and other polyunsaturated fatty acid substrates. Figure reproduced from Cornejo-Garcia, et al., 2012, Clin. & Exp. Allergy 42: 1772-81.

FIG. 2 shows data indicating ablation of ALOX15 activity for T560M. Figure reproduced from Horn, et al., 2013, Redox Biol. 1:566-77.

FIG. 3 shows data indicating a new ALOX15 variant associated with airway diseases is an expression quantitative trait locus for ALOX15. The data was obtained from the GTEx Portal.

FIG. 4 is a table showing expression of ALOX15 in nasal polyps. Figure reproduced from Rostkowska, et al., 2011 Auris Nasus Larynx 38(1):58-64.

FIG. 5 shows data indicating activity of ALOX15 in nasal polyps tissue. Figure reproduced from Smith et al., Int. Arch. Allergy Appl. Immunol. 82(1):83-8.

FIG. 6 is a clinical pathology image showing increased expression of ALOX15 in eosinophilic asthma. Figure reproduced from Chu et al., 2002 Nov, Clin Exp Allergy 32(11):1558-65.

FIG. 7 is a Manhattan plot showing that about 20 genomic loci that were significantly associated with diagnosis of nasal polyps.

FIGS. 8A and 8B show the levels of ALOX15 mRNA (FIG. 8A) and 15(S)-HETE (FIG. 8B) in A549 cells following administration of ALOX15 siRNA.

### DETAILED DESCRIPTION

The concept that chronic inflammation commonly affects both the upper and lower airways via similar mechanisms is now well established. Clinically, chronic airway inflammation often presents as allergic rhinitis (AR), non-allergic rhinitis (NAR) and chronic rhinosinusitis (CRS) in the upper airway, and as asthma, COPD and the asthma-COPD overlap syndrome (ACOS) in the lower airway. These observations have fostered increasingly strong support for the so-called unified airway hypothesis. The airway is a continuous structure lined with ciliated, pseudostratified columnar epithelium that extends from the nasal vestibule to the distal bronchioles. Its mucosal surface is constantly exposed to environmental insults and is thus highly adapted in its role as the first line of defense, instigated by the innate and adaptive arms of the immune system. Though these diseases are heterogeneous in terms of their presentation and disease course, comprising many endotypes, they all share a common endotype with patients displaying a Th2-dominant response characterized by airway inflammation with local and/or systemic eosinophilia, among other features. The epidemiological and pathophysiological observations have resulted in the established dogma that the eosinophilic endotypes of airway diseases benefit from similar therapeutic approaches, revolving around modulation of the dysregulated innate, adaptive and inflammatory responses that are characteristic of these diseases.

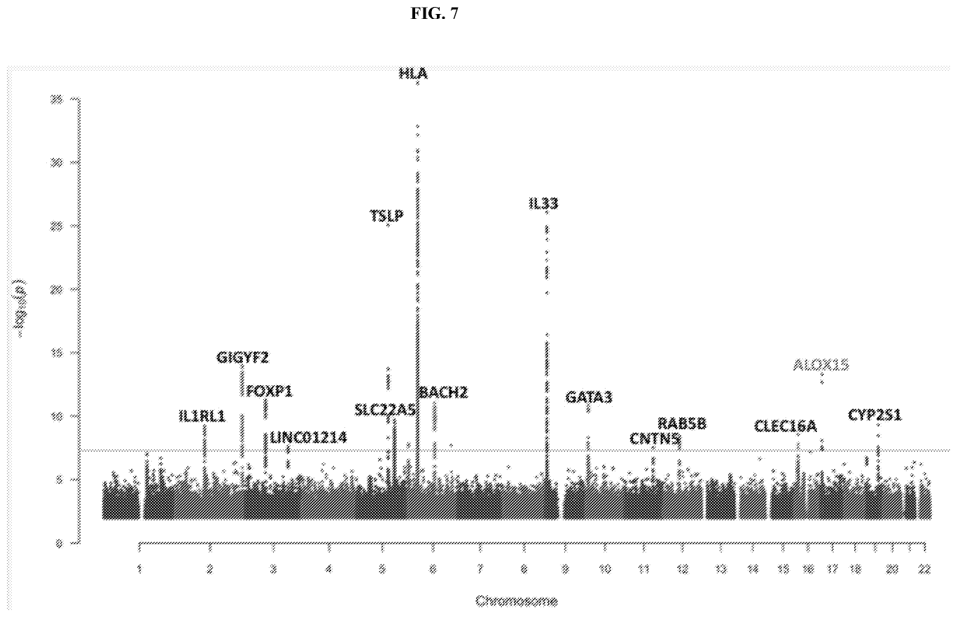

Applicants evaluated approximately 24,000,000 imputed and directly genotyped variants in ˜400,000 individuals for associations with a range of chronic airway diseases in which eosinophilic endotypes are prevalent and with blood eosinophil counts. As described herein in Example 1, about 20 genomic loci were determined to be significantly associated with diagnosis of nasal polyps (see FIG. 7).

One cluster of association was at chromosome 17p13.2, encompassing the ALOX15 gene. The most significantly associated variant (rs34210653) at this locus was a low frequency missense variant (minor allele frequency ˜1.7%) in exon 13 of the ALOX15 gene which was consistently associated with the evaluated phenotypes (see Example 1). This variant was associated with reduced risk of nasal polyps; carriers of the minor allele of this variant had less than half the risk of nasal polyps as non-carriers (p=2×10\hat{ }−15; OR=0.38). This variant was also associated with reduced risk of chronic rhinosinusitis (p=7×10\hat{ }−12; OR=0.65), allergic rhinitis (p=5×10\hat{ }−9; OR=0.80), asthma (p=9×10\hat{ }−6; OR=0.93) and reduced risk of undergoing sinus surgery including nasal polypectomy (p=5×10\hat{ }−11; OR=0.46); This variant was also associated with reduced blood eosinophil counts (p=2×10\hat{ }−65; beta=−0.02).

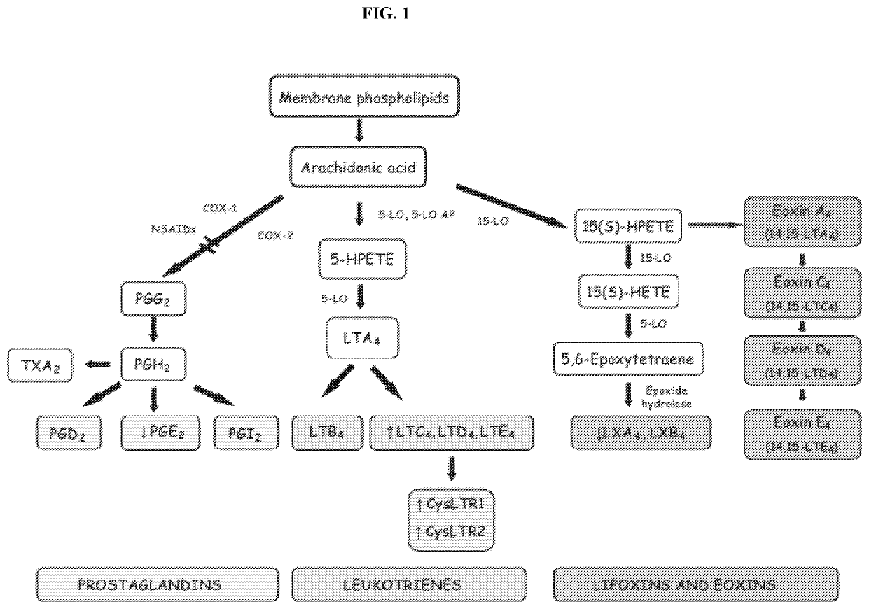

ALOX15 is one of five (ALOX5/12/12B/15/15B) human lipoxygenases and is involved in the metabolism of arachidonic acid and other polyunsaturated fatty acid substrates (FIG. 1). 15-HETE is its major arachidonic acid-derived metabolite, which is then further metabolized to eoxins, 5-oxo-15-hydroxy-ETE and other metabolites. ALOX15 metabolites are largely pro-inflammatory and have been shown to induce airway epithelial injury and promote goblet cell hyperplasia/mucus hypersecretion (15-HETE), increase vascular permeability (eoxin C4) and are potent eosinophil chemoattractants (5-oxo-15-hydroxy-ETE). ALOX15 is highly expressed in the airway and is induced in vitro by IL-13, a central mediator of the Th2 response.

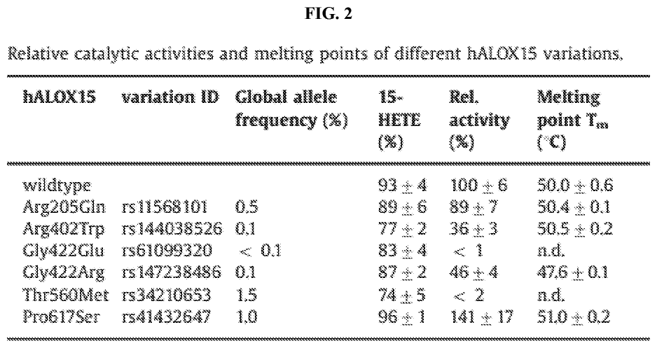

The rs34210653 variant results in a threonine to methionine change at amino acid 560 (T560M). Reports demonstrate that this T560M exchange results in near complete ablation of ALOX15 catalytic activity (FIG. 2), as measured by 15-HETE production from arachidonic acid (PMIDs: 17959182, 24282679, 21558275).

By inference, these data indicate that loss of function of ALOX15 protects against the development of nasal polyps, chronic rhinosinusitis, allergic rhinitis and asthma and strongly suggest therapeutic inhibition of ALOX15 as a novel, genetically-informed method of treatment for these diseases.

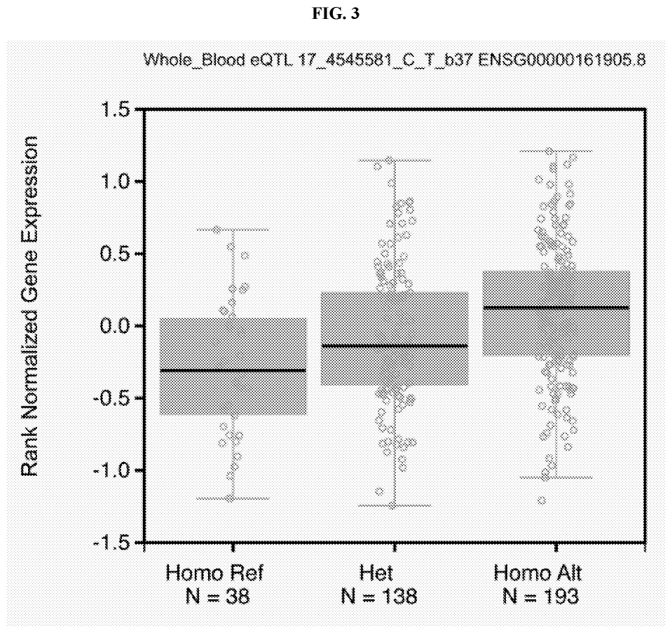

In addition to the protective LOF variant outlined above, Applicants discovered that a separate, independent ALOX15 variant is associated with risk of these diseases. This variant, rs2255888, is an ALOX15 regulatory variant that is associated with increased expression of ALOX15 in whole blood (see Example 1, FIG. 3).

In an analysis conditioning on the LOF variant, the T allele of rs2255888, which is associated with increased expression of ALOX15 in whole blood, is associated with increased risk of nasal polyps (p=7×10\hat{ }−5; OR=1.2) and increased blood eosinophil counts (p=2×10\hat{ }−22; beta=0.004). In combination with the T560M loss of function variant, Applicants have therefore identified an ALOX15 allelic series that modulates risk for nasal polyps and blood eosinophils. This allelic series consist of an ALOX15 loss of function variant (rs34210653) that is associated with decreased risk of nasal polyps, chronic rhinosinusitis, allergic rhinitis, asthma and decreased blood eosinophil counts, and an ALOX15 regulatory variant that increases ALOX15 expression (rs2255888) that is associated with increased risk of nasal polyps and increased blood eosinophil counts, further suggesting therapeutic inhibition of ALOX15 as a novel, genetically-informed method of treatment of nasal polyps and related eosinophilic diseases of the airway.

Applicants also identified three additional rare variants with evidence for being protein truncating or damaging to the protein and analyzed them collectively in a gene burden test (see Example 1). In aggregate these variants are associated with decreased risk of nasal polyps (p=0.0008; OR=0.53) and decreased blood eosinophil counts (p=1.4×10\hat{ }−9; beta=−0.016).

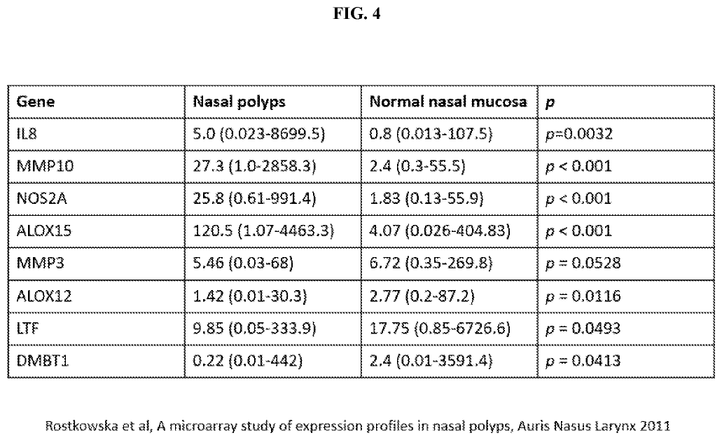

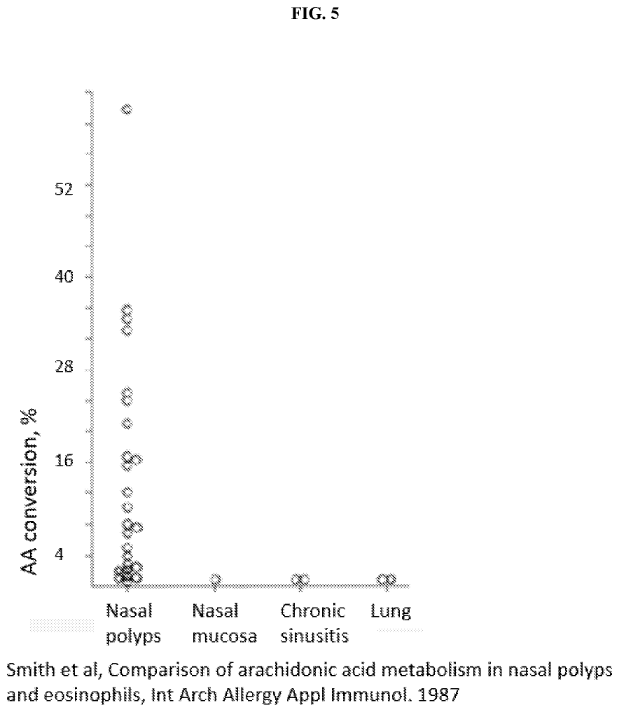

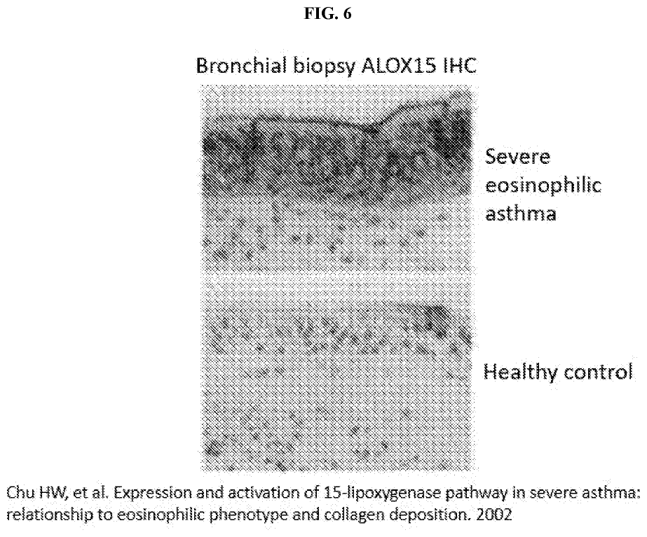

In the context of these novel genetic findings, it is notable that previous expression studies have found that ALOX15 is highly expressed in airway epithelial cells, eosinophils and particularly in nasal polyps tissue. Both expression (FIG. 4) and activity (FIG. 5) of ALOX15 are ˜30 times greater in nasal polyps tissue than in normal mucosa. ALOX15 expression is also increased in lung granulocytes and bronchial biopsies of asthmatic patients (FIG. 6), and in bronchial biopsies of patients with COPD. Together with Applicants' genetic findings, these data very strongly suggest that ALOX15 inhibition will have therapeutic benefit in the treatment of nasal polyps and related eosinophilic diseases of the airway.

Applicants propose that therapies designed to inhibit the production of ALOX15 protein delivered locally to the nasal epithelium, via inhalation or systemically, will be efficacious in treating nasal polyposis and related disorders of the upper and lower airway, including chronic rhinosinusitis, asthma, allergic rhinitis and NSAID-exacerbated respiratory disease. Our therapeutic mechanism utilizes RNA interference (RNAi) approaches designed to inhibit translation of or degrade ALOX15 mRNA. The RNAi based therapeutic modalities include siRNA targeting of ALOX15 resulting in RISC mediated mRNA cleavage and exonuclease degradation, or miRNA targeting resulting in inhibition of translation and/or degradation by exonucleases.

Provided herein are double-stranded RNAi (dsRNA) agents capable of inhibiting the expression of ALOX15. The dsRNA (dsRNA) agents, in some embodiments, are small interfering RNAs (siRNAs). dsRNA agents as described herein may comprise therapeutic use. For example, dsRNA agents are used for the treatment of various diseases involving ALOX-15 gene products.

The dsRNA agent, in some embodiments, comprises a sense strand and an antisense strand. Each strand of the dsRNA agent can range from 12-30 nucleotides in length. For example, each strand can be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. In some embodiments, each strand is from about 10 to about 50 nucleotides in length. In some instances, each strand is from about 10 to about 30, from about 15 to about 30, from about 18 to about 25, form about 18 to about 24, from about 19 to about 23, or from about 20 to about 22 nucleotides in length. In some embodiments, each strand is about 50 nucleotides in length. In some instances, each strand is about 45 nucleotides in length. In some instances, each strand is about 40 nucleotides in length. In some instances, each strand is about 35 nucleotides in length. In some instances, each strand is about 30 nucleotides in length. In some instances, each strand is about 25 nucleotides in length. In some instances, each strand is about 21 nucleotides in length. In some instances, each strand is about 20 nucleotides in length. In some instances, each strand is about 19 nucleotides in length. In some instances, each strand is about 18 nucleotides in length. In some instances, each strand is about 17 nucleotides in length. In some instances, each strand is about 16 nucleotides in length. In some instances, each strand is about 15 nucleotides in length. In some instances, each strand is about 14 nucleotides in length. In some instances, each strand is about 13 nucleotides in length. In some instances, each strand is about 12 nucleotides in length. In some instances, each strand is about 11 nucleotides in length. In some instances, each strand is about 10 nucleotides in length. In some instances, each strand is between about 10 and about 50 nucleotides in length. In some instances, each strand is between about 10 and about 45 nucleotides in length. In some instances, each strand is between about 10 and about 40 nucleotides in length. In some instances, each strand is between about 10 and about 35 nucleotides in length. In some instances, each strand is between about 10 and about 30 nucleotides in length. In some instances, each strand is between about 10 and about 25 nucleotides in length. In some instances, each strand is between about 10 and about 20 nucleotides in length. In some instances, each strand is between about 15 and about 25 nucleotides in length. In some instances, each strand is between about 15 and about 30 nucleotides in length. In some instances, each strand is between about 12 and about 30 nucleotides in length.

The sense strand and antisense strand typically form a duplex dsRNA. The duplex region of a dsRNA agent may be 12-30 nucleotide pairs in length. For example, the duplex region can be between 14-30 nucleotide pairs in length, 17-30 nucleotide pairs in length, 25-30 nucleotides in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In some embodiments, the duplex region is selected from 15, 16, 17, 18, 19, 20, 21, 22, 23, 24, 25, 26, and 27. In some embodiments, the duplex region is from about 10 to about 50 nucleotides in length. In some instances, the duplex region is from about 10 to about 30, from about 15 to about 30, from about 18 to about 25, form about 18 to about 24, from about 19 to about 23, or from about 20 to about 22 nucleotides in length. In some embodiments, the duplex region is about 50 nucleotides in length. In some instances, the duplex region is about 45 nucleotides in length. In some instances, the duplex region is about 40 nucleotides in length. In some instances, the duplex region is about 35 nucleotides in length. In some instances, the duplex region is about 30 nucleotides in length. In some instances, the duplex region is about 25 nucleotides in length. In some instances, the duplex region is about 21 nucleotides in length. In some instances, the duplex region is about 20 nucleotides in length. In some instances, the duplex region is about 19 nucleotides in length. In some instances, the duplex region is about 18 nucleotides in length. In some instances, the duplex region is about 17 nucleotides in length. In some instances, the duplex region is about 16 nucleotides in length. In some instances, the duplex region is about 15 nucleotides in length. In some instances, the duplex region is about 14 nucleotides in length. In some instances, the duplex region is about 13 nucleotides in length. In some instances, the duplex region is about 12 nucleotides in length. In some instances, the duplex region is about 11 nucleotides in length. In some instances, the duplex region is about 10 nucleotides in length. In some instances, the duplex region is between about 10 and about 50 nucleotides in length. In some instances, the duplex region is between about 10 and about 45 nucleotides in length. In some instances, the duplex region is between about 10 and about 40 nucleotides in length. In some instances, the duplex region is between about 10 and about 35 nucleotides in length. In some instances, the duplex region is between about 10 and about 30 nucleotides in length. In some instances, the duplex region is between about 10 and about 25 nucleotides in length. In some instances, the duplex region is between about 10 and about 20 nucleotides in length. In some instances, the duplex region is between about 15 and about 25 nucleotides in length. In some instances, the duplex region is between about 15 and about 30 nucleotides in length. In some instances, the duplex region is between about 12 and about 30 nucleotides in length.

In some embodiments, the dsRNA agent described herein comprises one or more overhang regions and/or capping groups of dsRNA agent at the 3′-end, or 5′-end or both ends of a strand. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be other sequence. The first and second strands can also be joined, e.g., by additional bases to form a hairpin, or by other non-base linkers.

In some embodiments, the nucleotides in the overhang region of the dsRNA agent described herein can each independently be a modified or unmodified nucleotide including, but no limited to 2′-sugar modified, such as, 2-F 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA agent described herein may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

Described herein are double-stranded RNAi (dsRNA) agents capable of inhibiting the expression of ALOX15. The dsRNA agent may comprise a sense strand and an antisense strand, each strand having 14 to 30 nucleotides.

### Nucleic Acids

In some aspects, described herein are dsRNA comprising a sense strand comprising at least about 80%, 85%, 90%, or 95% identity to any one of SEQ ID NOS: 5568-5971, and an antisense strand that is complementary to the sense strand. In some aspects, described herein are dsRNA comprising a sense strand comprising at least about 80%, 85%, 90%, or 95% identity to any one of SEQ ID NOS: 6000-6038, and an antisense strand that is complementary to the sense strand. In some aspects, described herein are dsRNA comprising a sense strand comprising at least about 80%, 85%, 90%, or 95% identity to any one of SEQ ID NOS: 6050-6074, and an antisense strand that is complementary to the sense strand. In some aspects, described herein are dsRNA comprising a sense strand comprising at least about 19 nucleotides of any one of SEQ ID NOS: 1-5349, and an antisense strand that is complementary to the sense strand. In some aspects, described herein are dsRNA comprising a sense strand comprising at least about 80%, 85%, 90%, or 95% identity to any one of SEQ ID NOS: 1-5349, and an antisense strand that is complementary to the sense strand. In some cases, complementarity of a sense strand and antisense strand means at least about 70%, 75%, 80%, 85%, 90%, or 95% of the nucleotides in each strand are capable of forming a base pair with the other strand. For instance, for dsRNA comprising 19 or 21 nucleotides, the sense strand and antisense strand are capable of forming at least about 15, 16, 17, 18, or 19 base pairs. In some embodiments, the sense strand comprises one or more sequences selected from SEQ ID NOS: 23, 31, 33, 37, 39, 41, 43, 51, 195, 197, 199, 251, 257, 263, 273, 275, 279, 2389, 2391, 2397, 2399, 2409, 2415, 2419, 2421, 2423, 2425, 2427, 2431, 2437, 2439, 2769, 2771, 2773, 2881, 2899, 2903, 2905, 2909, 2913, 2915, 3013, 3015, 3017, 3019, 3027, 3029, 3037, 3039, 3041, 3047, 3053, 3055, 3063, 3065, 3183, 3231, 3233, 3243, 3439, 3443, 3449, 3457, 3461, 3463, 3573, 3703, 3705, 3875, 3889, 3981, 4089, 4265, 4321, 4339, 4347, 4507, 53, 2627, 2629, 3355, 3365, 87, 2329, 2379, 2381, 2521, 2525, 2607, 3057, 3061, 3067, 3501, 3503, 3873, 4179, 5568-5668, 5770-5870. In some embodiments, the antisense strand comprises one or more sequences selected from SEQ ID NOS: 24, 32, 34, 38, 40, 42, 44, 52, 196, 198, 200, 252, 258, 264, 274, 276, 280, 2390, 2392, 2398, 2400, 2410, 2416, 2420, 2422, 2424, 2426, 2428, 2432, 2438, 2440, 2770, 2772, 2774, 2882, 2900, 2904, 2906, 2910, 2914, 2916, 3014, 3016, 3018, 3020, 3028, 3030, 3038, 3040, 3042, 3048, 3054, 3056, 3064, 3066, 3184, 3232, 3234, 3244, 3440, 3444, 3450, 3458, 3462, 3464, 3574, 3704, 3706, 3876, 3890, 3982, 4090, 4266, 4322, 4340, 4348, 4508, 54, 2628, 2630, 3356, 3366, 88, 2330, 2380, 2382, 2522, 2526, 2608, 3058, 3062, 3068, 3502, 3504, 3874, 4180, 5669-5769, 5871-5971. An exemplary dsRNA comprises SEQ ID NOS: 5770 and 5871. Another exemplary dsRNA comprises SEQ ID NOS: 5771 and 5872. Another exemplary dsRNA comprises SEQ ID NOS: 5772 and 5873. Another exemplary dsRNA comprises SEQ ID NOS: 5773 and 5874. Another exemplary dsRNA comprises SEQ ID NOS: 5774 and 5875. Another exemplary dsRNA comprises SEQ ID NOS: 5775 and 5876. Another exemplary dsRNA comprises SEQ ID NOS: 5776 and 5877. Another exemplary dsRNA comprises SEQ ID NOS: 5777 and 5878. Another exemplary dsRNA comprises SEQ ID NOS: 5778 and 5879. Another exemplary dsRNA comprises SEQ ID NOS: 5779 and 5880. Another exemplary dsRNA comprises SEQ ID NOS: 5780 and 5881. Another exemplary dsRNA comprises SEQ ID NOS: 5781 and 5882. Another exemplary dsRNA comprises SEQ ID NOS: 5782 and 5883. Another exemplary dsRNA comprises SEQ ID NOS: 5783 and 5884. Another exemplary dsRNA comprises SEQ ID NOS: 5784 and 5885. Another exemplary dsRNA comprises SEQ ID NOS: 5785 and 5886. Another exemplary dsRNA comprises SEQ ID NOS: 5786 and 5887. Another exemplary dsRNA comprises SEQ ID NOS: 5787 and 5888. Another exemplary dsRNA comprises SEQ ID NOS: 5788 and 5889. Another exemplary dsRNA comprises SEQ ID NOS: 5789 and 5890. Another exemplary dsRNA comprises SEQ ID NOS: 5790 and 5891. Another exemplary dsRNA comprises SEQ ID NOS: 5791 and 5892. Another exemplary dsRNA comprises SEQ ID NOS: 5792 and 5893. Another exemplary dsRNA comprises SEQ ID NOS: 5793 and 5894. Another exemplary dsRNA comprises SEQ ID NOS: 5794 and 5895. Another exemplary dsRNA comprises SEQ ID NOS: 5795 and 5896. Another exemplary dsRNA comprises SEQ ID NOS: 5796 and 5897. Another exemplary dsRNA comprises SEQ ID NOS: 5797 and 5898. Another exemplary dsRNA comprises SEQ ID NOS: 5798 and 5899. Another exemplary dsRNA comprises SEQ ID NOS: 5799 and 5900. Another exemplary dsRNA comprises SEQ ID NOS: 5800 and 5901. Another exemplary dsRNA comprises SEQ ID NOS: 5801 and 5902. Another exemplary dsRNA comprises SEQ ID NOS: 5802 and 5903. Another exemplary dsRNA comprises SEQ ID NOS: 5803 and 5904. Another exemplary dsRNA comprises SEQ ID NOS: 5804 and 5905. Another exemplary dsRNA comprises SEQ ID NOS: 5805 and 5906. Another exemplary dsRNA comprises SEQ ID NOS: 5806 and 5907. Another exemplary dsRNA comprises SEQ ID NOS: 5807 and 5908. Another exemplary dsRNA comprises SEQ ID NOS: 5808 and 5909. Another exemplary dsRNA comprises SEQ ID NOS: 5809 and 5910. Another exemplary dsRNA comprises SEQ ID NOS: 5810 and 5911. Another exemplary dsRNA comprises SEQ ID NOS: 5811 and 5912. Another exemplary dsRNA comprises SEQ ID NOS: 5812 and 5913. Another exemplary dsRNA comprises SEQ ID NOS: 5813 and 5914. Another exemplary dsRNA comprises SEQ ID NOS: 5814 and 5915. Another exemplary dsRNA comprises SEQ ID NOS: 5815 and 5916. Another exemplary dsRNA comprises SEQ ID NOS: 5816 and 5917. Another exemplary dsRNA comprises SEQ ID NOS: 5817 and 5918. Another exemplary dsRNA comprises SEQ ID NOS: 5818 and 5919. Another exemplary dsRNA comprises SEQ ID NOS: 5819 and 5920. Another exemplary dsRNA comprises SEQ ID NOS: 5820 and 5921. Another exemplary dsRNA comprises SEQ ID NOS: 5821 and 5922. Another exemplary dsRNA comprises SEQ ID NOS: 5822 and 5923. Another exemplary dsRNA comprises SEQ ID NOS: 5823 and 5924. Another exemplary dsRNA comprises SEQ ID NOS: 5824 and 5925. Another exemplary dsRNA comprises SEQ ID NOS: 5825 and 5926. Another exemplary dsRNA comprises SEQ ID NOS: 5826 and 5927. Another exemplary dsRNA comprises SEQ ID NOS: 5827 and 5928. Another exemplary dsRNA comprises SEQ ID NOS: 5828 and 5929. Another exemplary dsRNA comprises SEQ ID NOS: 5829 and 5930. Another exemplary dsRNA comprises SEQ ID NOS: 5830 and 5931. Another exemplary dsRNA comprises SEQ ID NOS: 5831 and 5932. Another exemplary dsRNA comprises SEQ ID NOS: 5832 and 5933. Another exemplary dsRNA comprises SEQ ID NOS: 5833 and 5934. Another exemplary dsRNA comprises SEQ ID NOS: 5834 and 5935. Another exemplary dsRNA comprises SEQ ID NOS: 5835 and 5936. Another exemplary dsRNA comprises SEQ ID NOS: 5836 and 5937. Another exemplary dsRNA comprises SEQ ID NOS: 5837 and 5938. Another exemplary dsRNA comprises SEQ ID NOS: 5838 and 5939. Another exemplary dsRNA comprises SEQ ID NOS: 5839 and 5940. Another exemplary dsRNA comprises SEQ ID NOS: 5840 and 5941. Another exemplary dsRNA comprises SEQ ID NOS: 5841 and 5942. Another exemplary dsRNA comprises SEQ ID NOS: 5842 and 5943. Another exemplary dsRNA comprises SEQ ID NOS: 5843 and 5944. Another exemplary dsRNA comprises SEQ ID NOS: 5844 and 5945. Another exemplary dsRNA comprises SEQ ID NOS: 5845 and 5946. Another exemplary dsRNA comprises SEQ ID NOS: 5846 and 5947. Another exemplary dsRNA comprises SEQ ID NOS: 5847 and 5948. Another exemplary dsRNA comprises SEQ ID NOS: 5848 and 5949. Another exemplary dsRNA comprises SEQ ID NOS: 5849 and 5950. Another exemplary dsRNA comprises SEQ ID NOS: 5850 and 5951. Another exemplary dsRNA comprises SEQ ID NOS: 5851 and 5952. Another exemplary dsRNA comprises SEQ ID NOS: 5852 and 5953. Another exemplary dsRNA comprises SEQ ID NOS: 5853 and 5954. Another exemplary dsRNA comprises SEQ ID NOS: 5854 and 5955. Another exemplary dsRNA comprises SEQ ID NOS: 5855 and 5956. Another exemplary dsRNA comprises SEQ ID NOS: 5856 and 5957. Another exemplary dsRNA comprises SEQ ID NOS: 5857 and 5958. Another exemplary dsRNA comprises SEQ ID NOS: 5858 and 5959. Another exemplary dsRNA comprises SEQ ID NOS: 5859 and 5960. Another exemplary dsRNA comprises SEQ ID NOS: 5860 and 5961. Another exemplary dsRNA comprises SEQ ID NOS: 5861 and 5962. Another exemplary dsRNA comprises SEQ ID NOS: 5862 and 5963. Another exemplary dsRNA comprises SEQ ID NOS: 5863 and 5964. Another exemplary dsRNA comprises SEQ ID NOS: 5864 and 5965. Another exemplary dsRNA comprises SEQ ID NOS: 5865 and 5966. Another exemplary dsRNA comprises SEQ ID NOS: 5866 and 5967. Another exemplary dsRNA comprises SEQ ID NOS: 5867 and 5968. Another exemplary dsRNA comprises SEQ ID NOS: 5868 and 5969. Another exemplary dsRNA comprises SEQ ID NOS: 5869 and 5970. Another exemplary dsRNA comprises SEQ ID NOS: 5870 and 5971. In some aspects, any of the dsRNA are modified, such as described elsewhere herein. Any of the dsRNA described herein may comprise a pattern, e.g., as described herein. For instance, the sense strand may comprise pattern 1S, 2S, 3S, 4S, or 5S. The antisense strand may comprise pattern 1AS, 2AS, 3AS, or 4AS.

In some aspects, described herein are nucleic acid sequences comprising Formula IA, IB, IIA, IIB, IIIA, IIIB, IVA, IVB, VA, VB, VIA, VIB, VIIA, VIIB, VIIIA, or VIIIB, or a combination thereof. In some aspects, a dsRNA described herein comprises a sense strand comprising a nucleic acid sequence of Formula IA. In some aspects, a dsRNA described herein comprises a sense strand comprising a nucleic acid sequence of Formula IIA. In some aspects, a dsRNA described herein comprises a sense strand comprising a nucleic acid sequence of Formula IIIA. In some aspects, a dsRNA described herein comprises a sense strand comprising a nucleic acid sequence of Formula IVA. In some aspects, a dsRNA described herein comprises a sense strand comprising a nucleic acid sequence of Formula VA. In some aspects, a dsRNA described herein comprises a sense strand comprising a nucleic acid sequence of Formula VIA. In some aspects, a dsRNA described herein comprises a sense strand comprising a nucleic acid sequence of Formula VIIA. In some aspects, a dsRNA described herein comprises a sense strand comprising a nucleic acid sequence of Formula VIIIA.

In some aspects, a dsRNA described herein comprises an antisense strand comprising a nucleic acid sequence of Formula IB. In some aspects, a dsRNA described herein comprises an antisense strand comprising a nucleic acid sequence of Formula IIB. In some aspects, a dsRNA described herein comprises an antisense strand comprising a nucleic acid sequence of Formula IIIB. In some aspects, a dsRNA described herein comprises an antisense strand comprising a nucleic acid sequence of Formula IVB. In some aspects, a dsRNA described herein comprises an antisense strand comprising a nucleic acid sequence of Formula VB. In some aspects, a dsRNA described herein comprises an antisense strand comprising a nucleic acid sequence of Formula VIB. In some aspects, a dsRNA described herein comprises an antisense strand comprising a nucleic acid sequence of Formula VIIB. In some aspects, a dsRNA described herein comprises an antisense strand comprising a nucleic acid sequence of Formula VIIIB.

Formula IA comprises 5′ Z1-U-Z2-G-Z3-U-C-Z4-A-U-U-Z5-L 3′, wherein Z1 represents 0, 3, 5, 11, or 13 nucleosides; Z2 represents 1 nucleoside; Z3 represents 0, 2, 5, 8, or 10 nucleosides; Z4 represents 0, 2, 3, 5, 8, or 11 nucleosides; Z5 represents 0 or 3 nucleosides; and L represents an optional lipid. In some embodiments, Z1 is 0 nucleosides. In some embodiments, Z1 is 3, 5, 11, or 13 nucleosides. In some embodiments, Z1 comprises UGGUG, CAGGU, CAGGUUUGCCACU (SEQ ID NO: 6007), GCACCUUUUCA (SEQ ID NO: 6008), or AGC, or a nucleic acid sequence thereof having about 1 or 2 nucleoside substitutions, additions, or deletions. In some embodiments, Z2 comprises C, G, or U. In some embodiments, Z3 is 0 nucleosides. In some embodiments, Z3 is 2, 5, 8, or 10 nucleosides. In some embodiments, Z3 comprises UG, UGUCGAGAGA (SEQ ID NO: 6009), AGAGA, AGAGA, CCACUUUG, or AGCCU, or a nucleic acid sequence thereof having about 1 or 2 nucleoside substitutions, additions, or deletions. In some embodiments, Z4 is 0 nucleosides. In some embodiments, Z4 is 2, 3, 5, 8, or 11 nucleosides. In some embodiments, Z4 comprises GAGAGAUCACU (SEQ ID NO: 6010), ACU, ACUGAAAU, UC, or CUAAC, or a nucleic acid sequence thereof having about 1 or 2 nucleoside substitutions, additions, or deletions. In some embodiments, Z5 represents 0 nucleosides. In some embodiments, Z5 represents 3 nucleosides. In some embodiments, Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z5 nucleosides comprise a deoxyribose. The one or more may be one, two, or three. In some embodiments, Formula IA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, Z1-U-Z2-G-Z3-U-C-Z4 comprises UGGUGUCGAGAGAUCACU (SEQ ID NO: 6011), or a nucleic acid sequence thereof having about 1 or 2 nucleoside substitutions, additions, or deletions. In some embodiments, Z1-U-Z2-G-Z3-U-C-Z4 comprises UCGAGAGAUCACUGAAAU (SEQ ID NO: 6012), or a nucleic acid sequence thereof having about 1 or 2 nucleoside substitutions, additions, or deletions. In some embodiments, Z1-U-Z2-G-Z3-U-C-Z4 comprises CAGGUUUGCCACUUUGUC (SEQ ID NO: 6013), or a nucleic acid sequence thereof having about 1 or 2 nucleoside substitutions, additions, or deletions. In some embodiments, Z1-U-Z2-G-Z3-U-C-Z4 comprises GCACCUUUUCAUGGUCUC (SEQ ID NO: 6014), or a nucleic acid sequence thereof having about 1 or 2 nucleoside substitutions, additions, or deletions. In some embodiments, Z1-U-Z2-G-Z3-U-C-Z4 comprises AGCUGGAGCCUUCCUAAC (SEQ ID NO: 15), or a nucleic acid sequence thereof having about 1 or 2 nucleoside substitutions, additions, or deletions. In some embodiments, the Z1-U-Z2-G-Z3-U-C-Z4-A-U-U sequence of Formula 1A is twenty-one nucleosides in length. In some embodiments, the antisense strand comprises the reserve complement of Z1-U-Z2-G-Z3-U-C-Z4.

Formula IB comprises 5′ U-Z6-G-A-Z7-C-Z8-A-Z9-U-U 3′, wherein Z6 represents 0, 2, 3, 5, 8, or 11 nucleosides; Z7 represents 0, 2, 5, 8, or 10 nucleosides; Z8 represents 1 nucleoside; Z9 represents 0, 3, 5, 11, or 13 nucleosides; and each “-” is independently a phosphodiester or modified internucleoside linkage. In some embodiments, Z6 is 0 nucleosides. In some embodiments, Z6 is 0, 2, 3, 5, 8, or 11 nucleosides. In some embodiments, Z6 comprises AGU, AGUGAUCUCUC (SEQ ID NO: 6016), AUUUCAGU, GA, or GUUAG, or a nucleic acid sequence thereof having about 1 or 2 nucleoside substitutions, additions, or deletions. In some embodiments, Z7 is 0 nucleosides. In some embodiments, Z7 is 0, 2, 5, 8, or 10 nucleosides. In some embodiments, Z7 comprises UCUCU, CA, UCUCU, CAAAGUGG, GA, or AGGCU, or a nucleic acid sequence thereof having about 1 or 2 nucleoside substitutions, additions, or deletions. In some embodiments, Z8 comprises C, G, or A. In some embodiments, Z9 is 0 nucleosides. In some embodiments, Z9 is 3, 5, 11, or 13 nucleosides. In some embodiments, Z9 comprises CACCA, AGUGGCAAACCUG (SEQ ID NO: 6017), ACCUG, UGAAAAGGUGC (SEQ ID NO: 6018), or GCU, or a nucleic acid sequence thereof having about 1 or 2 nucleoside substitutions, additions, or deletions. In some embodiments, Z6-G-A-Z7-C-Z8-A-Z9 comprises AGUGAUCUCUCGACACCA (SEQ ID NO: 6019), or a nucleic acid sequence thereof having about 1 or 2 nucleoside substitutions, additions, or deletions. In some embodiments, Z6-G-A-Z7-C-Z8-A-Z9 comprises AUUUCAGUGAUCUCUCGA (SEQ ID NO: 6020), or a nucleic acid sequence thereof having about 1 or 2 nucleoside substitutions, additions, or deletions. In some embodiments, Z6-G-A-Z7-C-Z8-A-Z9 comprises GACAAAGUGGCAAACCUG (SEQ ID NO: 6021), or a nucleic acid sequence thereof having about 1 or 2 nucleoside substitutions, additions, or deletions. In some embodiments, Z6-G-A-Z7-C-Z8-A-Z9 comprises GAGACCAUGAAAAGGUGC (SEQ ID NO: 6022), or a nucleic acid sequence thereof having about 1 or 2 nucleoside substitutions, additions, or deletions. In some embodiments, Z6-G-A-Z7-C-Z8-A-Z9 comprises GUUAGGAAGGCUCCAGCU (SEQ ID NO: 6023), or a nucleic acid sequence thereof having about 1 or 2 nucleoside substitutions, additions, or deletions. In some embodiments, the U-Z6-G-A-Z7-C-Z8-A-Z9-U-U is 21 nucleosides in length.

Formula IIA comprises 5′ Z1-U-Z2-G-Z3-C-U-Z4-A-U-U-Z5-L 3′, wherein Z1 represents 0, 3, 5, or 11 nucleosides; Z2 represents 1 nucleoside; Z3 represents 1, 3, 7, 8, or 13 nucleosides; Z4 represents 0, 1, 3, 5, or 7 nucleosides; Z5 represents 0 or 3 nucleosides; and L comprises an optional lipid. In some cases, Z1 comprises UGGUG, CAGGU, GCACCUUUUCA (SEQ ID NO: 6008), or AGC, or a nucleic acid sequence thereof having about 1 or 2 nucleoside substitutions, additions, or deletions. In some cases, Z2 comprises G, C, or U. In some cases, Z3 comprises UGUCGAGAGAUCA (SEQ ID NO: 6024), AGAGAUCA, AGAGAUCA, CCA, U, AGC, or AGCCUUC, or a nucleic acid sequence thereof having about 1 or 2 nucleoside substitutions, additions, or deletions. In some cases, Z4 comprises GAAAU, UUGUC, C, UCCUAAC, or AAC, or a nucleic acid sequence thereof having about 1 or 2 nucleoside substitutions, additions, or deletions. In some embodiments, Z5 represents 0 nucleosides. In some embodiments, Z5 represents 3 nucleosides. In some embodiments, Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z5 nucleosides comprise a deoxyribose. The one or more may be one, two, or three. In some embodiments, Formula IIA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula IIB comprises 5′ U-Z6-A-G-Z7-C-Z8-A-Z9-U-U 3′, wherein Z6 represents 0, 1, 3, 5, or 7 nucleosides; Z7 represents 1, 3, 7, 8, or 13 nucleosides; Z8 represents 1 nucleoside; and Z9 represents 0, 3, 5, or 11 nucleosides. In some cases, Z6 comprises AUUUC, GACAA, G, GUU, or a nucleic acid sequence thereof having about 1 or 2 nucleoside substitutions, additions, or deletions. In some cases, Z7 comprises UGAUCUCU, UGAUCUCU, UGG, A, GAAGGCU, GCU, or a nucleic acid sequence thereof having about 1 or 2 nucleoside substitutions, additions, or deletions. In some cases, Z8 comprises G, C, or A. In some cases, Z9 comprises CACCA, ACCUG, UGAAAAGGUGC (SEQ ID NO: 6018), GCU, or a nucleic acid sequence thereof having about 1 or 2 nucleoside substitutions, additions, or deletions.

Formula IIIA comprises 5′ Z1-U-Z2-G-Z3-U-Z4-C-Z5-A-U-U-Z6-L 3′, wherein Z1 represents 0, 3, 5, or 11 nucleosides; Z2 represents 1 nucleoside; Z3 represents 0, 4, 5, 6, 8, or 10 nucleosides; Z4 represents 2 nucleosides; Z5 represents 1, 4, 6, or 11 nucleosides; Z6 represents 0 or 3 nucleosides; and L comprises an optional lipid. In some cases, Z1 comprises UGGUG, CAGGU, GCACCUUUUCA (SEQ ID NO: 6008), AGC, or a nucleic acid sequence thereof having about 1 or 2 nucleoside substitutions, additions, or deletions. In some cases, Z2 comprises G, C, or U. In some cases, Z3 comprises UGUCGAGAGA (SEQ ID NO: 6009), AGAGA, CCACUU, AGCC, AGCCUUCC, or a nucleic acid sequence thereof having about 1 or 2 nucleoside substitutions, additions, or deletions. In some cases, Z4 comprises GU, CA, CU, UC, or AA. In some cases, Z5 comprises GAGAGAUCACU (SEQ ID NO: 6010), U, UGAAAU, UAAC, or a nucleic acid sequence thereof having about 1 or 2 nucleoside substitutions, additions, or deletions. In some embodiments, Z6 represents 0 nucleosides. In some embodiments, Z5 represents 3 nucleosides. In some embodiments, Z6 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z6 nucleosides comprise a deoxyribose. The one or more may be one, two, or three. In some embodiments, Formula IIIA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula IIIB comprises 5′ U-Z7-G-Z8-A-Z9-C-Z10-A-Z11-U-U 3′, wherein Z7 represents 0, 1, 4, 6, or 11 nucleosides; Z8 represents 2 nucleosides; Z9 represents 0, 4, 5, 6, 8 or 10 nucleosides; Z10 represents 1 nucleoside; and Z11 represents 0, 3, 5, or 11 nucleosides. In some cases, Z7 comprises A, AGUGAUCUCUC (SEQ ID NO: 6016), AUUUCA, GUUA, or a nucleic acid sequence thereof having about 1 or 2 nucleoside substitutions, additions, or deletions. In some cases, Z8 comprises UG, AC, AG, UU, or GA. In some cases, Z9 comprises UCUCU, UCUCUCGACA (SEQ ID NO: 6025), UCUCU, AAGUGG, GGAAGGCU, or GGCU or a nucleic acid sequence thereof having about 1 or 2 nucleoside substitutions, additions, or deletions. In some cases, Z10 comprises G, C, or A. In some cases, Z11 comprises CACCA, ACCUG, UGAAAAGGUGC (SEQ ID NO: 6018), GCU, or a nucleic acid sequence thereof having about 1 or 2 nucleoside substitutions, additions, or deletions.

Formula IVA comprises 5′ Z1-G-Z2-U-Z3-C-U-Z4-A-U-U-Z5-L 3′, wherein Z1 represents 1, 2, 3, 6, 11, or 12 nucleosides; Z2 represents 1 nucleoside; Z3 represents 0, 2, 5, 6, 9, or 12 nucleosides; Z4 represents 0, 1, 3, or 5 nucleosides; Z5 represents 0 or 3 nucleosides; and L comprises an optional lipid. In some cases, Z1 comprises U, UGGUGUCGAGA (SEQ ID NO: 6026), UCGAGA, CA, A, GCACCUUUUCAU (SEQ ID NO: 6027), or a nucleic acid sequence thereof having about 1 or 2 nucleoside substitutions, additions, or deletions. In some cases, Z2 comprises G, C, A, or U. In some cases, Z3 comprises GUCGAGAGAUCA (SEQ ID NO: 6028), CA, UGCCA, GGAGC, GGAGCCUUC, or a nucleic acid sequence thereof having about 1 or 2 nucleoside substitutions, additions, or deletions. In some cases, Z4 comprises GAAAU, UUGUC, C, UCCUAAC, or AAC, or a nucleic acid sequence thereof having about 1 or 2 nucleoside substitutions, additions, or deletions. In some embodiments, Z5 represents 0 nucleosides. In some embodiments, Z5 represents 3 nucleosides. In some embodiments, Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z5 nucleosides comprise a deoxyribose. The one or more may be one, two, or three. In some embodiments, Formula IVA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula IVB comprises 5′ U-Z6-A-G-Z7-A-Z8-C-Z9-U-U 3′, wherein Z6 represents 0, 1, 3, or 5 nucleosides; Z7 represents 0, 2, 5, 6, 9, or 12 nucleosides; Z8 represents 1 nucleoside; and Z9 represents 1, 2, 3, 6, 11, or 12 nucleosides. In some cases, Z6 comprises AUUUC, GACAA, A, GUU, GUUAGGA, or a nucleic acid sequence thereof having about 1 or 2 nucleoside substitutions, additions, or deletions. In some cases, Z7 comprises UG, UGAUCUCUCGAC (SEQ ID NO: 6029), UGGCA, GAAGGCUCC, GCUCC, UGGCAA, or a nucleic acid sequence thereof having about 1 or 2 nucleoside substitutions, additions, or deletions. In some cases, Z8 comprises U, G, C, or A. In some cases, Z9 comprises UCUCGACACCA (SEQ ID NO: 6030), A, UG, U, UCUCGA, AUGAAAAGGUGC (SEQ ID NO: 6031), or a nucleic acid sequence thereof having about 1 or 2 nucleoside substitutions, additions, or deletions.

Formula VA comprises 5′ Z1-C-Z2-G-Z3-U-C-Z4-A-U-U-Z5-L 3′, wherein Z1 represents 0, 1, 2, 6, or 9 nucleosides; Z2 represents 2 nucleosides; Z3 represents 1, 3, 5, or 12 nucleosides; Z4 represents 0, 2, 3, 5, 8 nucleosides; Z5 represents 0 or 3 nucleosides; and L comprises an optional lipid. In some cases, Z1 comprises UGGUGU, U, AG, or GCACCUUUU or a nucleic acid sequence thereof having about 1 or 2 nucleoside substitutions, additions, or deletions. In some cases, Z2 comprises GA, AG, AU, or UG. In some cases, Z3 comprises AGA, G, UUUGCCACUUUG (SEQ ID NO: 6032), AGCCU, or a nucleic acid sequence thereof having about 1 or 2 nucleoside substitutions, additions, or deletions. In some cases, Z4 comprises ACU, ACUGAAAU, UC, CUAAC, GUC, or a nucleic acid sequence thereof having about 1 or 2 nucleoside substitutions, additions, or deletions. In some embodiments, Z5 represents 0 nucleosides. In some embodiments, Z5 represents 3 nucleosides. In some embodiments, Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z5 nucleosides comprise a deoxyribose. The one or more may be one, two, or three. In some embodiments, Formula VA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula VB comprises 5′ U-Z6-G-A-Z7-C-Z8-G-Z9-U-U 3′, wherein Z6 represents 0, 2, 3, 5, or 8 nucleosides; Z7 represents 1, 3, 5, or 12 nucleosides; Z8 represents 2 nucleosides; and Z9 represents 0, 1, 2, 6, or 9 nucleosides. In some cases, Z6 comprises AGU, AUUUCAGU, GA, GUUAG, or a nucleic acid sequence thereof having about 1 or 2 nucleoside substitutions, additions, or deletions. In some cases, Z7 comprises UCU, C, CAAAGUGGCAAA (SEQ ID NO: 6033), AGGCU, or a nucleic acid sequence thereof having about 1 or 2 nucleoside substitutions, additions, or deletions. In some cases, Z8 comprises UC, CU, AU, or CA. In some cases, Z9 comprises ACACCA, A, AAAAGGUGC, or CU, or a nucleic acid sequence thereof having about 1 or 2 nucleoside substitutions, additions, or deletions.

Formula VIA comprises 5′ Z1-C-Z2-G-Z3-C-U-Z4-A-U-U-Z5-L 3′, wherein Z1 represents 0, 1, 2, 6, or 9 nucleosides; Z2 represents 2 nucleosides; Z3 represents 2, 3, 6, 7 nucleosides; Z4 represents 0, 1, 3, 5, or 7 nucleosides; Z5 represents 0 or 3 nucleosides; and L comprises an optional lipid. In some cases, Z1 comprises UGGUGU, U, AG, or GCACCUUUU or a nucleic acid sequence thereof having about 1 or 2 nucleoside substitutions, additions, or deletions. In some cases, Z2 comprises GA, AG, AU, or UG. In some cases, Z3 comprises AGAUCA, UUUGCCA, GU, AGC, or AGCCUUC, or a nucleic acid sequence thereof having about 1 or 2 nucleoside substitutions, additions, or deletions. In some cases, Z4 comprises GAAAU, UUGUC, C, AAC, or UCCUAAC, or a nucleic acid sequence thereof having about 1 or 2 nucleoside substitutions, additions, or deletions. In some embodiments, Z5 represents 0 nucleosides. In some embodiments, Z5 represents 3 nucleosides. In some embodiments, Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z5 nucleosides comprise a deoxyribose. The one or more may be one, two, or three. In some embodiments, Formula VIA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula VIB comprises 5′ U-Z6-A-G-Z7-C-Z8-G-Z9-U-U 3′, wherein Z6 represents 0, 1, 3, 5, or 7 nucleosides; Z7 represents 2, 3, 6, or 7 nucleosides; Z8 represents 2 nucleosides; and Z9 represents 0, 1, 2, 6, or 9 nucleosides. In some cases, Z6 comprises AUUUC, GACAA, G, GUU, or GUUAGGA or a nucleic acid sequence thereof having about 1 or 2 nucleoside substitutions, additions, or deletions. In some cases, Z7 comprises UGAUCU, AC, GAAGGCU, or GCU, or a nucleic acid sequence thereof having about 1 or 2 nucleoside substitutions, additions, or deletions. In some cases, Z8 comprises UC, CU, AU, or CA. In some cases, Z9 comprises ACACCA, A, CU, AAAAGGUGC, or a nucleic acid sequence thereof having about 1 or 2 nucleoside substitutions, additions, or deletions.

Formula VIIA comprises 5′ Z1-C-Z2-G-Z3-U-Z4-C-Z5-A-U-U-Z6-L 3′, wherein Z1 represents 0, 1, 2, 6, or 9 nucleosides; Z2 represents 2 nucleosides; Z3 represents 1, 2, 3, 4, 8, or 10 nucleosides; Z4 represents 2 nucleosides; Z5 represents 0, 1, 4, 6, 8, or 9 nucleosides; Z6 represents 0 or 3 nucleosides; and L comprises an optional lipid. In some cases, Z1 comprises UGGUGU, U, GCACCUUUU, AGCUGGAGCCUUCC (SEQ ID NO: 6034), CAGGUU, or AG, or a nucleic acid sequence thereof having about 1 or 2 nucleoside substitutions, additions, or deletions. In some cases, Z2 comprises GA, AG, AU, or UG. In some cases, Z3 comprises AGA, U, G, AGCC, UUUGCCACUU (SEQ ID NO: 6035), AGCCUUCC, or a nucleic acid sequence thereof having about 1 or 2 nucleoside substitutions, additions, or deletions. In some cases, Z4 comprises CA, UG, GC, GU, CU, UC, or AA. In some cases, Z5 comprises U, UGAAAU, ACUUUGUC, CACUUUGUC, UAAC, or a nucleic acid sequence thereof having about 1 or 2 nucleoside substitutions, additions, or deletions. In some embodiments, Z6 represents 0 nucleosides. In some embodiments, Z6 represents 3 nucleosides. In some embodiments, Z6 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z6 nucleosides comprise a deoxyribose. The one or more may be one, two, or three. In some embodiments, Formula VIIA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula VIIB comprises 5′ U-Z7-G-Z8-A-Z9-C-Z10-G-Z11-U-U 3′, wherein Z7 represents 0, 1, 4, 6, 8, or 9 nucleosides; Z8 represents 2 nucleosides; Z9 represents 1, 2, 3, 4, 8, or 10 nucleosides; Z10 represents 2 nucleosides; and Z11 represents 0, 1, 2, 6, or 9 nucleosides. In some cases, Z7 comprises A, AUUUCA, GACAAAGU, GACAAAGUG, GUUA, or a nucleic acid sequence thereof having about 1 or 2 nucleoside substitutions, additions, or deletions. In some cases, Z8 comprises UG, GC, CA, AG, UU, or GA. In some cases, Z9 comprises UCU, A, AAGUGGCAAA (SEQ ID NO: 6036), C, GGCU, GGAAGGCU, or a nucleic acid sequence thereof having about 1 or 2 nucleoside substitutions, additions, or deletions. In some cases, Z10 comprises UC, CU, AU, or CA. In some cases, Z11 comprises ACACCA, A, CU, AAAAGGUGC, or a nucleic acid sequence thereof having about 1 or 2 nucleoside substitutions, additions, or deletions.

Formula VIIIA comprises 5′ Z1-A-Z2-U-Z3-C-U-Z4-A-U-U-Z5-L 3′, wherein Z1 represents 0, 1, 2, 5, or 10 nucleosides; Z2 represents 2 nucleosides; Z3 represents 2, 5, 6, or 9 nucleosides; Z4 represents 0, 1, 3, 5, or 7 nucleosides; Z5 represents 0 or 3 nucleosides; and L comprises an optional lipid. In some cases, Z1 comprises UGGUGUCGAG (SEQ ID NO: 6037), UCGAG, C, GC, or a nucleic acid sequence thereof having about 1 or 2 nucleoside substitutions, additions, or deletions. In some cases, Z2 comprises GA, GG, CC, or GC. In some cases, Z3 comprises CA, UUGCCA, UUUCAUGGU, GGAGC, GGAGCCUUC, or a nucleic acid sequence thereof having about 1 or 2 nucleoside substitutions, additions, or deletions. In some cases, Z4 comprises GAAAU, UUGUC, C, AAC, or UCCUAAC, or a nucleic acid sequence thereof having about 1 or 2 nucleoside substitutions, additions, or deletions. In some embodiments, Z5 represents 0 nucleosides. In some embodiments, Z5 represents 3 nucleosides. In some embodiments, Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. In some embodiments, one or more of the Z5 nucleosides comprise a deoxyribose. The one or more may be one, two, or three. In some embodiments, Formula VIIIA comprises the lipid. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

Formula VIIIB comprises 5′ U-Z6-A-G-Z7-A-Z8-U-Z9-U-U 3′, wherein Z6 represents 0, 1, 3, 5, or 7 nucleosides; Z7 represents 2, 5, 6, or 9 nucleosides; Z8 represents 2 nucleosides; and Z9 represents 0, 1, 2, 5, or 10 nucleosides. In some cases, Z6 comprises AUUUC, GACAA, G, GUU, or GUUAGGA, or a nucleic acid sequence thereof having about 1 or 2 nucleoside substitutions, additions, or deletions. In some cases, Z7 comprises UG, UGGCAA, ACCAUGAAA, GAAGGCUCC, GCUCC, or a nucleic acid sequence thereof having about 1 or 2 nucleoside substitutions, additions, or deletions. In some cases, Z8 comprises UC, CC, GG, or GC. In some cases, Z9 comprises CUCGACACCA (SEQ ID NO: 6038), CUCGA, G, or GC or a nucleic acid sequence thereof having about 1 or 2 nucleoside substitutions, additions, or deletions.

In some embodiments, the sense strand comprises mUsmGsmGmUfGmUfCfGfAmGmAmGmAmUmCmAmCmUmAsmUsmU (SEQ ID NO: 5558), or a nucleic acid sequence thereof having about 1 or 2 nucleoside substitutions, additions, or deletions. In some embodiments, the sense strand comprises mUsmCsmGmAfGmAfGfAfUmCmAmCmUmGmAmAmAmUmAsmUsmU (SEQ ID NO: 5560), or a nucleic acid sequence thereof having about 1 or 2 nucleoside substitutions, additions, or deletions. In some embodiments, the sense strand comprises mCsmAsmGmGfUmUfUfGfCmCmAmCmUmUmUmGmUmCmAsmUsmU (SEQ ID NO: 5562), or a nucleic acid sequence thereof having about 1 or 2 nucleoside substitutions, additions, or deletions. In some embodiments, the sense strand comprises mGsmCsmAmCfCmUfUfUfUmCmAmUmGmGmUmCmUmCmAsmUsmU (SEQ ID NO: 5564), or a nucleic acid sequence thereof having about 1 or 2 nucleoside substitutions, additions, or deletions. In some embodiments, the sense strand comprises mAsmGsmCmUfGmGfAfGfCmCmUmUmCmCmUmAmAmCmAsmUsmU (SEQ ID NO: 5566), or a nucleic acid sequence thereof having about 1 or 2 nucleoside substitutions, additions, or deletions.

In some embodiments, the sense strand comprises fUsmGsfGmUfGmUfCfGfAmGfAmGfAmUfCmAfCmUfAsmUsmU (SEQ ID NO: 5456), or a nucleic acid sequence thereof having about 1 or 2 nucleoside substitutions, additions, or deletions. In some embodiments, the sense strand comprises fUsmCsfGmAfGmAfGfAfUmCfAmCfUmGfAmAfAmUfAsmUsmU (SEQ ID NO: 5460), or a nucleic acid sequence thereof having about 1 or 2 nucleoside substitutions, additions, or deletions. In some embodiments, the sense strand comprises fCsmAsfGmGfUmUfUfGfCmCfAmCfUmUfUmGfUmCfAsmUsmU (SEQ ID NO: 5476), or a nucleic acid sequence thereof having about 1 or 2 nucleoside substitutions, additions, or deletions. In some embodiments, the sense strand comprises fGsmCsfAmCfCmUfUfUfUmCfAmUfGmGfUmCfUmCfAsmUsmU (SEQ ID NO: 5510), or a nucleic acid sequence thereof having about 1 or 2 nucleoside substitutions, additions, or deletions. In some embodiments, the sense strand comprises fAsmGsfCmUfGmGfAfGfCmCfUmUfCmCfUmAfAmCfAsmUsmU (SEQ ID NO: 5524), or a nucleic acid sequence thereof having about 1 or 2 nucleoside substitutions, additions, or deletions.

In some embodiments, the sense strand comprises mUsmGsmGmUfGmUfCmGfAmGmAmGmAmUmCmAmCmUmAsmUsmU (SEQ ID NO: 6050). In some embodiments, the sense strand comprises mUsmCsmGmAfGmAfGmAfUmCmAmCmUmGmAmAmAmUmAsmUsmU (SEQ ID NO: 6051), or a nucleic acid sequence thereof having about 1 or 2 nucleoside substitutions, additions, or deletions. In some embodiments, the sense strand comprises mCsmAsmGmGfUmUfUmGfCmCmAmCmUmUmUmGmUmCmAsmUsmU (SEQ ID NO: 6052), or a nucleic acid sequence thereof having about 1 or 2 nucleoside substitutions, additions, or deletions. In some embodiments, the sense strand comprises mGsmCsmAmCfCmUfUmUfUmCmAmUmGmGmUmCmUmCmAsmUsmU (SEQ ID NO: 6053), or a nucleic acid sequence thereof having about 1 or 2 nucleoside substitutions, additions, or deletions. In some embodiments, the sense strand comprises mAsmGsmCmUfGmGfAmGfCmCmUmUmCmCmUmAmAmCmAsmUsmU (SEQ ID NO: 6054), or a nucleic acid sequence thereof having about 1 or 2 nucleoside substitutions, additions, or deletions.

In some embodiments, the sense strand comprises fUsmGsfGmUfGmUfCfGfAmGfAmGfAmUfCmAfCmUfAsmUsmUNNN-lipid (wherein each N is independently a nucleobase such as A, U, C, or G) (SEQ ID NO: 6055), or a nucleic acid sequence thereof having about 1 or 2 nucleoside substitutions, additions, or deletions. In some embodiments, the sense strand comprises fUsmCsfGmAfGmAfGfAfUmCfAmCfUmGfAmAfAmUfAsmUsmUNNN-lipid (wherein each N is independently a nucleobase such as A, U, C, or G) (SEQ ID NO: 6056), or a nucleic acid sequence thereof having about 1 or 2 nucleoside substitutions, additions, or deletions. In some embodiments, the sense strand comprises fCsmAsfGmGfUmUfUfGfCmCfAmCfUmUfUmGfUmCfAsmUsmUNNN-lipid (wherein each N is independently a nucleobase such as A, U, C, or G) (SEQ ID NO: 6057), or a nucleic acid sequence thereof having about 1 or 2 nucleoside substitutions, additions, or deletions. In some embodiments, the sense strand comprises fGsmCsfAmCfCmUfUfUfUmCfAmUfGmGfUmCfUmCfAsmUsmUNNN-lipid (wherein each N is independently a nucleobase such as A, U, C, or G) (SEQ ID NO: 6058), or a nucleic acid sequence thereof having about 1 or 2 nucleoside substitutions, additions, or deletions. In some embodiments, the sense strand comprises fAsmGsfCmUfGmGfAfGfCmCfUmUfCmCfUmAfAmCfAsmUsmUNNN-lipid (wherein each N is independently a nucleobase such as A, U, C, or G) (SEQ ID NO: 6059), or a nucleic acid sequence thereof having about 1 or 2 nucleoside substitutions, additions, or deletions.

In some embodiments, the sense strand comprises mUsmGsmGmUfGmUfCfGfAmGmAmGmAmUmCmAmCmUmAsmUsmUNNN-lipid (wherein each N is independently a nucleobase such as A, U, C, or G) (SEQ ID NO: 6060), or a nucleic acid sequence thereof having about 1 or 2 nucleoside substitutions, additions, or deletions. In some embodiments, the sense strand comprises mUsmCsmGmAfGmAfGfAfUmCmAmCmUmGmAmAmAmUmAsmUsmUNNN-lipid (wherein each N is independently a nucleobase such as A, U, C, or G) (SEQ ID NO: 6061), or a nucleic acid sequence thereof having about 1 or 2 nucleoside substitutions, additions, or deletions. In some embodiments, the sense strand comprises mCsmAsmGmGfUmUfUfGfCmCmAmCmUmUmUmGmUmCmAsmUsmUNNN-lipid (wherein each N is independently a nucleobase such as A, U, C, or G) (SEQ ID NO: 6062), or a nucleic acid sequence thereof having about 1 or 2 nucleoside substitutions, additions, or deletions. In some embodiments, the sense strand comprises mGsmCsmAmCfCmUfUfUfUmCmAmUmGmGmUmCmUmCmAsmUsmUNNN-lipid (wherein each N is independently a nucleobase such as A, U, C, or G) (SEQ ID NO: 6063), or a nucleic acid sequence thereof having about 1 or 2 nucleoside substitutions, additions, or deletions. In some embodiments, the sense strand comprises mAsmGsmCmUfGmGfAfGfCmCmUmUmCmCmUmAmAmCmAsmUsmUNNN-lipid (wherein each N is independently a nucleobase such as A, U, C, or G) (SEQ ID NO: 6064), or a nucleic acid sequence thereof having about 1 or 2 nucleoside substitutions, additions, or deletions.

In some embodiments, the antisense strand comprises mUsfAsmGmUmGfAmUmCmUmCmUmCmGfAmCfAmCmCmAsmUsmU (SEQ ID NO: 5559), or a nucleic acid sequence thereof having about 1 or 2 nucleoside substitutions, additions, or deletions. In some embodiments, the antisense strand comprises mUsfAsmUmUmUfCmAmGmUmGmAmUmCfUmCfUmCmGmAsmUsmU (SEQ ID NO: 5561), or a nucleic acid sequence thereof having about 1 or 2 nucleoside substitutions, additions, or deletions. In some embodiments, the antisense strand comprises mUsfGsmAmCmAfAmAmGmUmGmGmCmAfAmAfCmCmUmGsmUsmU (SEQ ID NO: 5563), or a nucleic acid sequence thereof having about 1 or 2 nucleoside substitutions, additions, or deletions. In some embodiments, the antisense strand comprises mUsfGsmAmGmAfCmCmAmUmGmAmAmAfAmGfGmUmGmCsmUsmU (SEQ ID NO: 5565), or a nucleic acid sequence thereof having about 1 or 2 nucleoside substitutions, additions, or deletions. In some embodiments, the antisense strand comprises mUsfGsmUmUmAfGmGmAmAmGmGmCmUfCmCfAmGmCmUsmUsmU (SEQ ID NO: 5567), or a nucleic acid sequence thereof having about 1 or 2 nucleoside substitutions, additions, or deletions.

In some embodiments, the antisense strand comprises mUsfAsmGfUmGfAmUfCmUfCmUmCmGfAmCfAmCfCmAsmUsmU (SEQ ID NO: 5457), or a nucleic acid sequence thereof having about 1 or 2 nucleoside substitutions, additions, or deletions. In some embodiments, the antisense strand comprises mUsfAsmUfUmUfCmAfGmUfGmAmUmCfUmCfUmCfGmAsmUsmU (SEQ ID NO: 5461), or a nucleic acid sequence thereof having about 1 or 2 nucleoside substitutions, additions, or deletions. In some embodiments, the antisense strand comprises mUsfGsmAfCmAfAmAfGmUfGmGmCmAfAmAfCmCfUmGsmUsmU (SEQ ID NO: 5477), or a nucleic acid sequence thereof having about 1 or 2 nucleoside substitutions, additions, or deletions. In some embodiments, the antisense strand comprises mUsfGsmAfGmAfCmCfAmUfGmAmAmAfAmGfGmUfGmCsmUsmU (SEQ ID NO: 5511), or a nucleic acid sequence thereof having about 1 or 2 nucleoside substitutions, additions, or deletions. In some embodiments, the antisense strand comprises mUsfGsmUfUmAfGmGfAmAfGmGmCmUfCmCfAmGfCmUsmUsmU (SEQ ID NO: 5525), or a nucleic acid sequence thereof having about 1 or 2 nucleoside substitutions, additions, or deletions.

In some embodiments, the antisense strand comprises mUsfAsmGmUmGfAmUfCfUmCmUmCmGfAmCfAmCmCmAsmUsmU (SEQ ID NO: 6065), or a nucleic acid sequence thereof having about 1 or 2 nucleoside substitutions, additions, or deletions. In some embodiments, the antisense strand comprises mUsfAsmUmUmUfCmAfGfUmGmAmUmCfUmCfUmCmGmAsmUsmU (SEQ ID NO: 6066), or a nucleic acid sequence thereof having about 1 or 2 nucleoside substitutions, additions, or deletions. In some embodiments, the antisense strand comprises mUsfGsmAmCmAfAmAfGfUmGmGmCmAfAmAfCmCmUmGsmUsmU (SEQ ID NO: 6067), or a nucleic acid sequence thereof having about 1 or 2 nucleoside substitutions, additions, or deletions. In some embodiments, the antisense strand comprises mUsfGsmAmGmAfCmCfAfUmGmAmAmAfAmGfGmUmGmCsmUsmU (SEQ ID NO: 6068), or a nucleic acid sequence thereof having about 1 or 2 nucleoside substitutions, additions, or deletions. In some embodiments, the antisense strand comprises mUsfGsmUmUmAfGmGfAfAmGmGmCmUfCmCfAmGmCmUsmUsmU (SEQ ID NO: 6069), or a nucleic acid sequence thereof having about 1 or 2 nucleoside substitutions, additions, or deletions.

In some embodiments, the antisense strand comprises mUsfAsmGfUmGfAmUmCmUmCmUmCmGfAmCfAmCmCmAsmUsmU (SEQ ID NO: 6070), or a nucleic acid sequence thereof having about 1 or 2 nucleoside substitutions, additions, or deletions. In some embodiments, the antisense strand comprises mUsfAsmUfUmUfCmAmGmUmGmAmUmCfUmCfUmCmGmAsmUsmU (SEQ ID NO: 6071), or a nucleic acid sequence thereof having about 1 or 2 nucleoside substitutions, additions, or deletions. In some embodiments, the antisense strand comprises mUsfGsmAfCmAfAmAmGmUmGmGmCmAfAmAfCmCmUmGsmUsmU (SEQ ID NO: 6072), or a nucleic acid sequence thereof having about 1 or 2 nucleoside substitutions, additions, or deletions. In some embodiments, the antisense strand comprises mUsfGsmAfGmAfCmCmAmUmGmAmAmAfAmGfGmUmGmCsmUsmU (SEQ ID NO: 6073), or a nucleic acid sequence thereof having about 1 or 2 nucleoside substitutions, additions, or deletions. In some embodiments, the antisense strand comprises mUsfGsmUfUmAfGmGmAmAmGmGmCmUfCmCfAmGmCmUsmUsmU (SEQ ID NO: 6074), or a nucleic acid sequence thereof having about 1 or 2 nucleoside substitutions, additions, or deletions.

A dsRNA may comprise any sense strand described herein in combination with any antisense strand described herein. In some embodiments, the sense strand comprises a nucleic acid comprising pattern 1S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 1AS. In some embodiments, the sense strand comprises a nucleic acid comprising pattern 1S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 2AS. In some embodiments, the sense strand comprises a nucleic acid comprising pattern 1S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 3AS. In some embodiments, the sense strand comprises a nucleic acid comprising pattern 1S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 4AS. In some embodiments, the sense strand comprises a sequence from SEQ ID NOS: 1-5349, 5366-5971, 6000-6038, 6050-6074.

A dsRNA may comprise any sense strand described herein in combination with any antisense strand described herein. In some embodiments, the sense strand comprises a nucleic acid comprising pattern 2S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 1AS. In some embodiments, the sense strand comprises a nucleic acid comprising pattern 2S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 2AS. In some embodiments, the sense strand comprises a nucleic acid comprising pattern 2S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 3AS. In some embodiments, the sense strand comprises a nucleic acid comprising pattern 2S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 4AS. In some embodiments, the sense strand comprises a sequence from SEQ ID NOS: 1-5349, 5366-5971, 6000-6038, 6050-6074.

A dsRNA may comprise any sense strand described herein in combination with any antisense strand described herein. In some embodiments, the sense strand comprises a nucleic acid comprising pattern 3S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 1AS. In some embodiments, the sense strand comprises a nucleic acid comprising pattern 3S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 2AS. In some embodiments, the sense strand comprises a nucleic acid comprising pattern 3S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 3AS. In some embodiments, the sense strand comprises a nucleic acid comprising pattern 3S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 4AS. In some embodiments, the sense strand comprises a sequence from SEQ ID NOS: 1-5349, 5366-5971, 6000-6038, 6050-6074.

A dsRNA may comprise any sense strand described herein in combination with any antisense strand described herein. In some embodiments, the sense strand comprises a nucleic acid comprising pattern 4S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 1AS. In some embodiments, the sense strand comprises a nucleic acid comprising pattern 4S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 2AS. In some embodiments, the sense strand comprises a nucleic acid comprising pattern 4S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 3AS. In some embodiments, the sense strand comprises a nucleic acid comprising pattern 4S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 4AS. In some embodiments, the sense strand comprises a sequence from SEQ ID NOS: 1-5349, 5366-5971, 6000-6038, 6050-6074.

A dsRNA may comprise any sense strand described herein in combination with any antisense strand described herein. In some embodiments, the sense strand comprises a nucleic acid comprising pattern 5S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 1AS. In some embodiments, the sense strand comprises a nucleic acid comprising pattern 5S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 2AS. In some embodiments, the sense strand comprises a nucleic acid comprising pattern 5S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 3AS. In some embodiments, the sense strand comprises a nucleic acid comprising pattern 5S and the antisense strand comprises the reverse complement of at least about 10, 11, 12, 13, 14, 15, 16, 17, 18, 19, 20, or 21 nucleosides of the sense strand (e.g., about 18 nucleosides), and the pattern 4AS. In some embodiments, the sense strand comprises a sequence from SEQ ID NOS: 1-5349, 5366-5971, 6000-6038, 6050-6074.

In some aspects, provided herein is a dsRNA, wherein the sense strand comprises UGGUGUCGAGAGAUCACUG (SEQ ID NO: 5664) or a nucleic acid sequence thereof having about 1 or 2 nucleoside substitutions, additions, or deletions; and the antisense strand comprises CAGUGAUCUCUCGACACCA (SEQ ID NO: 5765) or a nucleic acid sequence thereof having about 1 or 2 nucleoside substitutions, additions, or deletions. In some aspects, provided herein is a dsRNA, wherein the sense strand comprises UGGUGUCGAGAGAUCACUA (SEQ ID NO: 5866) or a nucleic acid sequence thereof having about 1 or 2 nucleoside substitutions, additions, or deletions; and the antisense strand comprises UAGUGAUCUCUCGACACCA (SEQ ID NO: 5967) or a nucleic acid sequence thereof having about 1 or 2 nucleoside substitutions, additions, or deletions. In some aspects, provided herein is a dsRNA, wherein the sense strand comprises mUsmGsmGmUfGmUfCfGfAmGmAmGmAmUmCmAmCmUmAsmUsmU (SEQ ID NO: 5558) or a nucleic acid sequence thereof having about 1 or 2 nucleoside substitutions, additions, or deletions; and the antisense strand comprises mUsfAsmGmUmGfAmUmCmUmCmUmCmGfAmCfAmCmCmAsmUsmU (SEQ ID NO: 5559) or a nucleic acid sequence thereof having about 1 or 2 nucleoside substitutions, additions, or deletions. In some aspects, provided herein is a dsRNA, wherein the sense strand comprises UCGAGAGAUCACUGAAAUC (SEQ ID NO: 5665) or a nucleic acid sequence thereof having about 1 or 2 nucleoside substitutions, additions, or deletions; and the antisense strand comprises GAUUUCAGUGAUCUCUCGA (SEQ ID NO: 5766) or a nucleic acid sequence thereof having about 1 or 2 nucleoside substitutions, additions, or deletions. In some aspects, provided herein is a dsRNA, wherein the sense strand comprises UCGAGAGAUCACUGAAAUA (SEQ ID NO: 5867) or a nucleic acid sequence thereof having about 1 or 2 nucleoside substitutions, additions, or deletions; and the antisense strand comprises UAUUUCAGUGAUCUCUCGA (SEQ ID NO: 5968) or a nucleic acid sequence thereof having about 1 or 2 nucleoside substitutions, additions, or deletions. In some aspects, provided herein is a dsRNA, wherein the sense strand comprises mUsmCsmGmAfGmAfGfAfUmCmAmCmUmGmAmAmAmUmAsmUsmU (SEQ ID NO: 5560) or a nucleic acid sequence thereof having about 1 or 2 nucleoside substitutions, additions, or deletions; and the antisense strand comprises mUsfAsmUmUmUfCmAmGmUmGmAmUmCfUmCfUmCmGmAsmUsmU (SEQ ID NO: 5561) or a nucleic acid sequence thereof having about 1 or 2 nucleoside substitutions, additions, or deletions. In some aspects, provided herein is a dsRNA, wherein the sense strand comprises CAGGUUUGCCACUUUGUCA (SEQ ID NO: 5868) or a nucleic acid sequence thereof having about 1 or 2 nucleoside substitutions, additions, or deletions; and the antisense strand comprises UGACAAAGUGGCAAACCUG (SEQ ID NO: 5969) or a nucleic acid sequence thereof having about 1 or 2 nucleoside substitutions, additions, or deletions. In some aspects, provided herein is a dsRNA, wherein the sense strand comprises CAGGUUUGCCACUUUGUCA (SEQ ID NO: 5868) or a nucleic acid sequence thereof having about 1 or 2 nucleoside substitutions, additions, or deletions; and the antisense strand comprises UGACAAAGUGGCAAACCUG (SEQ ID NO: 5969) or a nucleic acid sequence thereof having about 1 or 2 nucleoside substitutions, additions, or deletions. In some aspects, provided herein is a dsRNA, wherein the sense strand comprises mCsmAsmGmGfUmUfUfGfCmCmAmCmUmUmUmGmUmCmAsmUsmU (SEQ ID NO: 5562) or a nucleic acid sequence thereof having about 1 or 2 nucleoside substitutions, additions, or deletions; and the antisense strand comprises mUsfGsmAmCmAfAmAmGmUmGmGmCmAfAmAfCmCmUmGsmUsmU (SEQ ID NO: 5563) or a nucleic acid sequence thereof having about 1 or 2 nucleoside substitutions, additions, or deletions. In some aspects, provided herein is a dsRNA, wherein the sense strand comprises GCACCUUUUCAUGGUCUCU (SEQ ID NO: 5667) or a nucleic acid sequence thereof having about 1 or 2 nucleoside substitutions, additions, or deletions; and the antisense strand comprises AGAGACCAUGAAAAGGUGC (SEQ ID NO: 5768) or a nucleic acid sequence thereof having about 1 or 2 nucleoside substitutions, additions, or deletions. In some aspects, provided herein is a dsRNA, wherein the sense strand comprises GCACCUUUUCAUGGUCUCA (SEQ ID NO: 5869) or a nucleic acid sequence thereof having about 1 or 2 nucleoside substitutions, additions, or deletions; and the antisense strand comprises UGAGACCAUGAAAAGGUGC (SEQ ID NO: 5970) or a nucleic acid sequence thereof having about 1 or 2 nucleoside substitutions, additions, or deletions. In some aspects, provided herein is a dsRNA, wherein the sense strand comprises mGsmCsmAmCfCmUfUfUfUmCmAmUmGmGmUmCmUmCmAsmUsmU (SEQ ID NO: 5564) or a nucleic acid sequence thereof having about 1 or 2 nucleoside substitutions, additions, or deletions; and the antisense strand comprises mUsfGsmAmGmAfCmCmAmUmGmAmAmAfAmGfGmUmGmCsmUsmU (SEQ ID NO: 5565) or a nucleic acid sequence thereof having about 1 or 2 nucleoside substitutions, additions, or deletions. In some aspects, provided herein is a dsRNA, wherein the sense strand comprises AGCUGGAGCCUUCCUAACC (SEQ ID NO: 5668) or a nucleic acid sequence thereof having about 1 or 2 nucleoside substitutions, additions, or deletions; and the antisense strand comprises GGUUAGGAAGGCUCCAGCU (SEQ ID NO: 5769) or a nucleic acid sequence thereof having about 1 or 2 nucleoside substitutions, additions, or deletions. In some aspects, provided herein is a dsRNA, wherein the sense strand comprises AGCUGGAGCCUUCCUAACA (SEQ ID NO: 5870) or a nucleic acid sequence thereof having about 1 or 2 nucleoside substitutions, additions, or deletions; and the antisense strand comprises UGUUAGGAAGGCUCCAGCU (SEQ ID NO: 5971) or a nucleic acid sequence thereof having about 1 or 2 nucleoside substitutions, additions, or deletions. In some aspects, provided herein is a dsRNA, wherein the sense strand comprises mAsmGsmCmUfGmGfAfGfCmCmUmUmCmCmUmAmAmCmAsmUsmU (SEQ ID NO: 5566) or a nucleic acid sequence thereof having about 1 or 2 nucleoside substitutions, additions, or deletions; and the antisense strand comprises mUsfGsmUmUmAfGmGmAmAmGmGmCmUfCmCfAmGmCmUsmUsmU (SEQ ID NO: 5567) or a nucleic acid sequence thereof having about 1 or 2 nucleoside substitutions, additions, or deletions.

In some aspects, provided herein is a dsRNA comprising a sense strand comprising one or more sequences selected from SEQ ID NOS: 1-5349, 5366-5971, 6000-6038, and 6050-6074; or a nucleic acid sequence thereof having about 1 or 2 nucleoside substitutions, additions, or deletions. In some cases, the dsRNA further comprises an antisense strand comprising the reverse complement of the sense strand. In some aspects, provided herein is a dsRNA comprising a sense strand comprising the first 19 nucleobases of one or more sequences selected from SEQ ID NOS: 1-5349, 5366-5971, 6000-6038, and 6050-6074; or a nucleic acid sequence thereof having about 1 or 2 nucleoside substitutions, additions, or deletions. In some cases, the dsRNA further comprises an antisense strand comprising the reverse complement of the sense strand.

In some embodiments, a dsRNA is provided that inhibits the expression of ALOX15, wherein the siRNA comprises a sense strand and an antisense strand, each strand is independently 14-30 nucleosides in length, and at least one of the sense strand and the antisense strand comprises a nucleoside sequence comprising at least 14 contiguous nucleosides of SEQ ID NO: 6000 (C AGAGCTGCAG ACCTGGTGTC GAGAGATCAC TGAAATCGGG CTGCAAGGGG CCCAGGACCG AGGGTTTCCT GTCTCTTTAC AGGCTCGGGA CCAGGTTTGC CACTTTGTCA CCATGTGTAT CTTCACCTGC ACCGGCCAAC ACGCCTCTGT). In some cases, the sense strand comprises at least 14 contiguous nucleosides of SEQ ID NO: 6001 (C AGAGCTGCAG ACCTGGTGTC GAGAGATCAC TGAAATCGGG CTGCAAGGGG). In some cases, the sense strand comprises at least 14 contiguous nucleosides of SEQ ID NO: 6002 (CTTTAC AGGCTCGGGA CCAGGTTTGC CACTTTGTCA CCATGTGTAT CTTCA).

In some embodiments, a dsRNA is provided that inhibits the expression of ALOX15, wherein the siRNA comprises a sense strand and an antisense strand, each strand is independently 14-30 nucleosides in length, and at least one of the sense strand and the antisense strand comprises a nucleoside sequence comprising at least 14 contiguous nucleosides of SEQ ID NO: 6003 (A CCCTCTTCCC ATGTCCCACC CTCCCTAGAG GGGCACCTTT TCATGGTCTC TGCACCCAGT GAACACATTT TACTCTAGAG GCATCACCTG GGACCTTACT). In some embodiments, the sense strand and/or antisense strand comprises at least 14 contiguous nucleosides of SEQ ID NO: 6004 (TAGAG GGGCACCTTT TCATGGTCTC TGCACCCAGT GAACACATTT TACTC).

In some embodiments, a dsRNA is provided that inhibits the expression of ALOX15, wherein the siRNA comprises a sense strand and an antisense strand, each strand is independently 14-30 nucleosides in length, and at least one of the sense strand and the antisense strand comprises a nucleoside sequence comprising at least 14 contiguous nucleosides of SEQ ID NO: 6005 (G ACATGGGAAT TTTCGACCAG ATAATGAGCA CTGGTGGGGG AGGCCACGTG CAGCTGCTCA AGCAAGCTGG AGCCTTCCTA ACCTACAGCT CCTTCTGTCC CCCTGATGAC TTGGCCGACC GGGGGCTCCT GGGAGTGAAG TCTTCCTTCT). In some cases, the sense strand and/or antisense strand comprises at least 14 contiguous nucleosides of SEQ ID NO: 6006 (G CAGCTGCTCA AGCAAGCTGG AGCCTTCCTA ACCTACAGCT CCTTCTGTCC).

### Modifications

Described herein, in some embodiments, are dsRNA comprising a modification. Any of the sense strands and/or antisense strands described elsewhere herein may comprise a modification. Non-limiting examples of sense strand modifications include patterns 1S, 2S, 3S, 4S, and 5S. Pattern 1S comprises 5′ fN s mN s fN-mN-fN-mN-fN-fN-fN-mN-fN-mN-fN-mN-fN-mN-fN-mN-fN s mN s mN 3′. Pattern 2S comprises 5′ mN s mN s mN-mN-fN-mN-fN-fN-fN-mN-mN-mN-mN-mN-mN-mN-mN-mN-mN s mN s mN 3′. Pattern 3S comprises 5′ mN s mN s mN-mN-fN-mN-fN-mN-fN-mN-mN-mN-mN-mN-mN-mN-mN-mN-mN s mN s mN 3′. Pattern 4S comprises 5′ fN s mN s fN-mN-fN-mN-fN-fN-fN-mN-fN-mN-fN-mN-fN-mN-fN-mN-fN s mN s mN-N-N-N-Lipid 3′. Pattern 5S comprises 5′ mN s mN s mN-mN-fN-mN-fN-fN-fN-mN-mN-mN-mN-mN-mN-mN-mN-mN-mN s mN s mN-N-N-N-Lipid 3′. The “N” can be any nucleoside (for example ribose, deoxyribose, or derivatives thereof), “fN” is a 2′ fluoro-modified nucleoside, “m” is a 2′ O-methyl modified nucleoside, “-” is a phosphodiester and “s” is a phosphorothioate, and the lipid can comprise any molecule containing more than 14 sp3 hybridized carbons, e.g., cholesterol, myristoyl, palmitoyl, stearoyl, lithocholoyl, docosanoyl, docosahexaenoyl, myristyl, palmityl stearyl, α-tocopherol, or a combination thereof. In some embodiments, the N at position 1, 2, 3, 4, 5, 6, 7, 8, 9, 10, 11, 12, 13, 14, 15, 16, 17, 18, 19, 20, or 21, or a combination thereof, is a ribose.

In some embodiments, the sense strand comprises the pattern mNfNmNfN, where each mN may be connected to each fN by a phosphodiester or phosphorothioate bond. In some cases, the mNfNmNfN is at positions 4-7 of the sense strand. In some cases, the sense strand comprises fNfNfN, where each fN may be connected by a phosphodiester or phosphorothioate bond. In some cases, the sense strand comprises fNfNfNfNfNfNfNfNfNfNfNfN, where each fN may be connected by a phosphodiester or phosphorothioate bond.

Non-limiting examples of antisense strand modifications include patterns 1AS, 2AS, 3AS, and 4AS. Pattern 1AS comprises 5′ mN s fN s mN-fN-mN-fN-mN-fN-mN-fN-mN-mN-mN-fN-mN-fN-mN-fN-mN s mN s mN 3′. Pattern 2AS comprises 5′ mN s fN s mN-mN-mN-fN-mN-fN-fN-mN-mN-mN-mN-fN-mN-fN-mN-mN-mN s mN s mN 3′. Pattern 3AS comprises 5′ mN s fN s mN-mN-mN-fN-mN-mN-mN-mN-mN-mN-mN-fN-mN-fN-mN-mN-mN s mN s mN 3′. Pattern 4AS comprises 5′ mN s fN s mN-fN-mN-fN-mN-mN-mN-mN-mN-mN-mN-fN-mN-fN-mN-mN-mN s mN s mN 3′.

In some embodiments, the antisense strand comprises at least two patterns of mNmNmN, where each mN may be connected by a phosphodiester or phosphorothioate bond. For instance, the antisense strand may comprise 2 mNmNmN patterns. In some cases, the antisense strand comprises a mNmNmNmN pattern and a mNmNmNmNmN pattern. In some cases, the antisense strand comprises a mNmNmNmNmNmNmN pattern and a mNmNmNmNmN pattern. In some embodiments, the antisense strand comprises mNfNmNfN. In some cases, the mNfNmNfN is at positions 13-16 of the antisense strand, in a 5′ to 3′ direction.

In some embodiments, a dsRNA comprises a sense strand comprising pattern 1S and an antisense strand comprising pattern 1AS. In some embodiments, a dsRNA comprises a sense strand comprising pattern 2S and an antisense strand comprising pattern 2AS. In some embodiments, a dsRNA comprises a sense strand comprising pattern 3S and an antisense strand comprising pattern 3AS. In some embodiments, a dsRNA comprises a sense strand comprising pattern 4S and an antisense strand comprising pattern 4AS.

In some embodiments, a dsRNA comprising Formula IA comprises pattern 1S. In some embodiments, a dsRNA comprising Formula IIA comprises pattern 1S. In some embodiments, a dsRNA comprising Formula IIIA comprises pattern 1S. In some embodiments, a dsRNA comprising Formula IVA comprises pattern 1S. In some embodiments, a dsRNA comprising Formula VA comprises pattern 1S. In some embodiments, a dsRNA comprising Formula VIA comprises pattern 1S. In some embodiments, a dsRNA comprising Formula VIIA comprises pattern 1S. In some embodiments, a dsRNA comprising Formula VIIIA comprises pattern 1S.

In some embodiments, a dsRNA comprising Formula IA comprises pattern 2S. In some embodiments, a dsRNA comprising Formula IIA comprises pattern 2S. In some embodiments, a dsRNA comprising Formula IIIA comprises pattern 2S. In some embodiments, a dsRNA comprising Formula IVA comprises pattern 2S. In some embodiments, a dsRNA comprising Formula VA comprises pattern 2S. In some embodiments, a dsRNA comprising Formula VIA comprises pattern 2S. In some embodiments, a dsRNA comprising Formula VIIA comprises pattern 2S. In some embodiments, a dsRNA comprising Formula VIIIA comprises pattern 2S.

In some embodiments, a dsRNA comprising Formula IA comprises pattern 3S. In some embodiments, a dsRNA comprising Formula IIA comprises pattern 3S. In some embodiments, a dsRNA comprising Formula IIIA comprises pattern 3S. In some embodiments, a dsRNA comprising Formula IVA comprises pattern 3S. In some embodiments, a dsRNA comprising Formula VA comprises pattern 3S. In some embodiments, a dsRNA comprising Formula VIA comprises pattern 3S. In some embodiments, a dsRNA comprising Formula VIIA comprises pattern 3S. In some embodiments, a dsRNA comprising Formula VIIIA comprises pattern 3S.

In some embodiments, a dsRNA comprising Formula IA comprises pattern 4S. In some embodiments, a dsRNA comprising Formula IIA comprises pattern 4S. In some embodiments, a dsRNA comprising Formula IIIA comprises pattern 4S. In some embodiments, a dsRNA comprising Formula IVA comprises pattern 4S. In some embodiments, a dsRNA comprising Formula VA comprises pattern 4S. In some embodiments, a dsRNA comprising Formula VIA comprises pattern 4S. In some embodiments, a dsRNA comprising Formula VIIA comprises pattern 4S. In some embodiments, a dsRNA comprising Formula VIIIA comprises pattern 4S.

In some embodiments, a dsRNA comprising Formula IA comprises pattern 5S. In some embodiments, a dsRNA comprising Formula IIA comprises pattern 5S. In some embodiments, a dsRNA comprising Formula IIIA comprises pattern 5S. In some embodiments, a dsRNA comprising Formula IVA comprises pattern 5S. In some embodiments, a dsRNA comprising Formula VA comprises pattern 5S. In some embodiments, a dsRNA comprising Formula VIA comprises pattern 5S. In some embodiments, a dsRNA comprising Formula VIIA comprises pattern 5S. In some embodiments, a dsRNA comprising Formula VIIIA comprises pattern 5S.

In some embodiments, a dsRNA comprising Formula IB comprises pattern 1AS. In some embodiments, a dsRNA comprising Formula IIB comprises pattern 1AS. In some embodiments, a dsRNA comprising Formula IIIB comprises pattern 1AS. In some embodiments, a dsRNA comprising Formula IVB comprises pattern 1AS. In some embodiments, a dsRNA comprising Formula VB comprises pattern 1AS. In some embodiments, a dsRNA comprising Formula VIB comprises pattern 1AS. In some embodiments, a dsRNA comprising Formula VIIB comprises pattern 1AS. In some embodiments, a dsRNA comprising Formula VIIIB comprises pattern 1AS.

In some embodiments, a dsRNA comprising Formula IB comprises pattern 2AS. In some embodiments, a dsRNA comprising Formula IIB comprises pattern 2AS. In some embodiments, a dsRNA comprising Formula IIIB comprises pattern 2AS. In some embodiments, a dsRNA comprising Formula IVB comprises pattern 2AS. In some embodiments, a dsRNA comprising Formula VB comprises pattern 2AS. In some embodiments, a dsRNA comprising Formula VIB comprises pattern 2AS. In some embodiments, a dsRNA comprising Formula VIIB comprises pattern 2AS. In some embodiments, a dsRNA comprising Formula VIIIB comprises pattern 2AS.

In some embodiments, a dsRNA comprising Formula IB comprises pattern 3AS. In some embodiments, a dsRNA comprising Formula IIB comprises pattern 3AS. In some embodiments, a dsRNA comprising Formula IIIB comprises pattern 3AS. In some embodiments, a dsRNA comprising Formula IVB comprises pattern 3AS. In some embodiments, a dsRNA comprising Formula VB comprises pattern 3AS. In some embodiments, a dsRNA comprising Formula VIB comprises pattern 3AS. In some embodiments, a dsRNA comprising Formula VIIB comprises pattern 3AS. In some embodiments, a dsRNA comprising Formula VIIIB comprises pattern 3AS.

In some embodiments, a dsRNA comprising Formula IB comprises pattern 4AS. In some embodiments, a dsRNA comprising Formula IIB comprises pattern 4AS. In some embodiments, a dsRNA comprising Formula IIIB comprises pattern 4AS. In some embodiments, a dsRNA comprising Formula IVB comprises pattern 4AS. In some embodiments, a dsRNA comprising Formula VB comprises pattern 4AS. In some embodiments, a dsRNA comprising Formula VIB comprises pattern 4AS. In some embodiments, a dsRNA comprising Formula VIIB comprises pattern 4AS. In some embodiments, a dsRNA comprising Formula VIIIB comprises pattern 4AS.

In some embodiments, a dsRNA comprising Formula IB comprises pattern 5AS. In some embodiments, a dsRNA comprising Formula IIB comprises pattern 5AS. In some embodiments, a dsRNA comprising Formula IIIB comprises pattern 5AS. In some embodiments, a dsRNA comprising Formula IVB comprises pattern 5AS. In some embodiments, a dsRNA comprising Formula VB comprises pattern 5AS. In some embodiments, a dsRNA comprising Formula VIB comprises pattern 5AS. In some embodiments, a dsRNA comprising Formula VIIB comprises pattern 5AS. In some embodiments, a dsRNA comprising Formula VIIIB comprises pattern 5AS.

In some embodiments, a dsRNA comprising any one of SEQ ID NOS: 1-5349, 5366-5971, 6000-6038, 6050-6074, comprises pattern 1S. In some embodiments, a dsRNA comprising any one of SEQ ID NOS: 1-5349, 5366-5971, 6000-6038, 6050-6074, comprises pattern 1AS. In some embodiments, a dsRNA comprising any one of SEQ ID NOS: 1-5349, 5366-5971, 6000-6038, 6050-6074, comprises pattern 2S. In some embodiments, a dsRNA comprising any one of SEQ ID NOS: 1-5349, 5366-5971, 6000-6038, 6050-6074, comprises pattern 2AS. In some embodiments, a dsRNA comprising any one of SEQ ID NOS: 1-5349, 5366-5971, 6000-6038, 6050-6074, comprises pattern 3s. In some embodiments, a dsRNA comprising any one of SEQ ID NOS: 1-5349, 5366-5971, 6000-6038, 6050-6074, comprises pattern 3AS. In some embodiments, a dsRNA comprising any one of SEQ ID NOS: 1-5349, 5366-5971, 6000-6038, 6050-6074, comprises pattern 4S. In some embodiments, a dsRNA comprising any one of SEQ ID NOS: 1-5349, 5366-5971, 6000-6038, 6050-6074, comprises pattern 4AS.

In some embodiments, a dsRNA comprises a sense strand comprising any one of SEQ ID NOS: 1-5349, 5366-5971, 6000-6038, 6050-6074, and pattern 1S. In some instances, the dsRNA further comprises an antisense strand comprising the reverse complement of the sense strand, and pattern 1AS, 2A, 3AS, or 4AS. For instance, the antisense strand comprises 1AS.

In some embodiments, a dsRNA comprises a sense strand comprising any one of SEQ ID NOS: 1-5349, 5366-5971, 6000-6038, 6050-6074, and pattern 2S. In some instances, the dsRNA further comprises an antisense strand comprising the reverse complement of the sense strand, and pattern 1AS, 2A, 3AS, or 4AS. For instance, the antisense strand comprises 2AS.

In some embodiments, a dsRNA comprises a sense strand comprising any one of SEQ ID NOS: 1-5349, 5366-5971, 6000-6038, 6050-6074, and pattern 3S. In some instances, the dsRNA further comprises an antisense strand comprising the reverse complement of the sense strand, and pattern 1AS, 2A, 3AS, or 4AS. For instance, the antisense strand comprises 3AS.

In some embodiments, a dsRNA comprises a sense strand comprising any one of SEQ ID NOS: 1-5349, 5366-5971, 6000-6038, 6050-6074, and pattern 4S. In some instances, the dsRNA further comprises an antisense strand comprising the reverse complement of the sense strand, and pattern 1AS, 2A, 3AS, or 4AS. For instance, the antisense strand comprises 4AS.

In some embodiments, a dsRNA comprises a sense strand comprising any one of SEQ ID NOS: 1-5349, 5366-5971, 6000-6038, 6050-6074, and pattern 5S. In some instances, the dsRNA further comprises an antisense strand comprising the reverse complement of the sense strand, and pattern 1AS, 2A, 3AS, or 4AS. For instance, the antisense strand comprises 4AS.

Internucleoside Linkage Modifications

In some embodiments, the modification comprises a phosphodiester group. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some cases, the modified internucleoside linkage comprises one or more phosphorothioate linkages, where the substitution of one non-bridging oxygen of a phosphodiester with a sulfur atom creates the phosphorothioate (PS) linkage. A PS bond creates a new stereocenter in the nucleotide and when synthesized under standard achiral conditions creates diastereomeric mixtures of Rp and Sp at the phosphorous atom. In some cases, the one or more phosphorothioate linkages is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 phosphorothioate linkages.

In some embodiments, the sense strand of the siRNA comprises one or more phosphorothioate linkages. In some cases, the one or more phosphorothioate linkages of the sense strand is about 1, 2, 3, 4, 5, 6, 7, 8, 9, 10, 11, 12, 13, 14, 15, 16, 17, 18, 19, 20, or 21 phosphorothioate linkages. In some cases, the one or more phosphorothioate linkages of the sense strand is about 1, 2, 3, 4, or 5 phosphorothioate linkages. In some cases, the one or more phosphorothioate linkages of the sense strand is about 4 phosphorothioate linkages. In some cases, the sense strand comprises a phosphorothioate linkage between the first nucleoside and the second nucleoside of the sense strand, in a 5′ to 3′ direction. In some cases, the sense strand comprises a phosphorothioate linkage between the second nucleoside and the third nucleoside of the sense strand, in a 5′ to 3′ direction. In some cases, the sense strand comprises a phosphorothioate linkage between the nineteenth nucleoside and the twentieth nucleoside of the sense strand, in a 5′ to 3′ direction. In some cases, the sense strand comprises a phosphorothioate linkage between the twentieth nucleoside and the twenty-first nucleoside of the sense strand, in a 5′ to 3′ direction.

In some embodiments, the antisense strand of the siRNA comprises one or more phosphorothioate linkages. In some cases, the one or more phosphorothioate linkages of the antisense strand is about 1, 2, 3, 4, 5, 6, 7, 8, 9, 10, 11, 12, 13, 14, 15, 16, 17, 18, 19, 20, or 21 phosphorothioate linkages. In some cases, the one or more phosphorothioate linkages of the antisense strand is about 1, 2, 3, 4, or 5 phosphorothioate linkages. In some cases, the one or more phosphorothioate linkages of the antisense strand is about 4 phosphorothioate linkages. In some cases, the antisense strand comprises a phosphorothioate linkage between the first nucleoside and the second nucleoside of the antisense strand, in a 5′ to 3′ direction. In some cases, the antisense strand comprises a phosphorothioate linkage between the second nucleoside and the third nucleoside of the antisense strand, in a 5′ to 3′ direction. In some cases, the antisense strand comprises a phosphorothioate linkage between the nineteenth nucleoside and the twentieth nucleoside of the sense strand, in a 5′ to 3′ direction. In some cases, the antisense strand comprises a phosphorothioate linkage between the twentieth nucleoside and the twenty-first nucleoside of the sense strand, in a 5′ to 3′ direction.

There are other functional groups that have been identified as replacements of the phosphodiester group in the oligonucleotide. Like phosphates and phosphorothioates, there are a variety of functional groups that are negatively charged such as phosphorodithioate (PS2) and thio-phosphoramidates. There are number of analogues that are uncharged such as phosphorodiamidate morpholino oligomer (PMO), peptide nucleic acid (PNA), phosphotriesters, and phosphonates. It has been postulated that the uncharged analogues are not only nuclease resistant, but may also be more membrane permeable; however, the size and hydrophilicity of uncharged oligonucleotides still preclude their passive diffusion across membranes.

Morpholino oligos (PMOs) use a hydrolytically stable, uncharged phosphorodiamidate functional group.

Peptide nucleic acids (PNAs) are—as their name suggests—based upon the amide functional group.

Enemas and intramuscular, intravitreal, intrathecal injections have been used for the administration of a variety of oligonucleotides with and without PS bonds.

Nucleoside Modifications

In some embodiments, a dsRNA comprises a modified nucleoside. In some instances, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some instances, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

In some embodiments, the modification comprises nucleoside analogues that alter the structure of ribose. There are a variety of nucleotide mimics wherein the ribose or deoxyribose is modified to increase affinity for target and/or increase nuclease resistance. Modifications to all five positions of the ribose ring have been made; however, the modifications of the 2′ position of ribose have been the most studied.

1′ Position. The base. There are a few examples of base modifications that are designed to increase base pairing. One example is the G-clamp which is a cytidine mimic that is designed to have increased affinity for guanosine bases due to hydrogen bonding through an aminoethyl group. C-5 propynyl pyrimidines are known to form more stable duplexes; however, they appear to be more toxic as well.

2′ Modifications. In some embodiments, modifications of the hydroxyl group at the 2′ position of ribose are used to mimic the structure of the ribose ring while inhibiting ribonucleases that require the 2′OH group for hydrolysis of RNA. 2′-O-Methyl ribonucleic acids are naturally occurring nucleosides and have been shown to increase binding affinity to RNA itself while being resistant to ribonuclease. 2′-O-Methyl groups can be extensively substituted into RNAi triggers, and were the first nucleotide analogues used in “antagomirs.” 2′-O-Methoxyethyl (MOE) modification was designed to mimic the ribonuclease resistance of O-methyl, attenuate protein-oligonucleotide interactions and have increased affinity for RNA.

Fluorine is highly electronegative, and 2′-deoxy-2′-fluoro (2′-F) analogues of nucleosides adopt C3′-endo conformations characteristic of the sugars in RNA helices.

In some embodiments, the modified nucleoside comprises one or more 2′fluoro modified nucleosides. In some instances, the one or more 2′ fluoro modified nucleosides is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 2′ fluoro modified nucleosides.

In some instances, the sense strand of the siRNA comprises one or more 2′ fluoro modified nucleosides. In some instances, the one or more 2′ fluoro modified nucleosides of the sense strand is about 1, 2, 3, 4, 5, 6, 7, 8, 9, 10, 11, 12, 13, 14, 15, 16, 17, 18, 19, 20, or 21 2′ fluoro modified nucleosides. In some instances, the nucleoside at position 1, 2, 3, 4, 5, 6, 7, 8, 9, 10, 11, 12, 13, 14, 15, 16, 17, 18, 19, 20, or 21, or a combination thereof, of the sense strand comprises the 2′ fluoro modified nucleoside, in a 5′ to 3′ direction. In some instances, the nucleoside at position 1, 3, 5, 7, 8, 9, 11, 13, 15, 17, or 19, or a combination thereof, of the sense strand comprises the 2′ fluoro modified nucleoside, in a 5′ to 3′ direction. In some instances, the fifth, seventh, and ninth nucleosides of the sense strand comprises the 2′ fluoro modified nucleoside, in a 5′ to 3′ direction.

In certain embodiments, the sense strand comprises the pattern fN-Z1-fN-Z2-fN, wherein fN comprises the 2′ fluoro modified nucleoside and Z1 and Z2 are independently a 2′ O-methyl modified nucleoside or a 2′ fluoro modified nucleoside. In some instances, the fN-Z1-fN-Z2-fN corresponds to nucleosides five to nine of the sense strand, in a 5′ to 3′ direction. In some instances, the sense strand comprises at least two contiguous 2′ fluoro modified nucleosides. In some instances, the at least two contiguous 2′ fluoro modified nucleosides is two contiguous 2′ fluoro modified nucleosides. In some instances, the at least two contiguous 2′ fluoro modified nucleosides is three contiguous 2′ fluoro modified nucleosides.

In some embodiments, the antisense strand of the siRNA comprises one or more 2′ fluoro modified nucleosides. In some instances, the one or more 2′ fluoro modified nucleosides of the antisense strand is about 1, 2, 3, 4, 5, 6, 7, 8, 9, 10, 11, 12, 13, 14, 15, 16, 17, 18, 19, 20, or 21 2′ fluoro modified nucleosides. In some instances, the one or more 2′ fluoro modified nucleosides of the antisense strand is about 8, 6, 5, or 4 2′ fluoro modified nucleosides. In some cases, the nucleoside at position, 1, 2, 3, 4, 5, 6, 7, 8, 9, 10, 11, 12, 13, 14, 15, 16, 17, 18, 19, 20, or 21, or a combination thereof, of the antisense strand comprises the 2′ fluoro modified nucleoside, in a 5′ to 3′ direction. In some cases, the nucleoside at position, 2, 4, 6, 8, 9, 10, 14, 16, or 18, or a combination thereof, of the antisense strand comprises the 2′ fluoro modified nucleoside, in a 5′ to 3′ direction. In some cases, the second and fourteenth nucleosides of the antisense strand comprises the 2′ fluoro modified nucleoside, in a 5′ to 3′ direction. In some cases, the second, sixth, fourteenth, and sixteenth nucleosides of the antisense strand comprises the 2′ fluoro modified nucleoside, in a 5′ to 3′ direction.

In certain embodiments, the antisense strand comprises the pattern Z3-fN-Z4-fN, wherein fN comprises the 2′ fluoro modified nucleoside and Z3 and Z4 are independently a 2′ O-methyl modified nucleoside or a 2′ fluoro modified nucleoside. In some cases, the Z3-fN-Z4-fN corresponds to nucleosides thirteen to sixteen of the antisense strand, in a 5′ to 3′ direction.

In some embodiments, the modified nucleoside comprises a 2′ O-alkyl modified nucleoside. In some instances, the 2′-O-alkyl modified nucleoside comprises one or more 2′ O-methyl modified nucleosides. In some instances, the one or more 2′ O-methyl modified nucleosides is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 2′ O-methyl modified nucleosides.

In certain embodiments, the sense strand of the siRNA comprises one or more 2′ O-methyl modified nucleosides. In some instances, the one or more 2′ O-methyl modified nucleosides of the sense strand is about 1, 2, 3, 4, 5, 6, 7, 8, 9, 10, 11, 12, 13, 14, 15, 16, 17, 18, 19, 20, or 21 2′ O-methyl modified nucleosides. In some instances, the nucleoside at position 1, 2, 3, 4, 5, 6, 7, 8, 9, 10, 11, 12, 13, 14, 15, 16, 17, 18, 19, 20, 21, or a combination thereof, of the sense strand comprises the one or more 2′ O-methyl modified nucleosides. In some instances, the nucleoside at position 1, 2, 3, 4, 6, 8, 10, 11, 12, 13, 14, 15, 16, 17, 18, 19, 20, 21, or a combination thereof, of the sense strand comprises the one or more 2′ O-methyl modified nucleosides. In some instances, the second, fourth, sixth, tenth, twelfth, fourteenth, and sixteenth, eighteenth, twentieth, and twenty-first nucleosides of the sense strand comprises the 2′ O-methyl modified nucleoside, in a 5′ to 3′ direction.

In some embodiments, the sense strand comprises the pattern mN-Z5-mN-Z6, wherein mN comprises the 2′ O-methyl modified nucleoside and Z5 and Z6 are independently a 2′ O-methyl modified nucleoside or a 2′ fluoro modified nucleoside. In some instances, the mN-Z5-mN-Z6-mN corresponds to nucleosides four to seven of the sense strand, in a 5′ to 3′ direction. In some instances, Z5 is the 2′ fluoro modified nucleoside. In some instances, Z5 is the 2′ O-methyl modified nucleoside. In some instances, Z6 is the 2′ fluoro modified nucleoside. In some instances, Z6 is the 2′ O-methyl modified nucleoside.

In some embodiments, the sense strand comprises the pattern mN-Z5-mN-Z6-mN, wherein mN comprises the 2′ O-methyl modified nucleoside and Z5 and Z6 are independently a 2′ O-methyl modified nucleoside or a 2′ fluoro modified nucleoside. In some instances, the mN-Z5-mN-Z6-mN corresponds to nucleosides two to six of the sense strand, in a 5′ to 3′ direction. In some instances, the mN-Z5-mN-Z6-mN corresponds to nucleosides ten to fourteen of the sense strand, in a 5′ to 3′ direction. In some instances, the mN-Z5-mN-Z6-mN corresponds to nucleosides twelve to sixteen of the sense strand, in a 5′ to 3′ direction. In some instances, the mN-Z5-mN-Z6-mN corresponds to nucleosides fourteen to eighteen of the sense strand, in a 5′ to 3′ direction. In some instances, the mN-Z5-mN-Z6-mN corresponds to nucleosides sixteen to twenty of the sense strand, in a 5′ to 3′ direction.

In some embodiments, the sense strand comprises the pattern mN-Z5-mN-Z6-mN-Z7-mN, wherein Z7 is a 2′ O-methyl modified nucleoside or a 2′ fluoro modified nucleoside. In some instances, the mN-Z5-mN-Z6-mN-Z7-mN corresponds to nucleosides ten to sixteen of the sense strand, in a 5′ to 3′ direction. In some instances, the mN-Z5-mN-Z6-mN-Z7-mN corresponds to nucleosides twelve to eighteen of the sense strand, in a 5′ to 3′ direction. In some instances, the mN-Z5-mN-Z6-mN-Z7-mN corresponds to nucleosides fourteen to twenty of the sense strand, in a 5′ to 3′ direction.

In some embodiments, the sense strand comprises the pattern mN-Z5-mN-Z6-mN-Z7-mN-Z8-mN, wherein Z8 is a 2′ O-methyl modified nucleoside or a 2′ fluoro modified nucleoside. In some instances, the mN-Z5-mN-Z6-mN-Z7-mN-Z8-mN corresponds to nucleosides ten to eighteen of the sense strand, in a 5′ to 3′ direction. In some instances, the mN-Z5-mN-Z6-mN-Z7-mN-Z8-mN corresponds to nucleosides twelve to twenty of the sense strand, in a 5′ to 3′ direction. In some embodiments, the sense strand comprises the pattern mN-Z5-mN-Z6-mN-Z7-mN-Z8-mN-Z9-mN, wherein Z9 is a 2′ O-methyl modified nucleoside or a 2′ fluoro modified nucleoside. In some instances, the mN-Z5-mN-Z6-mN-Z7-mN-Z8-mN-Z9-mN corresponds to nucleosides ten to twenty of the sense strand, in a 5′ to 3′ direction.

In some embodiments, the sense strand comprises at least two contiguous 2′ O-methyl modified nucleosides. In some instances, the at least two contiguous 2′ O-methyl modified nucleosides is 3, 4, 5, 6, 7, 8, 9, 10, 11, 12 contiguous 2′ O-methyl modified nucleosides.

In some embodiments, the antisense strand of the siRNA comprises one or more 2′ O-methyl modified nucleosides. In some instances, the one or more 2′ O-methyl modified nucleosides of the antisense strand is about 1, 2, 3, 4, 5, 6, 7, 8, 9, 10, 11, 12, 13, 14, 15, 16, 17, 18, 19, 20, or 21 2′ O-methyl modified nucleosides. In some instances, the nucleotide at position 1, 2, 3, 4, 5, 6, 7, 8, 9, 10, 11, 12, 13, 14, 15, 16, 17, 18, 19, 20, or 21, or a combination thereof, of the antisense strand comprises the 2′ O-methyl modified nucleoside, in a 5′ to 3′ direction. In some instances, the nucleotide at position 1, 3, 4, 5, 7, 8, 9, 10, 11, 12, 13, 15, 17, 18, 19, 20, or 21, or a combination thereof, of the antisense strand comprises the 2′ O-methyl modified nucleoside, in a 5′ to 3′ direction. In some instances, the first, third, fifth, seventh, eleventh, twelfth, thirteenth, fifteenth, seventeenth, nineteenth, twentieth, and twenty-first nucleosides of the antisense strand comprises the 2′ O-methyl modified nucleoside, in a 5′ to 3′ direction.

In some embodiments, the antisense strand comprises at least two contiguous 2′ O-methyl modified nucleosides. In some instances, the at least two contiguous 2′ O-methyl modified nucleosides is 3, 4, 5, 6, or 7 contiguous 2′ O-methyl modified nucleosides.

In some embodiments, the antisense strand comprises a first sequence comprising at least two contiguous 2′ O-methyl modified nucleosides and a second sequence comprising at least two contiguous 2′ O-methyl modified nucleosides. In some instances, the first sequence comprises at least three contiguous 2′ O-methyl modified nucleosides, and the second sequence comprises at least three contiguous 2′ O-methyl modified nucleosides. In some instances, the first sequence comprises three contiguous 2′ O-methyl modified nucleosides, and the second sequence comprises three contiguous 2′ O-methyl modified nucleosides. In some instances, the first sequence comprises four contiguous 2′ O-methyl modified nucleosides, and the second sequence comprises five contiguous 2′ O-methyl modified nucleosides. In some instances, the first sequence comprises seven contiguous 2′ O-methyl modified nucleosides, and the second sequence comprises five contiguous 2′ O-methyl modified nucleosides. In some instances, the first sequence comprises at least four contiguous 2′ O-methyl modified nucleosides. In some instances, the first sequence comprises at least five contiguous 2′ O-methyl modified nucleosides. In some instances, the first sequence comprises at least six contiguous 2′ O-methyl modified nucleosides. In some instances, the first sequence comprises at least seven contiguous 2′ O-methyl modified nucleosides. In some instances, the second sequence comprises at least four contiguous 2′ O-methyl modified nucleosides. In some instances, the second sequence comprises at least five contiguous 2′ O-methyl modified nucleosides. In some instances, the second sequence comprises at least six contiguous 2′ O-methyl modified nucleosides. In some instances, the second sequence comprises at least seven contiguous 2′ O-methyl modified nucleosides.

4′- and 5′-modifications. Alkoxy substituents at the 4′ position of 2′deoxyribose mimic the conformation of ribose.

Bicyclic 2′-4′-modifications. There are a variety of ribose derivatives that lock the carbohydrate ring into the 3′ endo conformation by the formation of bicyclic structures with a bridge between the 2′ oxygen and the 4′ position. The original bicyclic structure can have a methylene bridging group; these are termed locked nucleic acids (LNAs). The bicyclic structure “locks” the ribose into its preferred 3′ endo conformation and increases base pairing affinity. The incorporation of LNAs into a DNA duplex can increase melting points up to 8° C. per LNA. Subsequently, a variety of bicyclic nucleotides have been developed such as Bridged Nucleic Acids (BNAs), Ethyl-bridged (ENAs), constrained ethyl (cEt) nucleic acids and tricyclic structures with varying affinity for target sites. LNAs can be incorporated into antagomirs, splice blocking oligonucleotides, either strand of an RNAi duplex; however, like other 3′ endo conformers, LNAs are not substrates for RNAse H.

Acyclic nucleic acid analogs: Nucleic acid analogs that have an alternative ribose ring structure have been developed. These include those in which the bond between 2′ and 3′ carbons in the ribose is absent, as well as those containing substitution of the ribose ring with a three-carbon backbone. Examples of acylic nucleic acid analogs include unlocked nucleic acid (UNA) and glycol nucleic acids (GNA). Incorporation of these analogs reduce the melting temperature of the RNAi duplex and can be incorporated into either strand. Incorporation at the 5′ end of the sense strand, or passenger strand, inhibits incorporation into this strand into RISC. Incorporation into the seed region of the antisense strand, or guide strand, can reduce off-target activity. Acyclic nucleic acid analogs may also increase resistance of the RNAi duplex to 3′-exonuclease activity.

Modification patterns: gapmer overall design. For RNAi duplexes, recognition by RISC requires RNA-like 3′-endo nucleotides and some patterns of RNA analogues. It was observed that a pattern of alternating 2′-O-methyl groups provides stability against nucleases, but not all permutations of alternating 2′-O-methyl are active RNAi agents. The fact that one may remove all 2′-hydroxy groups with alternating 2′-fluoro and 2′-O-methyl groups to produce duplexes that are resistant to nucleases and active in RNAi suggests the 2′-hydroxy group is not absolutely required for activity, but that some sites in the RNAi duplex are sensitive to the added steric bulk of the methyl group.

In some embodiments, the modified siRNA comprises modified nucleotides including, but not limited to, 2′OMe nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. In preferred embodiments, the modified siRNA comprises 2′OMe nucleotides (e.g., 2′OMe purine and/or pyrimidine nucleotides) such as, for example, 2′OMe-guanosine nucleotides, 2′OMe-uridine nucleotides, 2′OMe-adenosine nucleotides, 2′OMe-cytosine nucleotides, and mixtures thereof. In some instances, the modified siRNA does not comprise a modification. The modification, for example, comprises 2′OMe-cytosine nucleotides. In some embodiments, the modified siRNA comprises a hairpin loop structure.

In some embodiments, the modified siRNA comprises natural or synthetic or artificial nucleotide analogues or bases. In some cases, the modified siRNA comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some embodiments, nucleotide analogues or artificial nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso group, a nitrile group, a heterocycle (e.g., imidazole, hydrazino or hydroxylamino) group, an isocyanate or cyanate group, or a sulfur containing group (e.g., sulfoxide, sulfone, sulfide, and disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety.

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties.

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer.

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA.

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, nucleotide analogues comprise modified bases such as, but not limited to, 5-propynyluridine, 5-propynylcytidine, 6-methyladenine, 6-methylguanine, N, N, -dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five-member ribose ring is substituted with a six-member morpholino ring containing four carbons, one nitrogen and one oxygen. In some cases, the ribose monomers are linked by a phosphorodiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

In some embodiments, peptide nucleic acid (PNA) does not contain sugar ring or phosphate linkage and the bases are attached and appropriately spaced by oligoglycine-like molecules, therefore, eliminating a backbone charge.

In some embodiments, one or more modifications optionally occur at the internucleotide linkage. In some instances, modified internucleotide linkage include, but is not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′linkage or 2′-5′linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage.

In some instances, the modification is a methyl or thiol modification such as methylphosphonate or thiolphosphonate modification.

In some instances, a modified nucleotide includes, but is not limited to, 2′-fluoro N3-P5′-phosphoramidites.

In some instances, a modified nucleotide includes, but is not limited to, hexitol nucleic acid (or 1′, 5′-anhydrohexitol nucleic acids (HNA)).

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, the modified siRNA comprises one or more of the artificial nucleotide analogues described herein. In some instances, the modified siRNA comprises 1, 2, 3, 4, 5, 6, 7, 8, 9, 10, 11, 12, 13, 14, 15, 16, 17, 18, 20, 25, or more of the artificial nucleotide analogues described herein. In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the modified siRNA comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the modified siRNA comprises 1, 2, 3, 4, 5, 6, 7, 8, 9, 10, 11, 12, 13, 14, 15, 16, 17, 18, 20, 25, or more of 2′-O-methyl modified nucleotides. In some instances, the modified siRNA comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the modified siRNA comprises 1, 2, 3, 4, 5, 6, 7, 8, 9, 10, 11, 12, 13, 14, 15, 16, 17, 18, 20, 25, or more of thiolphosphonate nucleotides.

The modified siRNA, in some embodiments, has an IC50 less than or equal to ten-fold that of the corresponding unmodified siRNA (i.e., the modified siRNA has an IC50 that is less than or equal to ten-times the IC50 of the corresponding unmodified siRNA). In some embodiments, the modified siRNA has an IC50 less than or equal to three-fold that of the corresponding unmodified siRNA. In yet other embodiments, the modified siRNA preferably has an IC50 less than or equal to two-fold that of the corresponding unmodified siRNA. In some embodiments, the modified siRNA has an IC50 of less than or equal to 10-fold, 9-fold, 8-fold, 7-fold, 6-fold, 5-fold, 4-fold, 3-fold, 2-fold, or 1.5-fold compared to the IC50 of a corresponding unmodified siRNA. It will be readily apparent to those of skill in the art that a dose response curve can be generated and the IC50 values for the modified siRNA and the corresponding unmodified siRNA can be readily determined using methods known to those of skill in the art.

The modified siRNA described herein may have 3′ overhangs. In some embodiments, the 3′ overhangs comprises at least or about 1, 2, 3, 4, 5, 6, 7, 8, 9, 10 or more than 10 nucleotides on one or both sides of the double-stranded region. In some embodiments, the modified siRNA lacks overhangs (i.e., have blunt ends). In some embodiments, the modified siRNA has 3′ overhangs of two nucleotides on each side of the double-stranded region. In some embodiments, the 3′ overhang on the antisense strand has complementarity to the target sequence and the 3′ overhang on the sense strand has complementarity to the complementary strand of the target sequence. Alternatively, or in combination, the 3′ overhangs do not have complementarity to the target sequence or the complementary strand thereof. In some embodiments, the 3′ overhangs comprise at least or about 1, 2, 3, 4, 5, 6, 7, 8, 9, 10 or more than 10 nucleotides such as 2′-deoxy(2′H) nucleotides. In some embodiments, the 3′ overhangs comprise deoxythymidine (dT) nucleotides.

Typically, the modified siRNA comprises from about 1% to about 100% (e.g., about 1%, 5%, 10%, 15%, 20%, 25%, 30%, 35%, 40%, 45%, 50%, 55%, 60%, 65%, 70%, 75%, 80%, 85%, 90%, 95%, or 100%) modified nucleotides in the double-stranded region of the siRNA duplex. In preferred embodiments, less than about 30% (e.g., less than about 30%, 25%, 20%, 15%, 10%, or 5%) or from about 1% to about 30% (e.g., from about 1%-30%, 5%-30%, 10%-30%, 15%-30%, 20%-30%, or 25%-30%) of the nucleotides in the double-stranded region comprise modified nucleotides. In some instances, the modified siRNA comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some embodiments, the modified siRNA does not comprise phosphate backbone modifications, e.g., in the sense and/or antisense strand of the double-stranded region. In some embodiments, the modified siRNA does not comprise 2′-deoxy nucleotides, e.g., in the sense and/or antisense strand of the double-stranded region. In some embodiments, the nucleotide at the 3′-end of the double-stranded region in the sense and/or antisense strand is not a modified nucleotide. In certain other instances, the nucleotides near the 3′-end (e.g., within one, two, three, or four nucleotides of the 3′-end) of the double-stranded region in the sense and/or antisense strand are not modified nucleotides.

The modified siRNA described herein may have 3′ overhangs of one, two, three, four, or more nucleotides on one or both sides of the double-stranded region, or may lack overhangs (i.e., have blunt ends). Preferably, the modified siRNA has 3′ overhangs of two nucleotides on each side of the double-stranded region. In some embodiments, the 3′ overhangs comprise one, two, three, four, or more nucleotides such as 2′-deoxy(2′H) nucleotides. Preferably, the 3′ overhangs comprise deoxythymidine (dT) nucleotides.

The dsRNA may also have a blunt end, located at the 5′-end of the antisense strand (or the 3′-end of the sense strand) or vice versa. Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process.

In some embodiments, the dsRNA agent described herein may also have two blunt ends, at both ends of the dsRNA duplex.

In some embodiments, every nucleotide in the sense strand and antisense strand of the dsRNA agent, including the nucleotides that are part of the motifs, may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in some cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of a RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In some embodiments, the sense strand and antisense strand each contains two differently modified nucleotides selected from 2′-O-methyl or 2′-fluoro.

In some embodiments, each residue of the sense strand and antisense strand is independently modified with 2′-O-methyl nucleotide, 2′-deoxyfluoro nucleotide, 2-O—N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “AC AC AC . . . ” “BDBDBD . . . ” or “CDCDCD . . . ,” etc.

In some embodiments, the dsRNA agent described herein comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5 Of the strand within the duplex region. As another example, the alternating motif in the sense strand may start with “AABBAABB” from 5′-3′ of the strand and the alternating motif in the antisense strand may start with “BBAABBAA” from 3′-5 of the strand within the duplex region, so that there is a complete or partial shift of the modification patterns between the sense strand and the antisense strand.

In some embodiments, the dsRNA agent described herein comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification. The introduction of one or more motifs of three identical modifications on three consecutive nucleotides to the sense strand and/or antisense strand interrupts the initial modification pattern present in the sense strand and/or antisense strand. This interruption of the modification pattern of the sense and/or antisense strand by introducing one or more motifs of three identical modifications on three consecutive nucleotides to the sense and/or antisense strand surprisingly enhances the gene silencing activity to the target gene.

The dsRNA agent described herein may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments the sense strand of the dsRNA comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments the antisense strand of the dsRNA comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA described herein further comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA described herein further comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the dsRNA can optionally further comprise one or more phosphorothioate or methylphosphonate internucleotide linkage modification within 1-10 of the termini position(s).

In some embodiments, the dsRNA described herein further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA described herein further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA described herein further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA described herein further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end). In some embodiments, the dsRNA described herein further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA described herein further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA described herein further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA described herein further comprises one phosphorothioate internucleotide linkage modification within position 1-5 (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA described herein further comprises two phosphorothioate internucleotide linkage modifications within position 1-5 (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA described herein further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA described herein further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA described herein further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA described herein further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA described herein further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA described herein further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA described herein further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA described herein further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA described herein further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, the dsRNA agent described herein comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings. In some embodiments, the dsRNA agent described herein comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

The dsRNA agent that contains conjugations of one or more carbohydrate moieties to a dsRNA agent can optimize one or more properties of the dsRNA agent. In many cases, the carbohydrate moiety will be attached to a modified subunit of the dsRNA agent. E.g., the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide.

Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

In some embodiments the dsRNA described herein is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone. The double-stranded RNA (dsRNA) agent described herein may optionally be conjugated to one or more ligands. The ligand can be attached to the sense strand, antisense strand or both strands, at the 3′-end, 5′-end or both ends. For instance, the ligand may be conjugated to the sense strand, in particular, the 3′-end of the sense strand.

Non-limiting examples of ALOX15 siRNA sequences directed against human ALOX15 mRNA useful in the present methods include those described in Table 5A and SEQ ID NOS: 1-5349, 5366-5971, 6000-6038, 6050-6074. The sequences are listed in the 5′-3′ orientation. Sequence ID numbers are indicated prior to each sequence. Odd numbered sequences in each line are the sense, or passenger, strand. Even numbered sequences in each strand are the antisense, or guide, strand. Each sense and antisense strand can be annealed to produce the siRNA. In embodiments, at least the final two 3′ nucleotides in each strand are substituted with different nucleotides. In embodiments, one or more nucleotides in each sequence is modified.

Ligands

A wide variety of entities can be coupled to the oligonucleotides described herein. In some embodiments, the moieties are ligands, which are coupled, preferably covalently, either directly or indirectly via an intervening tether.

In preferred embodiments, a ligand alters the distribution, targeting or lifetime of the molecule into which it is incorporated. In preferred embodiments a ligand provides an enhanced affinity for a selected target, e.g., molecule, cell or cell type, compartment, receptor e.g., a cellular or organ compartment, tissue, organ or region of the body, as, e.g., compared to a species absent such a ligand. Ligands providing enhanced affinity for a selected target are also termed targeting ligands.

Some ligands can have endosomolytic properties. The endosomolytic ligands promote the lysis of the endosome and/or transport of the composition described herein, or its components, from the endosome to the cytoplasm of the cell. The endosomolytic ligand may be a polyanionic peptide or peptidomimetic which shows pH-dependent membrane activity and fusogenicity. In some embodiments, the endosomolytic ligand assumes its active conformation at endosomal pH. The “active” conformation is that conformation in which the endosomolytic ligand promotes lysis of the endosome and/or transport of the composition described herein, or its components, from the endosome to the cytoplasm of the cell. Exemplary endosomolytic ligands include the GALA peptide, the EALA peptide, and their derivatives. In some embodiments, the endosomolytic component may contain a chemical group (e.g., an amino acid) which will undergo a change in charge or protonation in response to a change in pH. The endosomolytic component may be linear or branched. Ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer. FIG. 7 shows some examples of targeting ligands and their associated receptors.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases or a chelator (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, an activator of p38 MAP kinase, or an activator of NF-KB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the oligonucleotide into the cell by activating an inflammatory response, for example. Exemplary ligands that would have such an effect include tumor necrosis factor alpha (TNF alpha), interleukin-1 beta, or gamma interferon.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include B vitamins, e.g., folic acid, B12, riboflavin, biotin, pyridoxal or other vitamins or nutrients taken up by cancer cells. Also included are HAS, low density lipoprotein (LDL) and high-density lipoprotein (HDL). In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF derived from human fibroblast growth factor 4 and having the amino acid sequence AAVALLPAVLLALLAP (SEQ ID NO: 5350). An RFGF analogue (e.g., amino acid sequence AALLPVLLAAP (SEQ ID NO: 5351)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 5352)) and the Drosophila Antennapedia protein (RQIKIWFQNRRMKWK (SEQ ID NO: 5353)) have been found to be capable of functioning as delivery peptides. A peptide or peptidomimetic can be encoded by a random sequence of DNA, such as a peptide identified from a phage-display library, or one-bead-one-compound (OBOC) combinatorial library. Preferably the peptide or peptidomimetic tethered to an iRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized. An RGD peptide moiety can be used to target a tumor cell, such as an endothelial tumor cell or a breast cancer tumor cell. An RGD peptide can facilitate targeting of an iRNA agent to tumors of a variety of other tissues, including the lung, kidney, spleen, or liver. Preferably, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing yB3. Peptides that target markers enriched in proliferating cells can be used. E.g., RGD containing peptides and peptidomimetics can target cancer cells, in particular cells that exhibit an integrin. Thus, one could use RGD peptides, cyclic peptides containing RGD, RGD peptides that include D-amino acids, as well as synthetic RGD mimics. In addition to RGD, one can use other moieties that target the integrin ligand. Generally, such ligands can be used to control proliferating cells and angiogenesis. Preferred conjugates of this type ligands that target PECAM-1, VEGF, or other cancer genes, e.g., a cancer gene described herein.

A “cell permeation peptide” is capable of permeating a cell, e.g., a microbial cell, such as a bacterial or fungal cell, or a mammalian cell, such as a human cell. A microbial cell-permeating peptide can be, for example, an a-helical linear peptide (e.g., LL-37 or Ceropin PI), a disulfide bond-containing peptide (e.g., a-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen.

In some embodiments, a targeting peptide can be an amphipathic alpha-helical peptide. Exemplary amphipathic α-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H2A peptides, Xenopus peptides, esculentinis-1, and caerins. A number of factors will preferably be considered to maintain the integrity of helix stability. For example, a maximum number of helix stabilization residues will be utilized (e.g., leu, ala, or lys), and a minimum number helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an apatamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein.

Endosomal release agents include imidazoles, poly or oligoimidazoles, PEIs, peptides, fusogenic peptides, polycaboxylates, polyacations, masked oligo or poly cations or anions, acetals, polyacetals, ketals/polyketyals, orthoesters, polymers with masked or unmasked cationic or anionic charges, dendrimers with masked or unmasked cationic or anionic charges.

PK modulator stands for pharmacokinetic modulator. PK modulator include lipophiles, bile acids, steroids, phospholipid analogues, peptides, protein binding agents, PEG, vitamins etc. Exemplary PK modulator include, but are not limited to, cholesterol, fatty acids, cholic acid, lithocholic acid, dialkylglycerides, diacylglyceride, phospholipids, sphingo lipids, naproxen, ibuprofen, vitamin E, biotin etc. Oligonucleotides that comprise a number of phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbone are also amenable as ligands (e.g. as PK modulating ligands).

In addition, aptamers that bind serum components (e.g. serum proteins) are also amenable as PK modulating ligands.

Other ligand conjugates amenable are described in U.S. patent application Ser. No. 10/916,185, filed Aug. 10, 2004; U.S. Ser. No. 10/946,873, filed Sep. 21, 2004; U.S. Ser. No. 10/833,934, filed Aug. 3, 2007; U.S. Ser. No. 11/115,989 filed Apr. 27, 2005 and U.S. Ser. No. 11/944,227 filed Nov. 21, 2007.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

Ligands can be coupled to the oligonucleotides at various places, for example, 3′-end, 5′-end, and/or at an internal position. In preferred embodiments, the ligand is attached to the oligonucleotides via an intervening tether, e.g. a carrier described herein. The ligand or tethered ligand may be present on a monomer when said monomer is incorporated into the growing strand. In some embodiments, the ligand may be incorporated via coupling to a “precursor” monomer after said “precursor” monomer has been incorporated into the growing strand. For example, a monomer having, e.g., an amino-terminated tether (i.e., having no associated ligand), e.g., TAP-(CH2)nNH2 may be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether. In another example, a monomer having a chemical group suitable for taking part in Click Chemistry reaction may be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands. In some embodiments, a double-stranded iRNA agent contains a ligand conjugated to the sense strand. In other embodiments, a double-stranded iRNA agent contains a ligand conjugated to the antisense strand.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The F position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

Any suitable ligand in the field of RNA interference may be used, although the ligand is typically a carbohydrate e.g. monosaccharide (such as GalNAc), disaccharide, trisaccharide, tetrasaccharide, polysaccharide. Linkers that conjugate the ligand to the nucleic acid include those discussed above. For example, the ligand can be one or more GalNAc (N-acetylglucosamine) derivatives attached through a bivalent or trivalent branched linker.

As used herein, the terms “dsRNA”, “siRNA”, and “iRNA agent” are used interchangeably to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an siRNA agent of 21 to 23 nucleotides.

As used herein, “specifically hybridizable” and “complementary” are terms which are used to indicate a sufficient degree of complementarity such that stable and specific binding occurs between a compound described herein and a target RNA molecule.

Specific binding requires a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences typically differ by at least 5 nucleotides.

In some embodiments, a dsRNA agent described herein is “sufficiently complementary” to a target RNA, e.g., a target mRNA, such that the dsRNA agent silences production of protein encoded by the target mRNA. In some embodiments, the dsRNA agent described herein is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA agent described herein specifically discriminates a single-nucleotide difference. In this case, the dsRNA agent only mediates RNAi if exact complementary is found in the region (e.g., within 7 nucleotides of) the single-nucleotide difference. As used herein, the term “oligonucleotide” refers to a nucleic acid molecule (RNA or DNA) for example of length less than 100, 200, 300, or 400 nucleotides.

The term “halo” refers to any radical of fluorine, chlorine, bromine or iodine. The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, Ci-Cio indicates that the group may have from 1 to 10 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl substituted with an amino. The term “mercapto” refers to an —SH radical. The term “thioalkoxy” refers to an —S-alkyl radical.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 1 1-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include trizolyl, tetrazolyl, piperazinyl, pyrrolidinyl, dioxanyl, morpholinyl, tetrahydrofuranyl, and the like.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylamino carbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent can be further substituted.

Cleavable Linking Groups

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood.

Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes. In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-Based Cleavable Linking Groups

Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-Based Linking Groups

Ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-Based Cleaving Groups

Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above. As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C5 and above (preferably C5-C8) sugars; di- and trisaccharides include sugars having two or three monosaccharide units (preferably C5-C8).

In still yet another aspect, the modified siRNA described herein is used in methods for silencing expression of a target sequence. In particular, it is an object of the present invention to provide in vitro and in vivo methods for treatment of a disease or disorder in a mammal by downregulating or silencing the transcription and/or translation of a target gene of interest. In some embodiments, described herein are methods for introducing an siRNA that silences expression (e.g., mRNA and/or protein levels) of a target sequence into a cell by contacting the cell with a modified siRNA described herein. In some embodiments, described herein are methods for in vivo delivery of a siRNA that silences expression of a target sequence by administering to a mammal a modified siRNA described herein. Administration of the modified siRNA can be by any route known in the art, such as, e.g., oral, intranasal, intravenous, intraperitoneal, intramuscular, intra-articular, intralesional, intratracheal, subcutaneous, or intradermal.

In certain embodiments, the modified siRNA further comprises a carrier system, e.g., to deliver the modified siRNA into a cell of a mammal. Non-limiting examples of carrier systems include, but are not limited to, nucleic acid-lipid particles, liposomes, micelles, virosomes, nucleic acid complexes, and mixtures thereof. In some embodiments, the modified siRNA molecule is complexed with a lipid such as a cationic lipid to form a lipoplex. In certain other instances, the modified siRNA molecule is complexed with a polymer such as a cationic polymer (e.g., polyethylenimine (PEI)) to form a polyplex. The modified siRNA molecule may also be complexed with cyclodextrin or a polymer thereof. Preferably, the modified siRNA molecule is encapsulated in a nucleic acid-lipid particle.

In some embodiments, described herein are methods for delivering dsRNA to a specific target in a subject by nasal administration.

### Additional Embodiments

In some embodiments, described herein are dsRNA agents capable of inhibiting the expression of a target gene. The dsRNA agent comprises a sense strand and an antisense strand, each strand having 14 to 30 nucleotides. Every nucleotide in the sense strand and antisense strand has been modified. The modifications on sense strand and antisense strand each independently comprises at least two different modifications.

In some embodiments, described herein are dsRNA agents capable of inhibiting the expression of a target gene. The dsRNA agent comprises a sense strand and an antisense strand, each strand having 14 to 30 nucleotides. The sense strand contains at least one motif of three identical modifications on three consecutive nucleotides, where at least one of the motifs occurs at or near the cleavage site in the antisense strand. The antisense strand contains at least one motif of three identical modifications on three consecutive nucleotides. The modification pattern of the antisense strand is shifted by one or more nucleotides relative to the modification pattern of the sense strand.

In some embodiments, described herein are dsRNA agents capable of inhibiting the expression of a target gene. The dsRNA agent comprises a sense strand and an antisense strand, each strand having 14 to 30 nucleotides. The sense strand contains at least two motifs of three identical modifications on three consecutive nucleotides, when at least one of the motifs occurs at the cleavage site in the strand and at least one of the motifs occurs at another portion of the strand that is separated from the motif at the cleavage site by at least one nucleotide. The antisense strand contains at least one motif of three identical modifications on three consecutive nucleotides, where at least one of the motifs occurs at or near the cleavage site in the strand and at least one of the motifs occurs at another portion of the strand that is separated from the motif at or near cleavage site by at least one nucleotide.

In some embodiments, described herein are dsRNA agents capable of inhibiting the expression of a target gene. The dsRNA agent comprises a sense strand and an antisense strand, each strand having 14 to 30 nucleotides. The sense strand contains at least two motifs of three identical modifications on three consecutive nucleotides, where at least one of the motifs occurs at the cleavage site in the strand and at least one of the motifs occurs at another portion of the strand that is separated from the motif at the cleavage site by at least one nucleotide. The antisense strand contains at least one motif of three identical modifications on three consecutive nucleotides, where at least one of the motifs occurs at or near the cleavage site in the strand and at least one of the motifs occurs at another portion of the strand that is separated from the motif at or near cleavage site by at least one nucleotide. The modification in the motif occurring at the cleavage site in the sense strand is different than the modification in the motif occurring at or near the cleavage site in the antisense strand. In some embodiments, described herein are dsRNA agents capable of inhibiting the expression of a target gene. The dsRNA agent comprises a sense strand and an antisense strand, each strand having 12 to 30 nucleotides. The sense strand contains at least one motif of three 2′-F modifications on three consecutive nucleotides, where at least one of the motifs occurs at the cleavage site in the strand. The antisense strand contains at least one motif of three 2′-O-methyl modifications on three consecutive nucleotides.

The sense strand may further comprises one or more motifs of three identical modifications on three consecutive nucleotides, where the one or more additional motifs occur at another portion of the strand that is separated from the three 2′-F modifications at the cleavage site by at least one nucleotide. The antisense strand may further comprises one or more motifs of three identical modifications on three consecutive nucleotides, where the one or more additional motifs occur at another portion of the strand that is separated from the three 2′-O-methyl modifications by at least one nucleotide. At least one of the nucleotides having a 2′-F modification may form a base pair with one of the nucleotides having a 2′-O-methyl modification.

In some embodiments, the dsRNA described herein is administered in buffer.

In some embodiments, siRNA compounds described herein can be formulated for administration to a subject. A formulated siRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A siRNA preparation can be formulated in combination with another agent, e.g., another therapeutic agent or an agent that stabilizes a siRNA, e.g., a protein that complexes with siRNA to form an iRNP. Still other agents include chelators, e.g., EDTA (e.g., to remove divalent cations such as Mg2+), salts, RNAse inhibitors (e.g., a broad specificity RNAse inhibitor such as RNAsin) and so forth.

In some embodiments, the siRNA preparation includes another siRNA compound, e.g., a second siRNA that can mediate RNAi with respect to a second gene, or with respect to the same gene. Still other preparation can include at least 3, 5, ten, twenty, fifty, or a hundred or more different siRNA species. Such siRNAs can mediate RNAi with respect to a similar number of different genes.

In some embodiments, the siRNA preparation includes at least a second therapeutic agent (e.g., an agent other than a RNA or a DNA). For example, a siRNA composition for the treatment of a viral disease, e.g., HIV, might include a known antiviral agent (e.g., a protease inhibitor or reverse transcriptase inhibitor). In another example, a siRNA composition for the treatment of a cancer might further comprise a chemotherapeutic agent.

Regarding the sequence listing in this specification, the last two 3′ bases in each oligonucleotide/siRNA can be substituted with any base (e.g., “NN”). Additionally, a 2′ deoxy can be a substitution at any position. In all of the oligonucleotides listed, any U (Uracil) can be substituted for a T (Thymine) as well as the reverse.

Further provided herein are the following embodiments: 1. A pharmaceutical composition comprising an siRNA molecule comprising a sense strand and an antisense strand, which targets SEQ ID NO: 5357 and when administered to a patient in an effective amount reduces eosinophil count, wherein the siRNA comprises a modification comprising a modified nucleoside and/or a modified internucleoside linkage; wherein the patient comprises nasal polyposis, chronic sinusitis, allergic rhinitis, or NSAID-exacerbated respiratory disease, or a combination thereof. 2. A pharmaceutical composition comprising an siRNA molecule comprising a sense strand and an antisense strand, which targets SEQ ID NO: 5357 and when administered to a patient in an effective amount reduces eosinophil count, and a pharmaceutically acceptable carrier; wherein the patient comprises nasal polyposis, chronic sinusitis, allergic rhinitis, or NSAID-exacerbated respiratory disease, or a combination thereof. 3. The pharmaceutical composition of embodiment 1 or embodiment 2, wherein the systemic or local eosinophil count is reduced by about 10% or more as compared to the eosinophil count prior to administration. 4. A pharmaceutical composition comprising an siRNA molecule which targets SEQ ID NO: 5357 and when administered in an effective amount to a patient comprising nasal polyps, the nasal polyps are reduced in number and/or size the patient, wherein the siRNA comprises a modification comprising a modified nucleoside and/or a modified internucleoside linkage. 5. A pharmaceutical composition comprising an siRNA molecule which targets SEQ ID NO: 5357 and when administered in an effective amount to a patient comprising nasal polyps, the nasal polyps are reduced in number and/or size in the patient; and a pharmaceutically acceptable carrier. 6. The pharmaceutical composition of embodiment 4 or embodiment 5, wherein the nasal polyps are reduced in number and/or size by about 10% or more by CT scan or endoscopic assessment, as compared to the number and/or size prior to administration. 7. A pharmaceutical composition comprising an siRNA molecule which targets SEQ ID NO: 5357 and when administered to a patient in an effective amount increases nasal inspiratory peak flow in the patient, wherein the siRNA comprises a modification comprising a modified nucleoside and/or a modified internucleoside linkage. 8. A pharmaceutical composition comprising an siRNA molecule which targets SEQ ID NO: 5357 and when administered to a patient in an effective amount increases nasal inspiratory peak flow in the patient, and a pharmaceutically acceptable carrier. 9. The pharmaceutical composition of embodiment 7 or embodiment 8, wherein the nasal inspiratory peak flow is increased by about 10% or more, as compared to prior to administration. 10. A pharmaceutical composition comprising an siRNA molecule which targets SEQ ID NO: 5357 and when administered to a patient in an effective amount reduces airway symptoms in the patient, wherein the patient comprises nasal polyposis, chronic sinusitis, allergic rhinitis, or NSAID-exacerbated respiratory disease, or a combination thereof, and wherein (i) the pharmaceutical composition comprises a pharmaceutically acceptable carrier, and/or (ii) the siRNA comprises a modification comprising a modified nucleoside and/or a modified internucleoside linkage. 11. The pharmaceutical composition of embodiment 10, wherein the airway inflammation symptoms are reduced by about 10% or more on a patient-reported outcome measure, as compared to prior to administration. 12. A pharmaceutical composition comprising an siRNA molecule which targets SEQ ID NO: 5357 and when administered to a patient in an effective amount improves sense of smell in the patient, wherein the patient comprises nasal polyposis, chronic sinusitis, allergic rhinitis, or NSAID-exacerbated respiratory disease, or a combination thereof, and wherein (i) the pharmaceutical composition comprises a pharmaceutically acceptable carrier, and/or (ii) the siRNA comprises a modification comprising a modified nucleoside and/or a modified internucleoside linkage. 13. The pharmaceutical composition of embodiment 12, wherein the sense of smell is improved by about 10% or more on a patient-reported outcome measure, as compared to prior to administration.

14. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6000 (C AGAGCTGCAG ACCTGGTGTC GAGAGATCAC TGAAATCGGG CTGCAAGGGG CCCAGGACCG AGGGTTTCCT GTCTCTTTAC AGGCTCGGGA CCAGGTTTGC CACTTTGTCA CCATGTGTAT CTTCACCTGC ACCGGCCAAC ACGCCTCTGT); and a pharmaceutically acceptable carrier. 15. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6000 (C AGAGCTGCAG ACCTGGTGTC GAGAGATCAC TGAAATCGGG CTGCAAGGGG CCCAGGACCG AGGGTTTCCT GTCTCTTTAC AGGCTCGGGA CCAGGTTTGC CACTTTGTCA CCATGTGTAT CTTCACCTGC ACCGGCCAAC ACGCCTCTGT); wherein the siRNA comprises a modification comprising a modified nucleoside and/or a modified internucleoside linkage. 16. The pharmaceutical composition of embodiment 14 or embodiment 15, wherein the sense strand comprises about 14-30 contiguous nucleosides of SEQ ID NO: 6001 (C AGAGCTGCAG ACCTGGTGTC GAGAGATCAC TGAAATCGGG CTGCAAGGGG) or SEQ ID NO: 6002 (CTTTAC AGGCTCGGGA CCAGGTTTGC CACTTTGTCA CCATGTGTAT CTTCA). 17. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6003 (A CCCTCTTCCC ATGTCCCACC CTCCCTAGAG GGGCACCTTT TCATGGTCTC TGCACCCAGT GAACACATTT TACTCTAGAG GCATCACCTG GGACCTTACT); and a pharmaceutically acceptable carrier. 18. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6003 (A CCCTCTTCCC ATGTCCCACC CTCCCTAGAG GGGCACCTTT TCATGGTCTC TGCACCCAGT GAACACATTT TACTCTAGAG GCATCACCTG GGACCTTACT); wherein the siRNA comprises a modification comprising a modified nucleoside and/or a modified internucleoside linkage. 19. The pharmaceutical composition of embodiment 17 or embodiment 18, wherein the sense strand comprises about 14-30 contiguous nucleosides of SEQ ID NO: 6004 (TAGAG GGGCACCTTT TCATGGTCTC TGCACCCAGT GAACACATTT TACTC). 20. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6005 (G ACATGGGAAT TTTCGACCAG ATAATGAGCA CTGGTGGGGG AGGCCACGTG CAGCTGCTCA AGCAAGCTGG AGCCTTCCTA ACCTACAGCT CCTTCTGTCC CCCTGATGAC TTGGCCGACC GGGGGCTCCT GGGAGTGAAG TCTTCCTTCT); and a pharmaceutically acceptable carrier. 21. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6005 (G ACATGGGAAT TTTCGACCAG ATAATGAGCA CTGGTGGGGG AGGCCACGTG CAGCTGCTCA AGCAAGCTGG AGCCTTCCTA ACCTACAGCT CCTTCTGTCC CCCTGATGAC TTGGCCGACC GGGGGCTCCT GGGAGTGAAG TCTTCCTTCT); wherein the siRNA comprises a modification comprising a modified nucleoside and/or a modified internucleoside linkage. 22. The pharmaceutical composition of embodiment 20 or embodiment 21, wherein the sense strand comprises about 14-30 contiguous nucleosides of SEQ ID NO: 6006 (G CAGCTGCTCA AGCAAGCTGG AGCCTTCCTA ACCTACAGCT CCTTCTGTCC).

23. The pharmaceutical composition of any one of embodiments 1, 3, 4, 6, 7, 9, 15, 16, 18, 19, 21, and 22 further comprising a pharmaceutically acceptable carrier. 24. The pharmaceutical composition of any one of embodiments 2, 3, 5, 6, 8, 9, 14, 16, 17, 19, 20, and 22 comprising a modified internucleoside linkage and/or a modified nucleoside. 25. The pharmaceutical composition of any one of embodiments 1, 3, 4, 6, 7, 9, 10, 11, 12, 13, 15, 16, 18, 19, 21, 22, 23, and 24 comprising the modified internucleoside linkage. 26. The pharmaceutical composition of embodiment 24 or embodiments 25, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. 27. The pharmaceutical composition of embodiment 26, wherein the modified internucleoside linkage comprises one or more phosphorothioate linkages. 28. The pharmaceutical composition of embodiment 27, wherein the one or more phosphorothioate linkages is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 phosphorothioate linkages.

29. The pharmaceutical composition of embodiment 27 or embodiment 28, wherein the sense strand of the siRNA comprises one or more phosphorothioate linkages. 30. The pharmaceutical composition of embodiment 29, wherein the one or more phosphorothioate linkages of the sense strand is about 1, 2, 3, 4, 5, 6, 7, 8, 9, 10, 11, 12, 13, 14, 15, 16, 17, 18, 19, 20, or 21 phosphorothioate linkages. 31. The pharmaceutical composition of embodiment 30, wherein the one or more phosphorothioate linkages of the sense strand is about 1, 2, 3, 4, or 5 phosphorothioate linkages. 32. The pharmaceutical composition of embodiment 31, wherein the one or more phosphorothioate linkages of the sense strand is about 4 phosphorothioate linkages. 33. The pharmaceutical composition of any one of embodiments 29-32, wherein the sense strand comprises a phosphorothioate linkage between the first nucleoside and the second nucleoside of the sense strand, in a 5′ to 3′ direction. 34. The pharmaceutical composition of any one of embodiments 29-33, wherein the sense strand comprises a phosphorothioate linkage between the second nucleoside and the third nucleoside of the sense strand, in a 5′ to 3′ direction. 35. The pharmaceutical composition of any one of embodiments 29-34, wherein the sense strand comprises a phosphorothioate linkage between the nineteenth nucleoside and the twentieth nucleoside of the sense strand, in a 5′ to 3′ direction. 36. The pharmaceutical composition of any one of embodiments 29-35, wherein the sense strand comprises a phosphorothioate linkage between the twentieth nucleoside and the twenty-first nucleoside of the sense strand, in a 5′ to 3′ direction.

37. The pharmaceutical composition of any one of embodiments 27-36, wherein the antisense strand of the siRNA comprises one or more phosphorothioate linkages. 38. The pharmaceutical composition of embodiment 37, wherein the one or more phosphorothioate linkages of the antisense strand is about 1, 2, 3, 4, 5, 6, 7, 8, 9, 10, 11, 12, 13, 14, 15, 16, 17, 18, 19, 20, or 21 phosphorothioate linkages. 39. The pharmaceutical composition of embodiment 38, wherein the one or more phosphorothioate linkages of the antisense strand is about 1, 2, 3, 4, or 5 phosphorothioate linkages. 40. The pharmaceutical composition of embodiment 39, wherein the one or more phosphorothioate linkages of the antisense strand is about 4 phosphorothioate linkages. 41. The pharmaceutical composition of any one of embodiments 37-40, wherein the antisense strand comprises a phosphorothioate linkage between the first nucleoside and the second nucleoside of the antisense strand, in a 5′ to 3′ direction. 42. The pharmaceutical composition of any one of embodiments 37-41, wherein the antisense strand comprises a phosphorothioate linkage between the second nucleoside and the third nucleoside of the antisense strand, in a 5′ to 3′ direction. 43. The pharmaceutical composition of any one of embodiments 37-42, wherein the antisense strand comprises a phosphorothioate linkage between the nineteenth nucleoside and the twentieth nucleoside of the sense strand, in a 5′ to 3′ direction. 44. The pharmaceutical composition of any one of embodiments 37-43, wherein the antisense strand comprises a phosphorothioate linkage between the twentieth nucleoside and the twenty-first nucleoside of the sense strand, in a 5′ to 3′ direction.

45. The pharmaceutical composition of any one of embodiments 1, 3, 4, 6, 7, 9, 10, 12, 14, 18, 21, 24 and 25-44, comprising the modified nucleoside. 46. The pharmaceutical composition of embodiment 45, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. 47. The pharmaceutical composition of embodiment 45 or embodiment 46, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

48. The pharmaceutical composition of any one of embodiments 45-47, wherein the modified nucleoside comprises one or more 2′fluoro modified nucleosides. 49. The pharmaceutical composition of embodiment 48, wherein the one or more 2′ fluoro modified nucleosides is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 2′ fluoro modified nucleosides.

50. The pharmaceutical composition of embodiment 48 or embodiment 49, wherein the sense strand of the siRNA comprises one or more 2′ fluoro modified nucleosides. 51. The pharmaceutical composition of embodiment 50, wherein the one or more 2′ fluoro modified nucleosides of the sense strand is about 1, 2, 3, 4, 5, 6, 7, 8, 9, 10, 11, 12, 13, 14, 15, 16, 17, 18, 19, 20, or 21 2′ fluoro modified nucleosides. 52. The pharmaceutical composition of embodiment 51, wherein the one or more 2′ fluoro modified nucleosides of the sense strand is about eleven 2′ fluoro modified nucleosides. 53. The pharmaceutical composition of embodiment 51, wherein the one or more 2′ fluoro modified nucleosides of the sense strand is about four 2′ fluoro modified nucleosides. 54. The pharmaceutical composition of embodiment 51, wherein the one or more 2′ fluoro modified nucleosides of the sense strand is about three 2′ fluoro modified nucleosides. 55. The pharmaceutical composition of any one of embodiments 50-54, wherein the first nucleoside of the sense strand comprises the 2′ fluoro modified nucleoside, in a 5′ to 3′ direction. 56. The pharmaceutical composition of any one of embodiments 50-55, wherein the third nucleoside of the sense strand comprises the 2′ fluoro modified nucleoside, in a 5′ to 3′ direction. 57. The pharmaceutical composition of any one of embodiments 50-56, wherein the fifth nucleoside of the sense strand comprises the 2′ fluoro modified nucleoside, in a 5′ to 3′ direction. 58. The pharmaceutical composition of any one of embodiments 50-57, wherein the seventh nucleoside of the sense strand comprises the 2′ fluoro modified nucleoside, in a 5′ to 3′ direction. 59. The pharmaceutical composition of any one of embodiments 50-58, wherein the eighth nucleoside of the sense strand comprises the 2′ fluoro modified nucleoside, in a 5′ to 3′ direction. 60. The pharmaceutical composition of any one of embodiments 50-59, wherein the ninth nucleoside of the sense strand comprises the 2′ fluoro modified nucleoside, in a 5′ to 3′ direction. 61. The pharmaceutical composition of any one of embodiments 50-60, wherein the eleventh nucleoside of the sense strand comprises the 2′ fluoro modified nucleoside, in a 5′ to 3′ direction. 62. The pharmaceutical composition of any one of embodiments 50-61, wherein the thirteenth nucleoside of the sense strand comprises the 2′ fluoro modified nucleoside, in a 5′ to 3′ direction. 63. The pharmaceutical composition of any one of embodiments 50-62, wherein the fifteenth nucleoside of the sense strand comprises the 2′ fluoro modified nucleoside, in a 5′ to 3′ direction. 64. The pharmaceutical composition of any one of embodiments 50-63, wherein the seventeenth nucleoside of the sense strand comprises the 2′ fluoro modified nucleoside, in a 5′ to 3′ direction. 65. The pharmaceutical composition of any one of embodiments 50-64, wherein the nineteenth nucleoside of the sense strand comprises the 2′ fluoro modified nucleoside, in a 5′ to 3′ direction. 66. The pharmaceutical composition of any one of embodiments 50-65, wherein the fifth, seventh, and ninth nucleosides of the sense strand comprises the 2′ fluoro modified nucleoside, in a 5′ to 3′ direction. 67. The pharmaceutical composition of any one of embodiments 50-66, wherein the sense strand comprises the pattern fN-Z1-fN-Z2-fN, wherein fN comprises the 2′ fluoro modified nucleoside and Z1 and Z2 are independently a 2′ O-methyl modified nucleoside or a 2′ fluoro modified nucleoside. 68. The pharmaceutical composition of embodiment 67, wherein the fN-Z1-fN-Z2-fN corresponds to nucleosides five to nine of the sense strand, in a 5′ to 3′ direction. 69. The pharmaceutical composition of any one of embodiments 50-68, wherein the sense strand comprises at least two contiguous 2′ fluoro modified nucleosides. 70. The pharmaceutical composition of embodiment 69, wherein the at least two contiguous 2′ fluoro modified nucleosides is two contiguous 2′ fluoro modified nucleosides. 71. The pharmaceutical composition of embodiment 69, wherein the at least two contiguous 2′ fluoro modified nucleosides is three contiguous 2′ fluoro modified nucleosides.

72. The pharmaceutical composition of any one of embodiments 48-71, wherein the antisense strand of the siRNA comprises one or more 2′ fluoro modified nucleosides. 73. The pharmaceutical composition of embodiment 72, wherein the one or more 2′ fluoro modified nucleosides of the antisense strand is about 1, 2, 3, 4, 5, 6, 7, 8, 9, 10, 11, 12, 13, 14, 15, 16, 17, 18, 19, 20, or 21 2′ fluoro modified nucleosides. 74. The pharmaceutical composition of embodiment 73, wherein the one or more 2′ fluoro modified nucleosides of the antisense strand is about eight 2′ fluoro modified nucleosides. 75. The pharmaceutical composition of embodiment 73, wherein the one or more 2′ fluoro modified nucleosides of the antisense strand is about six 2′ fluoro modified nucleosides. 76. The pharmaceutical composition of embodiment 73, wherein the one or more 2′ fluoro modified nucleosides of the antisense strand is about five 2′ fluoro modified nucleosides. 77. The pharmaceutical composition of embodiment 73, wherein the one or more 2′ fluoro modified nucleosides of the antisense strand is about four 2′ fluoro modified nucleosides. 78. The pharmaceutical composition of any one of embodiments 72-77, wherein the second nucleoside of the antisense strand comprises the 2′ fluoro modified nucleoside, in a 5′ to 3′ direction. 79. The pharmaceutical composition of any one of embodiments 72-78, wherein the fourth nucleoside of the antisense strand comprises the 2′ fluoro modified nucleoside, in a 5′ to 3′ direction. 80. The pharmaceutical composition of any one of embodiments 72-79, wherein the sixth nucleoside of the antisense strand comprises the 2′ fluoro modified nucleoside, in a 5′ to 3′ direction. 81. The pharmaceutical composition of any one of embodiments 72-80, wherein the eighth nucleoside of the antisense strand comprises the 2′ fluoro modified nucleoside, in a 5′ to 3′ direction. 82. The pharmaceutical composition of any one of embodiments 72-81, wherein the ninth nucleoside of the antisense strand comprises the 2′ fluoro modified nucleoside, in a 5′ to 3′ direction. 83. The pharmaceutical composition of any one of embodiments 72-82, wherein the tenth nucleoside of the antisense strand comprises the 2′ fluoro modified nucleoside, in a 5′ to 3′ direction. 84. The pharmaceutical composition of any one of embodiments 72-83, wherein the fourteenth nucleoside of the antisense strand comprises the 2′ fluoro modified nucleoside, in a 5′ to 3′ direction. 85. The pharmaceutical composition of any one of embodiments 72-84, wherein the sixteenth nucleoside of the antisense strand comprises the 2′ fluoro modified nucleoside, in a 5′ to 3′ direction. 86. The pharmaceutical composition of any one of embodiments 72-85, wherein the eighteenth nucleoside of the antisense strand comprises the 2′ fluoro modified nucleoside, in a 5′ to 3′ direction. 87. The pharmaceutical composition of any one of embodiments 72-86, wherein the second and fourteenth nucleosides of the antisense strand comprises the 2′ fluoro modified nucleoside, in a 5′ to 3′ direction. 88. The pharmaceutical composition of any one of embodiments 72-88, wherein the second, sixth, fourteenth, and sixteenth nucleosides of the antisense strand comprises the 2′ fluoro modified nucleoside, in a 5′ to 3′ direction. 89. The pharmaceutical composition of any one of embodiments 72-88, wherein the antisense strand comprises the pattern Z3-fN-Z4-fN, wherein fN comprises the 2′ fluoro modified nucleoside and Z3 and Z4 are independently a 2′ O-methyl modified nucleoside or a 2′ fluoro modified nucleoside. 90. The pharmaceutical composition of embodiment 89, wherein the Z3-fN-Z4-fN corresponds to nucleosides thirteen to sixteen of the antisense strand, in a 5′ to 3′ direction.

91. The pharmaceutical composition of any one of embodiments 45-90, wherein the modified nucleoside comprises a 2′ O-alkyl modified nucleoside. 92. The pharmaceutical composition of embodiment 91, wherein the 2′-O-alkyl modified nucleoside comprises one or more 2′ O-methyl modified nucleosides. 93. The pharmaceutical composition of embodiment 92, wherein the one or more 2′ O-methyl modified nucleosides is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 2′ O-methyl modified nucleosides.

94. The pharmaceutical composition of any one of embodiments 91-93, wherein the sense strand of the siRNA comprises one or more 2′ O-methyl modified nucleosides. 95. The pharmaceutical composition of embodiment 94, wherein the one or more 2′ O-methyl modified nucleosides of the sense strand is about 1, 2, 3, 4, 5, 6, 7, 8, 9, 10, 11, 12, 13, 14, 15, 16, 17, 18, 19, 20, or 21 2′ O-methyl modified nucleosides. 96. The pharmaceutical composition of embodiment 95, wherein the one or more 2′ O-methyl modified nucleosides of the sense strand is about ten 2′ O-methyl modified nucleosides. 97. The pharmaceutical composition of embodiment 95, wherein the one or more 2′ O-methyl modified nucleosides of the sense strand is about seventeen 2′ O-methyl modified nucleosides. 98. The pharmaceutical composition of embodiment 95, wherein the one or more 2′ O-methyl modified nucleosides of the sense strand is about eighteen 2′ O-methyl modified nucleosides. 99. The pharmaceutical composition of any one of embodiments 94-98, wherein the first nucleoside of the sense strand comprises the 2′ O-methyl modified nucleoside, in a 5′ to 3′ direction. 100. The pharmaceutical composition of any one of embodiments 94-99, wherein the second nucleoside of the sense strand comprises the 2′ O-methyl modified nucleoside, in a 5′ to 3′ direction. 101. The pharmaceutical composition of any one of embodiments 94-100, wherein the third nucleoside of the sense strand comprises the 2′ O-methyl modified nucleoside, in a 5′ to 3′ direction. 102. The pharmaceutical composition of any one of embodiments 94-101, wherein the fourth nucleoside of the sense strand comprises the 2′ O-methyl modified nucleoside, in a 5′ to 3′ direction. 103. The pharmaceutical composition of any one of embodiments 94-102, wherein the sixth nucleoside of the sense strand comprises the 2′ O-methyl modified nucleoside, in a 5′ to 3′ direction. 104. The pharmaceutical composition of any one of embodiments 94-103, wherein the eighth nucleoside of the sense strand comprises the 2′ O-methyl modified nucleoside, in a 5′ to 3′ direction. 105. The pharmaceutical composition of any one of embodiments 94-104, wherein the tenth nucleoside of the sense strand comprises the 2′ O-methyl modified nucleoside, in a 5′ to 3′ direction. 106. The pharmaceutical composition of any one of embodiments 94-105, wherein the eleventh nucleoside of the sense strand comprises the 2′ O-methyl modified nucleoside, in a 5′ to 3′ direction. 107. The pharmaceutical composition of any one of embodiments 94-106, wherein the twelfth nucleoside of the sense strand comprises the 2′ O-methyl modified nucleoside, in a 5′ to 3′ direction. 108. The pharmaceutical composition of any one of embodiments 94-107, wherein the thirteenth nucleoside of the sense strand comprises the 2′ O-methyl modified nucleoside, in a 5′ to 3′ direction. 109. The pharmaceutical composition of any one of embodiments 94-108, wherein the fourteenth nucleoside of the sense strand comprises the 2′ O-methyl modified nucleoside, in a 5′ to 3′ direction. 110 The pharmaceutical composition of any one of embodiments 94-109, wherein the fifteenth nucleoside of the sense strand comprises the 2′ O-methyl modified nucleoside, in a 5′ to 3′ direction. 111. The pharmaceutical composition of any one of embodiments 94-110, wherein the sixteenth nucleoside of the sense strand comprises the 2′ O-methyl modified nucleoside, in a 5′ to 3′ direction. 112. The pharmaceutical composition of any one of embodiments 94-111, wherein the seventeenth nucleoside of the sense strand comprises the 2′ O-methyl modified nucleoside, in a 5′ to 3′ direction. 113. The pharmaceutical composition of any one of embodiments 94-112, wherein the eighteenth nucleoside of the sense strand comprises the 2′ O-methyl modified nucleoside, in a 5′ to 3′ direction. 114. The pharmaceutical composition of any one of embodiments 94-113, wherein the nineteenth nucleoside of the sense strand comprises the 2′ O-methyl modified nucleoside, in a 5′ to 3′ direction. 115. The pharmaceutical composition of any one of embodiments 94-114, wherein the twentieth nucleoside of the sense strand comprises the 2′ O-methyl modified nucleoside, in a 5′ to 3′ direction. 116. The pharmaceutical composition of any one of embodiments 94-115, wherein the twenty-first nucleoside of the sense strand comprises the 2′ O-methyl modified nucleoside, in a 5′ to 3′ direction. 117. The pharmaceutical composition of any one of embodiments 94-116, wherein the second, fourth, sixth, tenth, twelfth, fourteenth, and sixteenth, eighteenth, twentieth, and twenty-first nucleosides of the sense strand comprises the 2′ O-methyl modified nucleoside, in a 5′ to 3′ direction. 118. The pharmaceutical composition of any one of embodiments 94-117, wherein the sense strand comprises the pattern mN-Z5-mN-Z6, wherein mN comprises the 2′ O-methyl modified nucleoside and Z5 and Z6 are independently a 2′ O-methyl modified nucleoside or a 2′ fluoro modified nucleoside. 119. The pharmaceutical composition of embodiment 118, wherein the mN-Z5-mN-Z6-mN corresponds to nucleosides four to seven of the sense strand, in a 5′ to 3′ direction. 120. The pharmaceutical composition of embodiment 118 or embodiment 119, wherein Z5 is the 2′ fluoro modified nucleoside. 121. The pharmaceutical composition of embodiment 118 or embodiment 119, wherein Z5 is the 2′ O-methyl modified nucleoside. 122. The pharmaceutical composition of any one of embodiments 118-121, wherein Z6 is the 2′ fluoro modified nucleoside. 123. The pharmaceutical composition of any one of embodiments 118-121, wherein Z6 is the 2′ O-methyl modified nucleoside. 124. The pharmaceutical composition of any one of embodiments 94-123, wherein the sense strand comprises the pattern mN-Z5-mN-Z6-mN, wherein mN comprises the 2′ O-methyl modified nucleoside and Z5 and Z6 are independently a 2′ O-methyl modified nucleoside or a 2′ fluoro modified nucleoside. 125. The pharmaceutical composition of embodiment 124, wherein the mN-Z5-mN-Z6-mN corresponds to nucleosides two to six of the sense strand, in a 5′ to 3′ direction. 126. The pharmaceutical composition of embodiment 124, wherein the mN-Z5-mN-Z6-mN corresponds to nucleosides ten to fourteen of the sense strand, in a 5′ to 3′ direction. 127. The pharmaceutical composition of embodiment 124, wherein the mN-Z5-mN-Z6-mN corresponds to nucleosides twelve to sixteen of the sense strand, in a 5′ to 3′ direction. 128. The pharmaceutical composition of embodiment 124, wherein the mN-Z5-mN-Z6-mN corresponds to nucleosides fourteen to eighteen of the sense strand, in a 5′ to 3′ direction. 129. The pharmaceutical composition of embodiment 124, wherein the mN-Z5-mN-Z6-mN corresponds to nucleosides sixteen to twenty of the sense strand, in a 5′ to 3′ direction. 130. The pharmaceutical composition of any one of embodiments 94-129, wherein the sense strand comprises the pattern mN-Z5-mN-Z6-mN-Z7-mN, wherein Z7 is a 2′ O-methyl modified nucleoside or a 2′ fluoro modified nucleoside. 131. The pharmaceutical composition of embodiment 130, wherein the mN-Z5-mN-Z6-mN-Z7-mN corresponds to nucleosides ten to sixteen of the sense strand, in a 5′ to 3′ direction. 132. The pharmaceutical composition of embodiment 130, wherein the mN-Z5-mN-Z6-mN-Z7-mN corresponds to nucleosides twelve to eighteen of the sense strand, in a 5′ to 3′ direction. 133. The pharmaceutical composition of embodiment 130, wherein the mN-Z5-mN-Z6-mN-Z7-mN corresponds to nucleosides fourteen to twenty of the sense strand, in a 5′ to 3′ direction. 134. The pharmaceutical composition of any one of embodiments 94-133, wherein the sense strand comprises the pattern mN-Z5-mN-Z6-mN-Z7-mN-Z8-mN, wherein Z8 is a 2′ O-methyl modified nucleoside or a 2′ fluoro modified nucleoside. 135. The pharmaceutical composition of embodiment 134, wherein the mN-Z5-mN-Z6-mN-Z7-mN-Z8-mN corresponds to nucleosides ten to eighteen of the sense strand, in a 5′ to 3′ direction. 136. The pharmaceutical composition of embodiment 134, wherein the mN-Z5-mN-Z6-mN-Z7-mN-Z8-mN corresponds to nucleosides twelve to twenty of the sense strand, in a 5′ to 3′ direction. 137. The pharmaceutical composition of any one of embodiments 94-136, wherein the sense strand comprises the pattern mN-Z5-mN-Z6-mN-Z7-mN-Z8-mN-Z9-mN, wherein Z9 is a 2′ O-methyl modified nucleoside or a 2′ fluoro modified nucleoside. 138. The pharmaceutical composition of embodiment 137, wherein the mN-Z5-mN-Z6-mN-Z7-mN-Z8-mN-Z9-mN corresponds to nucleosides ten to twenty of the sense strand, in a 5′ to 3′ direction. 139. The pharmaceutical composition of any one of embodiments 94-137, wherein the sense strand comprises at least two contiguous 2′ O-methyl modified nucleosides. 140. The pharmaceutical composition of embodiment 139, wherein the at least two contiguous 2′ O-methyl modified nucleosides is three contiguous 2′ O-methyl modified nucleosides. 141. The pharmaceutical composition of embodiment 139, wherein the at least two contiguous 2′ O-methyl modified nucleosides is four contiguous 2′ O-methyl modified nucleosides. 142. The pharmaceutical composition of embodiment 139, wherein the at least two contiguous 2′ O-methyl modified nucleosides is five contiguous 2′ O-methyl modified nucleosides. 143. The pharmaceutical composition of embodiment 139, wherein the at least two contiguous 2′ O-methyl modified nucleosides is six contiguous 2′ O-methyl modified nucleosides. 144. The pharmaceutical composition of embodiment 139, wherein the at least two contiguous 2′ O-methyl modified nucleosides is seven contiguous 2′ O-methyl modified nucleosides. 145. The pharmaceutical composition of embodiment 139, wherein the at least two contiguous 2′ O-methyl modified nucleosides is eight contiguous 2′ O-methyl modified nucleosides. 146. The pharmaceutical composition of embodiment 139, wherein the at least two contiguous 2′ O-methyl modified nucleosides is nine contiguous 2′ O-methyl modified nucleosides. 147. The pharmaceutical composition of embodiment 139, wherein the at least two contiguous 2′ O-methyl modified nucleosides is ten contiguous 2′ O-methyl modified nucleosides. 148. The pharmaceutical composition of embodiment 139, wherein the at least two contiguous 2′ O-methyl modified nucleosides is eleven contiguous 2′ O-methyl modified nucleosides. 149. The pharmaceutical composition of embodiment 139, wherein the at least two contiguous 2′ O-methyl modified nucleosides is twelve contiguous 2′ O-methyl modified nucleosides.

150. The pharmaceutical composition of any one of embodiments 91-149, wherein the antisense strand of the siRNA comprises one or more 2′ O-methyl modified nucleosides. 151. The pharmaceutical composition of embodiment 150, wherein the one or more 2′ O-methyl modified nucleosides of the antisense strand is about 1, 2, 3, 4, 5, 6, 7, 8, 9, 10, 11, 12, 13, 14, 15, 16, 17, 18, 19, 20, or 21 2′ O-methyl modified nucleosides. 152. The pharmaceutical composition of embodiment 151, wherein the one or more 2′ O-methyl modified nucleosides of the antisense strand is about thirteen 2′ O-methyl modified nucleosides. 153. The pharmaceutical composition of embodiment 151, wherein the one or more 2′ O-methyl modified nucleosides of the antisense strand is about fifteen 2′ O-methyl modified nucleosides. 154. The pharmaceutical composition of embodiment 151, wherein the one or more 2′ O-methyl modified nucleosides of the antisense strand is about seventeen 2′ O-methyl modified nucleosides. 155. The pharmaceutical composition of any one of embodiments 150-154, wherein the first nucleoside of the antisense strand comprises the 2′ O-methyl modified nucleoside, in a 5′ to 3′ direction. 156. The pharmaceutical composition of any one of embodiments 150-155, wherein the third nucleoside of the antisense strand comprises the 2′ O-methyl modified nucleoside, in a 5′ to 3′ direction. 157. The pharmaceutical composition of any one of embodiments 150-156, wherein the fourth nucleoside of the antisense strand comprises the 2′ O-methyl modified nucleoside, in a 5′ to 3′ direction. 158. The pharmaceutical composition of any one of embodiments 150-157, wherein the fifth nucleoside of the antisense strand comprises the 2′ O-methyl modified nucleoside, in a 5′ to 3′ direction. 159. The pharmaceutical composition of any one of embodiments 150-158, wherein the seventh nucleoside of the antisense strand comprises the 2′ O-methyl modified nucleoside, in a 5′ to 3′ direction. 160. The pharmaceutical composition of any one of embodiments 150-159, wherein the eighth nucleoside of the antisense strand comprises the 2′ O-methyl modified nucleoside, in a 5′ to 3′ direction. 161. The pharmaceutical composition of any one of embodiments 150-160, wherein the ninth nucleoside of the antisense strand comprises the 2′ O-methyl modified nucleoside, in a 5′ to 3′ direction. 162. The pharmaceutical composition of any one of embodiments 150-161, wherein the tenth nucleoside of the antisense strand comprises the 2′ O-methyl modified nucleoside, in a 5′ to 3′ direction. 163. The pharmaceutical composition of any one of embodiments 150-162, wherein the eleventh nucleoside of the antisense strand comprises the 2′ O-methyl modified nucleoside, in a 5′ to 3′ direction. 164. The pharmaceutical composition of any one of embodiments 150-163, wherein the twelfth nucleoside of the antisense strand comprises the 2′ O-methyl modified nucleoside, in a 5′ to 3′ direction. 165. The pharmaceutical composition of any one of embodiments 150-164, wherein the thirteenth nucleoside of the antisense strand comprises the 2′ O-methyl modified nucleoside, in a 5′ to 3′ direction. 166. The pharmaceutical composition of any one of embodiments 150-165, wherein the fifteenth nucleoside of the antisense strand comprises the 2′ O-methyl modified nucleoside, in a 5′ to 3′ direction. 167. The pharmaceutical composition of any one of embodiments 150-166, wherein the seventeenth nucleoside of the antisense strand comprises the 2′ O-methyl modified nucleoside, in a 5′ to 3′ direction. 168. The pharmaceutical composition of any one of embodiments 150-167, wherein the eighteenth nucleoside of the antisense strand comprises the 2′ O-methyl modified nucleoside, in a 5′ to 3′ direction. 169. The pharmaceutical composition of any one of embodiments 150-168, wherein the nineteenth nucleoside of the antisense strand comprises the 2′ O-methyl modified nucleoside, in a 5′ to 3′ direction. 170. The pharmaceutical composition of any one of embodiments 150-169, wherein the twentieth nucleoside of the antisense strand comprises the 2′ O-methyl modified nucleoside, in a 5′ to 3′ direction. 171. The pharmaceutical composition of any one of embodiments 150-170, wherein the twenty-first nucleoside of the antisense strand comprises the 2′ O-methyl modified nucleoside, in a 5′ to 3′ direction. 172. The pharmaceutical composition of any one of embodiments 150-171, wherein the first, third, fifth, seventh, eleventh, twelfth, thirteenth, fifteenth, seventeenth, nineteenth, twentieth, and twenty-first nucleosides of the antisense strand comprises the 2′ O-methyl modified nucleoside, in a 5′ to 3′ direction. 173. The pharmaceutical composition of any one of embodiments 150-172, wherein the antisense strand comprises at least two contiguous 2′ O-methyl modified nucleosides. 174. The pharmaceutical composition of embodiment 173, wherein the at least two contiguous 2′ O-methyl modified nucleosides is three contiguous 2′ O-methyl modified nucleosides. 175. The pharmaceutical composition of embodiment 173, wherein the at least two contiguous 2′ O-methyl modified nucleosides is four contiguous 2′ O-methyl modified nucleosides. 176. The pharmaceutical composition of embodiment 173, wherein the at least two contiguous 2′ O-methyl modified nucleosides is five contiguous 2′ O-methyl modified nucleosides. 177. The pharmaceutical composition of embodiment 173, wherein the at least two contiguous 2′ O-methyl modified nucleosides is six contiguous 2′ O-methyl modified nucleosides. 178. The pharmaceutical composition of embodiment 173, wherein the at least two contiguous 2′ O-methyl modified nucleosides is seven contiguous 2′ O-methyl modified nucleosides. 179. The pharmaceutical composition of any one of embodiments 150-178, wherein the antisense strand comprises a first sequence comprising at least two contiguous 2′ O-methyl modified nucleosides and a second sequence comprising at least two contiguous 2′ O-methyl modified nucleosides. 180. The pharmaceutical composition of embodiment 179, wherein the first sequence comprises at least three contiguous 2′ O-methyl modified nucleosides, and the second sequence comprises at least three contiguous 2′ O-methyl modified nucleosides. 181. The pharmaceutical composition of embodiment 180, wherein the first sequence comprises three contiguous 2′ O-methyl modified nucleosides, and the second sequence comprises three contiguous 2′ O-methyl modified nucleosides. 182. The pharmaceutical composition of embodiment 180, wherein the first sequence comprises four contiguous 2′ O-methyl modified nucleosides, and the second sequence comprises five contiguous 2′ O-methyl modified nucleosides. 183. The pharmaceutical composition of embodiment 180, wherein the first sequence comprises seven contiguous 2′ O-methyl modified nucleosides, and the second sequence comprises five contiguous 2′ O-methyl modified nucleosides. 184. The pharmaceutical composition of embodiment 180, wherein the first sequence comprises at least four contiguous 2′ O-methyl modified nucleosides. 185. The pharmaceutical composition of embodiment 180, wherein the first sequence comprises at least five contiguous 2′ O-methyl modified nucleosides. 186. The pharmaceutical composition of embodiment 180, wherein the first sequence comprises at least six contiguous 2′ O-methyl modified nucleosides. 187. The pharmaceutical composition of embodiment 180, wherein the first sequence comprises at least seven contiguous 2′ O-methyl modified nucleosides. 188. The pharmaceutical composition of any one of embodiments 184-187, wherein the second sequence comprises at least four contiguous 2′ O-methyl modified nucleosides. 189. The pharmaceutical composition of any one of embodiments 184-187, wherein the second sequence comprises at least five contiguous 2′ O-methyl modified nucleosides. 190. The pharmaceutical composition of any one of embodiments 184-187, wherein the second sequence comprises at least six contiguous 2′ O-methyl modified nucleosides. 191. The pharmaceutical composition of any one of embodiments 184-187, wherein the second sequence comprises at least seven contiguous 2′ O-methyl modified nucleosides.

192. The pharmaceutical composition of any one of embodiments 1-191, wherein the sense strand comprises a ribose. 193. The pharmaceutical composition of embodiment 192, wherein the sense strand comprises about 1, 2, 3, 4, 5, 6, 7, 8, 9, 10, 11, 12, 13, 14, 15, 16, 17, 18, 19, 20, or 21 ribose. 194. The pharmaceutical composition of embodiment 192 or embodiment 193, wherein the first nucleoside of the sense strand comprises the ribose, in a 5′ to 3′ direction. 195. The pharmaceutical composition of any one of embodiments 192-194, wherein the second nucleoside of the sense strand comprises the ribose, in a 5′ to 3′ direction. 196. The pharmaceutical composition of any one of embodiments 192-195, wherein the third nucleoside of the sense strand comprises the ribose, in a 5′ to 3′ direction. 197. The pharmaceutical composition of any one of embodiments 192-196, wherein the fourth nucleoside of the sense strand comprises the ribose, in a 5′ to 3′ direction. 198. The pharmaceutical composition of any one of embodiments 192-197, wherein the fifth nucleoside of the sense strand comprises the ribose, in a 5′ to 3′ direction. 199. The pharmaceutical composition of any one of embodiments 192-198, wherein the sixth nucleoside of the sense strand comprises the ribose, in a 5′ to 3′ direction. 200. The pharmaceutical composition of any one of embodiments 192-199, wherein the seventh nucleoside of the sense strand comprises the ribose, in a 5′ to 3′ direction. 201. The pharmaceutical composition of any one of embodiments 192-200, wherein the eighth nucleoside of the sense strand comprises the ribose, in a 5′ to 3′ direction. 202. The pharmaceutical composition of any one of embodiments 192-201, wherein the ninth nucleoside of the sense strand comprises the ribose, in a 5′ to 3′ direction. 203. The pharmaceutical composition of any one of embodiments 192-202, wherein the tenth nucleoside of the sense strand comprises the ribose, in a 5′ to 3′ direction. 204. The pharmaceutical composition of any one of embodiments 192-203, wherein the eleventh nucleoside of the sense strand comprises the ribose, in a 5′ to 3′ direction. 205. The pharmaceutical composition of any one of embodiments 192-204, wherein the twelfth nucleoside of the sense strand comprises the ribose, in a 5′ to 3′ direction. 206. The pharmaceutical composition of any one of embodiments 192-205, wherein the thirteenth nucleoside of the sense strand comprises the ribose, in a 5′ to 3′ direction. 207. The pharmaceutical composition of any one of embodiments 192-206, wherein the fourteenth nucleoside of the sense strand comprises the ribose, in a 5′ to 3′ direction. 208. The pharmaceutical composition of any one of embodiments 192-207, wherein the fifteenth nucleoside of the sense strand comprises the ribose, in a 5′ to 3′ direction. 209. The pharmaceutical composition of any one of embodiments 192-208, wherein the sixteenth nucleoside of the sense strand comprises the ribose, in a 5′ to 3′ direction. 210. The pharmaceutical composition of any one of embodiments 192-209, wherein the seventeenth nucleoside of the sense strand comprises the ribose, in a 5′ to 3′ direction. 211. The pharmaceutical composition of any one of embodiments 192-210, wherein the eighteenth nucleoside of the sense strand comprises the ribose, in a 5′ to 3′ direction. 212. The pharmaceutical composition of any one of embodiments 192-211, wherein the nineteenth nucleoside of the sense strand comprises the ribose, in a 5′ to 3′ direction. 213. The pharmaceutical composition of any one of embodiments 192-212, wherein the twentieth nucleoside of the sense strand comprises the ribose, in a 5′ to 3′ direction. 214. The pharmaceutical composition of any one of embodiments 192-213, wherein the twenty-first nucleoside of the sense strand comprises the ribose, in a 5′ to 3′ direction. 215. The pharmaceutical composition of any one of embodiments 1-214, wherein the antisense strand comprises a ribose. 216. The pharmaceutical composition of embodiment 215, wherein the antisense strand comprises about 1, 2, 3, 4, 5, 6, 7, 8, 9, 10, 11, 12, 13, 14, 15, 16, 17, 18, 19, 20, or 21 ribose. 217. The pharmaceutical composition of embodiment 215 or embodiment 216, wherein the first nucleoside of the antisense strand comprises the ribose, in a 5′ to 3′ direction. 218. The pharmaceutical composition of any one of embodiments 215-217, wherein the second nucleoside of the antisense strand comprises the ribose, in a 5′ to 3′ direction. 219. The pharmaceutical composition of any one of embodiments 215-218, wherein the third nucleoside of the antisense strand comprises the ribose, in a 5′ to 3′ direction. 220. The pharmaceutical composition of any one of embodiments 215-219, wherein the fourth nucleoside of the antisense strand comprises the ribose, in a 5′ to 3′ direction. 221. The pharmaceutical composition of any one of embodiments 215-220, wherein the fifth nucleoside of the antisense strand comprises the ribose, in a 5′ to 3′ direction. 222. The pharmaceutical composition of any one of embodiments 215-221, wherein the sixth nucleoside of the antisense strand comprises the ribose, in a 5′ to 3′ direction. 223. The pharmaceutical composition of any one of embodiments 215-222, wherein the seventh nucleoside of the antisense strand comprises the ribose, in a 5′ to 3′ direction. 224. The pharmaceutical composition of any one of embodiments 215-223, wherein the eighth nucleoside of the antisense strand comprises the ribose, in a 5′ to 3′ direction. 225. The pharmaceutical composition of any one of embodiments 215-224, wherein the ninth nucleoside of the antisense strand comprises the ribose, in a 5′ to 3′ direction. 226. The pharmaceutical composition of any one of embodiments 215-225, wherein the tenth nucleoside of the sense antisense comprises the ribose, in a 5′ to 3′ direction. 227. The pharmaceutical composition of any one of embodiments 215-226, wherein the eleventh nucleoside of the antisense strand comprises the ribose, in a 5′ to 3′ direction. 228. The pharmaceutical composition of any one of embodiments 215-227, wherein the twelfth nucleoside of the antisense strand comprises the ribose, in a 5′ to 3′ direction. 229. The pharmaceutical composition of any one of embodiments 215-228, wherein the thirteenth nucleoside of the antisense strand comprises the ribose, in a 5′ to 3′ direction. 230. The pharmaceutical composition of any one of embodiments 215-229, wherein the fourteenth nucleoside of the antisense strand comprises the ribose, in a 5′ to 3′ direction. 231. The pharmaceutical composition of any one of embodiments 215-230, wherein the fifteenth nucleoside of the antisense strand comprises the ribose, in a 5′ to 3′ direction. 232. The pharmaceutical composition of any one of embodiments 215-231, wherein the sixteenth nucleoside of the antisense strand comprises the ribose, in a 5′ to 3′ direction. 233. The pharmaceutical composition of any one of embodiments 215-232, wherein the seventeenth nucleoside of the antisense strand comprises the ribose, in a 5′ to 3′ direction. 234. The pharmaceutical composition of any one of embodiments 215-233, wherein the eighteenth nucleoside of the antisense strand comprises the ribose, in a 5′ to 3′ direction. 235. The pharmaceutical composition of any one of embodiments 215-234, wherein the nineteenth nucleoside of the antisense strand comprises the ribose, in a 5′ to 3′ direction. 236. The pharmaceutical composition of any one of embodiments 215-235, wherein the twentieth nucleoside of the antisense strand comprises the ribose, in a 5′ to 3′ direction. 237. The pharmaceutical composition of any one of embodiments 215-236, wherein the twenty-first nucleoside of the antisense strand comprises the ribose, in a 5′ to 3′ direction.

238. The pharmaceutical composition of any one of embodiments 1-237, further comprising a lipid attached at either 3′ or 5′ terminus of the sense strand and/or antisense strand of the siRNA. 239. The pharmaceutical composition of embodiment 238, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof. 240. The pharmaceutical composition of embodiment 238 or embodiment 239, wherein the lipid comprises a first lipid on the sense strand and a second lipid on the antisense strand. 241. The pharmaceutical composition of any one of embodiments 238-240, wherein the lipid is positioned on the sense strand. 242. The pharmaceutical composition of embodiment 241, wherein the lipid is positioned at the 5′ end of the sense strand. 243. The pharmaceutical composition of embodiment 241, wherein the lipid is positioned at the 3′ end of the sense strand. 244. The pharmaceutical composition of any one of embodiments 238-243, wherein the lipid is positioned on the antisense strand. 245. The pharmaceutical composition of embodiment 244, wherein the lipid is positioned at the 5′ end of the antisense strand. 246. The pharmaceutical composition of embodiment 244, wherein the lipid is positioned at the 3′ end of the antisense strand.

247. The pharmaceutical composition of any one of embodiments 1-246, wherein the sense strand and the antisense strand form a double-stranded RNA duplex. 248. The pharmaceutical composition of embodiment 247, wherein the double-stranded RNA duplex comprises from about 14 to about 30 nucleosides. 249. The pharmaceutical composition of embodiment 248, wherein the double-stranded RNA duplex comprises from about 17 to about 30 nucleosides. 250. The pharmaceutical composition of embodiment 249, wherein the double-stranded RNA duplex comprises about 21 nucleosides. 251. The pharmaceutical composition of any one of embodiments 247-250, wherein the double-stranded RNA duplex comprises at least one base pair. 252. The pharmaceutical composition of embodiment 251, wherein the first base pair of the double-stranded RNA duplex is an AU base pair.

253. The pharmaceutical composition of any one of embodiments 1-252, wherein the sense strand comprises pattern 1S: 5′ fN s mN s fN-mN-fN-mN-fN-fN-fN-mN-fN-mN-fN-mN-fN-mN-fN-mN-fN s mN s mN 3′, wherein “fN” is a 2′ fluoro-modified nucleoside, “mN” is a 2′ O-methyl modified nucleoside, “-” is a phosphodiester, and “s” is a phosphorothioate. 254. The pharmaceutical composition of any one of embodiments 1-252, wherein the sense strand comprises pattern 2S: 5′ mN s mN s mN-mN-fN-mN-fN-fN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 255. The pharmaceutical composition of any one of embodiments 1-252, wherein the sense strand comprises pattern 3S: 5′ mN s mN s mN-mN-fN-mN-fN-mN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 256. The pharmaceutical composition of any one of embodiments 1-252, wherein the sense strand comprises pattern 4S: 5′ fN s mN s fN-mN-fN-mN-fN-fN-fN-mN-fN-mN-fN-mN-fN-mN-fN-mN-fN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides. 257. The pharmaceutical composition of embodiment 256, wherein the one or more nucleosides is three nucleosides. 258. The pharmaceutical composition of embodiment 256 or embodiment 257, wherein each of the one or more nucleosides independently comprise a ribose or deoxyribose. 259. The pharmaceutical composition of any one of embodiments 1-252, wherein the sense strand comprises pattern 5S: 5′ mN s mN s mN-mN-fN-mN-fN-fN-fN-mN-mN-mN-mN-mN-mN-mN-mN-mN-mN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides. 260. The pharmaceutical composition of embodiment 259, wherein the one or more nucleosides is three nucleosides. 261. The pharmaceutical composition of embodiment 259 or embodiment 260, wherein each of the one or more nucleosides independently comprise a ribose or deoxyribose. 262. The pharmaceutical composition of any one of embodiments 1-261, wherein the antisense strand comprises pattern 1AS: 5′ mN s fN s mN-fN-mN-fN-mN-fN-mN-fN-mN-mN-mN-fN-mN-fN-mN-fN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 263. The pharmaceutical composition of any one of embodiments 1-261, wherein the antisense strand comprises pattern 2AS: 5′ mN s fN s mN-mN-mN-fN-mN-fN-fN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 264. The pharmaceutical composition of any one of embodiments 1-261, wherein the antisense strand comprises pattern 3AS: 5′ mN s fN s mN-mN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 265. The pharmaceutical composition of any one of embodiments 1-261, wherein the antisense strand comprises pattern 4AS: 5′ mN s fN s mN-fN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 266. The pharmaceutical composition of any one of embodiments 1-252, wherein the sense strand comprises pattern 1S and the antisense strand comprises pattern 1AS. 267. The pharmaceutical composition of any one of embodiments 1-252, wherein the sense strand comprises pattern 2S and the antisense strand comprises pattern 2AS. 268. The pharmaceutical composition of any one of embodiments 1-252, wherein the sense strand comprises pattern 3S and the antisense strand comprises pattern 3AS. 269. The pharmaceutical composition of any one of embodiments 1-252, wherein the sense strand comprises pattern 4S and the antisense strand comprises pattern 4AS.

270. The pharmaceutical composition of any one of embodiments 1-259, wherein the sense strand comprises a sequence of Formula IA: 5′ Z1-U-Z2-G-Z3-U-C-Z4-A-U-U-Z5-L 3′ wherein Z1 is 0, 3, 5, 11, or 13 nucleosides; Z2 is 1 nucleoside; Z3 is 0, 2, 5, 8, or 10 nucleosides; Z4 is 0, 2, 3, 5, 8, or 11 nucleosides; Z5 is 0 or 3 nucleosides; each “-” is independently a phosphodiester or modified internucleoside linkage; and L is an optional lipid. 271. The pharmaceutical composition of embodiment 270, wherein Z1 is 0 nucleosides. 272. The pharmaceutical composition of embodiment 270, wherein Z1 is 3, 5, 11, or 13 nucleosides. 273. The pharmaceutical composition of embodiment 272, wherein Z1 comprises UGGUG, CAGGU, CAGGUUUGCCACU (SEQ ID NO: 6007), GCACCUUUUCA (SEQ ID NO: 6008), or AGC, or a nucleic acid sequence thereof having about 1 or 2 nucleoside substitutions, additions, or deletions. 274. The pharmaceutical composition of any one of embodiments 270-273, wherein Z2 comprises C, G, or U. 275. The pharmaceutical composition of any one of embodiments 270-274, wherein Z3 is 0 nucleosides. 276. The pharmaceutical composition of any one of embodiments 270-274, wherein Z3 is 2, 5, 8, or 10 nucleosides. 277. The pharmaceutical composition of embodiment 276, wherein Z3 comprises UG, UGUCGAGAGA (SEQ ID NO: 6009), AGAGA, AGAGA, CCACUUUG, or AGCCU, or a nucleic acid sequence thereof having about 1 or 2 nucleoside substitutions, additions, or deletions. 278. The pharmaceutical composition of any one of embodiments 270-277, wherein Z4 is 0 nucleosides. 279. The pharmaceutical composition of any one of embodiments 270-277, wherein Z4 is 2, 3, 5, 8, or 11 nucleosides. 280. The pharmaceutical composition of embodiment 279, wherein Z4 comprises GAGAGAUCACU (SEQ ID NO: 6010), ACU, ACUGAAAU, UC, or CUAAC, or a nucleic acid sequence thereof having about 1 or 2 nucleoside substitutions, additions, or deletions. 281. The pharmaceutical composition of any one of embodiments 270-280, wherein Z5 represents 0 nucleosides. 282. The pharmaceutical composition of any one of embodiments 270-280, wherein Z5 represents 3 nucleosides. 283. The pharmaceutical composition of embodiment 282, wherein Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. 284. The pharmaceutical composition of any one of embodiments 270-283, wherein one or more of the Z5 nucleosides comprise a deoxyribose. 285. The pharmaceutical composition of embodiment 284, wherein the one or more is one. 286. The pharmaceutical composition of embodiment 284, wherein the one or more is two. 287. The pharmaceutical composition of embodiment 284, wherein the one or more is three. 288. The pharmaceutical composition of any one of embodiments 270-287, comprising the lipid. 289. The pharmaceutical composition of any one of embodiments 288, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof. 290. The pharmaceutical composition of any one of embodiments 270-289, wherein Z1-U-Z2-G-Z3-U-C-Z4 comprises UGGUGUCGAGAGAUCACU (SEQ ID NO: 6011), or a nucleic acid sequence thereof having about 1 or 2 nucleoside substitutions, additions, or deletions. 291. The pharmaceutical composition of any one of embodiments 270-289, wherein Z1-U-Z2-G-Z3-U-C-Z4 comprises UCGAGAGAUCACUGAAAU (SEQ ID NO: 6012), or a nucleic acid sequence thereof having about 1 or 2 nucleoside substitutions, additions, or deletions. 292. The pharmaceutical composition of any one of embodiments 270-289, wherein Z1-U-Z2-G-Z3-U-C-Z4 comprises CAGGUUUGCCACUUUGUC (SEQ ID NO: 6013), or a nucleic acid sequence thereof having about 1 or 2 nucleoside substitutions, additions, or deletions. 293. The pharmaceutical composition of any one of embodiments 270-289, wherein Z1-U-Z2-G-Z3-U-C-Z4 comprises GCACCUUUUCAUGGUCUC (SEQ ID NO: 6014), or a nucleic acid sequence thereof having about 1 or 2 nucleoside substitutions, additions, or deletions. 294. The pharmaceutical composition of any one of embodiments 270-289, wherein Z1-U-Z2-G-Z3-U-C-Z4 comprises AGCUGGAGCCUUCCUAAC (SEQ ID NO: 15), or a nucleic acid sequence thereof having about 1 or 2 nucleoside substitutions, additions, or deletions. 295. The pharmaceutical composition of any one of embodiments 270-289, wherein the sense strand comprises mUsmGsmGmUfGmUfCfGfAmGmAmGmAmUmCmAmCmUmAsmUsmU (SEQ ID NO: 5558), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 296. The pharmaceutical composition of any one of embodiments 270-289, wherein the sense strand comprises mUsmCsmGmAfGmAfGfAfUmCmAmCmUmGmAmAmAmUmAsmUsmU (SEQ ID NO: 5560), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 297. The pharmaceutical composition of any one of embodiments 270-289, wherein the sense strand comprises mCsmAsmGmGfUmUfUfGfCmCmAmCmUmUmUmGmUmCmAsmUsmU (SEQ ID NO: 5562), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 298. The pharmaceutical composition of any one of embodiments 270-289, wherein the sense strand comprises mGsmCsmAmCfCmUfUfUfUmCmAmUmGmGmUmCmUmCmAsmUsmU (SEQ ID NO: 5564), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 299. The pharmaceutical composition of any one of embodiments 270-289, wherein the sense strand comprises mAsmGsmCmUfGmGfAfGfCmCmUmUmCmCmUmAmAmCmAsmUsmU (SEQ ID NO: 5566), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 300. The pharmaceutical composition of any one of embodiments 270-299, wherein the Z1-U-Z2-G-Z3-U-C-Z4-A-U-U sequence of Formula 1A is twenty-one nucleosides in length. 301. The pharmaceutical composition of any one of embodiments 270-300, wherein the antisense strand comprises the reserve complement of Z1-U-Z2-G-Z3-U-C-Z4.

302. The pharmaceutical composition of any one of embodiments 1-301, wherein the antisense strand comprises Formula IB: 5′ U-Z6-G-A-Z7-C-Z8-A-Z9-U-U 3′ wherein Z6 represents 0, 2, 3, 5, 8, or 11 nucleosides; Z7 represents 0, 2, 5, 8, or 10 nucleosides; Z8 represents 1 nucleoside; Z9 represents 0, 3, 5, 11, or 13 nucleosides; and each “-” is independently a phosphodiester or modified internucleoside linkage. 303. The pharmaceutical composition of embodiment 302, wherein Z6 is 0 nucleosides. 304. The pharmaceutical composition of embodiment 302, wherein Z6 is 0, 2, 3, 5, 8, or 11 nucleosides. 305. The pharmaceutical composition of embodiment 304, wherein Z6 comprises AGU, AGUGAUCUCUC (SEQ ID NO: 6016), AUUUCAGU, GA, or GUUAG, or a nucleic acid sequence thereof having about 1 or 2 nucleoside substitutions, additions, or deletions. 306. The pharmaceutical composition of any one of embodiments 302-305, wherein Z7 is 0 nucleosides. 307. The pharmaceutical composition of any one of embodiments 302-305, wherein Z7 is 0, 2, 5, 8, or 10 nucleosides. 308. The pharmaceutical composition of embodiment 307, wherein Z7 comprises UCUCU, CA, UCUCU, CAAAGUGG, GA, or AGGCU, or a nucleic acid sequence thereof having about 1 or 2 nucleoside substitutions, additions, or deletions. 309. The pharmaceutical composition of any one of embodiments 302-308, wherein Z8 comprises C, G, or A. 310. The pharmaceutical composition of any one of embodiments 302-309, wherein Z9 is 0 nucleosides. 311. The pharmaceutical composition of any one of embodiments 302-309, wherein Z9 is 3, 5, 11, or 13 nucleosides. 312. The pharmaceutical composition of embodiment 311, wherein Z9 comprises CACCA, AGUGGCAAACCUG (SEQ ID NO: 6017), ACCUG, UGAAAAGGUGC (SEQ ID NO: 6018), or GCU, or a nucleic acid sequence thereof having about 1 or 2 nucleoside substitutions, additions, or deletions. 313. The pharmaceutical composition of any one of embodiments 302-312, wherein Z6-G-A-Z7-C-Z8-A-Z9 comprises AGUGAUCUCUCGACACCA (SEQ ID NO: 6019), or a nucleic acid sequence thereof having about 1 or 2 nucleoside substitutions, additions, or deletions. 314. The pharmaceutical composition of any one of embodiments 302-312, wherein Z6-G-A-Z7-C-Z8-A-Z9 comprises AUUUCAGUGAUCUCUCGA (SEQ ID NO: 6020), or a nucleic acid sequence thereof having about 1 or 2 nucleoside substitutions, additions, or deletions. 315. The pharmaceutical composition of any one of embodiments 302-312, wherein Z6-G-A-Z7-C-Z8-A-Z9 comprises GACAAAGUGGCAAACCUG (SEQ ID NO: 6021), or a nucleic acid sequence thereof having about 1 or 2 nucleoside substitutions, additions, or deletions. 316. The pharmaceutical composition of any one of embodiments 302-312, wherein Z6-G-A-Z7-C-Z8-A-Z9 comprises GAGACCAUGAAAAGGUGC (SEQ ID NO: 6022), or a nucleic acid sequence thereof having about 1 or 2 nucleoside substitutions, additions, or deletions. 317. The pharmaceutical composition of any one of embodiments 302-312, wherein Z6-G-A-Z7-C-Z8-A-Z9 comprises GUUAGGAAGGCUCCAGCU (SEQ ID NO: 6023), or a nucleic acid sequence thereof having about 1 or 2 nucleoside substitutions, additions, or deletions. 318. The pharmaceutical composition of any one of embodiments 302-312, wherein the antisense strand comprises mUsfAsmGmUmGfAmUmCmUmCmUmCmGfAmCfAmCmCmAsmUsmU (SEQ ID NO: 5559), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 319. The pharmaceutical composition of any one of embodiments 302-312, wherein the antisense strand comprises mUsfAsmUmUmUfCmAmGmUmGmAmUmCfUmCfUmCmGmAsmUsmU (SEQ ID NO: 5561), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 320. The pharmaceutical composition of any one of embodiments 302-312, wherein the antisense strand comprises mUsfGsmAmCmAfAmAmGmUmGmGmCmAfAmAfCmCmUmGsmUsmU (SEQ ID NO: 5563), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 321. The pharmaceutical composition of any one of embodiments 302-312, wherein the antisense strand comprises mUsfGsmAmGmAfCmCmAmUmGmAmAmAfAmGfGmUmGmCsmUsmU (SEQ ID NO: 5565), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 322. The pharmaceutical composition of any one of embodiments 302-312, wherein the antisense strand comprises mUsfGsmUmUmAfGmGmAmAmGmGmCmUfCmCfAmGmCmUsmUsmU (SEQ ID NO: 5567), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 323. The pharmaceutical composition of any one of embodiments 302-323, wherein the U-Z6-G-A-Z7-C-Z8-A-Z9-U-U is 21 nucleosides in length. 324. The pharmaceutical composition of any one of embodiments 1-269, wherein the sense strand comprises UGGUGUCGAGAGAUCACUG (SEQ ID NO: 5664) or a nucleic acid sequence thereof having about 1 or 2 nucleoside substitutions, additions, or deletions; and the antisense strand comprises CAGUGAUCUCUCGACACCA (SEQ ID NO: 5765) or a nucleic acid sequence thereof having about 1 or 2 nucleoside substitutions, additions, or deletions. 325. The pharmaceutical composition of any one of embodiments 1-269, wherein the sense strand comprises UGGUGUCGAGAGAUCACUA (SEQ ID NO: 5866) or a nucleic acid sequence thereof having about 1 or 2 nucleoside substitutions, additions, or deletions; and the antisense strand comprises UAGUGAUCUCUCGACACCA (SEQ ID NO: 5967) or a nucleic acid sequence thereof having about 1 or 2 nucleoside substitutions, additions, or deletions. 326. The pharmaceutical composition of any one of embodiments 1-269, wherein the sense strand comprises mUsmGsmGmUfGmUfCfGfAmGmAmGmAmUmCmAmCmUmAsmUsmU (SEQ ID NO: 5558) or a nucleic acid sequence thereof having about 1 or 2 nucleoside substitutions, additions, or deletions; and the antisense strand comprises mUsfAsmGmUmGfAmUmCmUmCmUmCmGfAmCfAmCmCmAsmUsmU (SEQ ID NO: 5559) or a nucleic acid sequence thereof having about 1 or 2 nucleoside substitutions, additions, or deletions. 327. The pharmaceutical composition of any one of embodiments 1-269, wherein the sense strand comprises UCGAGAGAUCACUGAAAUC (SEQ ID NO: 5665) or a nucleic acid sequence thereof having about 1 or 2 nucleoside substitutions, additions, or deletions; and the antisense strand comprises GAUUUCAGUGAUCUCUCGA (SEQ ID NO: 5766) or a nucleic acid sequence thereof having about 1 or 2 nucleoside substitutions, additions, or deletions. 328. The pharmaceutical composition of any one of embodiments 1-269, wherein the sense strand comprises UCGAGAGAUCACUGAAAUA (SEQ ID NO: 5867) or a nucleic acid sequence thereof having about 1 or 2 nucleoside substitutions, additions, or deletions; and the antisense strand comprises UAUUUCAGUGAUCUCUCGA (SEQ ID NO: 5968) or a nucleic acid sequence thereof having about 1 or 2 nucleoside substitutions, additions, or deletions. 329. The pharmaceutical composition of any one of embodiments 1-269, wherein the sense strand comprises mUsmCsmGmAfGmAfGfAfUmCmAmCmUmGmAmAmAmUmAsmUsmU (SEQ ID NO: 5560) or a nucleic acid sequence thereof having about 1 or 2 nucleoside substitutions, additions, or deletions; and the antisense strand comprises mUsfAsmUmUmUfCmAmGmUmGmAmUmCfUmCfUmCmGmAsmUsmU (SEQ ID NO: 5561) or a nucleic acid sequence thereof having about 1 or 2 nucleoside substitutions, additions, or deletions. 330. The pharmaceutical composition of any one of embodiments 1-269, wherein the sense strand comprises CAGGUUUGCCACUUUGUCA (SEQ ID NO: 5868) or a nucleic acid sequence thereof having about 1 or 2 nucleoside substitutions, additions, or deletions; and the antisense strand comprises UGACAAAGUGGCAAACCUG (SEQ ID NO: 5969) or a nucleic acid sequence thereof having about 1 or 2 nucleoside substitutions, additions, or deletions.

331. The pharmaceutical composition of any one of embodiments 1-269, wherein the sense strand comprises CAGGUUUGCCACUUUGUCA (SEQ ID NO: 5868) or a nucleic acid sequence thereof having about 1 or 2 nucleoside substitutions, additions, or deletions; and the antisense strand comprises UGACAAAGUGGCAAACCUG (SEQ ID NO: 5969) or a nucleic acid sequence thereof having about 1 or 2 nucleoside substitutions, additions, or deletions. 332. The pharmaceutical composition of any one of embodiments 1-269, wherein the sense strand comprises mCsmAsmGmGfUmUfUfGfCmCmAmCmUmUmUmGmUmCmAsmUsmU (SEQ ID NO: 5562) or a nucleic acid sequence thereof having about 1 or 2 nucleoside substitutions, additions, or deletions; and the antisense strand comprises mUsfGsmAmCmAfAmAmGmUmGmGmCmAfAmAfCmCmUmGsmUsmU (SEQ ID NO: 5563) or a nucleic acid sequence thereof having about 1 or 2 nucleoside substitutions, additions, or deletions. 333. The pharmaceutical composition of any one of embodiments 1-269, wherein the sense strand comprises GCACCUUUUCAUGGUCUCU (SEQ ID NO: 5667) or a nucleic acid sequence thereof having about 1 or 2 nucleoside substitutions, additions, or deletions; and the antisense strand comprises AGAGACCAUGAAAAGGUGC (SEQ ID NO: 5768) or a nucleic acid sequence thereof having about 1 or 2 nucleoside substitutions, additions, or deletions. 334. The pharmaceutical composition of any one of embodiments 1-269, wherein the sense strand comprises GCACCUUUUCAUGGUCUCA (SEQ ID NO: 5869) or a nucleic acid sequence thereof having about 1 or 2 nucleoside substitutions, additions, or deletions; and the antisense strand comprises UGAGACCAUGAAAAGGUGC (SEQ ID NO: 5970) or a nucleic acid sequence thereof having about 1 or 2 nucleoside substitutions, additions, or deletions. 335. The pharmaceutical composition of any one of embodiments 1-269, wherein the sense strand comprises mGsmCsmAmCfCmUfUfUfUmCmAmUmGmGmUmCmUmCmAsmUsmU (SEQ ID NO: 5564) or a nucleic acid sequence thereof having about 1 or 2 nucleoside substitutions, additions, or deletions; and the antisense strand comprises mUsfGsmAmGmAfCmCmAmUmGmAmAmAfAmGfGmUmGmCsmUsmU (SEQ ID NO: 5565) or a nucleic acid sequence thereof having about 1 or 2 nucleoside substitutions, additions, or deletions. 336. The pharmaceutical composition of any one of embodiments 1-269, wherein the sense strand comprises AGCUGGAGCCUUCCUAACC (SEQ ID NO: 5668) or a nucleic acid sequence thereof having about 1 or 2 nucleoside substitutions, additions, or deletions; and the antisense strand comprises GGUUAGGAAGGCUCCAGCU (SEQ ID NO: 5769) or a nucleic acid sequence thereof having about 1 or 2 nucleoside substitutions, additions, or deletions. 337. The pharmaceutical composition of any one of embodiments 1-269, wherein the sense strand comprises AGCUGGAGCCUUCCUAACA (SEQ ID NO: 5870) or a nucleic acid sequence thereof having about 1 or 2 nucleoside substitutions, additions, or deletions; and the antisense strand comprises UGUUAGGAAGGCUCCAGCU (SEQ ID NO: 5971) or a nucleic acid sequence thereof having about 1 or 2 nucleoside substitutions, additions, or deletions. 338. The pharmaceutical composition of any one of embodiments 1-269, wherein the sense strand comprises mAsmGsmCmUfGmGfAfGfCmCmUmUmCmCmUmAmAmCmAsmUsmU (SEQ ID NO: 5566) or a nucleic acid sequence thereof having about 1 or 2 nucleoside substitutions, additions, or deletions; and the antisense strand comprises mUsfGsmUmUmAfGmGmAmAmGmGmCmUfCmCfAmGmCmUsmUsmU (SEQ ID NO: 5567) or a nucleic acid sequence thereof having about 1 or 2 nucleoside substitutions, additions, or deletions. 339. The pharmaceutical composition of any one of embodiments 1-269, wherein the sense strand comprises SEQ ID NO: 23, the first 19 nucleobases of SEQ ID NO: 23, or a nucleic acid sequence thereof having about 1 or 2 nucleoside substitutions, additions, or deletions, and the antisense strand comprises SEQ ID NO: 24, the first 19 nucleobases of SEQ ID NO: 24, or a nucleic acid sequence thereof having about 1 or 2 nucleoside substitutions, additions, or deletions; or the sense strand comprises SEQ ID NO: 5366 or a nucleic acid sequence thereof having about 1 or 2 nucleoside substitutions, additions, or deletions, and the antisense strand comprises SEQ ID NO: 5367 or a nucleic acid sequence thereof having about 1 or 2 nucleoside substitutions, additions, or deletions. 340. The pharmaceutical composition of any one of embodiments 1-269, wherein the sense strand comprises SEQ ID NO: 31, the first 19 nucleobases of SEQ ID NO: 31, or a nucleic acid sequence thereof having about 1 or 2 nucleoside substitutions, additions, or deletions, and the antisense strand comprises SEQ ID NO: 32, the first 19 nucleobases of SEQ ID NO: 32, or a nucleic acid sequence thereof having about 1 or 2 nucleoside substitutions, additions, or deletions; or the sense strand comprises SEQ ID NO: 5368 or a nucleic acid sequence thereof having about 1 or 2 nucleoside substitutions, additions, or deletions, and the antisense strand comprises SEQ ID NO: 5369 or a nucleic acid sequence thereof having about 1 or 2 nucleoside substitutions, additions, or deletions. 341. The pharmaceutical composition of any one of embodiments 1-269, wherein the sense strand comprises SEQ ID NO: 33, the first 19 nucleobases of SEQ ID NO: 33, or a nucleic acid sequence thereof having about 1 or 2 nucleoside substitutions, additions, or deletions, and the antisense strand comprises SEQ ID NO: 34, the first 19 nucleobases of SEQ ID NO: 34, or a nucleic acid sequence thereof having about 1 or 2 nucleoside substitutions, additions, or deletions; or the sense strand comprises SEQ ID NO: 5370 or a nucleic acid sequence thereof having about 1 or 2 nucleoside substitutions, additions, or deletions, and the antisense strand comprises SEQ ID NO: 5371 or a nucleic acid sequence thereof having about 1 or 2 nucleoside substitutions, additions, or deletions. 342. The pharmaceutical composition of any one of embodiments 1-269, wherein the sense strand comprises SEQ ID NO: 37, the first 19 nucleobases of SEQ ID NO: 37, or a nucleic acid sequence thereof having about 1 or 2 nucleoside substitutions, additions, or deletions, and the antisense strand comprises SEQ ID NO: 38, the first 19 nucleobases of SEQ ID NO: 38, or a nucleic acid sequence thereof having about 1 or 2 nucleoside substitutions, additions, or deletions; or the sense strand comprises SEQ ID NO: 5372 or a nucleic acid sequence thereof having about 1 or 2 nucleoside substitutions, additions, or deletions, and the antisense strand comprises SEQ ID NO: 5373 or a nucleic acid sequence thereof having about 1 or 2 nucleoside substitutions, additions, or deletions. 343. The pharmaceutical composition of any one of embodiments 1-269, wherein the sense strand comprises SEQ ID NO: 39, the first 19 nucleobases of SEQ ID NO: 39, or a nucleic acid sequence thereof having about 1 or 2 nucleoside substitutions, additions, or deletions, and the antisense strand comprises SEQ ID NO: 40, the first 19 nucleobases of SEQ ID NO: 40, or a nucleic acid sequence thereof having about 1 or 2 nucleoside substitutions, additions, or deletions; or the sense strand comprises SEQ ID NO: 5374 or a nucleic acid sequence thereof having about 1 or 2 nucleoside substitutions, additions, or deletions, and the antisense strand comprises SEQ ID NO: 5375 or a nucleic acid sequence thereof having about 1 or 2 nucleoside substitutions, additions, or deletions. 344. The pharmaceutical composition of any one of embodiments 1-269, wherein the sense strand comprises SEQ ID NO: 41, the first 19 nucleobases of SEQ ID NO: 41, or a nucleic acid sequence thereof having about 1 or 2 nucleoside substitutions, additions, or deletions, and the antisense strand comprises SEQ ID NO: 42, the first 19 nucleobases of SEQ ID NO: 42, or a nucleic acid sequence thereof having about 1 or 2 nucleoside substitutions, additions, or deletions; or the sense strand comprises SEQ ID NO: 5376 or a nucleic acid sequence thereof having about 1 or 2 nucleoside substitutions, additions, or deletions, and the antisense strand comprises SEQ ID NO: 5377 or a nucleic acid sequence thereof having about 1 or 2 nucleoside substitutions, additions, or deletions. 345. The pharmaceutical composition of any one of embodiments 1-269, wherein the sense strand comprises SEQ ID NO: 43, the first 19 nucleobases of SEQ ID NO: 43, or a nucleic acid sequence thereof having about 1 or 2 nucleoside substitutions, additions, or deletions, and the antisense strand comprises SEQ ID NO: 44, the first 19 nucleobases of SEQ ID NO: 44, or a nucleic acid sequence thereof having about 1 or 2 nucleoside substitutions, additions, or deletions; or the sense strand comprises SEQ ID NO: 5378 or a nucleic acid sequence thereof having about 1 or 2 nucleoside substitutions, additions, or deletions, and the antisense strand comprises SEQ ID NO: 5379 or a nucleic acid sequence thereof having about 1 or 2 nucleoside substitutions, additions, or deletions. 346. The pharmaceutical composition of any one of embodiments 1-269, wherein the sense strand comprises SEQ ID NO: 51, the first 19 nucleobases of SEQ ID NO: 51, or a nucleic acid sequence thereof having about 1 or 2 nucleoside substitutions, additions, or deletions, and the antisense strand comprises SEQ ID NO: 52, the first 19 nucleobases of SEQ ID NO: 52, or a nucleic acid sequence thereof having about 1 or 2 nucleoside substitutions, additions, or deletions; or the sense strand comprises SEQ ID NO: 5380 or a nucleic acid sequence thereof having about 1 or 2 nucleoside substitutions, additions, or deletions, and the antisense strand comprises SEQ ID NO: 5381 or a nucleic acid sequence thereof having about 1 or 2 nucleoside substitutions, additions, or deletions. 347. The pharmaceutical composition of any one of embodiments 1-269, wherein the sense strand comprises SEQ ID NO: 195, the first 19 nucleobases of SEQ ID NO: 195, or a nucleic acid sequence thereof having about 1 or 2 nucleoside substitutions, additions, or deletions, and the antisense strand comprises SEQ ID NO: 196, the first 19 nucleobases of SEQ ID NO: 196, or a nucleic acid sequence thereof having about 1 or 2 nucleoside substitutions, additions, or deletions; or the sense strand comprises SEQ ID NO: 5382 or a nucleic acid sequence thereof having about 1 or 2 nucleoside substitutions, additions, or deletions, and the antisense strand comprises SEQ ID NO: 5383 or a nucleic acid sequence thereof having about 1 or 2 nucleoside substitutions, additions, or deletions. 348. The pharmaceutical composition of any one of embodiments 1-269, wherein the sense strand comprises SEQ ID NO: 197, the first 19 nucleobases of SEQ ID NO: 197, or a nucleic acid sequence thereof having about 1 or 2 nucleoside substitutions, additions, or deletions, and the antisense strand comprises SEQ ID NO: 198, the first 19 nucleobases of SEQ ID NO: 198, or a nucleic acid sequence thereof having about 1 or 2 nucleoside substitutions, additions, or deletions; or the sense strand comprises SEQ ID NO: 5384 or a nucleic acid sequence thereof having about 1 or 2 nucleoside substitutions, additions, or deletions, and the antisense strand comprises SEQ ID NO: 5385 or a nucleic acid sequence thereof having about 1 or 2 nucleoside substitutions, additions, or deletions. 349. The pharmaceutical composition of any one of embodiments 1-269, wherein the sense strand comprises SEQ ID NO: 199, the first 19 nucleobases of SEQ ID NO: 199, or a nucleic acid sequence thereof having about 1 or 2 nucleoside substitutions, additions, or deletions, and the antisense strand comprises SEQ ID NO: 200, the first 19 nucleobases of SEQ ID NO: 200, or a nucleic acid sequence thereof having about 1 or 2 nucleoside substitutions, additions, or deletions; or the sense strand comprises SEQ ID NO: 5386 or a nucleic acid sequence thereof having about 1 or 2 nucleoside substitutions, additions, or deletions, and the antisense strand comprises SEQ ID NO: 5387 or a nucleic acid sequence thereof having about 1 or 2 nucleoside substitutions, additions, or deletions. 350. The pharmaceutical composition of any one of embodiments 1-269, wherein the sense strand comprises SEQ ID NO: 251, the first 19 nucleobases of SEQ ID NO: 251, or a nucleic acid sequence thereof having about 1 or 2 nucleoside substitutions, additions, or deletions, and the antisense strand comprises SEQ ID NO: 252, the first 19 nucleobases of SEQ ID NO: 252, or a nucleic acid sequence thereof having about 1 or 2 nucleoside substitutions, additions, or deletions; or the sense strand comprises SEQ ID NO: 5388 or a nucleic acid sequence thereof having about 1 or 2 nucleoside substitutions, additions, or deletions, and the antisense strand comprises SEQ ID NO: 5389 or a nucleic acid sequence thereof having about 1 or 2 nucleoside substitutions, additions, or deletions. 351. The pharmaceutical composition of any one of embodiments 1-269, wherein the sense strand comprises SEQ ID NO: 257, the first 19 nucleobases of SEQ ID NO: 257, or a nucleic acid sequence thereof having about 1 or 2 nucleoside substitutions, additions, or deletions, and the antisense strand comprises SEQ ID NO: 258, the first 19 nucleobases of SEQ ID NO: 258, or a nucleic acid sequence thereof having about 1 or 2 nucleoside substitutions, additions, or deletions; or the sense strand comprises SEQ ID NO: 5390 or a nucleic acid sequence thereof having about 1 or 2 nucleoside substitutions, additions, or deletions, and the antisense strand comprises SEQ ID NO: 5391 or a nucleic acid sequence thereof having about 1 or 2 nucleoside substitutions, additions, or deletions. 352. The pharmaceutical composition of any one of embodiments 1-269, wherein the sense strand comprises SEQ ID NO: 263, the first 19 nucleobases of SEQ ID NO: 263, or a nucleic acid sequence thereof having about 1 or 2 nucleoside substitutions, additions, or deletions, and the antisense strand comprises SEQ ID NO: 264, the first 19 nucleobases of SEQ ID NO: 264, or a nucleic acid sequence thereof having about 1 or 2 nucleoside substitutions, additions, or deletions; or the sense strand comprises SEQ ID NO: 5392 or a nucleic acid sequence thereof having about 1 or 2 nucleoside substitutions, additions, or deletions, and the antisense strand comprises SEQ ID NO: 5393 or a nucleic acid sequence thereof having about 1 or 2 nucleoside substitutions, additions, or deletions. 353. The pharmaceutical composition of any one of embodiments 1-269, wherein the sense strand comprises SEQ ID NO: 273, the first 19 nucleobases of SEQ ID NO: 273, or a nucleic acid sequence thereof having about 1 or 2 nucleoside substitutions, additions, or deletions, and the antisense strand comprises SEQ ID NO: 274, the first 19 nucleobases of SEQ ID NO: 274, or a nucleic acid sequence thereof having about 1 or 2 nucleoside substitutions, additions, or deletions; or the sense strand comprises SEQ ID NO: 5394 or a nucleic acid sequence thereof having about 1 or 2 nucleoside substitutions, additions, or deletions, and the antisense strand comprises SEQ ID NO: 5395 or a nucleic acid sequence thereof having about 1 or 2 nucleoside substitutions, additions, or deletions. 354. The pharmaceutical composition of any one of embodiments 1-269, wherein the sense strand comprises SEQ ID NO: 275, the first 19 nucleobases of SEQ ID NO: 275, or a nucleic acid sequence thereof having about 1 or 2 nucleoside substitutions, additions, or deletions, and the antisense strand comprises SEQ ID NO: 276, the first 19 nucleobases of SEQ ID NO: 276, or a nucleic acid sequence thereof having about 1 or 2 nucleoside substitutions, additions, or deletions; or the sense strand comprises SEQ ID NO: 5396 or a nucleic acid sequence thereof having about 1 or 2 nucleoside substitutions, additions, or deletions, and the antisense strand comprises SEQ ID NO: 5397 or a nucleic acid sequence thereof having about 1 or 2 nucleoside substitutions, additions, or deletions. 355. The pharmaceutical composition of any one of embodiments 1-269, wherein the sense strand comprises SEQ ID NO: 279, the first 19 nucleobases of SEQ ID NO: 279, or a nucleic acid sequence thereof having about 1 or 2 nucleoside substitutions, additions, or deletions, and the antisense strand comprises SEQ ID NO: 280, the first 19 nucleobases of SEQ ID NO: 280, or a nucleic acid sequence thereof having about 1 or 2 nucleoside substitutions, additions, or deletions; or the sense strand comprises SEQ ID NO: 5398 or a nucleic acid sequence thereof having about 1 or 2 nucleoside substitutions, additions, or deletions, and the antisense strand comprises SEQ ID NO: 5399 or a nucleic acid sequence thereof having about 1 or 2 nucleoside substitutions, additions, or deletions. 356. The pharmaceutical composition of any one of embodiments 1-269, wherein the sense strand comprises SEQ ID NO: 2389, the first 19 nucleobases of SEQ ID NO: 2389, or a nucleic acid sequence thereof having about 1 or 2 nucleoside substitutions, additions, or deletions, and the antisense strand comprises SEQ ID NO: 2390, the first 19 nucleobases of SEQ ID NO: 2390, or a nucleic acid sequence thereof having about 1 or 2 nucleoside substitutions, additions, or deletions; or the sense strand comprises SEQ ID NO: 5400 or a nucleic acid sequence thereof having about 1 or 2 nucleoside substitutions, additions, or deletions, and the antisense strand comprises SEQ ID NO: 5401 or a nucleic acid sequence thereof having about 1 or 2 nucleoside substitutions, additions, or deletions. 357. The pharmaceutical composition of any one of embodiments 1-269, wherein the sense strand comprises SEQ ID NO: 2391, the first 19 nucleobases of SEQ ID NO: 2391, or a nucleic acid sequence thereof having about 1 or 2 nucleoside substitutions, additions, or deletions, and the antisense strand comprises SEQ ID NO: 2392, the first 19 nucleobases of SEQ ID NO: 2392, or a nucleic acid sequence thereof having about 1 or 2 nucleoside substitutions, additions, or deletions; or the sense strand comprises SEQ ID NO: 5402 or a nucleic acid sequence thereof having about 1 or 2 nucleoside substitutions, additions, or deletions, and the antisense strand comprises SEQ ID NO: 5403 or a nucleic acid sequence thereof having about 1 or 2 nucleoside substitutions, additions, or deletions. 358. The pharmaceutical composition of any one of embodiments 1-269, wherein the sense strand comprises SEQ ID NO: 2397, the first 19 nucleobases of SEQ ID NO: 2397, or a nucleic acid sequence thereof having about 1 or 2 nucleoside substitutions, additions, or deletions, and the antisense strand comprises SEQ ID NO: 2398, the first 19 nucleobases of SEQ ID NO: 2398, or a nucleic acid sequence thereof having about 1 or 2 nucleoside substitutions, additions, or deletions; or the sense strand comprises SEQ ID NO: 5404 or a nucleic acid sequence thereof having about 1 or 2 nucleoside substitutions, additions, or deletions, and the antisense strand comprises SEQ ID NO: 5405 or a nucleic acid sequence thereof having about 1 or 2 nucleoside substitutions, additions, or deletions. 359. The pharmaceutical composition of any one of embodiments 1-269, wherein the sense strand comprises SEQ ID NO: 2399, the first 19 nucleobases of SEQ ID NO: 2399, or a nucleic acid sequence thereof having about 1 or 2 nucleoside substitutions, additions, or deletions, and the antisense strand comprises SEQ ID NO: 2400, the first 19 nucleobases of SEQ ID NO: 2400, or a nucleic acid sequence thereof having about 1 or 2 nucleoside substitutions, additions, or deletions; or the sense strand comprises SEQ ID NO: 5406 or a nucleic acid sequence thereof having about 1 or 2 nucleoside substitutions, additions, or deletions, and the antisense strand comprises SEQ ID NO: 5407 or a nucleic acid sequence thereof having about 1 or 2 nucleoside substitutions, additions, or deletions. 360. The pharmaceutical composition of any one of embodiments 1-269, wherein the sense strand comprises SEQ ID NO: 2409, the first 19 nucleobases of SEQ ID NO: 2409, or a nucleic acid sequence thereof having about 1 or 2 nucleoside substitutions, additions, or deletions, and the antisense strand comprises SEQ ID NO: 2410, the first 19 nucleobases of SEQ ID NO: 2410, or a nucleic acid sequence thereof having about 1 or 2 nucleoside substitutions, additions, or deletions; or the sense strand comprises SEQ ID NO: 5408 or a nucleic acid sequence thereof having about 1 or 2 nucleoside substitutions, additions, or deletions, and the antisense strand comprises SEQ ID NO: 5409 or a nucleic acid sequence thereof having about 1 or 2 nucleoside substitutions, additions, or deletions. 361. The pharmaceutical composition of any one of embodiments 1-269, wherein the sense strand comprises SEQ ID NO: 2415, the first 19 nucleobases of SEQ ID NO: 2415, or a nucleic acid sequence thereof having about 1 or 2 nucleoside substitutions, additions, or deletions, and the antisense strand comprises SEQ ID NO: 2416, the first 19 nucleobases of SEQ ID NO: 2416, or a nucleic acid sequence thereof having about 1 or 2 nucleoside substitutions, additions, or deletions; or the sense strand comprises SEQ ID NO: 5410 or a nucleic acid sequence thereof having about 1 or 2 nucleoside substitutions, additions, or deletions, and the antisense strand comprises SEQ ID NO: 5411 or a nucleic acid sequence thereof having about 1 or 2 nucleoside substitutions, additions, or deletions. 362. The pharmaceutical composition of any one of embodiments 1-269, wherein the sense strand comprises SEQ ID NO: 2419, the first 19 nucleobases of SEQ ID NO: 2419, or a nucleic acid sequence thereof having about 1 or 2 nucleoside substitutions, additions, or deletions, and the antisense strand comprises SEQ ID NO: 2420, the first 19 nucleobases of SEQ ID NO: 2420, or a nucleic acid sequence thereof having about 1 or 2 nucleoside substitutions, additions, or deletions; or the sense strand comprises SEQ ID NO: 5412 or a nucleic acid sequence thereof having about 1 or 2 nucleoside substitutions, additions, or deletions, and the antisense strand comprises SEQ ID NO: 5413 or a nucleic acid sequence thereof having about 1 or 2 nucleoside substitutions, additions, or deletions. 363. The pharmaceutical composition of any one of embodiments 1-269, wherein the sense strand comprises SEQ ID NO: 2421, the first 19 nucleobases of SEQ ID NO: 2421, or a nucleic acid sequence thereof having about 1 or 2 nucleoside substitutions, additions, or deletions, and the antisense strand comprises SEQ ID NO: 2422, the first 19 nucleobases of SEQ ID NO: 2422, or a nucleic acid sequence thereof having about 1 or 2 nucleoside substitutions, additions, or deletions; or the sense strand comprises SEQ ID NO: 5414 or a nucleic acid sequence thereof having about 1 or 2 nucleoside substitutions, additions, or deletions, and the antisense strand comprises SEQ ID NO: 5415 or a nucleic acid sequence thereof having about 1 or 2 nucleoside substitutions, additions, or deletions. 364. The pharmaceutical composition of any one of embodiments 1-269, wherein the sense strand comprises SEQ ID NO: 2423, the first 19 nucleobases of SEQ ID NO: 2423, or a nucleic acid sequence thereof having about 1 or 2 nucleoside substitutions, additions, or deletions, and the antisense strand comprises SEQ ID NO: 2424, the first 19 nucleobases of SEQ ID NO: 2424, or a nucleic acid sequence thereof having about 1 or 2 nucleoside substitutions, additions, or deletions; or the sense strand comprises SEQ ID NO: 5416 or a nucleic acid sequence thereof having about 1 or 2 nucleoside substitutions, additions, or deletions, and the antisense strand comprises SEQ ID NO: 5417 or a nucleic acid sequence thereof having about 1 or 2 nucleoside substitutions, additions, or deletions. 365. The pharmaceutical composition of any one of embodiments 1-269, wherein the sense strand comprises SEQ ID NO: 2425, the first 19 nucleobases of SEQ ID NO: 2425, or a nucleic acid sequence thereof having about 1 or 2 nucleoside substitutions, additions, or deletions, and the antisense strand comprises SEQ ID NO: 2426, the first 19 nucleobases of SEQ ID NO: 2426, or a nucleic acid sequence thereof having about 1 or 2 nucleoside substitutions, additions, or deletions; or the sense strand comprises SEQ ID NO: 5418 or a nucleic acid sequence thereof having about 1 or 2 nucleoside substitutions, additions, or deletions, and the antisense strand comprises SEQ ID NO: 5419 or a nucleic acid sequence thereof having about 1 or 2 nucleoside substitutions, additions, or deletions. 366. The pharmaceutical composition of any one of embodiments 1-269, wherein the sense strand comprises SEQ ID NO: 2427, the first 19 nucleobases of SEQ ID NO: 2427, or a nucleic acid sequence thereof having about 1 or 2 nucleoside substitutions, additions, or deletions, and the antisense strand comprises SEQ ID NO: 2428, the first 19 nucleobases of SEQ ID NO: 2428, or a nucleic acid sequence thereof having about 1 or 2 nucleoside substitutions, additions, or deletions; or the sense strand comprises SEQ ID NO: 5420 or a nucleic acid sequence thereof having about 1 or 2 nucleoside substitutions, additions, or deletions, and the antisense strand comprises SEQ ID NO: 5421 or a nucleic acid sequence thereof having about 1 or 2 nucleoside substitutions, additions, or deletions. 367. The pharmaceutical composition of any one of embodiments 1-269, wherein the sense strand comprises SEQ ID NO: 2431, the first 19 nucleobases of SEQ ID NO: 2431, or a nucleic acid sequence thereof having about 1 or 2 nucleoside substitutions, additions, or deletions, and the antisense strand comprises SEQ ID NO: 2432, the first 19 nucleobases of SEQ ID NO: 2432, or a nucleic acid sequence thereof having about 1 or 2 nucleoside substitutions, additions, or deletions; or the sense strand comprises SEQ ID NO: 5422 or a nucleic acid sequence thereof having about 1 or 2 nucleoside substitutions, additions, or deletions, and the antisense strand comprises SEQ ID NO: 5423 or a nucleic acid sequence thereof having about 1 or 2 nucleoside substitutions, additions, or deletions. 368. The pharmaceutical composition of any one of embodiments 1-269, wherein the sense strand comprises SEQ ID NO: 2437, the first 19 nucleobases of SEQ ID NO: 2437, or a nucleic acid sequence thereof having about 1 or 2 nucleoside substitutions, additions, or deletions, and the antisense strand comprises SEQ ID NO: 2438, the first 19 nucleobases of SEQ ID NO: 2438, or a nucleic acid sequence thereof having about 1 or 2 nucleoside substitutions, additions, or deletions; or the sense strand comprises SEQ ID NO: 5424 or a nucleic acid sequence thereof having about 1 or 2 nucleoside substitutions, additions, or deletions, and the antisense strand comprises SEQ ID NO: 5425 or a nucleic acid sequence thereof having about 1 or 2 nucleoside substitutions, additions, or deletions. 369. The pharmaceutical composition of any one of embodiments 1-269, wherein the sense strand comprises SEQ ID NO: 2439, the first 19 nucleobases of SEQ ID NO: 2439, or a nucleic acid sequence thereof having about 1 or 2 nucleoside substitutions, additions, or deletions, and the antisense strand comprises SEQ ID NO: 2440, the first 19 nucleobases of SEQ ID NO: 2440, or a nucleic acid sequence thereof having about 1 or 2 nucleoside substitutions, additions, or deletions; or the sense strand comprises SEQ ID NO: 5426 or a nucleic acid sequence thereof having about 1 or 2 nucleoside substitutions, additions, or deletions, and the antisense strand comprises SEQ ID NO: 5427 or a nucleic acid sequence thereof having about 1 or 2 nucleoside substitutions, additions, or deletions. 370. The pharmaceutical composition of any one of embodiments 1-269, wherein the sense strand comprises SEQ ID NO: 2769, the first 19 nucleobases of SEQ ID NO: 2769, or a nucleic acid sequence thereof having about 1 or 2 nucleoside substitutions, additions, or deletions, and the antisense strand comprises SEQ ID NO: 2770, the first 19 nucleobases of SEQ ID NO: 2770, or a nucleic acid sequence thereof having about 1 or 2 nucleoside substitutions, additions, or deletions; or the sense strand comprises SEQ ID NO: 5428 or a nucleic acid sequence thereof having about 1 or 2 nucleoside substitutions, additions, or deletions, and the antisense strand comprises SEQ ID NO: 5429 or a nucleic acid sequence thereof having about 1 or 2 nucleoside substitutions, additions, or deletions. 371. The pharmaceutical composition of any one of embodiments 1-269, wherein the sense strand comprises SEQ ID NO: 2771, the first 19 nucleobases of SEQ ID NO: 2771, or a nucleic acid sequence thereof having about 1 or 2 nucleoside substitutions, additions, or deletions, and the antisense strand comprises SEQ ID NO: 2772, the first 19 nucleobases of SEQ ID NO: 2772, or a nucleic acid sequence thereof having about 1 or 2 nucleoside substitutions, additions, or deletions; or the sense strand comprises SEQ ID NO: 5430 or a nucleic acid sequence thereof having about 1 or 2 nucleoside substitutions, additions, or deletions, and the antisense strand comprises SEQ ID NO: 5431 or a nucleic acid sequence thereof having about 1 or 2 nucleoside substitutions, additions, or deletions. 372. The pharmaceutical composition of any one of embodiments 1-269, wherein the sense strand comprises SEQ ID NO: 2773, the first 19 nucleobases of SEQ ID NO: 2773, or a nucleic acid sequence thereof having about 1 or 2 nucleoside substitutions, additions, or deletions, and the antisense strand comprises SEQ ID NO: 2774, the first 19 nucleobases of SEQ ID NO: 2774, or a nucleic acid sequence thereof having about 1 or 2 nucleoside substitutions, additions, or deletions; or the sense strand comprises SEQ ID NO: 5432 or a nucleic acid sequence thereof having about 1 or 2 nucleoside substitutions, additions, or deletions, and the antisense strand comprises SEQ ID NO: 5433 or a nucleic acid sequence thereof having about 1 or 2 nucleoside substitutions, additions, or deletions. 373. The pharmaceutical composition of any one of embodiments 1-269, wherein the sense strand comprises SEQ ID NO: 2881, the first 19 nucleobases of SEQ ID NO: 2881, or a nucleic acid sequence thereof having about 1 or 2 nucleoside substitutions, additions, or deletions, and the antisense strand comprises SEQ ID NO: 2882, the first 19 nucleobases of SEQ ID NO: 2882, or a nucleic acid sequence thereof having about 1 or 2 nucleoside substitutions, additions, or deletions; or the sense strand comprises SEQ ID NO: 5434 or a nucleic acid sequence thereof having about 1 or 2 nucleoside substitutions, additions, or deletions, and the antisense strand comprises SEQ ID NO: 5435 or a nucleic acid sequence thereof having about 1 or 2 nucleoside substitutions, additions, or deletions. 374. The pharmaceutical composition of any one of embodiments 1-269, wherein the sense strand comprises SEQ ID NO: 2899, the first 19 nucleobases of SEQ ID NO: 2899, or a nucleic acid sequence thereof having about 1 or 2 nucleoside substitutions, additions, or deletions, and the antisense strand comprises SEQ ID NO: 2900, the first 19 nucleobases of SEQ ID NO: 2900, or a nucleic acid sequence thereof having about 1 or 2 nucleoside substitutions, additions, or deletions; or the sense strand comprises SEQ ID NO: 5436 or a nucleic acid sequence thereof having about 1 or 2 nucleoside substitutions, additions, or deletions, and the antisense strand comprises SEQ ID NO: 5437 or a nucleic acid sequence thereof having about 1 or 2 nucleoside substitutions, additions, or deletions. 375. The pharmaceutical composition of any one of embodiments 1-269, wherein the sense strand comprises SEQ ID NO: 2903, the first 19 nucleobases of SEQ ID NO: 2903, or a nucleic acid sequence thereof having about 1 or 2 nucleoside substitutions, additions, or deletions, and the antisense strand comprises SEQ ID NO: 2904, the first 19 nucleobases of SEQ ID NO: 2904, or a nucleic acid sequence thereof having about 1 or 2 nucleoside substitutions, additions, or deletions; or the sense strand comprises SEQ ID NO: 5438 or a nucleic acid sequence thereof having about 1 or 2 nucleoside substitutions, additions, or deletions, and the antisense strand comprises SEQ ID NO: 5439 or a nucleic acid sequence thereof having about 1 or 2 nucleoside substitutions, additions, or deletions. 376. The pharmaceutical composition of any one of embodiments 1-269, wherein the sense strand comprises SEQ ID NO: 2905, the first 19 nucleobases of SEQ ID NO: 2905, or a nucleic acid sequence thereof having about 1 or 2 nucleoside substitutions, additions, or deletions, and the antisense strand comprises SEQ ID NO: 2906, the first 19 nucleobases of SEQ ID NO: 2906, or a nucleic acid sequence thereof having about 1 or 2 nucleoside substitutions, additions, or deletions; or the sense strand comprises SEQ ID NO: 5440 or a nucleic acid sequence thereof having about 1 or 2 nucleoside substitutions, additions, or deletions, and the antisense strand comprises SEQ ID NO: 5441 or a nucleic acid sequence thereof having about 1 or 2 nucleoside substitutions, additions, or deletions. 377. The pharmaceutical composition of any one of embodiments 1-269, wherein the sense strand comprises SEQ ID NO: 2909, the first 19 nucleobases of SEQ ID NO: 2909, or a nucleic acid sequence thereof having about 1 or 2 nucleoside substitutions, additions, or deletions, and the antisense strand comprises SEQ ID NO: 2910, the first 19 nucleobases of SEQ ID NO: 2910, or a nucleic acid sequence thereof having about 1 or 2 nucleoside substitutions, additions, or deletions; or the sense strand comprises SEQ ID NO: 5442 or a nucleic acid sequence thereof having about 1 or 2 nucleoside substitutions, additions, or deletions, and the antisense strand comprises SEQ ID NO: 5443 or a nucleic acid sequence thereof having about 1 or 2 nucleoside substitutions, additions, or deletions. 378. The pharmaceutical composition of any one of embodiments 1-269, wherein the sense strand comprises SEQ ID NO: 2913, the first 19 nucleobases of SEQ ID NO: 2913, or a nucleic acid sequence thereof having about 1 or 2 nucleoside substitutions, additions, or deletions, and the antisense strand comprises SEQ ID NO: 2914, the first 19 nucleobases of SEQ ID NO: 2914, or a nucleic acid sequence thereof having about 1 or 2 nucleoside substitutions, additions, or deletions; or the sense strand comprises SEQ ID NO: 5444 or a nucleic acid sequence thereof having about 1 or 2 nucleoside substitutions, additions, or deletions, and the antisense strand comprises SEQ ID NO: 5445 or a nucleic acid sequence thereof having about 1 or 2 nucleoside substitutions, additions, or deletions. 379. The pharmaceutical composition of any one of embodiments 1-269, wherein the sense strand comprises SEQ ID NO: 2915, the first 19 nucleobases of SEQ ID NO: 2915, or a nucleic acid sequence thereof having about 1 or 2 nucleoside substitutions, additions, or deletions, and the antisense strand comprises SEQ ID NO: 2916, the first 19 nucleobases of SEQ ID NO: 2916, or a nucleic acid sequence thereof having about 1 or 2 nucleoside substitutions, additions, or deletions; or the sense strand comprises SEQ ID NO: 5446 or a nucleic acid sequence thereof having about 1 or 2 nucleoside substitutions, additions, or deletions, and the antisense strand comprises SEQ ID NO: 5447 or a nucleic acid sequence thereof having about 1 or 2 nucleoside substitutions, additions, or deletions. 380. The pharmaceutical composition of any one of embodiments 1-269, wherein the sense strand comprises SEQ ID NO: 3013, the first 19 nucleobases of SEQ ID NO: 3013, or a nucleic acid sequence thereof having about 1 or 2 nucleoside substitutions, additions, or deletions, and the antisense strand comprises SEQ ID NO: 3014, the first 19 nucleobases of SEQ ID NO: 3014, or a nucleic acid sequence thereof having about 1 or 2 nucleoside substitutions, additions, or deletions; or the sense strand comprises SEQ ID NO: 5448 or a nucleic acid sequence thereof having about 1 or 2 nucleoside substitutions, additions, or deletions, and the antisense strand comprises SEQ ID NO: 5449 or a nucleic acid sequence thereof having about 1 or 2 nucleoside substitutions, additions, or deletions. 381. The pharmaceutical composition of any one of embodiments 1-269, wherein the sense strand comprises SEQ ID NO: 3015, the first 19 nucleobases of SEQ ID NO: 3015, or a nucleic acid sequence thereof having about 1 or 2 nucleoside substitutions, additions, or deletions, and the antisense strand comprises SEQ ID NO: 3016, the first 19 nucleobases of SEQ ID NO: 3016, or a nucleic acid sequence thereof having about 1 or 2 nucleoside substitutions, additions, or deletions; or the sense strand comprises SEQ ID NO: 5450 or a nucleic acid sequence thereof having about 1 or 2 nucleoside substitutions, additions, or deletions, and the antisense strand comprises SEQ ID NO: 5451 or a nucleic acid sequence thereof having about 1 or 2 nucleoside substitutions, additions, or deletions. 382. The pharmaceutical composition of any one of embodiments 1-269, wherein the sense strand comprises SEQ ID NO: 3017, the first 19 nucleobases of SEQ ID NO: 3017, or a nucleic acid sequence thereof having about 1 or 2 nucleoside substitutions, additions, or deletions, and the antisense strand comprises SEQ ID NO: 3018, the first 19 nucleobases of SEQ ID NO: 3018, or a nucleic acid sequence thereof having about 1 or 2 nucleoside substitutions, additions, or deletions; or the sense strand comprises SEQ ID NO: 5452 or a nucleic acid sequence thereof having about 1 or 2 nucleoside substitutions, additions, or deletions, and the antisense strand comprises SEQ ID NO: 5453 or a nucleic acid sequence thereof having about 1 or 2 nucleoside substitutions, additions, or deletions. 383. The pharmaceutical composition of any one of embodiments 1-269, wherein the sense strand comprises SEQ ID NO: 3019, the first 19 nucleobases of SEQ ID NO: 3019, or a nucleic acid sequence thereof having about 1 or 2 nucleoside substitutions, additions, or deletions, and the antisense strand comprises SEQ ID NO: 3020, the first 19 nucleobases of SEQ ID NO: 3020, or a nucleic acid sequence thereof having about 1 or 2 nucleoside substitutions, additions, or deletions; or the sense strand comprises SEQ ID NO: 5454 or a nucleic acid sequence thereof having about 1 or 2 nucleoside substitutions, additions, or deletions, and the antisense strand comprises SEQ ID NO: 5455 or a nucleic acid sequence thereof having about 1 or 2 nucleoside substitutions, additions, or deletions. 384. The pharmaceutical composition of any one of embodiments 1-269, wherein the sense strand comprises SEQ ID NO: 3027, the first 19 nucleobases of SEQ ID NO: 3027, or a nucleic acid sequence thereof having about 1 or 2 nucleoside substitutions, additions, or deletions, and the antisense strand comprises SEQ ID NO: 3028, the first 19 nucleobases of SEQ ID NO: 3028, or a nucleic acid sequence thereof having about 1 or 2 nucleoside substitutions, additions, or deletions; or the sense strand comprises SEQ ID NO: 5456 or a nucleic acid sequence thereof having about 1 or 2 nucleoside substitutions, additions, or deletions, and the antisense strand comprises SEQ ID NO: 5457 or a nucleic acid sequence thereof having about 1 or 2 nucleoside substitutions, additions, or deletions. 385. The pharmaceutical composition of any one of embodiments 1-269, wherein the sense strand comprises SEQ ID NO: 3029, the first 19 nucleobases of SEQ ID NO: 3029, or a nucleic acid sequence thereof having about 1 or 2 nucleoside substitutions, additions, or deletions, and the antisense strand comprises SEQ ID NO: 3030, the first 19 nucleobases of SEQ ID NO: 3030, or a nucleic acid sequence thereof having about 1 or 2 nucleoside substitutions, additions, or deletions; or the sense strand comprises SEQ ID NO: 5458 or a nucleic acid sequence thereof having about 1 or 2 nucleoside substitutions, additions, or deletions, and the antisense strand comprises SEQ ID NO: 5459 or a nucleic acid sequence thereof having about 1 or 2 nucleoside substitutions, additions, or deletions. 386. The pharmaceutical composition of any one of embodiments 1-269, wherein the sense strand comprises SEQ ID NO: 3037, the first 19 nucleobases of SEQ ID NO: 3037, or a nucleic acid sequence thereof having about 1 or 2 nucleoside substitutions, additions, or deletions, and the antisense strand comprises SEQ ID NO: 3038, the first 19 nucleobases of SEQ ID NO: 3038, or a nucleic acid sequence thereof having about 1 or 2 nucleoside substitutions, additions, or deletions; or the sense strand comprises SEQ ID NO: 5460 or a nucleic acid sequence thereof having about 1 or 2 nucleoside substitutions, additions, or deletions, and the antisense strand comprises SEQ ID NO: 5461 or a nucleic acid sequence thereof having about 1 or 2 nucleoside substitutions, additions, or deletions. 387. The pharmaceutical composition of any one of embodiments 1-269, wherein the sense strand comprises SEQ ID NO: 3039, the first 19 nucleobases of SEQ ID NO: 3039, or a nucleic acid sequence thereof having about 1 or 2 nucleoside substitutions, additions, or deletions, and the antisense strand comprises SEQ ID NO: 3040, the first 19 nucleobases of SEQ ID NO: 3040, or a nucleic acid sequence thereof having about 1 or 2 nucleoside substitutions, additions, or deletions; or the sense strand comprises SEQ ID NO: 5462 or a nucleic acid sequence thereof having about 1 or 2 nucleoside substitutions, additions, or deletions, and the antisense strand comprises SEQ ID NO: 5463 or a nucleic acid sequence thereof having about 1 or 2 nucleoside substitutions, additions, or deletions. 388. The pharmaceutical composition of any one of embodiments 1-269, wherein the sense strand comprises SEQ ID NO: 3041, the first 19 nucleobases of SEQ ID NO: 3041, or a nucleic acid sequence thereof having about 1 or 2 nucleoside substitutions, additions, or deletions, and the antisense strand comprises SEQ ID NO: 3042, the first 19 nucleobases of SEQ ID NO: 3042, or a nucleic acid sequence thereof having about 1 or 2 nucleoside substitutions, additions, or deletions; or the sense strand comprises SEQ ID NO: 5464 or a nucleic acid sequence thereof having about 1 or 2 nucleoside substitutions, additions, or deletions, and the antisense strand comprises SEQ ID NO: 5465 or a nucleic acid sequence thereof having about 1 or 2 nucleoside substitutions, additions, or deletions. 389. The pharmaceutical composition of any one of embodiments 1-269, wherein the sense strand comprises SEQ ID NO: 3047, the first 19 nucleobases of SEQ ID NO: 3047, or a nucleic acid sequence thereof having about 1 or 2 nucleoside substitutions, additions, or deletions, and the antisense strand comprises SEQ ID NO: 3048, the first 19 nucleobases of SEQ ID NO: 3048, or a nucleic acid sequence thereof having about 1 or 2 nucleoside substitutions, additions, or deletions; or the sense strand comprises SEQ ID NO: 5466 or a nucleic acid sequence thereof having about 1 or 2 nucleoside substitutions, additions, or deletions, and the antisense strand comprises SEQ ID NO: 5467 or a nucleic acid sequence thereof having about 1 or 2 nucleoside substitutions, additions, or deletions. 390. The pharmaceutical composition of any one of embodiments 1-269, wherein the sense strand comprises SEQ ID NO: 3053, the first 19 nucleobases of SEQ ID NO: 3053, or a nucleic acid sequence thereof having about 1 or 2 nucleoside substitutions, additions, or deletions, and the antisense strand comprises SEQ ID NO: 3054, the first 19 nucleobases of SEQ ID NO: 3054, or a nucleic acid sequence thereof having about 1 or 2 nucleoside substitutions, additions, or deletions; or the sense strand comprises SEQ ID NO: 5468 or a nucleic acid sequence thereof having about 1 or 2 nucleoside substitutions, additions, or deletions, and the antisense strand comprises SEQ ID NO: 5469 or a nucleic acid sequence thereof having about 1 or 2 nucleoside substitutions, additions, or deletions. 391. The pharmaceutical composition of any one of embodiments 1-269, wherein the sense strand comprises SEQ ID NO: 3055, the first 19 nucleobases of SEQ ID NO: 3055, or a nucleic acid sequence thereof having about 1 or 2 nucleoside substitutions, additions, or deletions, and the antisense strand comprises SEQ ID NO: 3056, the first 19 nucleobases of SEQ ID NO: 3056, or a nucleic acid sequence thereof having about 1 or 2 nucleoside substitutions, additions, or deletions; or the sense strand comprises SEQ ID NO: 5470 or a nucleic acid sequence thereof having about 1 or 2 nucleoside substitutions, additions, or deletions, and the antisense strand comprises SEQ ID NO: 5471 or a nucleic acid sequence thereof having about 1 or 2 nucleoside substitutions, additions, or deletions. 392. The pharmaceutical composition of any one of embodiments 1-269, wherein the sense strand comprises SEQ ID NO: 3063, the first 19 nucleobases of SEQ ID NO: 3063, or a nucleic acid sequence thereof having about 1 or 2 nucleoside substitutions, additions, or deletions, and the antisense strand comprises SEQ ID NO: 3064, the first 19 nucleobases of SEQ ID NO: 3064, or a nucleic acid sequence thereof having about 1 or 2 nucleoside substitutions, additions, or deletions; or the sense strand comprises SEQ ID NO: 5472 or a nucleic acid sequence thereof having about 1 or 2 nucleoside substitutions, additions, or deletions, and the antisense strand comprises SEQ ID NO: 5473 or a nucleic acid sequence thereof having about 1 or 2 nucleoside substitutions, additions, or deletions. 393. The pharmaceutical composition of any one of embodiments 1-269, wherein the sense strand comprises SEQ ID NO: 3065, the first 19 nucleobases of SEQ ID NO: 3065, or a nucleic acid sequence thereof having about 1 or 2 nucleoside substitutions, additions, or deletions, and the antisense strand comprises SEQ ID NO: 3066, the first 19 nucleobases of SEQ ID NO: 3066, or a nucleic acid sequence thereof having about 1 or 2 nucleoside substitutions, additions, or deletions; or the sense strand comprises SEQ ID NO: 5474 or a nucleic acid sequence thereof having about 1 or 2 nucleoside substitutions, additions, or deletions, and the antisense strand comprises SEQ ID NO: 5475 or a nucleic acid sequence thereof having about 1 or 2 nucleoside substitutions, additions, or deletions. 394. The pharmaceutical composition of any one of embodiments 1-269, wherein the sense strand comprises SEQ ID NO: 3183, the first 19 nucleobases of SEQ ID NO: 3183, or a nucleic acid sequence thereof having about 1 or 2 nucleoside substitutions, additions, or deletions, and the antisense strand comprises SEQ ID NO: 3184, the first 19 nucleobases of SEQ ID NO: 3184, or a nucleic acid sequence thereof having about 1 or 2 nucleoside substitutions, additions, or deletions; or the sense strand comprises SEQ ID NO: 5476 or a nucleic acid sequence thereof having about 1 or 2 nucleoside substitutions, additions, or deletions, and the antisense strand comprises SEQ ID NO: 5477 or a nucleic acid sequence thereof having about 1 or 2 nucleoside substitutions, additions, or deletions. 395. The pharmaceutical composition of any one of embodiments 1-269, wherein the sense strand comprises SEQ ID NO: 3231, the first 19 nucleobases of SEQ ID NO: 3231, or a nucleic acid sequence thereof having about 1 or 2 nucleoside substitutions, additions, or deletions, and the antisense strand comprises SEQ ID NO: 3232, the first 19 nucleobases of SEQ ID NO: 3232, or a nucleic acid sequence thereof having about 1 or 2 nucleoside substitutions, additions, or deletions; or the sense strand comprises SEQ ID NO: 5478 or a nucleic acid sequence thereof having about 1 or 2 nucleoside substitutions, additions, or deletions, and the antisense strand comprises SEQ ID NO: 5479 or a nucleic acid sequence thereof having about 1 or 2 nucleoside substitutions, additions, or deletions. 396. The pharmaceutical composition of any one of embodiments 1-269, wherein the sense strand comprises SEQ ID NO: 3233, the first 19 nucleobases of SEQ ID NO: 3233, or a nucleic acid sequence thereof having about 1 or 2 nucleoside substitutions, additions, or deletions, and the antisense strand comprises SEQ ID NO: 3234, the first 19 nucleobases of SEQ ID NO: 3234, or a nucleic acid sequence thereof having about 1 or 2 nucleoside substitutions, additions, or deletions; or the sense strand comprises SEQ ID NO: 5480 or a nucleic acid sequence thereof having about 1 or 2 nucleoside substitutions, additions, or deletions, and the antisense strand comprises SEQ ID NO: 5481 or a nucleic acid sequence thereof having about 1 or 2 nucleoside substitutions, additions, or deletions. 397. The pharmaceutical composition of any one of embodiments 1-269, wherein the sense strand comprises SEQ ID NO: 3243, the first 19 nucleobases of SEQ ID NO: 3243, or a nucleic acid sequence thereof having about 1 or 2 nucleoside substitutions, additions, or deletions, and the antisense strand comprises SEQ ID NO: 3244, the first 19 nucleobases of SEQ ID NO: 3244, or a nucleic acid sequence thereof having about 1 or 2 nucleoside substitutions, additions, or deletions; or the sense strand comprises SEQ ID NO: 5482 or a nucleic acid sequence thereof having about 1 or 2 nucleoside substitutions, additions, or deletions, and the antisense strand comprises SEQ ID NO: 5483 or a nucleic acid sequence thereof having about 1 or 2 nucleoside substitutions, additions, or deletions. 398. The pharmaceutical composition of any one of embodiments 1-269, wherein the sense strand comprises SEQ ID NO: 3439, the first 19 nucleobases of SEQ ID NO: 3439, or a nucleic acid sequence thereof having about 1 or 2 nucleoside substitutions, additions, or deletions, and the antisense strand comprises SEQ ID NO: 3440, the first 19 nucleobases of SEQ ID NO: 3440, or a nucleic acid sequence thereof having about 1 or 2 nucleoside substitutions, additions, or deletions; or the sense strand comprises SEQ ID NO: 5484 or a nucleic acid sequence thereof having about 1 or 2 nucleoside substitutions, additions, or deletions, and the antisense strand comprises SEQ ID NO: 5485 or a nucleic acid sequence thereof having about 1 or 2 nucleoside substitutions, additions, or deletions. 399. The pharmaceutical composition of any one of embodiments 1-269, wherein the sense strand comprises SEQ ID NO: 3443, the first 19 nucleobases of SEQ ID NO: 3443, or a nucleic acid sequence thereof having about 1 or 2 nucleoside substitutions, additions, or deletions, and the antisense strand comprises SEQ ID NO: 3444, the first 19 nucleobases of SEQ ID NO: 3444, or a nucleic acid sequence thereof having about 1 or 2 nucleoside substitutions, additions, or deletions; or the sense strand comprises SEQ ID NO: 5486 or a nucleic acid sequence thereof having about 1 or 2 nucleoside substitutions, additions, or deletions, and the antisense strand comprises SEQ ID NO: 5487 or a nucleic acid sequence thereof having about 1 or 2 nucleoside substitutions, additions, or deletions. 400. The pharmaceutical composition of any one of embodiments 1-269, wherein the sense strand comprises SEQ ID NO: 3449, the first 19 nucleobases of SEQ ID NO: 3449, or a nucleic acid sequence thereof having about 1 or 2 nucleoside substitutions, additions, or deletions, and the antisense strand comprises SEQ ID NO: 3450, the first 19 nucleobases of SEQ ID NO: 3450, or a nucleic acid sequence thereof having about 1 or 2 nucleoside substitutions, additions, or deletions; or the sense strand comprises SEQ ID NO: 5488 or a nucleic acid sequence thereof having about 1 or 2 nucleoside substitutions, additions, or deletions, and the antisense strand comprises SEQ ID NO: 5489 or a nucleic acid sequence thereof having about 1 or 2 nucleoside substitutions, additions, or deletions. 401. The pharmaceutical composition of any one of embodiments 1-269, wherein the sense strand comprises SEQ ID NO: 3457, the first 19 nucleobases of SEQ ID NO: 3457, or a nucleic acid sequence thereof having about 1 or 2 nucleoside substitutions, additions, or deletions, and the antisense strand comprises SEQ ID NO: 3458, the first 19 nucleobases of SEQ ID NO: 3458, or a nucleic acid sequence thereof having about 1 or 2 nucleoside substitutions, additions, or deletions; or the sense strand comprises SEQ ID NO: 5490 or a nucleic acid sequence thereof having about 1 or 2 nucleoside substitutions, additions, or deletions, and the antisense strand comprises SEQ ID NO: 5491 or a nucleic acid sequence thereof having about 1 or 2 nucleoside substitutions, additions, or deletions. 402. The pharmaceutical composition of any one of embodiments 1-269, wherein the sense strand comprises SEQ ID NO: 3461, the first 19 nucleobases of SEQ ID NO: 3461, or a nucleic acid sequence thereof having about 1 or 2 nucleoside substitutions, additions, or deletions, and the antisense strand comprises SEQ ID NO: 3462, the first 19 nucleobases of SEQ ID NO: 3462, or a nucleic acid sequence thereof having about 1 or 2 nucleoside substitutions, additions, or deletions; or the sense strand comprises SEQ ID NO: 5492 or a nucleic acid sequence thereof having about 1 or 2 nucleoside substitutions, additions, or deletions, and the antisense strand comprises SEQ ID NO: 5493 or a nucleic acid sequence thereof having about 1 or 2 nucleoside substitutions, additions, or deletions. 403. The pharmaceutical composition of any one of embodiments 1-269, wherein the sense strand comprises SEQ ID NO: 3463, the first 19 nucleobases of SEQ ID NO: 3463, or a nucleic acid sequence thereof having about 1 or 2 nucleoside substitutions, additions, or deletions, and the antisense strand comprises SEQ ID NO: 3464, the first 19 nucleobases of SEQ ID NO: 3464, or a nucleic acid sequence thereof having about 1 or 2 nucleoside substitutions, additions, or deletions; or the sense strand comprises SEQ ID NO: 5494 or a nucleic acid sequence thereof having about 1 or 2 nucleoside substitutions, additions, or deletions, and the antisense strand comprises SEQ ID NO: 5495 or a nucleic acid sequence thereof having about 1 or 2 nucleoside substitutions, additions, or deletions. 404. The pharmaceutical composition of any one of embodiments 1-269, wherein the sense strand comprises SEQ ID NO: 3573, the first 19 nucleobases of SEQ ID NO: 3573, or a nucleic acid sequence thereof having about 1 or 2 nucleoside substitutions, additions, or deletions, and the antisense strand comprises SEQ ID NO: 3574, the first 19 nucleobases of SEQ ID NO: 3574, or a nucleic acid sequence thereof having about 1 or 2 nucleoside substitutions, additions, or deletions; or the sense strand comprises SEQ ID NO: 5496 or a nucleic acid sequence thereof having about 1 or 2 nucleoside substitutions, additions, or deletions, and the antisense strand comprises SEQ ID NO: 5497 or a nucleic acid sequence thereof having about 1 or 2 nucleoside substitutions, additions, or deletions. 405. The pharmaceutical composition of any one of embodiments 1-269, wherein the sense strand comprises SEQ ID NO: 3703, the first 19 nucleobases of SEQ ID NO: 3703, or a nucleic acid sequence thereof having about 1 or 2 nucleoside substitutions, additions, or deletions, and the antisense strand comprises SEQ ID NO: 3704, the first 19 nucleobases of SEQ ID NO: 3704, or a nucleic acid sequence thereof having about 1 or 2 nucleoside substitutions, additions, or deletions; or the sense strand comprises SEQ ID NO: 5498 or a nucleic acid sequence thereof having about 1 or 2 nucleoside substitutions, additions, or deletions, and the antisense strand comprises SEQ ID NO: 5499 or a nucleic acid sequence thereof having about 1 or 2 nucleoside substitutions, additions, or deletions. 406. The pharmaceutical composition of any one of embodiments 1-269, wherein the sense strand comprises SEQ ID NO: 3705, the first 19 nucleobases of SEQ ID NO: 3705, or a nucleic acid sequence thereof having about 1 or 2 nucleoside substitutions, additions, or deletions, and the antisense strand comprises SEQ ID NO: 3706, the first 19 nucleobases of SEQ ID NO: 3706, or a nucleic acid sequence thereof having about 1 or 2 nucleoside substitutions, additions, or deletions; or the sense strand comprises SEQ ID NO: 5500 or a nucleic acid sequence thereof having about 1 or 2 nucleoside substitutions, additions, or deletions, and the antisense strand comprises SEQ ID NO: 5501 or a nucleic acid sequence thereof having about 1 or 2 nucleoside substitutions, additions, or deletions. 407. The pharmaceutical composition of any one of embodiments 1-269, wherein the sense strand comprises SEQ ID NO: 3875, the first 19 nucleobases of SEQ ID NO: 3875, or a nucleic acid sequence thereof having about 1 or 2 nucleoside substitutions, additions, or deletions, and the antisense strand comprises SEQ ID NO: 3876, the first 19 nucleobases of SEQ ID NO: 3876, or a nucleic acid sequence thereof having about 1 or 2 nucleoside substitutions, additions, or deletions; or the sense strand comprises SEQ ID NO: 5502 or a nucleic acid sequence thereof having about 1 or 2 nucleoside substitutions, additions, or deletions, and the antisense strand comprises SEQ ID NO: 5503 or a nucleic acid sequence thereof having about 1 or 2 nucleoside substitutions, additions, or deletions. 408. The pharmaceutical composition of any one of embodiments 1-269, wherein the sense strand comprises SEQ ID NO: 3889, the first 19 nucleobases of SEQ ID NO: 3889, or a nucleic acid sequence thereof having about 1 or 2 nucleoside substitutions, additions, or deletions, and the antisense strand comprises SEQ ID NO: 3890, the first 19 nucleobases of SEQ ID NO: 3890, or a nucleic acid sequence thereof having about 1 or 2 nucleoside substitutions, additions, or deletions; or the sense strand comprises SEQ ID NO: 5504 or a nucleic acid sequence thereof having about 1 or 2 nucleoside substitutions, additions, or deletions, and the antisense strand comprises SEQ ID NO: 5505 or a nucleic acid sequence thereof having about 1 or 2 nucleoside substitutions, additions, or deletions. 409. The pharmaceutical composition of any one of embodiments 1-269, wherein the sense strand comprises SEQ ID NO: 3981, the first 19 nucleobases of SEQ ID NO: 3981, or a nucleic acid sequence thereof having about 1 or 2 nucleoside substitutions, additions, or deletions, and the antisense strand comprises SEQ ID NO: 3982, the first 19 nucleobases of SEQ ID NO: 3982, or a nucleic acid sequence thereof having about 1 or 2 nucleoside substitutions, additions, or deletions; or the sense strand comprises SEQ ID NO: 5506 or a nucleic acid sequence thereof having about 1 or 2 nucleoside substitutions, additions, or deletions, and the antisense strand comprises SEQ ID NO: 5507 or a nucleic acid sequence thereof having about 1 or 2 nucleoside substitutions, additions, or deletions. 410. The pharmaceutical composition of any one of embodiments 1-269, wherein the sense strand comprises SEQ ID NO: 4089, the first 19 nucleobases of SEQ ID NO: 4089, or a nucleic acid sequence thereof having about 1 or 2 nucleoside substitutions, additions, or deletions, and the antisense strand comprises SEQ ID NO: 4090, the first 19 nucleobases of SEQ ID NO: 4090, or a nucleic acid sequence thereof having about 1 or 2 nucleoside substitutions, additions, or deletions; or the sense strand comprises SEQ ID NO: 5508 or a nucleic acid sequence thereof having about 1 or 2 nucleoside substitutions, additions, or deletions, and the antisense strand comprises SEQ ID NO: 5509 or a nucleic acid sequence thereof having about 1 or 2 nucleoside substitutions, additions, or deletions. 411. The pharmaceutical composition of any one of embodiments 1-269, wherein the sense strand comprises SEQ ID NO: 4265, the first 19 nucleobases of SEQ ID NO: 4265, or a nucleic acid sequence thereof having about 1 or 2 nucleoside substitutions, additions, or deletions, and the antisense strand comprises SEQ ID NO: 4266, the first 19 nucleobases of SEQ ID NO: 4266, or a nucleic acid sequence thereof having about 1 or 2 nucleoside substitutions, additions, or deletions; or the sense strand comprises SEQ ID NO: 5510 or a nucleic acid sequence thereof having about 1 or 2 nucleoside substitutions, additions, or deletions, and the antisense strand comprises SEQ ID NO: 5511 or a nucleic acid sequence thereof having about 1 or 2 nucleoside substitutions, additions, or deletions. 412. The pharmaceutical composition of any one of embodiments 1-269, wherein the sense strand comprises SEQ ID NO: 4321, the first 19 nucleobases of SEQ ID NO: 4321, or a nucleic acid sequence thereof having about 1 or 2 nucleoside substitutions, additions, or deletions, and the antisense strand comprises SEQ ID NO: 4322, the first 19 nucleobases of SEQ ID NO: 4322, or a nucleic acid sequence thereof having about 1 or 2 nucleoside substitutions, additions, or deletions; or the sense strand comprises SEQ ID NO: 5512 or a nucleic acid sequence thereof having about 1 or 2 nucleoside substitutions, additions, or deletions, and the antisense strand comprises SEQ ID NO: 5513 or a nucleic acid sequence thereof having about 1 or 2 nucleoside substitutions, additions, or deletions. 413. The pharmaceutical composition of any one of embodiments 1-269, wherein the sense strand comprises SEQ ID NO: 4339, the first 19 nucleobases of SEQ ID NO: 4339, or a nucleic acid sequence thereof having about 1 or 2 nucleoside substitutions, additions, or deletions, and the antisense strand comprises SEQ ID NO: 4340, the first 19 nucleobases of SEQ ID NO: 4340, or a nucleic acid sequence thereof having about 1 or 2 nucleoside substitutions, additions, or deletions; or the sense strand comprises SEQ ID NO: 5514 or a nucleic acid sequence thereof having about 1 or 2 nucleoside substitutions, additions, or deletions, and the antisense strand comprises SEQ ID NO: 5515 or a nucleic acid sequence thereof having about 1 or 2 nucleoside substitutions, additions, or deletions. 414. The pharmaceutical composition of any one of embodiments 1-269, wherein the sense strand comprises SEQ ID NO: 4347, the first 19 nucleobases of SEQ ID NO: 4347, or a nucleic acid sequence thereof having about 1 or 2 nucleoside substitutions, additions, or deletions, and the antisense strand comprises SEQ ID NO: 4348, the first 19 nucleobases of SEQ ID NO: 4348, or a nucleic acid sequence thereof having about 1 or 2 nucleoside substitutions, additions, or deletions; or the sense strand comprises SEQ ID NO: 5516 or a nucleic acid sequence thereof having about 1 or 2 nucleoside substitutions, additions, or deletions, and the antisense strand comprises SEQ ID NO: 5517 or a nucleic acid sequence thereof having about 1 or 2 nucleoside substitutions, additions, or deletions. 415. The pharmaceutical composition of any one of embodiments 1-269, wherein the sense strand comprises SEQ ID NO: 4507, the first 19 nucleobases of SEQ ID NO: 4507, or a nucleic acid sequence thereof having about 1 or 2 nucleoside substitutions, additions, or deletions, and the antisense strand comprises SEQ ID NO: 4508, the first 19 nucleobases of SEQ ID NO: 4508, or a nucleic acid sequence thereof having about 1 or 2 nucleoside substitutions, additions, or deletions; or the sense strand comprises SEQ ID NO: 5518 or a nucleic acid sequence thereof having about 1 or 2 nucleoside substitutions, additions, or deletions, and the antisense strand comprises SEQ ID NO: 5519 or a nucleic acid sequence thereof having about 1 or 2 nucleoside substitutions, additions, or deletions. 416. The pharmaceutical composition of any one of embodiments 1-269, wherein the sense strand comprises SEQ ID NO: 53, the first 19 nucleobases of SEQ ID NO: 53, or a nucleic acid sequence thereof having about 1 or 2 nucleoside substitutions, additions, or deletions, and the antisense strand comprises SEQ ID NO: 54, the first 19 nucleobases of SEQ ID NO: 54, or a nucleic acid sequence thereof having about 1 or 2 nucleoside substitutions, additions, or deletions; or the sense strand comprises SEQ ID NO: 5520 or a nucleic acid sequence thereof having about 1 or 2 nucleoside substitutions, additions, or deletions, and the antisense strand comprises SEQ ID NO: 5521 or a nucleic acid sequence thereof having about 1 or 2 nucleoside substitutions, additions, or deletions. 417. The pharmaceutical composition of any one of embodiments 1-269, wherein the sense strand comprises SEQ ID NO: 2627, the first 19 nucleobases of SEQ ID NO: 2627, or a nucleic acid sequence thereof having about 1 or 2 nucleoside substitutions, additions, or deletions, and the antisense strand comprises SEQ ID NO: 2628, the first 19 nucleobases of SEQ ID NO: 2628, or a nucleic acid sequence thereof having about 1 or 2 nucleoside substitutions, additions, or deletions; or the sense strand comprises SEQ ID NO: 5522 or a nucleic acid sequence thereof having about 1 or 2 nucleoside substitutions, additions, or deletions, and the antisense strand comprises SEQ ID NO: 5523 or a nucleic acid sequence thereof having about 1 or 2 nucleoside substitutions, additions, or deletions. 418. The pharmaceutical composition of any one of embodiments 1-269, wherein the sense strand comprises SEQ ID NO: 2629, the first 19 nucleobases of SEQ ID NO: 2629, or a nucleic acid sequence thereof having about 1 or 2 nucleoside substitutions, additions, or deletions, and the antisense strand comprises SEQ ID NO: 2630, the first 19 nucleobases of SEQ ID NO: 2630, or a nucleic acid sequence thereof having about 1 or 2 nucleoside substitutions, additions, or deletions; or the sense strand comprises SEQ ID NO: 5524 or a nucleic acid sequence thereof having about 1 or 2 nucleoside substitutions, additions, or deletions, and the antisense strand comprises SEQ ID NO: 5525 or a nucleic acid sequence thereof having about 1 or 2 nucleoside substitutions, additions, or deletions. 419. The pharmaceutical composition of any one of embodiments 1-269, wherein the sense strand comprises SEQ ID NO: 3355, the first 19 nucleobases of SEQ ID NO: 3355, or a nucleic acid sequence thereof having about 1 or 2 nucleoside substitutions, additions, or deletions, and the antisense strand comprises SEQ ID NO: 3356, the first 19 nucleobases of SEQ ID NO: 3356, or a nucleic acid sequence thereof having about 1 or 2 nucleoside substitutions, additions, or deletions; or the sense strand comprises SEQ ID NO: 5526 or a nucleic acid sequence thereof having about 1 or 2 nucleoside substitutions, additions, or deletions, and the antisense strand comprises SEQ ID NO: 5527 or a nucleic acid sequence thereof having about 1 or 2 nucleoside substitutions, additions, or deletions. 420. The pharmaceutical composition of any one of embodiments 1-269, wherein the sense strand comprises SEQ ID NO: 3365, the first 19 nucleobases of SEQ ID NO: 3365, or a nucleic acid sequence thereof having about 1 or 2 nucleoside substitutions, additions, or deletions, and the antisense strand comprises SEQ ID NO: 3366, the first 19 nucleobases of SEQ ID NO: 3366, or a nucleic acid sequence thereof having about 1 or 2 nucleoside substitutions, additions, or deletions; or the sense strand comprises SEQ ID NO: 5528 or a nucleic acid sequence thereof having about 1 or 2 nucleoside substitutions, additions, or deletions, and the antisense strand comprises SEQ ID NO: 5529 or a nucleic acid sequence thereof having about 1 or 2 nucleoside substitutions, additions, or deletions. 421. The pharmaceutical composition of any one of embodiments 1-269, wherein the sense strand comprises SEQ ID NO: 87, the first 19 nucleobases of SEQ ID NO: 87, or a nucleic acid sequence thereof having about 1 or 2 nucleoside substitutions, additions, or deletions, and the antisense strand comprises SEQ ID NO: 88, the first 19 nucleobases of SEQ ID NO: 88, or a nucleic acid sequence thereof having about 1 or 2 nucleoside substitutions, additions, or deletions; or the sense strand comprises SEQ ID NO: 5530 or a nucleic acid sequence thereof having about 1 or 2 nucleoside substitutions, additions, or deletions, and the antisense strand comprises SEQ ID NO: 5531 or a nucleic acid sequence thereof having about 1 or 2 nucleoside substitutions, additions, or deletions. 422. The pharmaceutical composition of any one of embodiments 1-269, wherein the sense strand comprises SEQ ID NO: 2329, the first 19 nucleobases of SEQ ID NO: 2329, or a nucleic acid sequence thereof having about 1 or 2 nucleoside substitutions, additions, or deletions, and the antisense strand comprises SEQ ID NO: 2330, the first 19 nucleobases of SEQ ID NO: 2330, or a nucleic acid sequence thereof having about 1 or 2 nucleoside substitutions, additions, or deletions; or the sense strand comprises SEQ ID NO: 5532 or a nucleic acid sequence thereof having about 1 or 2 nucleoside substitutions, additions, or deletions, and the antisense strand comprises SEQ ID NO: 5533 or a nucleic acid sequence thereof having about 1 or 2 nucleoside substitutions, additions, or deletions. 423. The pharmaceutical composition of any one of embodiments 1-269, wherein the sense strand comprises SEQ ID NO: 2379, the first 19 nucleobases of SEQ ID NO: 2379, or a nucleic acid sequence thereof having about 1 or 2 nucleoside substitutions, additions, or deletions, and the antisense strand comprises SEQ ID NO: 2380, the first 19 nucleobases of SEQ ID NO: 2380, or a nucleic acid sequence thereof having about 1 or 2 nucleoside substitutions, additions, or deletions; or the sense strand comprises SEQ ID NO: 5534 or a nucleic acid sequence thereof having about 1 or 2 nucleoside substitutions, additions, or deletions, and the antisense strand comprises SEQ ID NO: 5535 or a nucleic acid sequence thereof having about 1 or 2 nucleoside substitutions, additions, or deletions. 424. The pharmaceutical composition of any one of embodiments 1-269, wherein the sense strand comprises SEQ ID NO: 2381, the first 19 nucleobases of SEQ ID NO: 2381, or a nucleic acid sequence thereof having about 1 or 2 nucleoside substitutions, additions, or deletions, and the antisense strand comprises SEQ ID NO: 2382, the first 19 nucleobases of SEQ ID NO: 2382, or a nucleic acid sequence thereof having about 1 or 2 nucleoside substitutions, additions, or deletions; or the sense strand comprises SEQ ID NO: 5536 or a nucleic acid sequence thereof having about 1 or 2 nucleoside substitutions, additions, or deletions, and the antisense strand comprises SEQ ID NO: 5537 or a nucleic acid sequence thereof having about 1 or 2 nucleoside substitutions, additions, or deletions. 425. The pharmaceutical composition of any one of embodiments 1-269, wherein the sense strand comprises SEQ ID NO: 2521, the first 19 nucleobases of SEQ ID NO: 2521, or a nucleic acid sequence thereof having about 1 or 2 nucleoside substitutions, additions, or deletions, and the antisense strand comprises SEQ ID NO: 2522, the first 19 nucleobases of SEQ ID NO: 2522, or a nucleic acid sequence thereof having about 1 or 2 nucleoside substitutions, additions, or deletions; or the sense strand comprises SEQ ID NO: 5538 or a nucleic acid sequence thereof having about 1 or 2 nucleoside substitutions, additions, or deletions, and the antisense strand comprises SEQ ID NO: 5539 or a nucleic acid sequence thereof having about 1 or 2 nucleoside substitutions, additions, or deletions. 426. The pharmaceutical composition of any one of embodiments 1-269, wherein the sense strand comprises SEQ ID NO: 2525, the first 19 nucleobases of SEQ ID NO: 2525, or a nucleic acid sequence thereof having about 1 or 2 nucleoside substitutions, additions, or deletions, and the antisense strand comprises SEQ ID NO: 2526, the first 19 nucleobases of SEQ ID NO: 2526, or a nucleic acid sequence thereof having about 1 or 2 nucleoside substitutions, additions, or deletions; or the sense strand comprises SEQ ID NO: 5540 or a nucleic acid sequence thereof having about 1 or 2 nucleoside substitutions, additions, or deletions, and the antisense strand comprises SEQ ID NO: 5541 or a nucleic acid sequence thereof having about 1 or 2 nucleoside substitutions, additions, or deletions. 427. The pharmaceutical composition of any one of embodiments 1-269, wherein the sense strand comprises SEQ ID NO: 2607, the first 19 nucleobases of SEQ ID NO: 2607, or a nucleic acid sequence thereof having about 1 or 2 nucleoside substitutions, additions, or deletions, and the antisense strand comprises SEQ ID NO: 2608, the first 19 nucleobases of SEQ ID NO: 2608, or a nucleic acid sequence thereof having about 1 or 2 nucleoside substitutions, additions, or deletions; or the sense strand comprises SEQ ID NO: 5542 or a nucleic acid sequence thereof having about 1 or 2 nucleoside substitutions, additions, or deletions, and the antisense strand comprises SEQ ID NO: 5543 or a nucleic acid sequence thereof having about 1 or 2 nucleoside substitutions, additions, or deletions. 428. The pharmaceutical composition of any one of embodiments 1-269, wherein the sense strand comprises SEQ ID NO: 3057, the first 19 nucleobases of SEQ ID NO: 3057, or a nucleic acid sequence thereof having about 1 or 2 nucleoside substitutions, additions, or deletions, and the antisense strand comprises SEQ ID NO: 3058, the first 19 nucleobases of SEQ ID NO: 3058, or a nucleic acid sequence thereof having about 1 or 2 nucleoside substitutions, additions, or deletions; or the sense strand comprises SEQ ID NO: 5544 or a nucleic acid sequence thereof having about 1 or 2 nucleoside substitutions, additions, or deletions, and the antisense strand comprises SEQ ID NO: 5545 or a nucleic acid sequence thereof having about 1 or 2 nucleoside substitutions, additions, or deletions. 429. The pharmaceutical composition of any one of embodiments 1-269, wherein the sense strand comprises SEQ ID NO: 3061, the first 19 nucleobases of SEQ ID NO: 3061, or a nucleic acid sequence thereof having about 1 or 2 nucleoside substitutions, additions, or deletions, and the antisense strand comprises SEQ ID NO: 3062, the first 19 nucleobases of SEQ ID NO: 3062, or a nucleic acid sequence thereof having about 1 or 2 nucleoside substitutions, additions, or deletions; or the sense strand comprises SEQ ID NO: 5546 or a nucleic acid sequence thereof having about 1 or 2 nucleoside substitutions, additions, or deletions, and the antisense strand comprises SEQ ID NO: 5547 or a nucleic acid sequence thereof having about 1 or 2 nucleoside substitutions, additions, or deletions. 430. The pharmaceutical composition of any one of embodiments 1-269, wherein the sense strand comprises SEQ ID NO: 3067, the first 19 nucleobases of SEQ ID NO: 3067, or a nucleic acid sequence thereof having about 1 or 2 nucleoside substitutions, additions, or deletions, and the antisense strand comprises SEQ ID NO: 3068, the first 19 nucleobases of SEQ ID NO: 3068, or a nucleic acid sequence thereof having about 1 or 2 nucleoside substitutions, additions, or deletions; or the sense strand comprises SEQ ID NO: 5548 or a nucleic acid sequence thereof having about 1 or 2 nucleoside substitutions, additions, or deletions, and the antisense strand comprises SEQ ID NO: 5549 or a nucleic acid sequence thereof having about 1 or 2 nucleoside substitutions, additions, or deletions. 431. The pharmaceutical composition of any one of embodiments 1-269, wherein the sense strand comprises SEQ ID NO: 3501, the first 19 nucleobases of SEQ ID NO: 3501, or a nucleic acid sequence thereof having about 1 or 2 nucleoside substitutions, additions, or deletions, and the antisense strand comprises SEQ ID NO: 3502, the first 19 nucleobases of SEQ ID NO: 3502, or a nucleic acid sequence thereof having about 1 or 2 nucleoside substitutions, additions, or deletions; or the sense strand comprises SEQ ID NO: 5550 or a nucleic acid sequence thereof having about 1 or 2 nucleoside substitutions, additions, or deletions, and the antisense strand comprises SEQ ID NO: 5551 or a nucleic acid sequence thereof having about 1 or 2 nucleoside substitutions, additions, or deletions. 432. The pharmaceutical composition of any one of embodiments 1-269, wherein the sense strand comprises SEQ ID NO: 3503, the first 19 nucleobases of SEQ ID NO: 3503, or a nucleic acid sequence thereof having about 1 or 2 nucleoside substitutions, additions, or deletions, and the antisense strand comprises SEQ ID NO: 3504, the first 19 nucleobases of SEQ ID NO: 3504, or a nucleic acid sequence thereof having about 1 or 2 nucleoside substitutions, additions, or deletions; or the sense strand comprises SEQ ID NO: 5552 or a nucleic acid sequence thereof having about 1 or 2 nucleoside substitutions, additions, or deletions, and the antisense strand comprises SEQ ID NO: 5553 or a nucleic acid sequence thereof having about 1 or 2 nucleoside substitutions, additions, or deletions. 433. The pharmaceutical composition of any one of embodiments 1-269, wherein the sense strand comprises SEQ ID NO: 3873, the first 19 nucleobases of SEQ ID NO: 3873, or a nucleic acid sequence thereof having about 1 or 2 nucleoside substitutions, additions, or deletions, and the antisense strand comprises SEQ ID NO: 3874, the first 19 nucleobases of SEQ ID NO: 3874, or a nucleic acid sequence thereof having about 1 or 2 nucleoside substitutions, additions, or deletions; or the sense strand comprises SEQ ID NO: 5554 or a nucleic acid sequence thereof having about 1 or 2 nucleoside substitutions, additions, or deletions, and the antisense strand comprises SEQ ID NO: 5555 or a nucleic acid sequence thereof having about 1 or 2 nucleoside substitutions, additions, or deletions. 434. The pharmaceutical composition of any one of embodiments 1-269, wherein the sense strand comprises SEQ ID NO: 4179, the first 19 nucleobases of SEQ ID NO: 4179, or a nucleic acid sequence thereof having about 1 or 2 nucleoside substitutions, additions, or deletions, and the antisense strand comprises SEQ ID NO: 4180, the first 19 nucleobases of SEQ ID NO: 4180, or a nucleic acid sequence thereof having about 1 or 2 nucleoside substitutions, additions, or deletions; or the sense strand comprises SEQ ID NO: 5556 or a nucleic acid sequence thereof having about 1 or 2 nucleoside substitutions, additions, or deletions, and the antisense strand comprises SEQ ID NO: 5557 or a nucleic acid sequence thereof having about 1 or 2 nucleoside substitutions, additions, or deletions. 435. The pharmaceutical composition of any one of embodiments 1-269, wherein the sense strand comprises SEQ ID NO: 3027, the first 19 nucleobases of SEQ ID NO: 3027, or a nucleic acid sequence thereof having about 1 or 2 nucleoside substitutions, additions, or deletions, and the antisense strand comprises SEQ ID NO: 3028, the first 19 nucleobases of SEQ ID NO: 3028, or a nucleic acid sequence thereof having about 1 or 2 nucleoside substitutions, additions, or deletions; or the sense strand comprises SEQ ID NO: 5558 or a nucleic acid sequence thereof having about 1 or 2 nucleoside substitutions, additions, or deletions, and the antisense strand comprises SEQ ID NO: 5559 or a nucleic acid sequence thereof having about 1 or 2 nucleoside substitutions, additions, or deletions. 436. The pharmaceutical composition of any one of embodiments 1-269, wherein the sense strand comprises SEQ ID NO: 3037, the first 19 nucleobases of SEQ ID NO: 3037, or a nucleic acid sequence thereof having about 1 or 2 nucleoside substitutions, additions, or deletions, and the antisense strand comprises SEQ ID NO: 3038, the first 19 nucleobases of SEQ ID NO: 3038, or a nucleic acid sequence thereof having about 1 or 2 nucleoside substitutions, additions, or deletions; or the sense strand comprises SEQ ID NO: 5560 or a nucleic acid sequence thereof having about 1 or 2 nucleoside substitutions, additions, or deletions, and the antisense strand comprises SEQ ID NO: 5561 or a nucleic acid sequence thereof having about 1 or 2 nucleoside substitutions, additions, or deletions. 437. The pharmaceutical composition of any one of embodiments 1-269, wherein the sense strand comprises SEQ ID NO: 3183, the first 19 nucleobases of SEQ ID NO: 3183, or a nucleic acid sequence thereof having about 1 or 2 nucleoside substitutions, additions, or deletions, and the antisense strand comprises SEQ ID NO: 3184, the first 19 nucleobases of SEQ ID NO: 3184, or a nucleic acid sequence thereof having about 1 or 2 nucleoside substitutions, additions, or deletions; or the sense strand comprises SEQ ID NO: 5562 or a nucleic acid sequence thereof having about 1 or 2 nucleoside substitutions, additions, or deletions, and the antisense strand comprises SEQ ID NO: 5563 or a nucleic acid sequence thereof having about 1 or 2 nucleoside substitutions, additions, or deletions. 438. The pharmaceutical composition of any one of embodiments 1-269, wherein the sense strand comprises SEQ ID NO: 4265, the first 19 nucleobases of SEQ ID NO: 4265, or a nucleic acid sequence thereof having about 1 or 2 nucleoside substitutions, additions, or deletions, and the antisense strand comprises SEQ ID NO: 4266, the first 19 nucleobases of SEQ ID NO: 4266, or a nucleic acid sequence thereof having about 1 or 2 nucleoside substitutions, additions, or deletions; or the sense strand comprises SEQ ID NO: 5564 or a nucleic acid sequence thereof having about 1 or 2 nucleoside substitutions, additions, or deletions, and the antisense strand comprises SEQ ID NO: 5565 or a nucleic acid sequence thereof having about 1 or 2 nucleoside substitutions, additions, or deletions. 439. The pharmaceutical composition of any one of embodiments 1-269, wherein the sense strand comprises SEQ ID NO: 2629, the first 19 nucleobases of SEQ ID NO: 2629, or a nucleic acid sequence thereof having about 1 or 2 nucleoside substitutions, additions, or deletions, and the antisense strand comprises SEQ ID NO: 2630, the first 19 nucleobases of SEQ ID NO: 2630, or a nucleic acid sequence thereof having about 1 or 2 nucleoside substitutions, additions, or deletions; or the sense strand comprises SEQ ID NO: 5566 or a nucleic acid sequence thereof having about 1 or 2 nucleoside substitutions, additions, or deletions, and the antisense strand comprises SEQ ID NO: 5567 or a nucleic acid sequence thereof having about 1 or 2 nucleoside substitutions, additions, or deletions. 440. The pharmaceutical composition of any one of embodiments 1-269, wherein the sense strand comprises one or more sequences selected from SEQ ID NOS: 1-5349, 5366-5971, 6000-6038, 6050-6074, or a nucleic acid sequence thereof having about 1 or 2 nucleoside substitutions, additions, or deletions. 441. The pharmaceutical composition of any one of embodiments 1-269, wherein the antisense strand comprises one or more sequences selected from SEQ ID NOS: 1-5349, 5366-5971, 6000-6038, 6050-6074, or a nucleic acid sequence thereof having about 1 or 2 nucleoside substitutions, additions, or deletions.

442. A pharmaceutical composition comprising a nucleic acid sequence comprising Formula IA: 5′ Z1-U-Z2-G-Z3-U-C-Z4-A-U-U-Z5-L 3′, wherein Z1 is 0, 3, 5, 11, or 13 nucleosides; Z2 is 1 nucleoside; Z3 is 0, 2, 5, 8, or 10 nucleosides; Z4 is 0, 2, 3, 5, 8, or 11 nucleosides; Z5 is 0 or 3 nucleosides; each “-” is independently a phosphodiester or modified internucleoside linkage; and L is an optional lipid; wherein the nucleic acid comprises a modified nucleoside and/or modified internucleoside linkage. 443. The pharmaceutical composition of embodiment 442, wherein Z1 is 0 nucleosides. 444. The pharmaceutical composition of embodiment 442, wherein Z1 is 3, 5, 11, or 13 nucleosides. 445. The pharmaceutical composition of embodiment 444, wherein Z1 comprises UGGUG, CAGGU, CAGGUUUGCCACU (SEQ ID NO: 6007), GCACCUUUUCA (SEQ ID NO: 6008), or AGC or a nucleic acid sequence thereof having about 1 or 2 nucleoside substitutions, additions, or deletions. 446. The pharmaceutical composition of any one of embodiments 442-445, wherein Z2 comprises C, G, or U. 447. The pharmaceutical composition of any one of embodiments 442-446, wherein Z3 is 0 nucleosides. 448. The pharmaceutical composition of any one of embodiments 442-446, wherein Z3 is 2, 5, 8, or 10 nucleosides. 449. The pharmaceutical composition of embodiment 448, wherein Z3 comprises UG, UGUCGAGAGA (SEQ ID NO: 6009), AGAGA, AGAGA, CCACUUUG, or AGCCU or a nucleic acid sequence thereof having about 1 or 2 nucleoside substitutions, additions, or deletions. 450. The pharmaceutical composition of any one of embodiments 442-449, wherein Z4 is 0 nucleosides. 451. The pharmaceutical composition of any one of embodiments 442-449, wherein Z4 is 2, 3, 5, 8, or 11 nucleosides. 452. The pharmaceutical composition of embodiment 451, wherein Z4 comprises GAGAGAUCACU (SEQ ID NO: 6010), ACU, ACUGAAAU, UC, or CUAAC or a nucleic acid sequence thereof having about 1 or 2 nucleoside substitutions, additions, or deletions. 453. The pharmaceutical composition of any one of embodiments 442-452, wherein Z5 represents 0 nucleosides. 454. The pharmaceutical composition of any one of embodiments 442-452, wherein Z5 represents 3 nucleosides. 455. The pharmaceutical composition of embodiment 454, wherein Z5 comprises UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG. 456. The pharmaceutical composition of any one of embodiments 442-455, wherein one or more of the Z5 nucleosides comprise a deoxyribose. 457. The pharmaceutical composition of embodiment 456, wherein the one or more is one. 458. The pharmaceutical composition of embodiment 456, wherein the one or more is two. 459. The pharmaceutical composition of embodiment 456, wherein the one or more is three. 460. The pharmaceutical composition of any one of embodiments 442-459, comprising the lipid. 461. The pharmaceutical composition of embodiment 460, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof. 462. The pharmaceutical composition of any one of embodiments 442-461, wherein Z1-U-Z2-G-Z3-U-C-Z4 comprises UGGUGUCGAGAGAUCACU (SEQ ID NO: 6011) or a nucleic acid sequence thereof having about 1 or 2 nucleoside substitutions, additions, or deletions. 463. The pharmaceutical composition of any one of embodiments 442-461, wherein Z1-U-Z2-G-Z3-U-C-Z4 comprises UCGAGAGAUCACUGAAAU (SEQ ID NO: 6012) or a nucleic acid sequence thereof having about 1 or 2 nucleoside substitutions, additions, or deletions. 464. The pharmaceutical composition of any one of embodiments 442-461, wherein Z1-U-Z2-G-Z3-U-C-Z4 comprises CAGGUUUGCCACUUUGUC (SEQ ID NO: 6013) or a nucleic acid sequence thereof having about 1 or 2 nucleoside substitutions, additions, or deletions. 465. The pharmaceutical composition of any one of embodiments 442-461, wherein Z1-U-Z2-G-Z3-U-C-Z4 comprises GCACCUUUUCAUGGUCUC (SEQ ID NO: 6014) or a nucleic acid sequence thereof having about 1 or 2 nucleoside substitutions, additions, or deletions. 466. The pharmaceutical composition of any one of embodiments 442-461, wherein Z1-U-Z2-G-Z3-U-C-Z4 comprises AGCUGGAGCCUUCCUAAC (SEQ ID NO: 15) or a nucleic acid sequence thereof having about 1 or 2 nucleoside substitutions, additions, or deletions. 467. The pharmaceutical composition of any one of embodiments 442-461, comprising mUsmGsmGmUfGmUfCfGfAmGmAmGmAmUmCmAmCmUmAsmUsmU (SEQ ID NO: 5558), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 468. The pharmaceutical composition of any one of embodiments 442-461, comprising mUsmCsmGmAfGmAfGfAfUmCmAmCmUmGmAmAmAmUmAsmUsmU (SEQ ID NO: 5560), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 469. The pharmaceutical composition of any one of embodiments 442-461, comprising mCsmAsmGmGfUmUfUfGfCmCmAmCmUmUmUmGmUmCmAsmUsmU (SEQ ID NO: 5562), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 470. The pharmaceutical composition of any one of embodiments 442-461, comprising mGsmCsmAmCfCmUfUfUfUmCmAmUmGmGmUmCmUmCmAsmUsmU (SEQ ID NO: 5564), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 471. The pharmaceutical composition of any one of embodiments 442-461, comprising mAsmGsmCmUfGmGfAfGfCmCmUmUmCmCmUmAmAmCmAsmUsmU (SEQ ID NO: 5566), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 472. The pharmaceutical composition of any one of embodiments 442-461, wherein the Z1-U-Z2-G-Z3-U-C-Z4-A-U-U sequence of Formula 1A is twenty-one nucleosides in length. 473. The pharmaceutical composition of any one of embodiments 442-461, wherein the antisense strand comprises the reserve complement of Z1-U-Z2-G-Z3-U-C-Z4.

474. A pharmaceutical composition comprising a nucleic acid sequence comprising Formula IB: 5′ U-Z6-G-A-Z7-C-Z8-A-Z9-U-U 3′, wherein Z6 represents 0, 2, 3, 5, 8, or 11 nucleosides; Z7 represents 0, 2, 5, 8, or 10 nucleosides; Z8 represents 1 nucleoside; Z9 represents 0, 3, 5, 11, or 13 nucleosides; and each “-” is independently a phosphodiester or modified internucleoside linkage. 475. The pharmaceutical composition of any one of embodiments 442-473 further comprising an anti-sense strand comprising Formula IB: 5′ U-Z6-G-A-Z7-C-Z8-A-Z9-U-U 3′, wherein Z6 represents 0, 2, 3, 5, 8, or 11 nucleosides; Z7 represents 0, 2, 5, 8, or 10 nucleosides; Z8 represents 1 nucleoside; Z9 represents 0, 3, 5, 11, or 13 nucleosides; and each “-” is independently a phosphodiester or modified internucleoside linkage. 476. The pharmaceutical composition of embodiment 474 or embodiment 475, wherein Z6 is 0 nucleosides. 477. The pharmaceutical composition of embodiment 474 or embodiment 475, wherein Z6 is 0, 2, 3, 5, 8, or 11 nucleosides. 478. The pharmaceutical composition of embodiment 477, wherein Z6 comprises AGU, AGUGAUCUCUC (SEQ ID NO: 6016), AUUUCAGU, GA, or GUUAG, or a nucleic acid sequence thereof having about 1 or 2 nucleoside substitutions, additions, or deletions. 479. The pharmaceutical composition of any one of embodiments 474-478, wherein Z7 is 0 nucleosides. 480. The pharmaceutical composition of any one of embodiments 474-478, wherein Z7 is 0, 2, 5, 8, or 10 nucleosides. 481. The pharmaceutical composition of embodiment 480, wherein Z7 comprises UCUCU, CA, UCUCU, CAAAGUGG, GA, or AGGCU, or a nucleic acid sequence thereof having about 1 or 2 nucleoside substitutions, additions, or deletions. 482. The pharmaceutical composition of any one of embodiments 474-481, wherein Z8 comprises C, G, or A. 483. The pharmaceutical composition of any one of embodiments 474-482, wherein Z9 is 0 nucleosides. 484. The pharmaceutical composition of any one of embodiments 474-482, wherein Z9 is 3, 5, 11, or 13 nucleosides. 485. The pharmaceutical composition of embodiment 484, wherein Z9 comprises CACCA, AGUGGCAAACCUG (SEQ ID NO: 6017), ACCUG, UGAAAAGGUGC (SEQ ID NO: 6018), or GCU, or a nucleic acid sequence thereof having about 1 or 2 nucleoside substitutions, additions, or deletions. 486. The pharmaceutical composition of any one of embodiments 474-485, wherein Z6-G-A-Z7-C-Z8-A-Z9 comprises AGUGAUCUCUCGACACCA (SEQ ID NO: 6019), or a nucleic acid sequence thereof having about 1 or 2 nucleoside substitutions, additions, or deletions. 487. The pharmaceutical composition of any one of embodiments 474-485, wherein Z6-G-A-Z7-C-Z8-A-Z9 comprises AUUUCAGUGAUCUCUCGA (SEQ ID NO: 6020), or a nucleic acid sequence thereof having about 1 or 2 nucleoside substitutions, additions, or deletions. 488. The pharmaceutical composition of any one of embodiments 474-485, wherein Z6-G-A-Z7-C-Z8-A-Z9 comprises GACAAAGUGGCAAACCUG (SEQ ID NO: 6021), or a nucleic acid sequence thereof having about 1 or 2 nucleoside substitutions, additions, or deletions. 489. The pharmaceutical composition of any one of embodiments 474-485, wherein Z6-G-A-Z7-C-Z8-A-Z9 comprises GAGACCAUGAAAAGGUGC (SEQ ID NO: 6022), or a nucleic acid sequence thereof having about 1 or 2 nucleoside substitutions, additions, or deletions. 490. The pharmaceutical composition of any one of embodiments 474-485, wherein Z6-G-A-Z7-C-Z8-A-Z9 comprises GUUAGGAAGGCUCCAGCU (SEQ ID NO: 6023), or a nucleic acid sequence thereof having about 1 or 2 nucleoside substitutions, additions, or deletions. 491. The pharmaceutical composition of any one of embodiments 474-485, wherein the antisense strand comprises mUsfAsmGmUmGfAmUmCmUmCmUmCmGfAmCfAmCmCmAsmUsmU (SEQ ID NO: 5559), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 492. The pharmaceutical composition of any one of embodiments 474-485, wherein the antisense strand comprises mUsfAsmUmUmUfCmAmGmUmGmAmUmCfUmCfUmCmGmAsmUsmU (SEQ ID NO: 5561), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 493. The pharmaceutical composition of any one of embodiments 474-485, wherein the antisense strand comprises mUsfGsmAmCmAfAmAmGmUmGmGmCmAfAmAfCmCmUmGsmUsmU (SEQ ID NO: 5563), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 494. The pharmaceutical composition of any one of embodiments 474-485, wherein the antisense strand comprises mUsfGsmAmGmAfCmCmAmUmGmAmAmAfAmGfGmUmGmCsmUsmU (SEQ ID NO: 5565), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 495. The pharmaceutical composition of any one of embodiments 474-485, wherein the antisense strand comprises mUsfGsmUmUmAfGmGmAmAmGmGmCmUfCmCfAmGmCmUsmUsmU (SEQ ID NO: 5567), or a nucleic acid sequence thereof having about 1 or 2 nucleoside substitutions, additions, or deletions; wherein “fN” is a 2′ fluoro-modified nucleoside, “mN” is a 2′ O-methyl modified nucleoside, “-” is a phosphodiester, and “s” is a phosphorothioate. 496. The pharmaceutical composition of any one of embodiments 474-485, wherein the U-Z6-G-A-Z7-C-Z8-A-Z9-U-U is 21 nucleosides in length.

497. A pharmaceutical composition comprising a sense strand and an antisense strand, wherein the sense strand comprises UGGUGUCGAGAGAUCACUG (SEQ ID NO: 5664) or a nucleic acid sequence thereof having about 1 or 2 nucleoside substitutions, additions, or deletions; and the antisense strand comprises CAGUGAUCUCUCGACACCA (SEQ ID NO: 5765) or a nucleic acid sequence thereof having about 1 or 2 nucleoside substitutions, additions, or deletions. 498. A pharmaceutical composition comprising a sense strand and an antisense strand, wherein the sense strand comprises UGGUGUCGAGAGAUCACUA (SEQ ID NO: 5866) or a nucleic acid sequence thereof having about 1 or 2 nucleoside substitutions, additions, or deletions; and the antisense strand comprises UAGUGAUCUCUCGACACCA (SEQ ID NO: 5967) or a nucleic acid sequence thereof having about 1 or 2 nucleoside substitutions, additions, or deletions. 499. A pharmaceutical composition comprising a sense strand and an antisense strand, wherein the sense strand comprises mUsmGsmGmUfGmUfCfGfAmGmAmGmAmUmCmAmCmUmAsmUsmU (SEQ ID NO: 5558) or a nucleic acid sequence thereof having about 1 or 2 nucleoside substitutions, additions, or deletions; and the antisense strand comprises mUsfAsmGmUmGfAmUmCmUmCmUmCmGfAmCfAmCmCmAsmUsmU (SEQ ID NO: 5559) or a nucleic acid sequence thereof having about 1 or 2 nucleoside substitutions, additions, or deletions.

500. A pharmaceutical composition comprising a sense strand and an antisense strand, wherein the sense strand comprises UCGAGAGAUCACUGAAAUC (SEQ ID NO: 5665) or a nucleic acid sequence thereof having about 1 or 2 nucleoside substitutions, additions, or deletions; and the antisense strand comprises GAUUUCAGUGAUCUCUCGA (SEQ ID NO: 5766) or a nucleic acid sequence thereof having about 1 or 2 nucleoside substitutions, additions, or deletions. 501. A pharmaceutical composition comprising a sense strand and an antisense strand, wherein the sense strand comprises UCGAGAGAUCACUGAAAUA (SEQ ID NO: 5867) or a nucleic acid sequence thereof having about 1 or 2 nucleoside substitutions, additions, or deletions; and the antisense strand comprises UAUUUCAGUGAUCUCUCGA (SEQ ID NO: 5968) or a nucleic acid sequence thereof having about 1 or 2 nucleoside substitutions, additions, or deletions. 502. A pharmaceutical composition comprising a sense strand and an antisense strand, wherein the sense strand comprises mUsmCsmGmAfGmAfGfAfUmCmAmCmUmGmAmAmAmUmAsmUsmU (SEQ ID NO: 5560) or a nucleic acid sequence thereof having about 1 or 2 nucleoside substitutions, additions, or deletions; and the antisense strand comprises mUsfAsmUmUmUfCmAmGmUmGmAmUmCfUmCfUmCmGmAsmUsmU (SEQ ID NO: 5561) or a nucleic acid sequence thereof having about 1 or 2 nucleoside substitutions, additions, or deletions. 503. A pharmaceutical composition comprising a sense strand and an antisense strand, wherein the sense strand comprises CAGGUUUGCCACUUUGUCA (SEQ ID NO: 5868) or a nucleic acid sequence thereof having about 1 or 2 nucleoside substitutions, additions, or deletions; and the antisense strand comprises UGACAAAGUGGCAAACCUG (SEQ ID NO: 5969) or a nucleic acid sequence thereof having about 1 or 2 nucleoside substitutions, additions, or deletions. 504. A pharmaceutical composition comprising a sense strand and an antisense strand, wherein the sense strand comprises CAGGUUUGCCACUUUGUCA (SEQ ID NO: 5868) or a nucleic acid sequence thereof having about 1 or 2 nucleoside substitutions, additions, or deletions; and the antisense strand comprises UGACAAAGUGGCAAACCUG (SEQ ID NO: 5969) or a nucleic acid sequence thereof having about 1 or 2 nucleoside substitutions, additions, or deletions. 505. A pharmaceutical composition comprising a sense strand and an antisense strand, wherein the sense strand comprises mCsmAsmGmGfUmUfUfGfCmCmAmCmUmUmUmGmUmCmAsmUsmU (SEQ ID NO: 5562) or a nucleic acid sequence thereof having about 1 or 2 nucleoside substitutions, additions, or deletions; and the antisense strand comprises mUsfGsmAmCmAfAmAmGmUmGmGmCmAfAmAfCmCmUmGsmUsmU (SEQ ID NO: 5563) or a nucleic acid sequence thereof having about 1 or 2 nucleoside substitutions, additions, or deletions. 506. A pharmaceutical composition comprising a sense strand and an antisense strand, wherein the sense strand comprises GCACCUUUUCAUGGUCUCU (SEQ ID NO: 5667) or a nucleic acid sequence thereof having about 1 or 2 nucleoside substitutions, additions, or deletions; and the antisense strand comprises AGAGACCAUGAAAAGGUGC (SEQ ID NO: 5768) or a nucleic acid sequence thereof having about 1 or 2 nucleoside substitutions, additions, or deletions. 507. A pharmaceutical composition comprising a sense strand and an antisense strand, wherein the sense strand comprises GCACCUUUUCAUGGUCUCA (SEQ ID NO: 5869) or a nucleic acid sequence thereof having about 1 or 2 nucleoside substitutions, additions, or deletions; and the antisense strand comprises UGAGACCAUGAAAAGGUGC (SEQ ID NO: 5970) or a nucleic acid sequence thereof having about 1 or 2 nucleoside substitutions, additions, or deletions. 508. A pharmaceutical composition comprising a sense strand and an antisense strand, wherein the sense strand comprises mGsmCsmAmCfCmUfUfUfUmCmAmUmGmGmUmCmUmCmAsmUsmU (SEQ ID NO: 5564) or a nucleic acid sequence thereof having about 1 or 2 nucleoside substitutions, additions, or deletions; and the antisense strand comprises mUsfGsmAmGmAfCmCmAmUmGmAmAmAfAmGfGmUmGmCsmUsmU (SEQ ID NO: 5565) or a nucleic acid sequence thereof having about 1 or 2 nucleoside substitutions, additions, or deletions. 509. A pharmaceutical composition comprising a sense strand and an antisense strand, wherein the sense strand comprises AGCUGGAGCCUUCCUAACC (SEQ ID NO: 5668) or a nucleic acid sequence thereof having about 1 or 2 nucleoside substitutions, additions, or deletions; and the antisense strand comprises GGUUAGGAAGGCUCCAGCU (SEQ ID NO: 5769) or a nucleic acid sequence thereof having about 1 or 2 nucleoside substitutions, additions, or deletions. 510. A pharmaceutical composition comprising a sense strand and an antisense strand, wherein the sense strand comprises AGCUGGAGCCUUCCUAACA (SEQ ID NO: 5870) or a nucleic acid sequence thereof having about 1 or 2 nucleoside substitutions, additions, or deletions; and the antisense strand comprises UGUUAGGAAGGCUCCAGCU (SEQ ID NO: 5971) or a nucleic acid sequence thereof having about 1 or 2 nucleoside substitutions, additions, or deletions. 511. A pharmaceutical composition comprising a sense strand and an antisense strand, wherein the sense strand comprises mAsmGsmCmUfGmGfAfGfCmCmUmUmCmCmUmAmAmCmAsmUsmU (SEQ ID NO: 5566) or a nucleic acid sequence thereof having about 1 or 2 nucleoside substitutions, additions, or deletions; and the antisense strand comprises mUsfGsmUmUmAfGmGmAmAmGmGmCmUfCmCfAmGmCmUsmUsmU (SEQ ID NO: 5567) or a nucleic acid sequence thereof having about 1 or 2 nucleoside substitutions, additions, or deletions.

512. A pharmaceutical composition comprising a sense strand and an antisense strand, wherein the sense strand comprises SEQ ID NO: 23, the first 19 nucleobases of SEQ ID NO: 23, or a nucleic acid sequence thereof having about 1 or 2 nucleoside substitutions, additions, or deletions, and the antisense strand comprises SEQ ID NO: 24, the first 19 nucleobases of SEQ ID NO: 24, or a nucleic acid sequence thereof having about 1 or 2 nucleoside substitutions, additions, or deletions; or the sense strand comprises SEQ ID NO: 5366 or a nucleic acid sequence thereof having about 1 or 2 nucleoside substitutions, additions, or deletions, and the antisense strand comprises SEQ ID NO: 5367 or a nucleic acid sequence thereof having about 1 or 2 nucleoside substitutions, additions, or deletions. 513. A pharmaceutical composition comprising a sense strand and an antisense strand, wherein the sense strand comprises SEQ ID NO: 31, the first 19 nucleobases of SEQ ID NO: 31, or a nucleic acid sequence thereof having about 1 or 2 nucleoside substitutions, additions, or deletions, and the antisense strand comprises SEQ ID NO: 32, the first 19 nucleobases of SEQ ID NO: 32, or a nucleic acid sequence thereof having about 1 or 2 nucleoside substitutions, additions, or deletions; or the sense strand comprises SEQ ID NO: 5368 or a nucleic acid sequence thereof having about 1 or 2 nucleoside substitutions, additions, or deletions, and the antisense strand comprises SEQ ID NO: 5369 or a nucleic acid sequence thereof having about 1 or 2 nucleoside substitutions, additions, or deletions. 514. A pharmaceutical composition comprising a sense strand and an antisense strand, wherein the sense strand comprises SEQ ID NO: 33, the first 19 nucleobases of SEQ ID NO: 33, or a nucleic acid sequence thereof having about 1 or 2 nucleoside substitutions, additions, or deletions, and the antisense strand comprises SEQ ID NO: 34, the first 19 nucleobases of SEQ ID NO: 34, or a nucleic acid sequence thereof having about 1 or 2 nucleoside substitutions, additions, or deletions; or the sense strand comprises SEQ ID NO: 5370 or a nucleic acid sequence thereof having about 1 or 2 nucleoside substitutions, additions, or deletions, and the antisense strand comprises SEQ ID NO: 5371 or a nucleic acid sequence thereof having about 1 or 2 nucleoside substitutions, additions, or deletions. 515. A pharmaceutical composition comprising a sense strand and an antisense strand, wherein the sense strand comprises SEQ ID NO: 37, the first 19 nucleobases of SEQ ID NO: 37, or a nucleic acid sequence thereof having about 1 or 2 nucleoside substitutions, additions, or deletions, and the antisense strand comprises SEQ ID NO: 38, the first 19 nucleobases of SEQ ID NO: 38, or a nucleic acid sequence thereof having about 1 or 2 nucleoside substitutions, additions, or deletions; or the sense strand comprises SEQ ID NO: 5372 or a nucleic acid sequence thereof having about 1 or 2 nucleoside substitutions, additions, or deletions, and the antisense strand comprises SEQ ID NO: 5373 or a nucleic acid sequence thereof having about 1 or 2 nucleoside substitutions, additions, or deletions. 516. A pharmaceutical composition comprising a sense strand and an antisense strand, wherein the sense strand comprises SEQ ID NO: 39, the first 19 nucleobases of SEQ ID NO: 39, or a nucleic acid sequence thereof having about 1 or 2 nucleoside substitutions, additions, or deletions, and the antisense strand comprises SEQ ID NO: 40, the first 19 nucleobases of SEQ ID NO: 40, or a nucleic acid sequence thereof having about 1 or 2 nucleoside substitutions, additions, or deletions; or the sense strand comprises SEQ ID NO: 5374 or a nucleic acid sequence thereof having about 1 or 2 nucleoside substitutions, additions, or deletions, and the antisense strand comprises SEQ ID NO: 5375 or a nucleic acid sequence thereof having about 1 or 2 nucleoside substitutions, additions, or deletions. 517. A pharmaceutical composition comprising a sense strand and an antisense strand, wherein the sense strand comprises SEQ ID NO: 41, the first 19 nucleobases of SEQ ID NO: 41, or a nucleic acid sequence thereof having about 1 or 2 nucleoside substitutions, additions, or deletions, and the antisense strand comprises SEQ ID NO: 42, the first 19 nucleobases of SEQ ID NO: 42, or a nucleic acid sequence thereof having about 1 or 2 nucleoside substitutions, additions, or deletions; or the sense strand comprises SEQ ID NO: 5376 or a nucleic acid sequence thereof having about 1 or 2 nucleoside substitutions, additions, or deletions, and the antisense strand comprises SEQ ID NO: 5377 or a nucleic acid sequence thereof having about 1 or 2 nucleoside substitutions, additions, or deletions. 518. A pharmaceutical composition comprising a sense strand and an antisense strand, wherein the sense strand comprises SEQ ID NO: 43, the first 19 nucleobases of SEQ ID NO: 43, or a nucleic acid sequence thereof having about 1 or 2 nucleoside substitutions, additions, or deletions, and the antisense strand comprises SEQ ID NO: 44, the first 19 nucleobases of SEQ ID NO: 44, or a nucleic acid sequence thereof having about 1 or 2 nucleoside substitutions, additions, or deletions; or the sense strand comprises SEQ ID NO: 5378 or a nucleic acid sequence thereof having about 1 or 2 nucleoside substitutions, additions, or deletions, and the antisense strand comprises SEQ ID NO: 5379 or a nucleic acid sequence thereof having about 1 or 2 nucleoside substitutions, additions, or deletions. 519. A pharmaceutical composition comprising a sense strand and an antisense strand, wherein the sense strand comprises SEQ ID NO: 51, the first 19 nucleobases of SEQ ID NO: 51, or a nucleic acid sequence thereof having about 1 or 2 nucleoside substitutions, additions, or deletions, and the antisense strand comprises SEQ ID NO: 52, the first 19 nucleobases of SEQ ID NO: 52, or a nucleic acid sequence thereof having about 1 or 2 nucleoside substitutions, additions, or deletions; or the sense strand comprises SEQ ID NO: 5380 or a nucleic acid sequence thereof having about 1 or 2 nucleoside substitutions, additions, or deletions, and the antisense strand comprises SEQ ID NO: 5381 or a nucleic acid sequence thereof having about 1 or 2 nucleoside substitutions, additions, or deletions. 520. A pharmaceutical composition comprising a sense strand and an antisense strand, wherein the sense strand comprises SEQ ID NO: 195, the first 19 nucleobases of SEQ ID NO: 195, or a nucleic acid sequence thereof having about 1 or 2 nucleoside substitutions, additions, or deletions, and the antisense strand comprises SEQ ID NO: 196, the first 19 nucleobases of SEQ ID NO: 196, or a nucleic acid sequence thereof having about 1 or 2 nucleoside substitutions, additions, or deletions; or the sense strand comprises SEQ ID NO: 5382 or a nucleic acid sequence thereof having about 1 or 2 nucleoside substitutions, additions, or deletions, and the antisense strand comprises SEQ ID NO: 5383 or a nucleic acid sequence thereof having about 1 or 2 nucleoside substitutions, additions, or deletions. 521. A pharmaceutical composition comprising a sense strand and an antisense strand, wherein the sense strand comprises SEQ ID NO: 197, the first 19 nucleobases of SEQ ID NO: 197, or a nucleic acid sequence thereof having about 1 or 2 nucleoside substitutions, additions, or deletions, and the antisense strand comprises SEQ ID NO: 198, the first 19 nucleobases of SEQ ID NO: 198, or a nucleic acid sequence thereof having about 1 or 2 nucleoside substitutions, additions, or deletions; or the sense strand comprises SEQ ID NO: 5384 or a nucleic acid sequence thereof having about 1 or 2 nucleoside substitutions, additions, or deletions, and the antisense strand comprises SEQ ID NO: 5385 or a nucleic acid sequence thereof having about 1 or 2 nucleoside substitutions, additions, or deletions. 522. A pharmaceutical composition comprising a sense strand and an antisense strand, wherein the sense strand comprises SEQ ID NO: 199, the first 19 nucleobases of SEQ ID NO: 199, or a nucleic acid sequence thereof having about 1 or 2 nucleoside substitutions, additions, or deletions, and the antisense strand comprises SEQ ID NO: 200, the first 19 nucleobases of SEQ ID NO: 200, or a nucleic acid sequence thereof having about 1 or 2 nucleoside substitutions, additions, or deletions; or the sense strand comprises SEQ ID NO: 5386 or a nucleic acid sequence thereof having about 1 or 2 nucleoside substitutions, additions, or deletions, and the antisense strand comprises SEQ ID NO: 5387 or a nucleic acid sequence thereof having about 1 or 2 nucleoside substitutions, additions, or deletions. 523. A pharmaceutical composition comprising a sense strand and an antisense strand, wherein the sense strand comprises SEQ ID NO: 251, the first 19 nucleobases of SEQ ID NO: 251, or a nucleic acid sequence thereof having about 1 or 2 nucleoside substitutions, additions, or deletions, and the antisense strand comprises SEQ ID NO: 252, the first 19 nucleobases of SEQ ID NO: 252, or a nucleic acid sequence thereof having about 1 or 2 nucleoside substitutions, additions, or deletions; or the sense strand comprises SEQ ID NO: 5388 or a nucleic acid sequence thereof having about 1 or 2 nucleoside substitutions, additions, or deletions, and the antisense strand comprises SEQ ID NO: 5389 or a nucleic acid sequence thereof having about 1 or 2 nucleoside substitutions, additions, or deletions. 524. A pharmaceutical composition comprising a sense strand and an antisense strand, wherein the sense strand comprises SEQ ID NO: 257, the first 19 nucleobases of SEQ ID NO: 257, or a nucleic acid sequence thereof having about 1 or 2 nucleoside substitutions, additions, or deletions, and the antisense strand comprises SEQ ID NO: 258, the first 19 nucleobases of SEQ ID NO: 258, or a nucleic acid sequence thereof having about 1 or 2 nucleoside substitutions, additions, or deletions; or the sense strand comprises SEQ ID NO: 5390 or a nucleic acid sequence thereof having about 1 or 2 nucleoside substitutions, additions, or deletions, and the antisense strand comprises SEQ ID NO: 5391 or a nucleic acid sequence thereof having about 1 or 2 nucleoside substitutions, additions, or deletions. 525. A pharmaceutical composition comprising a sense strand and an antisense strand, wherein the sense strand comprises SEQ ID NO: 263, the first 19 nucleobases of SEQ ID NO: 263, or a nucleic acid sequence thereof having about 1 or 2 nucleoside substitutions, additions, or deletions, and the antisense strand comprises SEQ ID NO: 264, the first 19 nucleobases of SEQ ID NO: 264, or a nucleic acid sequence thereof having about 1 or 2 nucleoside substitutions, additions, or deletions; or the sense strand comprises SEQ ID NO: 5392 or a nucleic acid sequence thereof having about 1 or 2 nucleoside substitutions, additions, or deletions, and the antisense strand comprises SEQ ID NO: 5393 or a nucleic acid sequence thereof having about 1 or 2 nucleoside substitutions, additions, or deletions. 526. A pharmaceutical composition comprising a sense strand and an antisense strand, wherein the sense strand comprises SEQ ID NO: 273, the first 19 nucleobases of SEQ ID NO: 273, or a nucleic acid sequence thereof having about 1 or 2 nucleoside substitutions, additions, or deletions, and the antisense strand comprises SEQ ID NO: 274, the first 19 nucleobases of SEQ ID NO: 274, or a nucleic acid sequence thereof having about 1 or 2 nucleoside substitutions, additions, or deletions; or the sense strand comprises SEQ ID NO: 5394 or a nucleic acid sequence thereof having about 1 or 2 nucleoside substitutions, additions, or deletions, and the antisense strand comprises SEQ ID NO: 5395 or a nucleic acid sequence thereof having about 1 or 2 nucleoside substitutions, additions, or deletions. 527. A pharmaceutical composition comprising a sense strand and an antisense strand, wherein the sense strand comprises SEQ ID NO: 275, the first 19 nucleobases of SEQ ID NO: 275, or a nucleic acid sequence thereof having about 1 or 2 nucleoside substitutions, additions, or deletions, and the antisense strand comprises SEQ ID NO: 276, the first 19 nucleobases of SEQ ID NO: 276, or a nucleic acid sequence thereof having about 1 or 2 nucleoside substitutions, additions, or deletions; or the sense strand comprises SEQ ID NO: 5396 or a nucleic acid sequence thereof having about 1 or 2 nucleoside substitutions, additions, or deletions, and the antisense strand comprises SEQ ID NO: 5397 or a nucleic acid sequence thereof having about 1 or 2 nucleoside substitutions, additions, or deletions. 528. A pharmaceutical composition comprising a sense strand and an antisense strand, wherein the sense strand comprises SEQ ID NO: 279, the first 19 nucleobases of SEQ ID NO: 279, or a nucleic acid sequence thereof having about 1 or 2 nucleoside substitutions, additions, or deletions, and the antisense strand comprises SEQ ID NO: 280, the first 19 nucleobases of SEQ ID NO: 280, or a nucleic acid sequence thereof having about 1 or 2 nucleoside substitutions, additions, or deletions; or the sense strand comprises SEQ ID NO: 5398 or a nucleic acid sequence thereof having about 1 or 2 nucleoside substitutions, additions, or deletions, and the antisense strand comprises SEQ ID NO: 5399 or a nucleic acid sequence thereof having about 1 or 2 nucleoside substitutions, additions, or deletions. 529. A pharmaceutical composition comprising a sense strand and an antisense strand, wherein the sense strand comprises SEQ ID NO: 2389, the first 19 nucleobases of SEQ ID NO: 2389, or a nucleic acid sequence thereof having about 1 or 2 nucleoside substitutions, additions, or deletions, and the antisense strand comprises SEQ ID NO: 2390, the first 19 nucleobases of SEQ ID NO: 2390, or a nucleic acid sequence thereof having about 1 or 2 nucleoside substitutions, additions, or deletions; or the sense strand comprises SEQ ID NO: 5400 or a nucleic acid sequence thereof having about 1 or 2 nucleoside substitutions, additions, or deletions, and the antisense strand comprises SEQ ID NO: 5401 or a nucleic acid sequence thereof having about 1 or 2 nucleoside substitutions, additions, or deletions. 530. A pharmaceutical composition comprising a sense strand and an antisense strand, wherein the sense strand comprises SEQ ID NO: 2391, the first 19 nucleobases of SEQ ID NO: 2391, or a nucleic acid sequence thereof having about 1 or 2 nucleoside substitutions, additions, or deletions, and the antisense strand comprises SEQ ID NO: 2392, the first 19 nucleobases of SEQ ID NO: 2392, or a nucleic acid sequence thereof having about 1 or 2 nucleoside substitutions, additions, or deletions; or the sense strand comprises SEQ ID NO: 5402 or a nucleic acid sequence thereof having about 1 or 2 nucleoside substitutions, additions, or deletions, and the antisense strand comprises SEQ ID NO: 5403 or a nucleic acid sequence thereof having about 1 or 2 nucleoside substitutions, additions, or deletions. 531. A pharmaceutical composition comprising a sense strand and an antisense strand, wherein the sense strand comprises SEQ ID NO: 2397, the first 19 nucleobases of SEQ ID NO: 2397, or a nucleic acid sequence thereof having about 1 or 2 nucleoside substitutions, additions, or deletions, and the antisense strand comprises SEQ ID NO: 2398, the first 19 nucleobases of SEQ ID NO: 2398, or a nucleic acid sequence thereof having about 1 or 2 nucleoside substitutions, additions, or deletions; or the sense strand comprises SEQ ID NO: 5404 or a nucleic acid sequence thereof having about 1 or 2 nucleoside substitutions, additions, or deletions, and the antisense strand comprises SEQ ID NO: 5405 or a nucleic acid sequence thereof having about 1 or 2 nucleoside substitutions, additions, or deletions. 532. A pharmaceutical composition comprising a sense strand and an antisense strand, wherein the sense strand comprises SEQ ID NO: 2399, the first 19 nucleobases of SEQ ID NO: 2399, or a nucleic acid sequence thereof having about 1 or 2 nucleoside substitutions, additions, or deletions, and the antisense strand comprises SEQ ID NO: 2400, the first 19 nucleobases of SEQ ID NO: 2400, or a nucleic acid sequence thereof having about 1 or 2 nucleoside substitutions, additions, or deletions; or the sense strand comprises SEQ ID NO: 5406 or a nucleic acid sequence thereof having about 1 or 2 nucleoside substitutions, additions, or deletions, and the antisense strand comprises SEQ ID NO: 5407 or a nucleic acid sequence thereof having about 1 or 2 nucleoside substitutions, additions, or deletions. 533. A pharmaceutical composition comprising a sense strand and an antisense strand, wherein the sense strand comprises SEQ ID NO: 2409, the first 19 nucleobases of SEQ ID NO: 2409, or a nucleic acid sequence thereof having about 1 or 2 nucleoside substitutions, additions, or deletions, and the antisense strand comprises SEQ ID NO: 2410, the first 19 nucleobases of SEQ ID NO: 2410, or a nucleic acid sequence thereof having about 1 or 2 nucleoside substitutions, additions, or deletions; or the sense strand comprises SEQ ID NO: 5408 or a nucleic acid sequence thereof having about 1 or 2 nucleoside substitutions, additions, or deletions, and the antisense strand comprises SEQ ID NO: 5409 or a nucleic acid sequence thereof having about 1 or 2 nucleoside substitutions, additions, or deletions. 534. A pharmaceutical composition comprising a sense strand and an antisense strand, wherein the sense strand comprises SEQ ID NO: 2415, the first 19 nucleobases of SEQ ID NO: 2415, or a nucleic acid sequence thereof having about 1 or 2 nucleoside substitutions, additions, or deletions, and the antisense strand comprises SEQ ID NO: 2416, the first 19 nucleobases of SEQ ID NO: 2416, or a nucleic acid sequence thereof having about 1 or 2 nucleoside substitutions, additions, or deletions; or the sense strand comprises SEQ ID NO: 5410 or a nucleic acid sequence thereof having about 1 or 2 nucleoside substitutions, additions, or deletions, and the antisense strand comprises SEQ ID NO: 5411 or a nucleic acid sequence thereof having about 1 or 2 nucleoside substitutions, additions, or deletions. 535. A pharmaceutical composition comprising a sense strand and an antisense strand, wherein the sense strand comprises SEQ ID NO: 2419, the first 19 nucleobases of SEQ ID NO: 2419, or a nucleic acid sequence thereof having about 1 or 2 nucleoside substitutions, additions, or deletions, and the antisense strand comprises SEQ ID NO: 2420, the first 19 nucleobases of SEQ ID NO: 2420, or a nucleic acid sequence thereof having about 1 or 2 nucleoside substitutions, additions, or deletions; or the sense strand comprises SEQ ID NO: 5412 or a nucleic acid sequence thereof having about 1 or 2 nucleoside substitutions, additions, or deletions, and the antisense strand comprises SEQ ID NO: 5413 or a nucleic acid sequence thereof having about 1 or 2 nucleoside substitutions, additions, or deletions. 536. A pharmaceutical composition comprising a sense strand and an antisense strand, wherein the sense strand comprises SEQ ID NO: 2421, the first 19 nucleobases of SEQ ID NO: 2421, or a nucleic acid sequence thereof having about 1 or 2 nucleoside substitutions, additions, or deletions, and the antisense strand comprises SEQ ID NO: 2422, the first 19 nucleobases of SEQ ID NO: 2422, or a nucleic acid sequence thereof having about 1 or 2 nucleoside substitutions, additions, or deletions; or the sense strand comprises SEQ ID NO: 5414 or a nucleic acid sequence thereof having about 1 or 2 nucleoside substitutions, additions, or deletions, and the antisense strand comprises SEQ ID NO: 5415 or a nucleic acid sequence thereof having about 1 or 2 nucleoside substitutions, additions, or deletions. 537. A pharmaceutical composition comprising a sense strand and an antisense strand, wherein the sense strand comprises SEQ ID NO: 2423, the first 19 nucleobases of SEQ ID NO: 2423, or a nucleic acid sequence thereof having about 1 or 2 nucleoside substitutions, additions, or deletions, and the antisense strand comprises SEQ ID NO: 2424, the first 19 nucleobases of SEQ ID NO: 2424, or a nucleic acid sequence thereof having about 1 or 2 nucleoside substitutions, additions, or deletions; or the sense strand comprises SEQ ID NO: 5416 or a nucleic acid sequence thereof having about 1 or 2 nucleoside substitutions, additions, or deletions, and the antisense strand comprises SEQ ID NO: 5417 or a nucleic acid sequence thereof having about 1 or 2 nucleoside substitutions, additions, or deletions. 538. A pharmaceutical composition comprising a sense strand and an antisense strand, wherein the sense strand comprises SEQ ID NO: 2425, the first 19 nucleobases of SEQ ID NO: 2425, or a nucleic acid sequence thereof having about 1 or 2 nucleoside substitutions, additions, or deletions, and the antisense strand comprises SEQ ID NO: 2426, the first 19 nucleobases of SEQ ID NO: 2426, or a nucleic acid sequence thereof having about 1 or 2 nucleoside substitutions, additions, or deletions; or the sense strand comprises SEQ ID NO: 5418 or a nucleic acid sequence thereof having about 1 or 2 nucleoside substitutions, additions, or deletions, and the antisense strand comprises SEQ ID NO: 5419 or a nucleic acid sequence thereof having about 1 or 2 nucleoside substitutions, additions, or deletions. 539. A pharmaceutical composition comprising a sense strand and an antisense strand, wherein the sense strand comprises SEQ ID NO: 2427, the first 19 nucleobases of SEQ ID NO: 2427, or a nucleic acid sequence thereof having about 1 or 2 nucleoside substitutions, additions, or deletions, and the antisense strand comprises SEQ ID NO: 2428, the first 19 nucleobases of SEQ ID NO: 2428, or a nucleic acid sequence thereof having about 1 or 2 nucleoside substitutions, additions, or deletions; or the sense strand comprises SEQ ID NO: 5420 or a nucleic acid sequence thereof having about 1 or 2 nucleoside substitutions, additions, or deletions, and the antisense strand comprises SEQ ID NO: 5421 or a nucleic acid sequence thereof having about 1 or 2 nucleoside substitutions, additions, or deletions. 540. A pharmaceutical composition comprising a sense strand and an antisense strand, wherein the sense strand comprises SEQ ID NO: 2431, the first 19 nucleobases of SEQ ID NO: 2431, or a nucleic acid sequence thereof having about 1 or 2 nucleoside substitutions, additions, or deletions, and the antisense strand comprises SEQ ID NO: 2432, the first 19 nucleobases of SEQ ID NO: 2432, or a nucleic acid sequence thereof having about 1 or 2 nucleoside substitutions, additions, or deletions; or the sense strand comprises SEQ ID NO: 5422 or a nucleic acid sequence thereof having about 1 or 2 nucleoside substitutions, additions, or deletions, and the antisense strand comprises SEQ ID NO: 5423 or a nucleic acid sequence thereof having about 1 or 2 nucleoside substitutions, additions, or deletions. 541. A pharmaceutical composition comprising a sense strand and an antisense strand, wherein the sense strand comprises SEQ ID NO: 2437, the first 19 nucleobases of SEQ ID NO: 2437, or a nucleic acid sequence thereof having about 1 or 2 nucleoside substitutions, additions, or deletions, and the antisense strand comprises SEQ ID NO: 2438, the first 19 nucleobases of SEQ ID NO: 2438, or a nucleic acid sequence thereof having about 1 or 2 nucleoside substitutions, additions, or deletions; or the sense strand comprises SEQ ID NO: 5424 or a nucleic acid sequence thereof having about 1 or 2 nucleoside substitutions, additions, or deletions, and the antisense strand comprises SEQ ID NO: 5425 or a nucleic acid sequence thereof having about 1 or 2 nucleoside substitutions, additions, or deletions. 542. A pharmaceutical composition comprising a sense strand and an antisense strand, wherein the sense strand comprises SEQ ID NO: 2439, the first 19 nucleobases of SEQ ID NO: 2439, or a nucleic acid sequence thereof having about 1 or 2 nucleoside substitutions, additions, or deletions, and the antisense strand comprises SEQ ID NO: 2440, the first 19 nucleobases of SEQ ID NO: 2440, or a nucleic acid sequence thereof having about 1 or 2 nucleoside substitutions, additions, or deletions; or the sense strand comprises SEQ ID NO: 5426 or a nucleic acid sequence thereof having about 1 or 2 nucleoside substitutions, additions, or deletions, and the antisense strand comprises SEQ ID NO: 5427 or a nucleic acid sequence thereof having about 1 or 2 nucleoside substitutions, additions, or deletions. 543. A pharmaceutical composition comprising a sense strand and an antisense strand, wherein the sense strand comprises SEQ ID NO: 2769, the first 19 nucleobases of SEQ ID NO: 2769, or a nucleic acid sequence thereof having about 1 or 2 nucleoside substitutions, additions, or deletions, and the antisense strand comprises SEQ ID NO: 2770, the first 19 nucleobases of SEQ ID NO: 2770, or a nucleic acid sequence thereof having about 1 or 2 nucleoside substitutions, additions, or deletions; or the sense strand comprises SEQ ID NO: 5428 or a nucleic acid sequence thereof having about 1 or 2 nucleoside substitutions, additions, or deletions, and the antisense strand comprises SEQ ID NO: 5429 or a nucleic acid sequence thereof having about 1 or 2 nucleoside substitutions, additions, or deletions. 544. A pharmaceutical composition comprising a sense strand and an antisense strand, wherein the sense strand comprises SEQ ID NO: 2771, the first 19 nucleobases of SEQ ID NO: 2771, or a nucleic acid sequence thereof having about 1 or 2 nucleoside substitutions, additions, or deletions, and the antisense strand comprises SEQ ID NO: 2772, the first 19 nucleobases of SEQ ID NO: 2772, or a nucleic acid sequence thereof having about 1 or 2 nucleoside substitutions, additions, or deletions; or the sense strand comprises SEQ ID NO: 5430 or a nucleic acid sequence thereof having about 1 or 2 nucleoside substitutions, additions, or deletions, and the antisense strand comprises SEQ ID NO: 5431 or a nucleic acid sequence thereof having about 1 or 2 nucleoside substitutions, additions, or deletions. 545. A pharmaceutical composition comprising a sense strand and an antisense strand, wherein the sense strand comprises SEQ ID NO: 2773, the first 19 nucleobases of SEQ ID NO: 2773, or a nucleic acid sequence thereof having about 1 or 2 nucleoside substitutions, additions, or deletions, and the antisense strand comprises SEQ ID NO: 2774, the first 19 nucleobases of SEQ ID NO: 2774, or a nucleic acid sequence thereof having about 1 or 2 nucleoside substitutions, additions, or deletions; or the sense strand comprises SEQ ID NO: 5432 or a nucleic acid sequence thereof having about 1 or 2 nucleoside substitutions, additions, or deletions, and the antisense strand comprises SEQ ID NO: 5433 or a nucleic acid sequence thereof having about 1 or 2 nucleoside substitutions, additions, or deletions. 546. A pharmaceutical composition comprising a sense strand and an antisense strand, wherein the sense strand comprises SEQ ID NO: 2881, the first 19 nucleobases of SEQ ID NO: 2881, or a nucleic acid sequence thereof having about 1 or 2 nucleoside substitutions, additions, or deletions, and the antisense strand comprises SEQ ID NO: 2882, the first 19 nucleobases of SEQ ID NO: 2882, or a nucleic acid sequence thereof having about 1 or 2 nucleoside substitutions, additions, or deletions; or the sense strand comprises SEQ ID NO: 5434 or a nucleic acid sequence thereof having about 1 or 2 nucleoside substitutions, additions, or deletions, and the antisense strand comprises SEQ ID NO: 5435 or a nucleic acid sequence thereof having about 1 or 2 nucleoside substitutions, additions, or deletions. 547. A pharmaceutical composition comprising a sense strand and an antisense strand, wherein the sense strand comprises SEQ ID NO: 2899, the first 19 nucleobases of SEQ ID NO: 2899, or a nucleic acid sequence thereof having about 1 or 2 nucleoside substitutions, additions, or deletions, and the antisense strand comprises SEQ ID NO: 2900, the first 19 nucleobases of SEQ ID NO: 2900, or a nucleic acid sequence thereof having about 1 or 2 nucleoside substitutions, additions, or deletions; or the sense strand comprises SEQ ID NO: 5436 or a nucleic acid sequence thereof having about 1 or 2 nucleoside substitutions, additions, or deletions, and the antisense strand comprises SEQ ID NO: 5437 or a nucleic acid sequence thereof having about 1 or 2 nucleoside substitutions, additions, or deletions. 548. A pharmaceutical composition comprising a sense strand and an antisense strand, wherein the sense strand comprises SEQ ID NO: 2903, the first 19 nucleobases of SEQ ID NO: 2903, or a nucleic acid sequence thereof having about 1 or 2 nucleoside substitutions, additions, or deletions, and the antisense strand comprises SEQ ID NO: 2904, the first 19 nucleobases of SEQ ID NO: 2904, or a nucleic acid sequence thereof having about 1 or 2 nucleoside substitutions, additions, or deletions; or the sense strand comprises SEQ ID NO: 5438 or a nucleic acid sequence thereof having about 1 or 2 nucleoside substitutions, additions, or deletions, and the antisense strand comprises SEQ ID NO: 5439 or a nucleic acid sequence thereof having about 1 or 2 nucleoside substitutions, additions, or deletions. 549. A pharmaceutical composition comprising a sense strand and an antisense strand, wherein the sense strand comprises SEQ ID NO: 2905, the first 19 nucleobases of SEQ ID NO: 2905, or a nucleic acid sequence thereof having about 1 or 2 nucleoside substitutions, additions, or deletions, and the antisense strand comprises SEQ ID NO: 2906, the first 19 nucleobases of SEQ ID NO: 2906, or a nucleic acid sequence thereof having about 1 or 2 nucleoside substitutions, additions, or deletions; or the sense strand comprises SEQ ID NO: 5440 or a nucleic acid sequence thereof having about 1 or 2 nucleoside substitutions, additions, or deletions, and the antisense strand comprises SEQ ID NO: 5441 or a nucleic acid sequence thereof having about 1 or 2 nucleoside substitutions, additions, or deletions. 550. A pharmaceutical composition comprising a sense strand and an antisense strand, wherein the sense strand comprises SEQ ID NO: 2909, the first 19 nucleobases of SEQ ID NO: 2909, or a nucleic acid sequence thereof having about 1 or 2 nucleoside substitutions, additions, or deletions, and the antisense strand comprises SEQ ID NO: 2910, the first 19 nucleobases of SEQ ID NO: 2910, or a nucleic acid sequence thereof having about 1 or 2 nucleoside substitutions, additions, or deletions; or the sense strand comprises SEQ ID NO: 5442 or a nucleic acid sequence thereof having about 1 or 2 nucleoside substitutions, additions, or deletions, and the antisense strand comprises SEQ ID NO: 5443 or a nucleic acid sequence thereof having about 1 or 2 nucleoside substitutions, additions, or deletions. 551. A pharmaceutical composition comprising a sense strand and an antisense strand, wherein the sense strand comprises SEQ ID NO: 2913, the first 19 nucleobases of SEQ ID NO: 2913, or a nucleic acid sequence thereof having about 1 or 2 nucleoside substitutions, additions, or deletions, and the antisense strand comprises SEQ ID NO: 2914, the first 19 nucleobases of SEQ ID NO: 2914, or a nucleic acid sequence thereof having about 1 or 2 nucleoside substitutions, additions, or deletions; or the sense strand comprises SEQ ID NO: 5444 or a nucleic acid sequence thereof having about 1 or 2 nucleoside substitutions, additions, or deletions, and the antisense strand comprises SEQ ID NO: 5445 or a nucleic acid sequence thereof having about 1 or 2 nucleoside substitutions, additions, or deletions. 552. A pharmaceutical composition comprising a sense strand and an antisense strand, wherein the sense strand comprises SEQ ID NO: 2915, the first 19 nucleobases of SEQ ID NO: 2915, or a nucleic acid sequence thereof having about 1 or 2 nucleoside substitutions, additions, or deletions, and the antisense strand comprises SEQ ID NO: 2916, the first 19 nucleobases of SEQ ID NO: 2916, or a nucleic acid sequence thereof having about 1 or 2 nucleoside substitutions, additions, or deletions; or the sense strand comprises SEQ ID NO: 5446 or a nucleic acid sequence thereof having about 1 or 2 nucleoside substitutions, additions, or deletions, and the antisense strand comprises SEQ ID NO: 5447 or a nucleic acid sequence thereof having about 1 or 2 nucleoside substitutions, additions, or deletions. 553. A pharmaceutical composition comprising a sense strand and an antisense strand, wherein the sense strand comprises SEQ ID NO: 3013, the first 19 nucleobases of SEQ ID NO: 3013, or a nucleic acid sequence thereof having about 1 or 2 nucleoside substitutions, additions, or deletions, and the antisense strand comprises SEQ ID NO: 3014, the first 19 nucleobases of SEQ ID NO: 3014, or a nucleic acid sequence thereof having about 1 or 2 nucleoside substitutions, additions, or deletions; or the sense strand comprises SEQ ID NO: 5448 or a nucleic acid sequence thereof having about 1 or 2 nucleoside substitutions, additions, or deletions, and the antisense strand comprises SEQ ID NO: 5449 or a nucleic acid sequence thereof having about 1 or 2 nucleoside substitutions, additions, or deletions. 554. A pharmaceutical composition comprising a sense strand and an antisense strand, wherein the sense strand comprises SEQ ID NO: 3015, the first 19 nucleobases of SEQ ID NO: 3015, or a nucleic acid sequence thereof having about 1 or 2 nucleoside substitutions, additions, or deletions, and the antisense strand comprises SEQ ID NO: 3016, the first 19 nucleobases of SEQ ID NO: 3016, or a nucleic acid sequence thereof having about 1 or 2 nucleoside substitutions, additions, or deletions; or the sense strand comprises SEQ ID NO: 5450 or a nucleic acid sequence thereof having about 1 or 2 nucleoside substitutions, additions, or deletions, and the antisense strand comprises SEQ ID NO: 5451 or a nucleic acid sequence thereof having about 1 or 2 nucleoside substitutions, additions, or deletions. 555. A pharmaceutical composition comprising a sense strand and an antisense strand, wherein the sense strand comprises SEQ ID NO: 3017, the first 19 nucleobases of SEQ ID NO: 3017, or a nucleic acid sequence thereof having about 1 or 2 nucleoside substitutions, additions, or deletions, and the antisense strand comprises SEQ ID NO: 3018, the first 19 nucleobases of SEQ ID NO: 3018, or a nucleic acid sequence thereof having about 1 or 2 nucleoside substitutions, additions, or deletions; or the sense strand comprises SEQ ID NO: 5452 or a nucleic acid sequence thereof having about 1 or 2 nucleoside substitutions, additions, or deletions, and the antisense strand comprises SEQ ID NO: 5453 or a nucleic acid sequence thereof having about 1 or 2 nucleoside substitutions, additions, or deletions. 556. A pharmaceutical composition comprising a sense strand and an antisense strand, wherein the sense strand comprises SEQ ID NO: 3019, the first 19 nucleobases of SEQ ID NO: 3019, or a nucleic acid sequence thereof having about 1 or 2 nucleoside substitutions, additions, or deletions, and the antisense strand comprises SEQ ID NO: 3020, the first 19 nucleobases of SEQ ID NO: 3020, or a nucleic acid sequence thereof having about 1 or 2 nucleoside substitutions, additions, or deletions; or the sense strand comprises SEQ ID NO: 5454 or a nucleic acid sequence thereof having about 1 or 2 nucleoside substitutions, additions, or deletions, and the antisense strand comprises SEQ ID NO: 5455 or a nucleic acid sequence thereof having about 1 or 2 nucleoside substitutions, additions, or deletions. 557. A pharmaceutical composition comprising a sense strand and an antisense strand, wherein the sense strand comprises SEQ ID NO: 3027, the first 19 nucleobases of SEQ ID NO: 3027, or a nucleic acid sequence thereof having about 1 or 2 nucleoside substitutions, additions, or deletions, and the antisense strand comprises SEQ ID NO: 3028, the first 19 nucleobases of SEQ ID NO: 3028, or a nucleic acid sequence thereof having about 1 or 2 nucleoside substitutions, additions, or deletions; or the sense strand comprises SEQ ID NO: 5456 or a nucleic acid sequence thereof having about 1 or 2 nucleoside substitutions, additions, or deletions, and the antisense strand comprises SEQ ID NO: 5457 or a nucleic acid sequence thereof having about 1 or 2 nucleoside substitutions, additions, or deletions. 558. A pharmaceutical composition comprising a sense strand and an antisense strand, wherein the sense strand comprises SEQ ID NO: 3029, the first 19 nucleobases of SEQ ID NO: 3029, or a nucleic acid sequence thereof having about 1 or 2 nucleoside substitutions, additions, or deletions, and the antisense strand comprises SEQ ID NO: 3030, the first 19 nucleobases of SEQ ID NO: 3030, or a nucleic acid sequence thereof having about 1 or 2 nucleoside substitutions, additions, or deletions; or the sense strand comprises SEQ ID NO: 5458 or a nucleic acid sequence thereof having about 1 or 2 nucleoside substitutions, additions, or deletions, and the antisense strand comprises SEQ ID NO: 5459 or a nucleic acid sequence thereof having about 1 or 2 nucleoside substitutions, additions, or deletions. 559. A pharmaceutical composition comprising a sense strand and an antisense strand, wherein the sense strand comprises SEQ ID NO: 3037, the first 19 nucleobases of SEQ ID NO: 3037, or a nucleic acid sequence thereof having about 1 or 2 nucleoside substitutions, additions, or deletions, and the antisense strand comprises SEQ ID NO: 3038, the first 19 nucleobases of SEQ ID NO: 3038, or a nucleic acid sequence thereof having about 1 or 2 nucleoside substitutions, additions, or deletions; or the sense strand comprises SEQ ID NO: 5460 or a nucleic acid sequence thereof having about 1 or 2 nucleoside substitutions, additions, or deletions, and the antisense strand comprises SEQ ID NO: 5461 or a nucleic acid sequence thereof having about 1 or 2 nucleoside substitutions, additions, or deletions. 560. A pharmaceutical composition comprising a sense strand and an antisense strand, wherein the sense strand comprises SEQ ID NO: 3039, the first 19 nucleobases of SEQ ID NO: 3039, or a nucleic acid sequence thereof having about 1 or 2 nucleoside substitutions, additions, or deletions, and the antisense strand comprises SEQ ID NO: 3040, the first 19 nucleobases of SEQ ID NO: 3040, or a nucleic acid sequence thereof having about 1 or 2 nucleoside substitutions, additions, or deletions; or the sense strand comprises SEQ ID NO: 5462 or a nucleic acid sequence thereof having about 1 or 2 nucleoside substitutions, additions, or deletions, and the antisense strand comprises SEQ ID NO: 5463 or a nucleic acid sequence thereof having about 1 or 2 nucleoside substitutions, additions, or deletions. 561. A pharmaceutical composition comprising a sense strand and an antisense strand, wherein the sense strand comprises SEQ ID NO: 3041, the first 19 nucleobases of SEQ ID NO: 3041, or a nucleic acid sequence thereof having about 1 or 2 nucleoside substitutions, additions, or deletions, and the antisense strand comprises SEQ ID NO: 3042, the first 19 nucleobases of SEQ ID NO: 3042, or a nucleic acid sequence thereof having about 1 or 2 nucleoside substitutions, additions, or deletions; or the sense strand comprises SEQ ID NO: 5464 or a nucleic acid sequence thereof having about 1 or 2 nucleoside substitutions, additions, or deletions, and the antisense strand comprises SEQ ID NO: 5465 or a nucleic acid sequence thereof having about 1 or 2 nucleoside substitutions, additions, or deletions. 562. A pharmaceutical composition comprising a sense strand and an antisense strand, wherein the sense strand comprises SEQ ID NO: 3047, the first 19 nucleobases of SEQ ID NO: 3047, or a nucleic acid sequence thereof having about 1 or 2 nucleoside substitutions, additions, or deletions, and the antisense strand comprises SEQ ID NO: 3048, the first 19 nucleobases of SEQ ID NO: 3048, or a nucleic acid sequence thereof having about 1 or 2 nucleoside substitutions, additions, or deletions; or the sense strand comprises SEQ ID NO: 5466 or a nucleic acid sequence thereof having about 1 or 2 nucleoside substitutions, additions, or deletions, and the antisense strand comprises SEQ ID NO: 5467 or a nucleic acid sequence thereof having about 1 or 2 nucleoside substitutions, additions, or deletions. 563. A pharmaceutical composition comprising a sense strand and an antisense strand, wherein the sense strand comprises SEQ ID NO: 3053, the first 19 nucleobases of SEQ ID NO: 3053, or a nucleic acid sequence thereof having about 1 or 2 nucleoside substitutions, additions, or deletions, and the antisense strand comprises SEQ ID NO: 3054, the first 19 nucleobases of SEQ ID NO: 3054, or a nucleic acid sequence thereof having about 1 or 2 nucleoside substitutions, additions, or deletions; or the sense strand comprises SEQ ID NO: 5468 or a nucleic acid sequence thereof having about 1 or 2 nucleoside substitutions, additions, or deletions, and the antisense strand comprises SEQ ID NO: 5469 or a nucleic acid sequence thereof having about 1 or 2 nucleoside substitutions, additions, or deletions. 564. A pharmaceutical composition comprising a sense strand and an antisense strand, wherein the sense strand comprises SEQ ID NO: 3055, the first 19 nucleobases of SEQ ID NO: 3055, or a nucleic acid sequence thereof having about 1 or 2 nucleoside substitutions, additions, or deletions, and the antisense strand comprises SEQ ID NO: 3056, the first 19 nucleobases of SEQ ID NO: 3056, or a nucleic acid sequence thereof having about 1 or 2 nucleoside substitutions, additions, or deletions; or the sense strand comprises SEQ ID NO: 5470 or a nucleic acid sequence thereof having about 1 or 2 nucleoside substitutions, additions, or deletions, and the antisense strand comprises SEQ ID NO: 5471 or a nucleic acid sequence thereof having about 1 or 2 nucleoside substitutions, additions, or deletions. 565. A pharmaceutical composition comprising a sense strand and an antisense strand, wherein the sense strand comprises SEQ ID NO: 3063, the first 19 nucleobases of SEQ ID NO: 3063, or a nucleic acid sequence thereof having about 1 or 2 nucleoside substitutions, additions, or deletions, and the antisense strand comprises SEQ ID NO: 3064, the first 19 nucleobases of SEQ ID NO: 3064, or a nucleic acid sequence thereof having about 1 or 2 nucleoside substitutions, additions, or deletions; or the sense strand comprises SEQ ID NO: 5472 or a nucleic acid sequence thereof having about 1 or 2 nucleoside substitutions, additions, or deletions, and the antisense strand comprises SEQ ID NO: 5473 or a nucleic acid sequence thereof having about 1 or 2 nucleoside substitutions, additions, or deletions. 566. A pharmaceutical composition comprising a sense strand and an antisense strand, wherein the sense strand comprises SEQ ID NO: 3065, the first 19 nucleobases of SEQ ID NO: 3065, or a nucleic acid sequence thereof having about 1 or 2 nucleoside substitutions, additions, or deletions, and the antisense strand comprises SEQ ID NO: 3066, the first 19 nucleobases of SEQ ID NO: 3066, or a nucleic acid sequence thereof having about 1 or 2 nucleoside substitutions, additions, or deletions; or the sense strand comprises SEQ ID NO: 5474 or a nucleic acid sequence thereof having about 1 or 2 nucleoside substitutions, additions, or deletions, and the antisense strand comprises SEQ ID NO: 5475 or a nucleic acid sequence thereof having about 1 or 2 nucleoside substitutions, additions, or deletions. 567. A pharmaceutical composition comprising a sense strand and an antisense strand, wherein the sense strand comprises SEQ ID NO: 3183, the first 19 nucleobases of SEQ ID NO: 3183, or a nucleic acid sequence thereof having about 1 or 2 nucleoside substitutions, additions, or deletions, and the antisense strand comprises SEQ ID NO: 3184, the first 19 nucleobases of SEQ ID NO: 3184, or a nucleic acid sequence thereof having about 1 or 2 nucleoside substitutions, additions, or deletions; or the sense strand comprises SEQ ID NO: 5476 or a nucleic acid sequence thereof having about 1 or 2 nucleoside substitutions, additions, or deletions, and the antisense strand comprises SEQ ID NO: 5477 or a nucleic acid sequence thereof having about 1 or 2 nucleoside substitutions, additions, or deletions. 568. A pharmaceutical composition comprising a sense strand and an antisense strand, wherein the sense strand comprises SEQ ID NO: 3231, the first 19 nucleobases of SEQ ID NO: 3231, or a nucleic acid sequence thereof having about 1 or 2 nucleoside substitutions, additions, or deletions, and the antisense strand comprises SEQ ID NO: 3232, the first 19 nucleobases of SEQ ID NO: 3232, or a nucleic acid sequence thereof having about 1 or 2 nucleoside substitutions, additions, or deletions; or the sense strand comprises SEQ ID NO: 5478 or a nucleic acid sequence thereof having about 1 or 2 nucleoside substitutions, additions, or deletions, and the antisense strand comprises SEQ ID NO: 5479 or a nucleic acid sequence thereof having about 1 or 2 nucleoside substitutions, additions, or deletions. 569. A pharmaceutical composition comprising a sense strand and an antisense strand, wherein the sense strand comprises SEQ ID NO: 3233, the first 19 nucleobases of SEQ ID NO: 3233, or a nucleic acid sequence thereof having about 1 or 2 nucleoside substitutions, additions, or deletions, and the antisense strand comprises SEQ ID NO: 3234, the first 19 nucleobases of SEQ ID NO: 3234, or a nucleic acid sequence thereof having about 1 or 2 nucleoside substitutions, additions, or deletions; or the sense strand comprises SEQ ID NO: 5480 or a nucleic acid sequence thereof having about 1 or 2 nucleoside substitutions, additions, or deletions, and the antisense strand comprises SEQ ID NO: 5481 or a nucleic acid sequence thereof having about 1 or 2 nucleoside substitutions, additions, or deletions. 570. A pharmaceutical composition comprising a sense strand and an antisense strand, wherein the sense strand comprises SEQ ID NO: 3243, the first 19 nucleobases of SEQ ID NO: 3243, or a nucleic acid sequence thereof having about 1 or 2 nucleoside substitutions, additions, or deletions, and the antisense strand comprises SEQ ID NO: 3244, the first 19 nucleobases of SEQ ID NO: 3244, or a nucleic acid sequence thereof having about 1 or 2 nucleoside substitutions, additions, or deletions; or the sense strand comprises SEQ ID NO: 5482 or a nucleic acid sequence thereof having about 1 or 2 nucleoside substitutions, additions, or deletions, and the antisense strand comprises SEQ ID NO: 5483 or a nucleic acid sequence thereof having about 1 or 2 nucleoside substitutions, additions, or deletions. 571. A pharmaceutical composition comprising a sense strand and an antisense strand, wherein the sense strand comprises SEQ ID NO: 3439, the first 19 nucleobases of SEQ ID NO: 3439, or a nucleic acid sequence thereof having about 1 or 2 nucleoside substitutions, additions, or deletions, and the antisense strand comprises SEQ ID NO: 3440, the first 19 nucleobases of SEQ ID NO: 3440, or a nucleic acid sequence thereof having about 1 or 2 nucleoside substitutions, additions, or deletions; or the sense strand comprises SEQ ID NO: 5484 or a nucleic acid sequence thereof having about 1 or 2 nucleoside substitutions, additions, or deletions, and the antisense strand comprises SEQ ID NO: 5485 or a nucleic acid sequence thereof having about 1 or 2 nucleoside substitutions, additions, or deletions. 572. A pharmaceutical composition comprising a sense strand and an antisense strand, wherein the sense strand comprises SEQ ID NO: 3443, the first 19 nucleobases of SEQ ID NO: 3443, or a nucleic acid sequence thereof having about 1 or 2 nucleoside substitutions, additions, or deletions, and the antisense strand comprises SEQ ID NO: 3444, the first 19 nucleobases of SEQ ID NO: 3444, or a nucleic acid sequence thereof having about 1 or 2 nucleoside substitutions, additions, or deletions; or the sense strand comprises SEQ ID NO: 5486 or a nucleic acid sequence thereof having about 1 or 2 nucleoside substitutions, additions, or deletions, and the antisense strand comprises SEQ ID NO: 5487 or a nucleic acid sequence thereof having about 1 or 2 nucleoside substitutions, additions, or deletions. 573. A pharmaceutical composition comprising a sense strand and an antisense strand, wherein the sense strand comprises SEQ ID NO: 3449, the first 19 nucleobases of SEQ ID NO: 3449, or a nucleic acid sequence thereof having about 1 or 2 nucleoside substitutions, additions, or deletions, and the antisense strand comprises SEQ ID NO: 3450, the first 19 nucleobases of SEQ ID NO: 3450, or a nucleic acid sequence thereof having about 1 or 2 nucleoside substitutions, additions, or deletions; or the sense strand comprises SEQ ID NO: 5488 or a nucleic acid sequence thereof having about 1 or 2 nucleoside substitutions, additions, or deletions, and the antisense strand comprises SEQ ID NO: 5489 or a nucleic acid sequence thereof having about 1 or 2 nucleoside substitutions, additions, or deletions. 574. A pharmaceutical composition comprising a sense strand and an antisense strand, wherein the sense strand comprises SEQ ID NO: 3457, the first 19 nucleobases of SEQ ID NO: 3457, or a nucleic acid sequence thereof having about 1 or 2 nucleoside substitutions, additions, or deletions, and the antisense strand comprises SEQ ID NO: 3458, the first 19 nucleobases of SEQ ID NO: 3458, or a nucleic acid sequence thereof having about 1 or 2 nucleoside substitutions, additions, or deletions; or the sense strand comprises SEQ ID NO: 5490 or a nucleic acid sequence thereof having about 1 or 2 nucleoside substitutions, additions, or deletions, and the antisense strand comprises SEQ ID NO: 5491 or a nucleic acid sequence thereof having about 1 or 2 nucleoside substitutions, additions, or deletions. 575. A pharmaceutical composition comprising a sense strand and an antisense strand, wherein the sense strand comprises SEQ ID NO: 3461, the first 19 nucleobases of SEQ ID NO: 3461, or a nucleic acid sequence thereof having about 1 or 2 nucleoside substitutions, additions, or deletions, and the antisense strand comprises SEQ ID NO: 3462, the first 19 nucleobases of SEQ ID NO: 3462, or a nucleic acid sequence thereof having about 1 or 2 nucleoside substitutions, additions, or deletions; or the sense strand comprises SEQ ID NO: 5492 or a nucleic acid sequence thereof having about 1 or 2 nucleoside substitutions, additions, or deletions, and the antisense strand comprises SEQ ID NO: 5493 or a nucleic acid sequence thereof having about 1 or 2 nucleoside substitutions, additions, or deletions. 576. A pharmaceutical composition comprising a sense strand and an antisense strand, wherein the sense strand comprises SEQ ID NO: 3463, the first 19 nucleobases of SEQ ID NO: 3463, or a nucleic acid sequence thereof having about 1 or 2 nucleoside substitutions, additions, or deletions, and the antisense strand comprises SEQ ID NO: 3464, the first 19 nucleobases of SEQ ID NO: 3464, or a nucleic acid sequence thereof having about 1 or 2 nucleoside substitutions, additions, or deletions; or the sense strand comprises SEQ ID NO: 5494 or a nucleic acid sequence thereof having about 1 or 2 nucleoside substitutions, additions, or deletions, and the antisense strand comprises SEQ ID NO: 5495 or a nucleic acid sequence thereof having about 1 or 2 nucleoside substitutions, additions, or deletions. 577. A pharmaceutical composition comprising a sense strand and an antisense strand, wherein the sense strand comprises SEQ ID NO: 3573, the first 19 nucleobases of SEQ ID NO: 3573, or a nucleic acid sequence thereof having about 1 or 2 nucleoside substitutions, additions, or deletions, and the antisense strand comprises SEQ ID NO: 3574, the first 19 nucleobases of SEQ ID NO: 3574, or a nucleic acid sequence thereof having about 1 or 2 nucleoside substitutions, additions, or deletions; or the sense strand comprises SEQ ID NO: 5496 or a nucleic acid sequence thereof having about 1 or 2 nucleoside substitutions, additions, or deletions, and the antisense strand comprises SEQ ID NO: 5497 or a nucleic acid sequence thereof having about 1 or 2 nucleoside substitutions, additions, or deletions. 578. A pharmaceutical composition comprising a sense strand and an antisense strand, wherein the sense strand comprises SEQ ID NO: 3703, the first 19 nucleobases of SEQ ID NO: 3703, or a nucleic acid sequence thereof having about 1 or 2 nucleoside substitutions, additions, or deletions, and the antisense strand comprises SEQ ID NO: 3704, the first 19 nucleobases of SEQ ID NO: 3704, or a nucleic acid sequence thereof having about 1 or 2 nucleoside substitutions, additions, or deletions; or the sense strand comprises SEQ ID NO: 5498 or a nucleic acid sequence thereof having about 1 or 2 nucleoside substitutions, additions, or deletions, and the antisense strand comprises SEQ ID NO: 5499 or a nucleic acid sequence thereof having about 1 or 2 nucleoside substitutions, additions, or deletions. 579. A pharmaceutical composition comprising a sense strand and an antisense strand, wherein the sense strand comprises SEQ ID NO: 3705, the first 19 nucleobases of SEQ ID NO: 3705, or a nucleic acid sequence thereof having about 1 or 2 nucleoside substitutions, additions, or deletions, and the antisense strand comprises SEQ ID NO: 3706, the first 19 nucleobases of SEQ ID NO: 3706, or a nucleic acid sequence thereof having about 1 or 2 nucleoside substitutions, additions, or deletions; or the sense strand comprises SEQ ID NO: 5500 or a nucleic acid sequence thereof having about 1 or 2 nucleoside substitutions, additions, or deletions, and the antisense strand comprises SEQ ID NO: 5501 or a nucleic acid sequence thereof having about 1 or 2 nucleoside substitutions, additions, or deletions. 580. A pharmaceutical composition comprising a sense strand and an antisense strand, wherein the sense strand comprises SEQ ID NO: 3875, the first 19 nucleobases of SEQ ID NO: 3875, or a nucleic acid sequence thereof having about 1 or 2 nucleoside substitutions, additions, or deletions, and the antisense strand comprises SEQ ID NO: 3876, the first 19 nucleobases of SEQ ID NO: 3876, or a nucleic acid sequence thereof having about 1 or 2 nucleoside substitutions, additions, or deletions; or the sense strand comprises SEQ ID NO: 5502 or a nucleic acid sequence thereof having about 1 or 2 nucleoside substitutions, additions, or deletions, and the antisense strand comprises SEQ ID NO: 5503 or a nucleic acid sequence thereof having about 1 or 2 nucleoside substitutions, additions, or deletions. 581. A pharmaceutical composition comprising a sense strand and an antisense strand, wherein the sense strand comprises SEQ ID NO: 3889, the first 19 nucleobases of SEQ ID NO: 3889, or a nucleic acid sequence thereof having about 1 or 2 nucleoside substitutions, additions, or deletions, and the antisense strand comprises SEQ ID NO: 3890, the first 19 nucleobases of SEQ ID NO: 3890, or a nucleic acid sequence thereof having about 1 or 2 nucleoside substitutions, additions, or deletions; or the sense strand comprises SEQ ID NO: 5504 or a nucleic acid sequence thereof having about 1 or 2 nucleoside substitutions, additions, or deletions, and the antisense strand comprises SEQ ID NO: 5505 or a nucleic acid sequence thereof having about 1 or 2 nucleoside substitutions, additions, or deletions. 582. A pharmaceutical composition comprising a sense strand and an antisense strand, wherein the sense strand comprises SEQ ID NO: 3981, the first 19 nucleobases of SEQ ID NO: 3981, or a nucleic acid sequence thereof having about 1 or 2 nucleoside substitutions, additions, or deletions, and the antisense strand comprises SEQ ID NO: 3982, the first 19 nucleobases of SEQ ID NO: 3982, or a nucleic acid sequence thereof having about 1 or 2 nucleoside substitutions, additions, or deletions; or the sense strand comprises SEQ ID NO: 5506 or a nucleic acid sequence thereof having about 1 or 2 nucleoside substitutions, additions, or deletions, and the antisense strand comprises SEQ ID NO: 5507 or a nucleic acid sequence thereof having about 1 or 2 nucleoside substitutions, additions, or deletions. 583. A pharmaceutical composition comprising a sense strand and an antisense strand, wherein the sense strand comprises SEQ ID NO: 4089, the first 19 nucleobases of SEQ ID NO: 4089, or a nucleic acid sequence thereof having about 1 or 2 nucleoside substitutions, additions, or deletions, and the antisense strand comprises SEQ ID NO: 4090, the first 19 nucleobases of SEQ ID NO: 4090, or a nucleic acid sequence thereof having about 1 or 2 nucleoside substitutions, additions, or deletions; or the sense strand comprises SEQ ID NO: 5508 or a nucleic acid sequence thereof having about 1 or 2 nucleoside substitutions, additions, or deletions, and the antisense strand comprises SEQ ID NO: 5509 or a nucleic acid sequence thereof having about 1 or 2 nucleoside substitutions, additions, or deletions. 584. A pharmaceutical composition comprising a sense strand and an antisense strand, wherein the sense strand comprises SEQ ID NO: 4265, the first 19 nucleobases of SEQ ID NO: 4265, or a nucleic acid sequence thereof having about 1 or 2 nucleoside substitutions, additions, or deletions, and the antisense strand comprises SEQ ID NO: 4266, the first 19 nucleobases of SEQ ID NO: 4266, or a nucleic acid sequence thereof having about 1 or 2 nucleoside substitutions, additions, or deletions; or the sense strand comprises SEQ ID NO: 5510 or a nucleic acid sequence thereof having about 1 or 2 nucleoside substitutions, additions, or deletions, and the antisense strand comprises SEQ ID NO: 5511 or a nucleic acid sequence thereof having about 1 or 2 nucleoside substitutions, additions, or deletions. 585. A pharmaceutical composition comprising a sense strand and an antisense strand, wherein the sense strand comprises SEQ ID NO: 4321, the first 19 nucleobases of SEQ ID NO: 4321, or a nucleic acid sequence thereof having about 1 or 2 nucleoside substitutions, additions, or deletions, and the antisense strand comprises SEQ ID NO: 4322, the first 19 nucleobases of SEQ ID NO: 4322, or a nucleic acid sequence thereof having about 1 or 2 nucleoside substitutions, additions, or deletions; or the sense strand comprises SEQ ID NO: 5512 or a nucleic acid sequence thereof having about 1 or 2 nucleoside substitutions, additions, or deletions, and the antisense strand comprises SEQ ID NO: 5513 or a nucleic acid sequence thereof having about 1 or 2 nucleoside substitutions, additions, or deletions. 586. A pharmaceutical composition comprising a sense strand and an antisense strand, wherein the sense strand comprises SEQ ID NO: 4339, the first 19 nucleobases of SEQ ID NO: 4339, or a nucleic acid sequence thereof having about 1 or 2 nucleoside substitutions, additions, or deletions, and the antisense strand comprises SEQ ID NO: 4340, the first 19 nucleobases of SEQ ID NO: 4340, or a nucleic acid sequence thereof having about 1 or 2 nucleoside substitutions, additions, or deletions; or the sense strand comprises SEQ ID NO: 5514 or a nucleic acid sequence thereof having about 1 or 2 nucleoside substitutions, additions, or deletions, and the antisense strand comprises SEQ ID NO: 5515 or a nucleic acid sequence thereof having about 1 or 2 nucleoside substitutions, additions, or deletions. 587. A pharmaceutical composition comprising a sense strand and an antisense strand, wherein the sense strand comprises SEQ ID NO: 4347, the first 19 nucleobases of SEQ ID NO: 4347, or a nucleic acid sequence thereof having about 1 or 2 nucleoside substitutions, additions, or deletions, and the antisense strand comprises SEQ ID NO: 4348, the first 19 nucleobases of SEQ ID NO: 4348, or a nucleic acid sequence thereof having about 1 or 2 nucleoside substitutions, additions, or deletions; or the sense strand comprises SEQ ID NO: 5516 or a nucleic acid sequence thereof having about 1 or 2 nucleoside substitutions, additions, or deletions, and the antisense strand comprises SEQ ID NO: 5517 or a nucleic acid sequence thereof having about 1 or 2 nucleoside substitutions, additions, or deletions. 588. A pharmaceutical composition comprising a sense strand and an antisense strand, wherein the sense strand comprises SEQ ID NO: 4507, the first 19 nucleobases of SEQ ID NO: 4507, or a nucleic acid sequence thereof having about 1 or 2 nucleoside substitutions, additions, or deletions, and the antisense strand comprises SEQ ID NO: 4508, the first 19 nucleobases of SEQ ID NO: 4508, or a nucleic acid sequence thereof having about 1 or 2 nucleoside substitutions, additions, or deletions; or the sense strand comprises SEQ ID NO: 5518 or a nucleic acid sequence thereof having about 1 or 2 nucleoside substitutions, additions, or deletions, and the antisense strand comprises SEQ ID NO: 5519 or a nucleic acid sequence thereof having about 1 or 2 nucleoside substitutions, additions, or deletions. 589. A pharmaceutical composition comprising a sense strand and an antisense strand, wherein the sense strand comprises SEQ ID NO: 53, the first 19 nucleobases of SEQ ID NO: 53, or a nucleic acid sequence thereof having about 1 or 2 nucleoside substitutions, additions, or deletions, and the antisense strand comprises SEQ ID NO: 54, the first 19 nucleobases of SEQ ID NO: 54, or a nucleic acid sequence thereof having about 1 or 2 nucleoside substitutions, additions, or deletions; or the sense strand comprises SEQ ID NO: 5520 or a nucleic acid sequence thereof having about 1 or 2 nucleoside substitutions, additions, or deletions, and the antisense strand comprises SEQ ID NO: 5521 or a nucleic acid sequence thereof having about 1 or 2 nucleoside substitutions, additions, or deletions. 590. A pharmaceutical composition comprising a sense strand and an antisense strand, wherein the sense strand comprises SEQ ID NO: 2627, the first 19 nucleobases of SEQ ID NO: 2627, or a nucleic acid sequence thereof having about 1 or 2 nucleoside substitutions, additions, or deletions, and the antisense strand comprises SEQ ID NO: 2628, the first 19 nucleobases of SEQ ID NO: 2628, or a nucleic acid sequence thereof having about 1 or 2 nucleoside substitutions, additions, or deletions; or the sense strand comprises SEQ ID NO: 5522 or a nucleic acid sequence thereof having about 1 or 2 nucleoside substitutions, additions, or deletions, and the antisense strand comprises SEQ ID NO: 5523 or a nucleic acid sequence thereof having about 1 or 2 nucleoside substitutions, additions, or deletions. 591. A pharmaceutical composition comprising a sense strand and an antisense strand, wherein the sense strand comprises SEQ ID NO: 2629, the first 19 nucleobases of SEQ ID NO: 2629, or a nucleic acid sequence thereof having about 1 or 2 nucleoside substitutions, additions, or deletions, and the antisense strand comprises SEQ ID NO: 2630, the first 19 nucleobases of SEQ ID NO: 2630, or a nucleic acid sequence thereof having about 1 or 2 nucleoside substitutions, additions, or deletions; or the sense strand comprises SEQ ID NO: 5524 or a nucleic acid sequence thereof having about 1 or 2 nucleoside substitutions, additions, or deletions, and the antisense strand comprises SEQ ID NO: 5525 or a nucleic acid sequence thereof having about 1 or 2 nucleoside substitutions, additions, or deletions. 592. A pharmaceutical composition comprising a sense strand and an antisense strand, wherein the sense strand comprises SEQ ID NO: 3355, the first 19 nucleobases of SEQ ID NO: 3355, or a nucleic acid sequence thereof having about 1 or 2 nucleoside substitutions, additions, or deletions, and the antisense strand comprises SEQ ID NO: 3356, the first 19 nucleobases of SEQ ID NO: 3356, or a nucleic acid sequence thereof having about 1 or 2 nucleoside substitutions, additions, or deletions; or the sense strand comprises SEQ ID NO: 5526 or a nucleic acid sequence thereof having about 1 or 2 nucleoside substitutions, additions, or deletions, and the antisense strand comprises SEQ ID NO: 5527 or a nucleic acid sequence thereof having about 1 or 2 nucleoside substitutions, additions, or deletions. 593. A pharmaceutical composition comprising a sense strand and an antisense strand, wherein the sense strand comprises SEQ ID NO: 3365, the first 19 nucleobases of SEQ ID NO: 3365, or a nucleic acid sequence thereof having about 1 or 2 nucleoside substitutions, additions, or deletions, and the antisense strand comprises SEQ ID NO: 3366, the first 19 nucleobases of SEQ ID NO: 3366, or a nucleic acid sequence thereof having about 1 or 2 nucleoside substitutions, additions, or deletions; or the sense strand comprises SEQ ID NO: 5528 or a nucleic acid sequence thereof having about 1 or 2 nucleoside substitutions, additions, or deletions, and the antisense strand comprises SEQ ID NO: 5529 or a nucleic acid sequence thereof having about 1 or 2 nucleoside substitutions, additions, or deletions. 594. A pharmaceutical composition comprising a sense strand and an antisense strand, wherein the sense strand comprises SEQ ID NO: 87, the first 19 nucleobases of SEQ ID NO: 87, or a nucleic acid sequence thereof having about 1 or 2 nucleoside substitutions, additions, or deletions, and the antisense strand comprises SEQ ID NO: 88, the first 19 nucleobases of SEQ ID NO: 88, or a nucleic acid sequence thereof having about 1 or 2 nucleoside substitutions, additions, or deletions; or the sense strand comprises SEQ ID NO: 5530 or a nucleic acid sequence thereof having about 1 or 2 nucleoside substitutions, additions, or deletions, and the antisense strand comprises SEQ ID NO: 5531 or a nucleic acid sequence thereof having about 1 or 2 nucleoside substitutions, additions, or deletions. 595. A pharmaceutical composition comprising a sense strand and an antisense strand, wherein the sense strand comprises SEQ ID NO: 2329, the first 19 nucleobases of SEQ ID NO: 2329, or a nucleic acid sequence thereof having about 1 or 2 nucleoside substitutions, additions, or deletions, and the antisense strand comprises SEQ ID NO: 2330, the first 19 nucleobases of SEQ ID NO: 2330, or a nucleic acid sequence thereof having about 1 or 2 nucleoside substitutions, additions, or deletions; or the sense strand comprises SEQ ID NO: 5532 or a nucleic acid sequence thereof having about 1 or 2 nucleoside substitutions, additions, or deletions, and the antisense strand comprises SEQ ID NO: 5533 or a nucleic acid sequence thereof having about 1 or 2 nucleoside substitutions, additions, or deletions. 596. A pharmaceutical composition comprising a sense strand and an antisense strand, wherein the sense strand comprises SEQ ID NO: 2379, the first 19 nucleobases of SEQ ID NO: 2379, or a nucleic acid sequence thereof having about 1 or 2 nucleoside substitutions, additions, or deletions, and the antisense strand comprises SEQ ID NO: 2380, the first 19 nucleobases of SEQ ID NO: 2380, or a nucleic acid sequence thereof having about 1 or 2 nucleoside substitutions, additions, or deletions; or the sense strand comprises SEQ ID NO: 5534 or a nucleic acid sequence thereof having about 1 or 2 nucleoside substitutions, additions, or deletions, and the antisense strand comprises SEQ ID NO: 5535 or a nucleic acid sequence thereof having about 1 or 2 nucleoside substitutions, additions, or deletions. 597. A pharmaceutical composition comprising a sense strand and an antisense strand, wherein the sense strand comprises SEQ ID NO: 2381, the first 19 nucleobases of SEQ ID NO: 2381, or a nucleic acid sequence thereof having about 1 or 2 nucleoside substitutions, additions, or deletions, and the antisense strand comprises SEQ ID NO: 2382, the first 19 nucleobases of SEQ ID NO: 2382, or a nucleic acid sequence thereof having about 1 or 2 nucleoside substitutions, additions, or deletions; or the sense strand comprises SEQ ID NO: 5536 or a nucleic acid sequence thereof having about 1 or 2 nucleoside substitutions, additions, or deletions, and the antisense strand comprises SEQ ID NO: 5537 or a nucleic acid sequence thereof having about 1 or 2 nucleoside substitutions, additions, or deletions. 598. A pharmaceutical composition comprising a sense strand and an antisense strand, wherein the sense strand comprises SEQ ID NO: 2521, the first 19 nucleobases of SEQ ID NO: 2521, or a nucleic acid sequence thereof having about 1 or 2 nucleoside substitutions, additions, or deletions, and the antisense strand comprises SEQ ID NO: 2522, the first 19 nucleobases of SEQ ID NO: 2522, or a nucleic acid sequence thereof having about 1 or 2 nucleoside substitutions, additions, or deletions; or the sense strand comprises SEQ ID NO: 5538 or a nucleic acid sequence thereof having about 1 or 2 nucleoside substitutions, additions, or deletions, and the antisense strand comprises SEQ ID NO: 5539 or a nucleic acid sequence thereof having about 1 or 2 nucleoside substitutions, additions, or deletions. 599. A pharmaceutical composition comprising a sense strand and an antisense strand, wherein the sense strand comprises SEQ ID NO: 2525, the first 19 nucleobases of SEQ ID NO: 2525, or a nucleic acid sequence thereof having about 1 or 2 nucleoside substitutions, additions, or deletions, and the antisense strand comprises SEQ ID NO: 2526, the first 19 nucleobases of SEQ ID NO: 2526, or a nucleic acid sequence thereof having about 1 or 2 nucleoside substitutions, additions, or deletions; or the sense strand comprises SEQ ID NO: 5540 or a nucleic acid sequence thereof having about 1 or 2 nucleoside substitutions, additions, or deletions, and the antisense strand comprises SEQ ID NO: 5541 or a nucleic acid sequence thereof having about 1 or 2 nucleoside substitutions, additions, or deletions. 600. A pharmaceutical composition comprising a sense strand and an antisense strand, wherein the sense strand comprises SEQ ID NO: 2607, the first 19 nucleobases of SEQ ID NO: 2607, or a nucleic acid sequence thereof having about 1 or 2 nucleoside substitutions, additions, or deletions, and the antisense strand comprises SEQ ID NO: 2608, the first 19 nucleobases of SEQ ID NO: 2608, or a nucleic acid sequence thereof having about 1 or 2 nucleoside substitutions, additions, or deletions; or the sense strand comprises SEQ ID NO: 5542 or a nucleic acid sequence thereof having about 1 or 2 nucleoside substitutions, additions, or deletions, and the antisense strand comprises SEQ ID NO: 5543 or a nucleic acid sequence thereof having about 1 or 2 nucleoside substitutions, additions, or deletions. 601. A pharmaceutical composition comprising a sense strand and an antisense strand, wherein the sense strand comprises SEQ ID NO: 3057, the first 19 nucleobases of SEQ ID NO: 3057, or a nucleic acid sequence thereof having about 1 or 2 nucleoside substitutions, additions, or deletions, and the antisense strand comprises SEQ ID NO: 3058, the first 19 nucleobases of SEQ ID NO: 3058, or a nucleic acid sequence thereof having about 1 or 2 nucleoside substitutions, additions, or deletions; or the sense strand comprises SEQ ID NO: 5544 or a nucleic acid sequence thereof having about 1 or 2 nucleoside substitutions, additions, or deletions, and the antisense strand comprises SEQ ID NO: 5545 or a nucleic acid sequence thereof having about 1 or 2 nucleoside substitutions, additions, or deletions. 602. A pharmaceutical composition comprising a sense strand and an antisense strand, wherein the sense strand comprises SEQ ID NO: 3061, the first 19 nucleobases of SEQ ID NO: 3061, or a nucleic acid sequence thereof having about 1 or 2 nucleoside substitutions, additions, or deletions, and the antisense strand comprises SEQ ID NO: 3062, the first 19 nucleobases of SEQ ID NO: 3062, or a nucleic acid sequence thereof having about 1 or 2 nucleoside substitutions, additions, or deletions; or the sense strand comprises SEQ ID NO: 5546 or a nucleic acid sequence thereof having about 1 or 2 nucleoside substitutions, additions, or deletions, and the antisense strand comprises SEQ ID NO: 5547 or a nucleic acid sequence thereof having about 1 or 2 nucleoside substitutions, additions, or deletions. 603. A pharmaceutical composition comprising a sense strand and an antisense strand, wherein the sense strand comprises SEQ ID NO: 3067, the first 19 nucleobases of SEQ ID NO: 3067, or a nucleic acid sequence thereof having about 1 or 2 nucleoside substitutions, additions, or deletions, and the antisense strand comprises SEQ ID NO: 3068, the first 19 nucleobases of SEQ ID NO: 3068, or a nucleic acid sequence thereof having about 1 or 2 nucleoside substitutions, additions, or deletions; or the sense strand comprises SEQ ID NO: 5548 or a nucleic acid sequence thereof having about 1 or 2 nucleoside substitutions, additions, or deletions, and the antisense strand comprises SEQ ID NO: 5549 or a nucleic acid sequence thereof having about 1 or 2 nucleoside substitutions, additions, or deletions. 604. A pharmaceutical composition comprising a sense strand and an antisense strand, wherein the sense strand comprises SEQ ID NO: 3501, the first 19 nucleobases of SEQ ID NO: 3501, or a nucleic acid sequence thereof having about 1 or 2 nucleoside substitutions, additions, or deletions, and the antisense strand comprises SEQ ID NO: 3502, the first 19 nucleobases of SEQ ID NO: 3502, or a nucleic acid sequence thereof having about 1 or 2 nucleoside substitutions, additions, or deletions; or the sense strand comprises SEQ ID NO: 5550 or a nucleic acid sequence thereof having about 1 or 2 nucleoside substitutions, additions, or deletions, and the antisense strand comprises SEQ ID NO: 5551 or a nucleic acid sequence thereof having about 1 or 2 nucleoside substitutions, additions, or deletions. 605. A pharmaceutical composition comprising a sense strand and an antisense strand, wherein the sense strand comprises SEQ ID NO: 3503, the first 19 nucleobases of SEQ ID NO: 3503, or a nucleic acid sequence thereof having about 1 or 2 nucleoside substitutions, additions, or deletions, and the antisense strand comprises SEQ ID NO: 3504, the first 19 nucleobases of SEQ ID NO: 3504, or a nucleic acid sequence thereof having about 1 or 2 nucleoside substitutions, additions, or deletions; or the sense strand comprises SEQ ID NO: 5552 or a nucleic acid sequence thereof having about 1 or 2 nucleoside substitutions, additions, or deletions, and the antisense strand comprises SEQ ID NO: 5553 or a nucleic acid sequence thereof having about 1 or 2 nucleoside substitutions, additions, or deletions. 606. A pharmaceutical composition comprising a sense strand and an antisense strand, wherein the sense strand comprises SEQ ID NO: 3873, the first 19 nucleobases of SEQ ID NO: 3873, or a nucleic acid sequence thereof having about 1 or 2 nucleoside substitutions, additions, or deletions, and the antisense strand comprises SEQ ID NO: 3874, the first 19 nucleobases of SEQ ID NO: 3874, or a nucleic acid sequence thereof having about 1 or 2 nucleoside substitutions, additions, or deletions; or the sense strand comprises SEQ ID NO: 5554 or a nucleic acid sequence thereof having about 1 or 2 nucleoside substitutions, additions, or deletions, and the antisense strand comprises SEQ ID NO: 5555 or a nucleic acid sequence thereof having about 1 or 2 nucleoside substitutions, additions, or deletions. 607. A pharmaceutical composition comprising a sense strand and an antisense strand, wherein the sense strand comprises SEQ ID NO: 4179, the first 19 nucleobases of SEQ ID NO: 4179, or a nucleic acid sequence thereof having about 1 or 2 nucleoside substitutions, additions, or deletions, and the antisense strand comprises SEQ ID NO: 4180, the first 19 nucleobases of SEQ ID NO: 4180, or a nucleic acid sequence thereof having about 1 or 2 nucleoside substitutions, additions, or deletions; or the sense strand comprises SEQ ID NO: 5556 or a nucleic acid sequence thereof having about 1 or 2 nucleoside substitutions, additions, or deletions, and the antisense strand comprises SEQ ID NO: 5557 or a nucleic acid sequence thereof having about 1 or 2 nucleoside substitutions, additions, or deletions. 608. A pharmaceutical composition comprising a sense strand and an antisense strand, wherein the sense strand comprises SEQ ID NO: 3027, the first 19 nucleobases of SEQ ID NO: 3027, or a nucleic acid sequence thereof having about 1 or 2 nucleoside substitutions, additions, or deletions, and the antisense strand comprises SEQ ID NO: 3028, the first 19 nucleobases of SEQ ID NO: 3028, or a nucleic acid sequence thereof having about 1 or 2 nucleoside substitutions, additions, or deletions; or the sense strand comprises SEQ ID NO: 5558 or a nucleic acid sequence thereof having about 1 or 2 nucleoside substitutions, additions, or deletions, and the antisense strand comprises SEQ ID NO: 5559 or a nucleic acid sequence thereof having about 1 or 2 nucleoside substitutions, additions, or deletions. 609. A pharmaceutical composition comprising a sense strand and an antisense strand, wherein the sense strand comprises SEQ ID NO: 3037, the first 19 nucleobases of SEQ ID NO: 3037, or a nucleic acid sequence thereof having about 1 or 2 nucleoside substitutions, additions, or deletions, and the antisense strand comprises SEQ ID NO: 3038, the first 19 nucleobases of SEQ ID NO: 3038, or a nucleic acid sequence thereof having about 1 or 2 nucleoside substitutions, additions, or deletions; or the sense strand comprises SEQ ID NO: 5560 or a nucleic acid sequence thereof having about 1 or 2 nucleoside substitutions, additions, or deletions, and the antisense strand comprises SEQ ID NO: 5561 or a nucleic acid sequence thereof having about 1 or 2 nucleoside substitutions, additions, or deletions. 610. A pharmaceutical composition comprising a sense strand and an antisense strand, wherein the sense strand comprises SEQ ID NO: 3183, the first 19 nucleobases of SEQ ID NO: 3183, or a nucleic acid sequence thereof having about 1 or 2 nucleoside substitutions, additions, or deletions, and the antisense strand comprises SEQ ID NO: 3184, the first 19 nucleobases of SEQ ID NO: 3184, or a nucleic acid sequence thereof having about 1 or 2 nucleoside substitutions, additions, or deletions; or the sense strand comprises SEQ ID NO: 5562 or a nucleic acid sequence thereof having about 1 or 2 nucleoside substitutions, additions, or deletions, and the antisense strand comprises SEQ ID NO: 5563 or a nucleic acid sequence thereof having about 1 or 2 nucleoside substitutions, additions, or deletions. 611. A pharmaceutical composition comprising a sense strand and an antisense strand, wherein the sense strand comprises SEQ ID NO: 4265, the first 19 nucleobases of SEQ ID NO: 4265, or a nucleic acid sequence thereof having about 1 or 2 nucleoside substitutions, additions, or deletions, and the antisense strand comprises SEQ ID NO: 4266, the first 19 nucleobases of SEQ ID NO: 4266, or a nucleic acid sequence thereof having about 1 or 2 nucleoside substitutions, additions, or deletions; or the sense strand comprises SEQ ID NO: 5564 or a nucleic acid sequence thereof having about 1 or 2 nucleoside substitutions, additions, or deletions, and the antisense strand comprises SEQ ID NO: 5565 or a nucleic acid sequence thereof having about 1 or 2 nucleoside substitutions, additions, or deletions. 612. A pharmaceutical composition comprising a sense strand and an antisense strand, wherein the sense strand comprises SEQ ID NO: 2629, the first 19 nucleobases of SEQ ID NO: 2629, or a nucleic acid sequence thereof having about 1 or 2 nucleoside substitutions, additions, or deletions, and the antisense strand comprises SEQ ID NO: 2630, the first 19 nucleobases of SEQ ID NO: 2630, or a nucleic acid sequence thereof having about 1 or 2 nucleoside substitutions, additions, or deletions; or the sense strand comprises SEQ ID NO: 5566 or a nucleic acid sequence thereof having about 1 or 2 nucleoside substitutions, additions, or deletions, and the antisense strand comprises SEQ ID NO: 5567 or a nucleic acid sequence thereof having about 1 or 2 nucleoside substitutions, additions, or deletions.

613. A pharmaceutical composition comprising a sense strand and an antisense strand, wherein the sense strand comprises one or more sequences selected from SEQ ID NOS: 1-5349, 5366-5971, 6000-6038, 6050-6074, or a nucleic acid sequence thereof having about 1 or 2 nucleoside substitutions, additions, or deletions. 614. The pharmaceutical composition comprising a sense strand and an antisense strand, wherein the antisense strand comprises one or more sequences selected from SEQ ID NOS: 1-5349, 5366-5971, 6000-6038, 6050-6074, or a nucleic acid sequence thereof having about 1 or 2 nucleoside substitutions, additions, or deletions.

615. The pharmaceutical composition of any one of embodiments 442-614, comprising the modified internucleoside linkage. 616. The pharmaceutical composition of embodiment 615, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. 617. The pharmaceutical composition of embodiment 616, wherein the modified internucleoside linkage comprises one or more phosphorothioate linkages. 618. The pharmaceutical composition of embodiment 617, wherein the one or more phosphorothioate linkages is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 phosphorothioate linkages. 619. The pharmaceutical composition of embodiment 617 or embodiment 618, wherein the sense strand of the siRNA comprises one or more phosphorothioate linkages. 620. The pharmaceutical composition of embodiment 619, wherein the one or more phosphorothioate linkages of the sense strand is about 1, 2, 3, 4, 5, 6, 7, 8, 9, 10, 11, 12, 13, 14, 15, 16, 17, 18, 19, 20, or 21 phosphorothioate linkages. 621. The pharmaceutical composition of embodiment 620, wherein the one or more phosphorothioate linkages of the sense strand is about 1, 2, 3, 4, or 5 phosphorothioate linkages. 622. The pharmaceutical composition of embodiment 621, wherein the one or more phosphorothioate linkages of the sense strand is about 4 phosphorothioate linkages. 623. The pharmaceutical composition of any one of embodiments 619-622, wherein the sense strand comprises a phosphorothioate linkage between the first nucleoside and the second nucleoside of the sense strand, in a 5′ to 3′ direction. 624. The pharmaceutical composition of any one of embodiments 619-623, wherein the sense strand comprises a phosphorothioate linkage between the second nucleoside and the third nucleoside of the sense strand, in a 5′ to 3′ direction. 625. The pharmaceutical composition of any one of embodiments 619-624, wherein the sense strand comprises a phosphorothioate linkage between the nineteenth nucleoside and the twentieth nucleoside of the sense strand, in a 5′ to 3′ direction. 626. The pharmaceutical composition of any one of embodiments 619-624, wherein the sense strand comprises a phosphorothioate linkage between the twentieth nucleoside and the twenty-first nucleoside of the sense strand, in a 5′ to 3′ direction. 627. The pharmaceutical composition of any one of embodiments 617-626, wherein the antisense strand of the siRNA comprises one or more phosphorothioate linkages. 628. The pharmaceutical composition of embodiment 627, wherein the one or more phosphorothioate linkages of the antisense strand is about 1, 2, 3, 4, 5, 6, 7, 8, 9, 10, 11, 12, 13, 14, 15, 16, 17, 18, 19, 20, or 21 phosphorothioate linkages. 629. The pharmaceutical composition of embodiment 628, wherein the one or more phosphorothioate linkages of the antisense strand is about 1, 2, 3, 4, or 5 phosphorothioate linkages. 630. The pharmaceutical composition of 629, wherein the one or more phosphorothioate linkages of the antisense strand is about 4 phosphorothioate linkages. 631. The pharmaceutical composition of any one of embodiments 627-630, wherein the antisense strand comprises a phosphorothioate linkage between the first nucleoside and the second nucleoside of the antisense strand, in a 5′ to 3′ direction. 632. The pharmaceutical composition of any one of embodiments 627-631, wherein the antisense strand comprises a phosphorothioate linkage between the second nucleoside and the third nucleoside of the antisense strand, in a 5′ to 3′ direction. 633. The pharmaceutical composition of any one of embodiments 627-632, wherein the antisense strand comprises a phosphorothioate linkage between the nineteenth nucleoside and the twentieth nucleoside of the sense strand, in a 5′ to 3′ direction. 634. The pharmaceutical composition of any one of embodiments 627-633, wherein the antisense strand comprises a phosphorothioate linkage between the twentieth nucleoside and the twenty-first nucleoside of the sense strand, in a 5′ to 3′ direction.

635. The pharmaceutical composition of any one of embodiments 442-634, comprising the modified nucleoside. 636. The pharmaceutical composition of embodiment 635, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. 637. The pharmaceutical composition of embodiment 635 or embodiment 636, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. 638. The pharmaceutical composition of any one of embodiments 635-637, wherein the modified nucleoside comprises one or more 2′fluoro modified nucleosides. 639. The pharmaceutical composition of embodiment 638, wherein the one or more 2′ fluoro modified nucleosides is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 2′ fluoro modified nucleosides. 640. The pharmaceutical composition of embodiment 638 or embodiment 639, wherein the sense strand of the siRNA comprises one or more 2′ fluoro modified nucleosides. 641. The pharmaceutical composition of embodiment 640, wherein the one or more 2′ fluoro modified nucleosides of the sense strand is about 1, 2, 3, 4, 5, 6, 7, 8, 9, 10, 11, 12, 13, 14, 15, 16, 17, 18, 19, 20, or 21 2′ fluoro modified nucleosides. 642. The pharmaceutical composition of embodiment 641, wherein the one or more 2′ fluoro modified nucleosides of the sense strand is about eleven 2′ fluoro modified nucleosides. 643. The pharmaceutical composition of embodiment 641, wherein the one or more 2′ fluoro modified nucleosides of the sense strand is about four 2′ fluoro modified nucleosides. 644. The pharmaceutical composition of embodiment 641, wherein the one or more 2′ fluoro modified nucleosides of the sense strand is about three 2′ fluoro modified nucleosides. 645. The pharmaceutical composition of any one of embodiments 640-644, wherein the first nucleoside of the sense strand comprises the 2′ fluoro modified nucleoside, in a 5′ to 3′ direction. 646. The pharmaceutical composition of any one of embodiments 640-645, wherein the third nucleoside of the sense strand comprises the 2′ fluoro modified nucleoside, in a 5′ to 3′ direction. 647. The pharmaceutical composition of any one of embodiments 640-646, wherein the fifth nucleoside of the sense strand comprises the 2′ fluoro modified nucleoside, in a 5′ to 3′ direction. 648. The pharmaceutical composition of any one of embodiments 640-647, wherein the seventh nucleoside of the sense strand comprises the 2′ fluoro modified nucleoside, in a 5′ to 3′ direction. 649. The pharmaceutical composition of any one of embodiments 640-648, wherein the eighth nucleoside of the sense strand comprises the 2′ fluoro modified nucleoside, in a 5′ to 3′ direction. 650. The pharmaceutical composition of any one of embodiments 640-649, wherein the ninth nucleoside of the sense strand comprises the 2′ fluoro modified nucleoside, in a 5′ to 3′ direction. 651. The pharmaceutical composition of any one of embodiments 640-650, wherein the eleventh nucleoside of the sense strand comprises the 2′ fluoro modified nucleoside, in a 5′ to 3′ direction. 652. The pharmaceutical composition of any one of embodiments 640-651, wherein the thirteenth nucleoside of the sense strand comprises the 2′ fluoro modified nucleoside, in a 5′ to 3′ direction. 653. The pharmaceutical composition of any one of embodiments 640-652, wherein the fifteenth nucleoside of the sense strand comprises the 2′ fluoro modified nucleoside, in a 5′ to 3′ direction. 654. The pharmaceutical composition of any one of embodiments 640-653, wherein the seventeenth nucleoside of the sense strand comprises the 2′ fluoro modified nucleoside, in a 5′ to 3′ direction. 655. The pharmaceutical composition of any one of embodiments 640-654, wherein the nineteenth nucleoside of the sense strand comprises the 2′ fluoro modified nucleoside, in a 5′ to 3′ direction. 656. The pharmaceutical composition of any one of embodiments 640-655, wherein the fifth, seventh, and ninth nucleosides of the sense strand comprises the 2′ fluoro modified nucleoside, in a 5′ to 3′ direction. 657. The pharmaceutical composition of any one of embodiments 640-656, wherein the sense strand comprises the pattern fN-Z1-fN-Z2-fN, wherein fN comprises the 2′ fluoro modified nucleoside and Z1 and Z2 are independently a 2′ O-methyl modified nucleoside or a 2′ fluoro modified nucleoside. 658. The pharmaceutical composition of embodiment 657, wherein the fN-Z1-fN-Z2-fN corresponds to nucleosides five to nine of the sense strand, in a 5′ to 3′ direction. 659. The pharmaceutical composition of any one of embodiments 640-658, wherein the sense strand comprises at least two contiguous 2′ fluoro modified nucleosides. 660. The pharmaceutical composition of embodiment 659, wherein the at least two contiguous 2′ fluoro modified nucleosides is two contiguous 2′ fluoro modified nucleosides. 661. The pharmaceutical composition of embodiment 659, wherein the at least two contiguous 2′ fluoro modified nucleosides is three contiguous 2′ fluoro modified nucleosides. 662. The pharmaceutical composition of any one of embodiments 638-661, wherein the antisense strand of the siRNA comprises one or more 2′ fluoro modified nucleosides. 663. The pharmaceutical composition of embodiment 662, wherein the one or more 2′ fluoro modified nucleosides of the antisense strand is about 1, 2, 3, 4, 5, 6, 7, 8, 9, 10, 11, 12, 13, 14, 15, 16, 17, 18, 19, 20, or 21 2′ fluoro modified nucleosides. 664. The pharmaceutical composition of embodiment 663, wherein the one or more 2′ fluoro modified nucleosides of the antisense strand is about eight 2′ fluoro modified nucleosides. 665. The pharmaceutical composition of embodiment 663, wherein the one or more 2′ fluoro modified nucleosides of the antisense strand is about six 2′ fluoro modified nucleosides. 666. The pharmaceutical composition of embodiment 663, wherein the one or more 2′ fluoro modified nucleosides of the antisense strand is about five 2′ fluoro modified nucleosides. 667. The pharmaceutical composition of embodiment 663, wherein the one or more 2′ fluoro modified nucleosides of the antisense strand is about four 2′ fluoro modified nucleosides. 668. The pharmaceutical composition of any one of embodiments 662-667, wherein the second nucleoside of the antisense strand comprises the 2′ fluoro modified nucleoside, in a 5′ to 3′ direction. 669. The pharmaceutical composition of any one of embodiments 662-668, wherein the fourth nucleoside of the antisense strand comprises the 2′ fluoro modified nucleoside, in a 5′ to 3′ direction. 670. The pharmaceutical composition of any one of embodiments 662-669, wherein the sixth nucleoside of the antisense strand comprises the 2′ fluoro modified nucleoside, in a 5′ to 3′ direction. 671. The pharmaceutical composition of any one of embodiments 662-670, wherein the eighth nucleoside of the antisense strand comprises the 2′ fluoro modified nucleoside, in a 5′ to 3′ direction. 672. The pharmaceutical composition of any one of embodiments 662-671, wherein the ninth nucleoside of the antisense strand comprises the 2′ fluoro modified nucleoside, in a 5′ to 3′ direction. 673. The pharmaceutical composition of any one of embodiments 662-672, wherein the tenth nucleoside of the antisense strand comprises the 2′ fluoro modified nucleoside, in a 5′ to 3′ direction. 674. The pharmaceutical composition of any one of embodiments 662-673, wherein the fourteenth nucleoside of the antisense strand comprises the 2′ fluoro modified nucleoside, in a 5′ to 3′ direction. 675. The pharmaceutical composition of any one of embodiments 662-674, wherein the sixteenth nucleoside of the antisense strand comprises the 2′ fluoro modified nucleoside, in a 5′ to 3′ direction. 676. The pharmaceutical composition of any one of embodiments 662-675, wherein the eighteenth nucleoside of the antisense strand comprises the 2′ fluoro modified nucleoside, in a 5′ to 3′ direction. 677. The pharmaceutical composition of any one of embodiments 662-676, wherein the second and fourteenth nucleosides of the antisense strand comprises the 2′ fluoro modified nucleoside, in a 5′ to 3′ direction. 678. The pharmaceutical composition of any one of embodiments 662-677, wherein the second, sixth, fourteenth, and sixteenth nucleosides of the antisense strand comprises the 2′ fluoro modified nucleoside, in a 5′ to 3′ direction. 679. The pharmaceutical composition of any one of embodiments 662-678, wherein the antisense strand comprises the pattern Z3-fN-Z4-fN, wherein fN comprises the 2′ fluoro modified nucleoside and Z3 and Z4 are independently a 2′ O-methyl modified nucleoside or a 2′ fluoro modified nucleoside. 680. The pharmaceutical composition of embodiment 679, wherein the Z3-fN-Z4-fN corresponds to nucleosides thirteen to sixteen of the antisense strand, in a 5′ to 3′ direction.

681. The pharmaceutical composition of any one of embodiments 635-680, wherein the modified nucleoside comprises a 2′ O-alkyl modified nucleoside. 682. The pharmaceutical composition of embodiment 681, wherein the 2′-O-alkyl modified nucleoside comprises one or more 2′ O-methyl modified nucleosides. 683. The pharmaceutical composition of embodiment 682, wherein the one or more 2′ O-methyl modified nucleosides is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 2′ O-methyl modified nucleosides. 684. The pharmaceutical composition of any one of embodiments 681-683, wherein the sense strand of the siRNA comprises one or more 2′ O-methyl modified nucleosides. 685. The pharmaceutical composition of embodiment 684, wherein the one or more 2′ O-methyl modified nucleosides of the sense strand is about 1, 2, 3, 4, 5, 6, 7, 8, 9, 10, 11, 12, 13, 14, 15, 16, 17, 18, 19, 20, or 21 2′ O-methyl modified nucleosides. 686. The pharmaceutical composition of embodiment 685, wherein the one or more 2′ O-methyl modified nucleosides of the sense strand is about ten 2′ O-methyl modified nucleosides. 687. The pharmaceutical composition of embodiment 685, wherein the one or more 2′ O-methyl modified nucleosides of the sense strand is about seventeen 2′ O-methyl modified nucleosides. 688. The pharmaceutical composition of embodiment 685, wherein the one or more 2′ O-methyl modified nucleosides of the sense strand is about eighteen 2′ O-methyl modified nucleosides. 689. The pharmaceutical composition of any one of embodiments 684-688, wherein the first nucleoside of the sense strand comprises the 2′ O-methyl modified nucleoside, in a 5′ to 3′ direction. 690. The pharmaceutical composition of any one of embodiments 684-689, wherein the second nucleoside of the sense strand comprises the 2′ O-methyl modified nucleoside, in a 5′ to 3′ direction. 691. The pharmaceutical composition of any one of embodiments 684-690, wherein the third nucleoside of the sense strand comprises the 2′ O-methyl modified nucleoside, in a 5′ to 3′ direction. 692. The pharmaceutical composition of any one of embodiments 684-691, wherein the fourth nucleoside of the sense strand comprises the 2′ O-methyl modified nucleoside, in a 5′ to 3′ direction. 693. The pharmaceutical composition of any one of embodiments 684-692, wherein the sixth nucleoside of the sense strand comprises the 2′ O-methyl modified nucleoside, in a 5′ to 3′ direction. 694. The pharmaceutical composition of any one of embodiments 684-693, wherein the eighth nucleoside of the sense strand comprises the 2′ O-methyl modified nucleoside, in a 5′ to 3′ direction. 695. The pharmaceutical composition of any one of embodiments 684-694, wherein the tenth nucleoside of the sense strand comprises the 2′ O-methyl modified nucleoside, in a 5′ to 3′ direction. 696. The pharmaceutical composition of any one of embodiments 684-695, wherein the eleventh nucleoside of the sense strand comprises the 2′ O-methyl modified nucleoside, in a 5′ to 3′ direction. 697. The pharmaceutical composition of any one of embodiments 684-696, wherein the twelfth nucleoside of the sense strand comprises the 2′ O-methyl modified nucleoside, in a 5′ to 3′ direction. 698. The pharmaceutical composition of any one of embodiments 684-697, wherein the thirteenth nucleoside of the sense strand comprises the 2′ O-methyl modified nucleoside, in a 5′ to 3′ direction. 699. The pharmaceutical composition of any one of embodiments 684-698, wherein the fourteenth nucleoside of the sense strand comprises the 2′ O-methyl modified nucleoside, in a 5′ to 3′ direction. 700. The pharmaceutical composition of any one of embodiments 684-699, wherein the fifteenth nucleoside of the sense strand comprises the 2′ O-methyl modified nucleoside, in a 5′ to 3′ direction. 701. The pharmaceutical composition of any one of embodiments 684-700, wherein the sixteenth nucleoside of the sense strand comprises the 2′ O-methyl modified nucleoside, in a 5′ to 3′ direction. 702. The pharmaceutical composition of any one of embodiments 684-701, wherein the seventeenth nucleoside of the sense strand comprises the 2′ O-methyl modified nucleoside, in a 5′ to 3′ direction. 703. The pharmaceutical composition of any one of embodiments 684-702, wherein the eighteenth nucleoside of the sense strand comprises the 2′ O-methyl modified nucleoside, in a 5′ to 3′ direction. 704. The pharmaceutical composition of any one of embodiments 684-703, wherein the nineteenth nucleoside of the sense strand comprises the 2′ O-methyl modified nucleoside, in a 5′ to 3′ direction. 705. The pharmaceutical composition of any one of embodiments 684-704, wherein the twentieth nucleoside of the sense strand comprises the 2′ O-methyl modified nucleoside, in a 5′ to 3′ direction. 706. The pharmaceutical composition of any one of embodiments 684-705, wherein the twenty-first nucleoside of the sense strand comprises the 2′ O-methyl modified nucleoside, in a 5′ to 3′ direction. 707. The pharmaceutical composition of any one of embodiments 684-706, wherein the second, fourth, sixth, tenth, twelfth, fourteenth, and sixteenth, eighteenth, twentieth, and twenty-first nucleosides of the sense strand comprises the 2′ O-methyl modified nucleoside, in a 5′ to 3′ direction. 708. The pharmaceutical composition of any one of embodiments 684-707, wherein the sense strand comprises the pattern mN-Z5-mN-Z6, wherein mN comprises the 2′ O-methyl modified nucleoside and Z5 and Z6 are independently a 2′ O-methyl modified nucleoside or a 2′ fluoro modified nucleoside. 709. The pharmaceutical composition of embodiment 708, wherein the mN-Z5-mN-Z6-mN corresponds to nucleosides four to seven of the sense strand, in a 5′ to 3′ direction. 710. The pharmaceutical composition of embodiment 708 or embodiment 709, wherein Z5 is the 2′ fluoro modified nucleoside. 711. The pharmaceutical composition of embodiment 708 or embodiment 709, wherein Z5 is the 2′ O-methyl modified nucleoside. 712. The pharmaceutical composition of any one of embodiments 708-711, wherein Z6 is the 2′ fluoro modified nucleoside. 713. The pharmaceutical composition of any one of embodiments 708-711, wherein Z6 is the 2′ O-methyl modified nucleoside. 714. The pharmaceutical composition of any one of embodiments 684-713, wherein the sense strand comprises the pattern mN-Z5-mN-Z6-mN, wherein mN comprises the 2′ O-methyl modified nucleoside and Z5 and Z6 are independently a 2′ O-methyl modified nucleoside or a 2′ fluoro modified nucleoside. 715. The pharmaceutical composition of embodiment 715, wherein the mN-Z5-mN-Z6-mN corresponds to nucleosides two to six of the sense strand, in a 5′ to 3′ direction. 716. The pharmaceutical composition of embodiment 715, wherein the mN-Z5-mN-Z6-mN corresponds to nucleosides ten to fourteen of the sense strand, in a 5′ to 3′ direction. 717. The pharmaceutical composition of embodiment 715, wherein the mN-Z5-mN-Z6-mN corresponds to nucleosides twelve to sixteen of the sense strand, in a 5′ to 3′ direction. 718. The pharmaceutical composition of embodiment 715, wherein the mN-Z5-mN-Z6-mN corresponds to nucleosides fourteen to eighteen of the sense strand, in a 5′ to 3′ direction. 719. The pharmaceutical composition of embodiment 715, wherein the mN-Z5-mN-Z6-mN corresponds to nucleosides sixteen to twenty of the sense strand, in a 5′ to 3′ direction. 720. The pharmaceutical composition of any one of embodiments 684-719, wherein the sense strand comprises the pattern mN-Z5-mN-Z6-mN-Z7-mN, wherein Z7 is a 2′ O-methyl modified nucleoside or a 2′ fluoro modified nucleoside. 721. The pharmaceutical composition of embodiment 720, wherein the mN-Z5-mN-Z6-mN-Z7-mN corresponds to nucleosides ten to sixteen of the sense strand, in a 5′ to 3′ direction. 722. The pharmaceutical composition of embodiment 720, wherein the mN-Z5-mN-Z6-mN-Z7-mN corresponds to nucleosides twelve to eighteen of the sense strand, in a 5′ to 3′ direction. 723. The pharmaceutical composition of embodiment 720, wherein the mN-Z5-mN-Z6-mN-Z7-mN corresponds to nucleosides fourteen to twenty of the sense strand, in a 5′ to 3′ direction. 724. The pharmaceutical composition of any one of embodiments 684-723, wherein the sense strand comprises the pattern mN-Z5-mN-Z6-mN-Z7-mN-Z8-mN, wherein Z8 is a 2′ O-methyl modified nucleoside or a 2′ fluoro modified nucleoside. 725. The pharmaceutical composition of embodiment 724, wherein the mN-Z5-mN-Z6-mN-Z7-mN-Z8-mN corresponds to nucleosides ten to eighteen of the sense strand, in a 5′ to 3′ direction. 726. The pharmaceutical composition of embodiment 724, wherein the mN-Z5-mN-Z6-mN-Z7-mN-Z8-mN corresponds to nucleosides twelve to twenty of the sense strand, in a 5′ to 3′ direction. 727. The pharmaceutical composition of any one of embodiments 684-726, wherein the sense strand comprises the pattern mN-Z5-mN-Z6-mN-Z7-mN-Z8-mN-Z9-mN, wherein Z9 is a 2′ O-methyl modified nucleoside or a 2′ fluoro modified nucleoside. 728. The pharmaceutical composition of embodiment 727, wherein the mN-Z5-mN-Z6-mN-Z7-mN-Z8-mN-Z9-mN corresponds to nucleosides ten to twenty of the sense strand, in a 5′ to 3′ direction. 729. The pharmaceutical composition of any one of embodiments 684-728, wherein the sense strand comprises at least two contiguous 2′ O-methyl modified nucleosides. 730. The pharmaceutical composition of embodiment 729, wherein the at least two contiguous 2′ O-methyl modified nucleosides is three contiguous 2′ O-methyl modified nucleosides. 731. The pharmaceutical composition of embodiment 729, wherein the at least two contiguous 2′ O-methyl modified nucleosides is four contiguous 2′ O-methyl modified nucleosides. 732. The pharmaceutical composition of embodiment 729, wherein the at least two contiguous 2′ O-methyl modified nucleosides is five contiguous 2′ O-methyl modified nucleosides. 733. The pharmaceutical composition of embodiment 729, wherein the at least two contiguous 2′ O-methyl modified nucleosides is six contiguous 2′ O-methyl modified nucleosides. 734. The pharmaceutical composition of embodiment 729, wherein the at least two contiguous 2′ O-methyl modified nucleosides is seven contiguous 2′ O-methyl modified nucleosides. 735. The pharmaceutical composition of embodiment 729, wherein the at least two contiguous 2′ O-methyl modified nucleosides is eight contiguous 2′ O-methyl modified nucleosides. 736. The pharmaceutical composition of embodiment 729, wherein the at least two contiguous 2′ O-methyl modified nucleosides is nine contiguous 2′ O-methyl modified nucleosides. 737. The pharmaceutical composition of embodiment 729, wherein the at least two contiguous 2′ O-methyl modified nucleosides is ten contiguous 2′ O-methyl modified nucleosides. 738. The pharmaceutical composition of embodiment 729, wherein the at least two contiguous 2′ O-methyl modified nucleosides is eleven contiguous 2′ O-methyl modified nucleosides. 739. The pharmaceutical composition of embodiment 729, wherein the at least two contiguous 2′ O-methyl modified nucleosides is twelve contiguous 2′ O-methyl modified nucleosides. 740. The pharmaceutical composition of any one of embodiments 681-739, wherein the antisense strand of the siRNA comprises one or more 2′ O-methyl modified nucleosides. 741. The pharmaceutical composition of embodiment 740, wherein the one or more 2′ O-methyl modified nucleosides of the antisense strand is about 1, 2, 3, 4, 5, 6, 7, 8, 9, 10, 11, 12, 13, 14, 15, 16, 17, 18, 19, 20, or 21 2′ O-methyl modified nucleosides. 742. The pharmaceutical composition of embodiment 741, wherein the one or more 2′ O-methyl modified nucleosides of the antisense strand is about thirteen 2′ O-methyl modified nucleosides. 743. The pharmaceutical composition of embodiment 741, wherein the one or more 2′ O-methyl modified nucleosides of the antisense strand is about fifteen 2′ O-methyl modified nucleosides. 744. The pharmaceutical composition of embodiment 741, wherein the one or more 2′ O-methyl modified nucleosides of the antisense strand is about seventeen 2′ O-methyl modified nucleosides. 745. The pharmaceutical composition of any one of embodiments 740-744, wherein the first nucleoside of the antisense strand comprises the 2′ O-methyl modified nucleoside, in a 5′ to 3′ direction. 746. The pharmaceutical composition of any one of embodiments 740-745, wherein the third nucleoside of the antisense strand comprises the 2′ O-methyl modified nucleoside, in a 5′ to 3′ direction. 747. The pharmaceutical composition of any one of embodiments 740-746, wherein the fourth nucleoside of the antisense strand comprises the 2′ O-methyl modified nucleoside, in a 5′ to 3′ direction. 748. The pharmaceutical composition of any one of embodiments 740-747, wherein the fifth nucleoside of the antisense strand comprises the 2′ O-methyl modified nucleoside, in a 5′ to 3′ direction. 749. The pharmaceutical composition of any one of embodiments 740-748, wherein the seventh nucleoside of the antisense strand comprises the 2′ O-methyl modified nucleoside, in a 5′ to 3′ direction. 750. The pharmaceutical composition of any one of embodiments 740-749, wherein the eighth nucleoside of the antisense strand comprises the 2′ O-methyl modified nucleoside, in a 5′ to 3′ direction. 751. The pharmaceutical composition of any one of embodiments 740-750, wherein the ninth nucleoside of the antisense strand comprises the 2′ O-methyl modified nucleoside, in a 5′ to 3′ direction. 752. The pharmaceutical composition of any one of embodiments 740-751, wherein the tenth nucleoside of the antisense strand comprises the 2′ O-methyl modified nucleoside, in a 5′ to 3′ direction. 753. The pharmaceutical composition of any one of embodiments 740-752, wherein the eleventh nucleoside of the antisense strand comprises the 2′ O-methyl modified nucleoside, in a 5′ to 3′ direction. 754. The pharmaceutical composition of any one of embodiments 740-753, wherein the twelfth nucleoside of the antisense strand comprises the 2′ O-methyl modified nucleoside, in a 5′ to 3′ direction. 755. The pharmaceutical composition of any one of embodiments 740-754, wherein the thirteenth nucleoside of the antisense strand comprises the 2′ O-methyl modified nucleoside, in a 5′ to 3′ direction. 756. The pharmaceutical composition of any one of embodiments 740-755, wherein the fifteenth nucleoside of the antisense strand comprises the 2′ O-methyl modified nucleoside, in a 5′ to 3′ direction. 757. The pharmaceutical composition of any one of embodiments 740-756, wherein the seventeenth nucleoside of the antisense strand comprises the 2′ O-methyl modified nucleoside, in a 5′ to 3′ direction. 758. The pharmaceutical composition of any one of embodiments 740-757, wherein the eighteenth nucleoside of the antisense strand comprises the 2′ O-methyl modified nucleoside, in a 5′ to 3′ direction. 759. The pharmaceutical composition of any one of embodiments 740-758, wherein the nineteenth nucleoside of the antisense strand comprises the 2′ O-methyl modified nucleoside, in a 5′ to 3′ direction. 760. The pharmaceutical composition of any one of embodiments 740-759, wherein the twentieth nucleoside of the antisense strand comprises the 2′ O-methyl modified nucleoside, in a 5′ to 3′ direction. 761. The pharmaceutical composition of any one of embodiments 740-760, wherein the twenty-first nucleoside of the antisense strand comprises the 2′ O-methyl modified nucleoside, in a 5′ to 3′ direction. 762. The pharmaceutical composition of any one of embodiments 740-761, wherein the first, third, fifth, seventh, eleventh, twelfth, thirteenth, fifteenth, seventeenth, nineteenth, twentieth, and twenty-first nucleosides of the antisense strand comprises the 2′ O-methyl modified nucleoside, in a 5′ to 3′ direction. 763. The pharmaceutical composition of any one of embodiments 740-762, wherein the antisense strand comprises at least two contiguous 2′ O-methyl modified nucleosides. 764. The pharmaceutical composition of embodiment 763, wherein the at least two contiguous 2′ O-methyl modified nucleosides is three contiguous 2′ O-methyl modified nucleosides. 765. The pharmaceutical composition of embodiment 763, wherein the at least two contiguous 2′ O-methyl modified nucleosides is four contiguous 2′ O-methyl modified nucleosides. 766. The pharmaceutical composition of embodiment 763, wherein the at least two contiguous 2′ O-methyl modified nucleosides is five contiguous 2′ O-methyl modified nucleosides. 767. The pharmaceutical composition of embodiment 763, wherein the at least two contiguous 2′ O-methyl modified nucleosides is six contiguous 2′ O-methyl modified nucleosides. 768. The pharmaceutical composition of embodiment 763, wherein the at least two contiguous 2′ O-methyl modified nucleosides is seven contiguous 2′ O-methyl modified nucleosides. 769. The pharmaceutical composition of any one of embodiments 740-768, wherein the antisense strand comprises a first sequence comprising at least two contiguous 2′ O-methyl modified nucleosides and a second sequence comprising at least two contiguous 2′ O-methyl modified nucleosides. 770. The pharmaceutical composition of embodiment 769, wherein the first sequence comprises at least three contiguous 2′ O-methyl modified nucleosides, and the second sequence comprises at least three contiguous 2′ O-methyl modified nucleosides. 771. The pharmaceutical composition of embodiment 770, wherein the first sequence comprises three contiguous 2′ O-methyl modified nucleosides, and the second sequence comprises three contiguous 2′ O-methyl modified nucleosides. 772. The pharmaceutical composition of embodiment 770, wherein the first sequence comprises four contiguous 2′ O-methyl modified nucleosides, and the second sequence comprises five contiguous 2′ O-methyl modified nucleosides. 773. The pharmaceutical composition of embodiment 770, wherein the first sequence comprises seven contiguous 2′ O-methyl modified nucleosides, and the second sequence comprises five contiguous 2′ O-methyl modified nucleosides. 774. The pharmaceutical composition of embodiment 770, wherein the first sequence comprises at least four contiguous 2′ O-methyl modified nucleosides. 775. The pharmaceutical composition of embodiment 770, wherein the first sequence comprises at least five contiguous 2′ O-methyl modified nucleosides. 776. The pharmaceutical composition of embodiment 770, wherein the first sequence comprises at least six contiguous 2′ O-methyl modified nucleosides. 777. The pharmaceutical composition of embodiment 770, wherein the first sequence comprises at least seven contiguous 2′ O-methyl modified nucleosides. 778. The pharmaceutical composition of any one of embodiments 769-777, wherein the second sequence comprises at least four contiguous 2′ O-methyl modified nucleosides. 779. The pharmaceutical composition of any one of embodiments 769-777, wherein the second sequence comprises at least five contiguous 2′ O-methyl modified nucleosides. 780. The pharmaceutical composition of any one of embodiments 769-777, wherein the second sequence comprises at least six contiguous 2′ O-methyl modified nucleosides. 781. The pharmaceutical composition of any one of embodiments 769-777, wherein the second sequence comprises at least seven contiguous 2′ O-methyl modified nucleosides.

782. The pharmaceutical composition of any one of embodiments 442-781, wherein the sense strand comprises a ribose. 783. The pharmaceutical composition of embodiment 782, wherein the sense strand comprises about 1, 2, 3, 4, 5, 6, 7, 8, 9, 10, 11, 12, 13, 14, 15, 16, 17, 18, 19, 20, or 21 ribose. 784. The pharmaceutical composition of embodiment 782 or embodiment 783, wherein the nucleoside at position 1, 2, 3, 4, 5, 6, 7, 8, 9, 10, 11, 12, 13, 14, 15, 16, 17, 18, 19, 20, or 21, or any combination thereof, comprises the ribose, in a 5′ to 3′ direction. 785. The pharmaceutical composition of any one of embodiments 442-784, wherein the antisense strand comprises a ribose. 786. The pharmaceutical composition of embodiment 785, wherein the antisense strand comprises about 1, 2, 3, 4, 5, 6, 7, 8, 9, 10, 11, 12, 13, 14, 15, 16, 17, 18, 19, 20, or 21 ribose. 787. The pharmaceutical composition of embodiment 785 or embodiment 786, wherein the nucleoside at position 1, 2, 3, 4, 5, 6, 7, 8, 9, 10, 11, 12, 13, 14, 15, 16, 17, 18, 19, 20, or 21, or any combination thereof, comprises the ribose, in a 5′ to 3′ direction. 788. The pharmaceutical composition of any one of embodiments 442-787, further comprising a lipid attached at either 3′ or 5′ terminus of the sense strand and/or antisense strand. 789. The pharmaceutical composition of embodiment 788, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof. 790. The pharmaceutical composition of embodiment 788 or embodiment 789, wherein the lipid comprises a first lipid on the sense strand and a second lipid on the antisense strand. 791. The pharmaceutical composition of any one of embodiments 788-790, wherein the lipid is positioned on the sense strand. 792. The pharmaceutical composition of embodiment 791, wherein the lipid is positioned at the 5′ end of the sense strand. 793. The pharmaceutical composition of embodiment 791, wherein the lipid is positioned at the 3′ end of the sense strand. 794. The pharmaceutical composition of any one of embodiments 788-793, wherein the lipid is positioned on the antisense strand. 795. The pharmaceutical composition of embodiment 794, wherein the lipid is positioned at the 5′ end of the antisense strand. 796. The pharmaceutical composition of embodiment 794, wherein the lipid is positioned at the 3′ end of the antisense strand. 797. The pharmaceutical composition of any one of embodiments 442-796, wherein the sense strand and the antisense strand form a double-stranded RNA duplex. 798. The pharmaceutical composition of embodiment 797, wherein the double-stranded RNA duplex comprises from about 14 to about 30 nucleosides. 799. The pharmaceutical composition of embodiment 798, wherein the double-stranded RNA duplex comprises from about 17 to about 30 nucleosides. 800. The pharmaceutical composition of embodiment 799, wherein the double-stranded RNA duplex comprises about 21 nucleosides. 801. The pharmaceutical composition of any one of embodiments 797-800, wherein the double-stranded RNA duplex comprises at least one base pair. 802. The pharmaceutical composition of embodiment 801, wherein the first base pair of the double-stranded RNA duplex is an AU base pair. 803. The pharmaceutical composition of any one of embodiments 442-802, wherein the sense strand comprises pattern 1S: 5′ fN s mN s fN-mN-fN-mN-fN-fN-fN-mN-fN-mN-fN-mN-fN-mN-fN-mN-fN s mN s mN 3′, wherein “fN” is a 2′ fluoro-modified nucleoside, “mN” is a 2′ O-methyl modified nucleoside, “-” is a phosphodiester, and “s” is a phosphorothioate.

804. The pharmaceutical composition of any one of embodiments 442-802, wherein the sense strand comprises pattern 2S: 5′ mN s mN s mN-mN-fN-mN-fN-fN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 805. The pharmaceutical composition of any one of embodiments 442-802, wherein the sense strand comprises pattern 3S: 5′ mN s mN s mN-mN-fN-mN-fN-mN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 806. The pharmaceutical composition of any one of embodiments 442-802, wherein the sense strand comprises pattern 4S: 5′ fN s mN s fN-mN-fN-mN-fN-fN-fN-mN-fN-mN-fN-mN-fN-mN-fN-mN-fN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides. 807. The pharmaceutical composition of embodiment 806, wherein the one or more nucleosides is three nucleosides. 808. The pharmaceutical composition of embodiment 806 of embodiment 807, wherein each of the one or more nucleosides independently comprise a ribose or deoxyribose. 809. The pharmaceutical composition of any one of embodiments 442-802, wherein the sense strand comprises pattern 5S: 5′ mN s mN s mN-mN-fN-mN-fN-fN-fN-mN-mN-mN-mN-mN-mN-mN-mN-mN-mN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides. 810. The pharmaceutical composition of embodiment 809, wherein the one or more nucleosides is three nucleosides. 811. The pharmaceutical composition of embodiment 809 of embodiment 810, wherein each of the one or more nucleosides independently comprise a ribose or deoxyribose. 812. The pharmaceutical composition of any one of embodiments 442-811, wherein the antisense strand comprises pattern 1AS: 5′ mN s fN s mN-fN-mN-fN-mN-fN-mN-fN-mN-mN-mN-fN-mN-fN-mN-fN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 813. The pharmaceutical composition of any one of embodiments 442-811, wherein the antisense strand comprises pattern 2AS: 5′ mN s fN s mN-mN-mN-fN-mN-fN-fN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 814. The pharmaceutical composition of any one of embodiments 442-811, wherein the antisense strand comprises pattern 3AS: 5′ mN s fN s mN-mN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate. 815. The pharmaceutical composition of any one of embodiments 442-811, wherein the antisense strand comprises pattern 4AS: 5′ mN s fN s mN-fN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

816. The pharmaceutical composition of any one of embodiments 442-802, wherein the sense strand comprises mUsmGsmGmUfGmUfCfGfAmGmAmGmAmUmCmAmCmUmAsmUsmU (SEQ ID NO: 5558), or a nucleic acid sequence thereof having about 1 or 2 nucleoside substitutions, additions, or deletions. 817. The pharmaceutical composition of any one of embodiments 442-802, wherein the sense strand comprises mUsmCsmGmAfGmAfGfAfUmCmAmCmUmGmAmAmAmUmAsmUsmU (SEQ ID NO: 5560), or a nucleic acid sequence thereof having about 1 or 2 nucleoside substitutions, additions, or deletions. 818. The pharmaceutical composition of any one of embodiments 442-802, wherein the sense strand comprises mCsmAsmGmGfUmUfUfGfCmCmAmCmUmUmUmGmUmCmAsmUsmU (SEQ ID NO: 5562), or a nucleic acid sequence thereof having about 1 or 2 nucleoside substitutions, additions, or deletions. 819. The pharmaceutical composition of any one of embodiments 442-802, wherein the sense strand comprises mGsmCsmAmCfCmUfUfUfUmCmAmUmGmGmUmCmUmCmAsmUsmU (SEQ ID NO: 5564), or a nucleic acid sequence thereof having about 1 or 2 nucleoside substitutions, additions, or deletions. 820. The pharmaceutical composition of any one of embodiments 442-802, wherein the sense strand comprises mAsmGsmCmUfGmGfAfGfCmCmUmUmCmCmUmAmAmCmAsmUsmU (SEQ ID NO: 5566), or a nucleic acid sequence thereof having about 1 or 2 nucleoside substitutions, additions, or deletions. 821. The pharmaceutical composition of any one of embodiments 442-802, wherein the sense strand comprises fUsmGsfGmUfGmUfCfGfAmGfAmGfAmUfCmAfCmUfAsmUsmU (SEQ ID NO: 5456), or a nucleic acid sequence thereof having about 1 or 2 nucleoside substitutions, additions, or deletions. 822. The pharmaceutical composition of any one of embodiments 442-802, wherein the sense strand comprises fUsmCsfGmAfGmAfGfAfUmCfAmCfUmGfAmAfAmUfAsmUsmU (SEQ ID NO: 5460), or a nucleic acid sequence thereof having about 1 or 2 nucleoside substitutions, additions, or deletions. 823. The pharmaceutical composition of any one of embodiments 442-802, wherein the sense strand comprises fCsmAsfGmGfUmUfUfGfCmCfAmCfUmUfUmGfUmCfAsmUsmU (SEQ ID NO: 5476), or a nucleic acid sequence thereof having about 1 or 2 nucleoside substitutions, additions, or deletions. 824. The pharmaceutical composition of any one of embodiments 442-802, wherein the sense strand comprises fGsmCsfAmCfCmUfUfUfUmCfAmUfGmGfUmCfUmCfAsmUsmU (SEQ ID NO: 5510), or a nucleic acid sequence thereof having about 1 or 2 nucleoside substitutions, additions, or deletions. 825. The pharmaceutical composition of any one of embodiments 442-802, wherein the sense strand comprises fAsmGsfCmUfGmGfAfGfCmCfUmUfCmCfUmAfAmCfAsmUsmU (SEQ ID NO: 5524), or a nucleic acid sequence thereof having about 1 or 2 nucleoside substitutions, additions, or deletions. 826. The pharmaceutical composition of any one of embodiments 442-802, wherein the sense strand comprises mUsmGsmGmUfGmUfCmGfAmGmAmGmAmUmCmAmCmUmAsmUsmU (SEQ ID NO: 6050). 827. The pharmaceutical composition of any one of embodiments 442-802, wherein the sense strand comprises mUsmCsmGmAfGmAfGmAfUmCmAmCmUmGmAmAmAmUmAsmUsmU (SEQ ID NO: 6051), or a nucleic acid sequence thereof having about 1 or 2 nucleoside substitutions, additions, or deletions. 828. The pharmaceutical composition of any one of embodiments 442-802, wherein the sense strand comprises mCsmAsmGmGfUmUfUmGfCmCmAmCmUmUmUmGmUmCmAsmUsmU (SEQ ID NO: 6052), or a nucleic acid sequence thereof having about 1 or 2 nucleoside substitutions, additions, or deletions. 829. The pharmaceutical composition of any one of embodiments 442-802, wherein the sense strand comprises mGsmCsmAmCfCmUfUmUfUmCmAmUmGmGmUmCmUmCmAsmUsmU (SEQ ID NO: 6053), or a nucleic acid sequence thereof having about 1 or 2 nucleoside substitutions, additions, or deletions. 830. The pharmaceutical composition of any one of embodiments 442-802, wherein the sense strand comprises mAsmGsmCmUfGmGfAmGfCmCmUmUmCmCmUmAmAmCmAsmUsmU (SEQ ID NO: 6054), or a nucleic acid sequence thereof having about 1 or 2 nucleoside substitutions, additions, or deletions. 831. The pharmaceutical composition of any one of embodiments 442-802, wherein the sense strand comprises fUsmGsfGmUfGmUfCfGfAmGfAmGfAmUfCmAfCmUfAsmUsmUNNN-lipid (wherein each N is independently a nucleobase such as A, U, C, or G) (SEQ ID NO: 6055), or a nucleic acid sequence thereof having about 1 or 2 nucleoside substitutions, additions, or deletions. 832. The pharmaceutical composition of any one of embodiments 442-802, wherein the sense strand comprises fUsmCsfGmAfGmAfGfAfUmCfAmCfUmGfAmAfAmUfAsmUsmUNNN-lipid (wherein each N is independently a nucleobase such as A, U, C, or G) (SEQ ID NO: 6056), or a nucleic acid sequence thereof having about 1 or 2 nucleoside substitutions, additions, or deletions. 833. The pharmaceutical composition of any one of embodiments 442-802, wherein the sense strand comprises fCsmAsfGmGfUmUfUfGfCmCfAmCfUmUfUmGfUmCfAsmUsmUNNN-lipid (wherein each N is independently a nucleobase such as A, U, C, or G) (SEQ ID NO: 6057), or a nucleic acid sequence thereof having about 1 or 2 nucleoside substitutions, additions, or deletions. 834. The pharmaceutical composition of any one of embodiments 442-802, wherein the sense strand comprises fGsmCsfAmCfCmUfUfUfUmCfAmUfGmGfUmCfUmCfAsmUsmUNNN-lipid (wherein each N is independently a nucleobase such as A, U, C, or G) (SEQ ID NO: 6058), or a nucleic acid sequence thereof having about 1 or 2 nucleoside substitutions, additions, or deletions. 835. The pharmaceutical composition of any one of embodiments 442-802, wherein the sense strand comprises fAsmGsfCmUfGmGfAfGfCmCfUmUfCmCfUmAfAmCfAsmUsmUNNN-lipid (wherein each N is independently a nucleobase such as A, U, C, or G) (SEQ ID NO: 6059), or a nucleic acid sequence thereof having about 1 or 2 nucleoside substitutions, additions, or deletions. 836. The pharmaceutical composition of any one of embodiments 442-802, wherein the sense strand comprises mUsmGsmGmUfGmUfCfGfAmGmAmGmAmUmCmAmCmUmAsmUsmUNNN-lipid (wherein each N is independently a nucleobase such as A, U, C, or G) (SEQ ID NO: 6060), or a nucleic acid sequence thereof having about 1 or 2 nucleoside substitutions, additions, or deletions. 837. The pharmaceutical composition of any one of embodiments 442-802, wherein the sense strand comprises mUsmCsmGmAfGmAfGfAfUmCmAmCmUmGmAmAmAmUmAsmUsmUNNN-lipid (wherein each N is independently a nucleobase such as A, U, C, or G) (SEQ ID NO: 6061), or a nucleic acid sequence thereof having about 1 or 2 nucleoside substitutions, additions, or deletions. 838. The pharmaceutical composition of any one of embodiments 442-802, wherein the sense strand comprises mCsmAsmGmGfUmUfUfGfCmCmAmCmUmUmUmGmUmCmAsmUsmUNNN-lipid (wherein each N is independently a nucleobase such as A, U, C, or G) (SEQ ID NO: 6062), or a nucleic acid sequence thereof having about 1 or 2 nucleoside substitutions, additions, or deletions. 839. The pharmaceutical composition of any one of embodiments 442-802, wherein the sense strand comprises mGsmCsmAmCfCmUfUfUfUmCmAmUmGmGmUmCmUmCmAsmUsmUNNN-lipid (wherein each N is independently a nucleobase such as A, U, C, or G) (SEQ ID NO: 6063), or a nucleic acid sequence thereof having about 1 or 2 nucleoside substitutions, additions, or deletions. 840. The pharmaceutical composition of any one of embodiments 442-802, wherein the sense strand comprises mAsmGsmCmUfGmGfAfGfCmCmUmUmCmCmUmAmAmCmAsmUsmUNNN-lipid (wherein each N is independently a nucleobase such as A, U, C, or G) (SEQ ID NO: 6064), or a nucleic acid sequence thereof having about 1 or 2 nucleoside substitutions, additions, or deletions. 841. The pharmaceutical composition of any one of embodiments 816-840, wherein the antisense strand comprises mUsfAsmGmUmGfAmUmCmUmCmUmCmGfAmCfAmCmCmAsmUsmU (SEQ ID NO: 5559), or a nucleic acid sequence thereof having about 1 or 2 nucleoside substitutions, additions, or deletions. 842. The pharmaceutical composition of any one of embodiments 816-840, wherein the antisense strand comprises mUsfAsmUmUmUfCmAmGmUmGmAmUmCfUmCfUmCmGmAsmUsmU (SEQ ID NO: 5561), or a nucleic acid sequence thereof having about 1 or 2 nucleoside substitutions, additions, or deletions. 843. The pharmaceutical composition of any one of embodiments 816-840, wherein the antisense strand comprises mUsfGsmAmCmAfAmAmGmUmGmGmCmAfAmAfCmCmUmGsmUsmU (SEQ ID NO: 5563), or a nucleic acid sequence thereof having about 1 or 2 nucleoside substitutions, additions, or deletions. 844. The pharmaceutical composition of any one of embodiments 816-840, wherein the antisense strand comprises mUsfGsmAmGmAfCmCmAmUmGmAmAmAfAmGfGmUmGmCsmUsmU (SEQ ID NO: 5565), or a nucleic acid sequence thereof having about 1 or 2 nucleoside substitutions, additions, or deletions. 845. The pharmaceutical composition of any one of embodiments 816-840, wherein the antisense strand comprises mUsfGsmUmUmAfGmGmAmAmGmGmCmUfCmCfAmGmCmUsmUsmU (SEQ ID NO: 5567), or a nucleic acid sequence thereof having about 1 or 2 nucleoside substitutions, additions, or deletions. 846. The pharmaceutical composition of any one of embodiments 816-840, wherein the antisense strand comprises mUsfAsmGfUmGfAmUfCmUfCmUmCmGfAmCfAmCfCmAsmUsmU (SEQ ID NO: 5457), or a nucleic acid sequence thereof having about 1 or 2 nucleoside substitutions, additions, or deletions. 847. The pharmaceutical composition of any one of embodiments 816-840, wherein the antisense strand comprises mUsfAsmUfUmUfCmAfGmUfGmAmUmCfUmCfUmCfGmAsmUsmU (SEQ ID NO: 5461), or a nucleic acid sequence thereof having about 1 or 2 nucleoside substitutions, additions, or deletions. 848. The pharmaceutical composition of any one of embodiments 816-840, wherein the antisense strand comprises mUsfGsmAfCmAfAmAfGmUfGmGmCmAfAmAfCmCfUmGsmUsmU (SEQ ID NO: 5477), or a nucleic acid sequence thereof having about 1 or 2 nucleoside substitutions, additions, or deletions. 849. The pharmaceutical composition of any one of embodiments 816-840, wherein the antisense strand comprises mUsfGsmAfGmAfCmCfAmUfGmAmAmAfAmGfGmUfGmCsmUsmU (SEQ ID NO: 5511), or a nucleic acid sequence thereof having about 1 or 2 nucleoside substitutions, additions, or deletions. 850. The pharmaceutical composition of any one of embodiments 816-840, wherein the antisense strand comprises mUsfGsmUfUmAfGmGfAmAfGmGmCmUfCmCfAmGfCmUsmUsmU (SEQ ID NO: 5525), or a nucleic acid sequence thereof having about 1 or 2 nucleoside substitutions, additions, or deletions. 851. The pharmaceutical composition of any one of embodiments 816-840, wherein the antisense strand comprises mUsfAsmGmUmGfAmUfCfUmCmUmCmGfAmCfAmCmCmAsmUsmU (SEQ ID NO: 6065), or a nucleic acid sequence thereof having about 1 or 2 nucleoside substitutions, additions, or deletions. 852. The pharmaceutical composition of any one of embodiments 816-840, wherein the antisense strand comprises mUsfAsmUmUmUfCmAfGfUmGmAmUmCfUmCfUmCmGmAsmUsmU (SEQ ID NO: 6066), or a nucleic acid sequence thereof having about 1 or 2 nucleoside substitutions, additions, or deletions. 853. The pharmaceutical composition of any one of embodiments 816-840, wherein the antisense strand comprises mUsfGsmAmCmAfAmAfGfUmGmGmCmAfAmAfCmCmUmGsmUsmU (SEQ ID NO: 6067), or a nucleic acid sequence thereof having about 1 or 2 nucleoside substitutions, additions, or deletions. 854. The pharmaceutical composition of any one of embodiments 816-840, wherein the antisense strand comprises mUsfGsmAmGmAfCmCfAfUmGmAmAmAfAmGfGmUmGmCsmUsmU (SEQ ID NO: 6068), or a nucleic acid sequence thereof having about 1 or 2 nucleoside substitutions, additions, or deletions. 855. The pharmaceutical composition of any one of embodiments 816-840, wherein the antisense strand comprises mUsfGsmUmUmAfGmGfAfAmGmGmCmUfCmCfAmGmCmUsmUsmU (SEQ ID NO: 6069), or a nucleic acid sequence thereof having about 1 or 2 nucleoside substitutions, additions, or deletions 856. The pharmaceutical composition of any one of embodiments 816-840, wherein the antisense strand comprises mUsfAsmGfUmGfAmUmCmUmCmUmCmGfAmCfAmCmCmAsmUsmU (SEQ ID NO: 6070), or a nucleic acid sequence thereof having about 1 or 2 nucleoside substitutions, additions, or deletions. 857. The pharmaceutical composition of any one of embodiments 816-840, wherein the antisense strand comprises mUsfAsmUfUmUfCmAmGmUmGmAmUmCfUmCfUmCmGmAsmUsmU (SEQ ID NO: 6071), or a nucleic acid sequence thereof having about 1 or 2 nucleoside substitutions, additions, or deletions. 858. The pharmaceutical composition of any one of embodiments 816-840, wherein the antisense strand comprises mUsfGsmAfCmAfAmAmGmUmGmGmCmAfAmAfCmCmUmGsmUsmU (SEQ ID NO: 6072), or a nucleic acid sequence thereof having about 1 or 2 nucleoside substitutions, additions, or deletions. 859. The pharmaceutical composition of any one of embodiments 816-840, wherein the antisense strand comprises mUsfGsmAfGmAfCmCmAmUmGmAmAmAfAmGfGmUmGmCsmUsmU (SEQ ID NO: 6073), or a nucleic acid sequence thereof having about 1 or 2 nucleoside substitutions, additions, or deletions. 860. The pharmaceutical composition of any one of embodiments 816-840, wherein the antisense strand comprises mUsfGsmUfUmAfGmGmAmAmGmGmCmUfCmCfAmGmCmUsmUsmU (SEQ ID NO: 6074), or a nucleic acid sequence thereof having about 1 or 2 nucleoside substitutions, additions, or deletions.

861. The pharmaceutical composition of any one of embodiments 442-802, wherein the sense strand comprises pattern 1S and the antisense strand comprises pattern 1AS. 862. The pharmaceutical composition of any one of embodiments 442-802, wherein the sense strand comprises pattern 2S and the antisense strand comprises pattern 2AS. 863. The pharmaceutical composition of any one of embodiments 442-802, wherein the sense strand comprises pattern 3S and the antisense strand comprises pattern 3AS. 864. The pharmaceutical composition of any one of embodiments 442-802, wherein the sense strand comprises pattern 4S and the antisense strand comprises pattern 4AS. 865. A method of treating one or more disorders of the upper and lower airway in a patient in need thereof, the method comprising administering to the patient an siRNA that targets a portion of mRNA encoding ALOX15, wherein the siRNA comprises a sense strand and an antisense strand, and a double-stranded nucleic acid region, and wherein there is greater than 90% sequence identity or greater than 90% sequence complementarity between the double-stranded nucleic acid region of the siRNA and the portion of mRNA encoding ALOX15 that is targeted by the siRNA; wherein the one or more disorders of the upper and lower airways comprises nasal polyposis, chronic sinusitis, allergic rhinitis, or NSAID-exacerbated respiratory disease, or a combination thereof. 866. The method of embodiment 865, wherein the portion comprises from about 14 to about 30 contiguous nucleosides of the mRNA encoding ALOX15.

867. A method of treating one or more disorders of the upper and lower airway in a patient in need thereof, the method comprising administering to the patient an agent comprising a siRNA directed towards a ALOX15 target sequence, the siRNA comprising: a) a sense sequence comprising a nucleic acid sequence identical to or substantially identical to the ALOX15 target sequence, wherein the sense strand that is substantially identical to the ALOX15 target sequence differs in identity from the ALOX15 target sequence by no more than five nucleosides; and b) an anti-sense sequence comprising a nucleic acid sequence complementary to or substantially complementary to the ALOX15 target sequence, wherein the antisense strand that is substantially complementary to the ALOX15 target sequence differs in complementarity from the ALOX15 target sequence by no more than five nucleosides; wherein the method is effective in treating the one or more disorders, and the one or more disorders comprises nasal polyposis, chronic sinusitis, allergic rhinitis, or NSAID-exacerbated respiratory disease, or a combination thereof. 868. The method of embodiment 867, wherein the ALOX15 target sequence comprises from about 14 to about 30 contiguous nucleosides of SEQ ID NO: 5357. 869. The method of any one of embodiments 865-868, wherein the siRNA is administered in an effective amount to reduce eosinophil count in the patient. 870. The method of any one of embodiments 865-869, wherein the siRNA is administered in an effective amount to reduce number and/or size of nasal polyps in the patient. 871. The method of any one of embodiments 865-870, wherein the siRNA is administered in an effective amount to increase nasal inspiratory peak in the patient. 872. The method of any one of embodiments 865-871, wherein the siRNA is administered in an effective amount to reduce airway symptoms in the patient. 873. The method of any one of embodiments 865-872, wherein the siRNA comprises any pharmaceutical composition of embodiments 1-864.

### Pharmaceutical Compositions and Formulations

In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof. In some instances, a nanoparticle includes a core or a core and a shell, as in a core-shell nanoparticle. In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin or dextrin or cyclodextrin. In some instances, a nanoparticle comprises a graphene-coated nanoparticle. In some cases, a nanoparticle has at least one dimension of less than about 500 nm, 400 nm, 300 nm, 200 nm, or 100 nm. In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some embodiments, the pharmaceutical formulation comprises a delivery vector, e.g., a recombinant vector, for the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

The pharmacokinetics and biodistribution of nanoparticles are dependent upon their size and charge. Upon iv administration, large (>200 nm) and/or highly positively charged (surface charge >20 mV) are primarily distributed among endothelial tissues and macrophages in the liver and spleen and have a half-life of circulation less than 2 hours. Reduction in size (<100 nm) and surface charge (˜0 mV) results increased circulation times. Local administration of positively charged polyplexes results in association with cells at site of application such as epithelial cells.

Strategies for cytoplasmic delivery. There are a variety of strategies to facilitate cytoplasmic delivery of oligonucleotides including endosomal buffering (i.e. proton sponge), titratable amphiphiles, cell penetrating peptides and masked membrane lytic polymers. The mechanism of endosomal buffering (i.e. proton sponge) to facilitate endosomolysis relies on the ability of agents such as polyamines to buffer endosomal/lysosomal compartments. The resistance to acidification is postulated to result in increased osmotic pressure that results in lysis of the lysosomal compartment. Titratable amphiphiles are polymers/peptides whose structure is pH-dependent in such a way that at acidic pH they are hydrophobic and membrane disruptive. Typically, titratable amphiphiles are polyanionic polymers or peptides composed of carboxylic acids that become neutral and membrane disruptive upon acidification. Cell penetrating peptides (CPPs) are cationic peptides, with a high propensity of guanidinium groups, that enter cells without any apparent membrane lysis. Masked lytic polymers are membrane disruptive polymers whose membrane interactivity is attenuated by reversible covalent modification. Like titratable amphiphiles, the mechanism of endosomolysis by masked polymers relies on the use of amphipathic polymers whose ability to lyse membranes is controlled such that the activity is only functional in the acidic environment of the endosome/lysosome. In the case of titratable amphiphiles, the mechanism of control is a reversible protonation of carboxylic acids. In the case of masked polymers, the control of membrane activity is the irreversible cleavage of a group that inhibits membrane interactivity of the polymer.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts also include sodium chloride, potassium chloride, sodium thiosulfate, sodium bisulfite and ammonium sulfate.

In some instances, the pharmaceutical formulations further include a diluent which used to stabilize compounds because it provides a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Conjugated oligonucleotides: oligonucleotides may have groups conjugated—via covalent bonds—that prolong circulation, provide targeting to tissues and facilitate intracellular delivery. In some embodiments, circulation is prolonged by the conjugation of polyethylene glycol (PEG). Conjugation of PEG can prevent clearance by two mechanisms: the increase in molecular weight above threshold for renal clearance and the prevention of non-specific interactions with extracellular surfaces and serum components. PEG can be incorporated into nucleic acid delivery vehicles by attachment to components that non-covalently associate with the nucleic acids, e.g. PEGylated lipids and polymers. In some embodiments, conjugation comprises direct PEG conjugation to increase nucleic acid circulation times, decrease nonspecific interactions and alter biodistribution; however, the targeting is passive and the potency of the nucleic may be compromised as PEG MW increases.

A commonly invoked tumor targeting mechanism is the Enhanced Permeability and Retention (EPR) effect, which is when nanoparticles accumulate in tumor tissue much more than they do in normal tissues due to the leaky disorganized vasculature associated with tumor tissues and their lack of lymphatic drainage. EPR-based targeting requires long circulating particles.

Polymer based delivery vehicles: Like lipoplexes, polymer-based transfection vehicles (polyplexes) provide nuclease protection and condensation of larger nucleic acids. Polyplexes are based upon cationic polymers that form electrostatic complexes with anionic nucleic acids. Polycations may be purely synthetic (such as polyethyleneimine), naturally occurring (such as histones, protamine, spermine and spermidine) or synthetic polymers based upon cationic amino acids such as ornithine, lysine and arginine.

Polycations form electrostatic complexes with polyanionic nucleic acids. The strength of the association is dependent upon the size of the nucleic acid and the size and charge density of the polycation.

There are three common strategies to improve the stability and surface charge of polyplexes to improve the circulation and targeting of ability of polyplexes: crosslinking of polycation, addition of a synthetic polyanion and conjugation of PEG.

Crosslinking, also called lateral stabilization and caging, is the formation of covalent polyamine-polyamine bonds after complexation/condensation of the nucleic acid. The crosslinking is accomplished by the addition of bifunctional, amine-reactive reagents that form a 3-D network of bonds around the nucleic acid, thereby making the polyplex resistant to displacement by salts and polyelectrolytes. The stability of the polyplexes is such that the nucleic acid is no longer active unless a mechanism of reversibility is introduced to allow for release of the nucleic acid. A common way to introduce reversibility is the use of disulfide-containing crosslinking reagent that can be reduced in the cytoplasm allowing release of nucleic acid therapeutic.

An exemplary method to reduce the surface charge of a polyplex is the conjugation of PEG, a method commonly known as steric stabilization. The resulting PEG modified polyplexes have prolonged circulation in vivo. PEG modifications can be added to the size chains of polyamines-either before or after polyplex formation- or at the end of the polymer as a block copolymer of PEG and polycation.

Crosslinking and PEGylation are often combined to make stabilized polyplexes of reduced surface charge for systemic administration that can either be passively or actively targeted. As observed for lipoplexes, a variety of small molecule (such as GalNAc, RGD and folate) and biologic targeting ligands (such as transferrin and antibodies) have been conjugated to PEG-modified polyplexes for tissues selective targeting.

The most commonly used polymer for polyplexes- and the originator of the proton sponge mechanism—is polyethylenimine (PEI). PEI's high density of amine groups endows it with high charge density and a continuum of amine pKa's that buffer in the entire pH range of the endosome. The buffering capacity of PEI has been mimicked by the addition of weakly basic imidazole groups.

Oligonucleotide vehicle formulation. The solution conditions in which the oligonucleotide is dissolved, or its delivery vehicle is dispersed may play a role in its delivery. Hypotonic and hypertonic solution conditions may aid in cytoplasmic delivery for systemic and locally administration.

Another class of molecules that can be conjugated in order to increases circulation times is the attachment of lipophilic groups such as cholesterol or other lipophilic moiety with >12 carbons which interact with serum components such as albumen and lipoproteins thereby increasing circulation times and passive accumulation in the liver. It should be remembered that extensive PS modification increases circulation times through associations with serum components, with roughly 10 PS groups required for serum binding.

Delivery Vehicles based upon complexation of nucleic acid. Complexation of oligonucleotide therapeutics with cationic agents inhibits nuclease from degrading the oligonucleotide by forming a steric barrier and by inhibiting nuclease binding by neutralizing anionic charge. The process of forming compact particles of nucleic acids from their extended chains is called condensation, which may be achieved by the addition of multiply-charged cationic species. Multiple positive charges can either be covalently attached to one another in a polycation or non-covalently associated with one another in a complex such as the surface of a cationic liposome. The resulting polycation-polyanion interaction is a colloidal dispersion where the nucleic acid particles vary in size and shape depending on the nucleic acid and the condensing cation. In general, the particles are greater than 20 nm in size, and—in the absence of agents to modulate surface charge such as polyethylene glycol (PEG)-have surface charges >20 mV.

Liposomes

Nucleic acids entrapped in lipids (lipoplexes) may be used as vehicle for the delivery of nucleic acids. Cationic lipids form electrostatic complexes between nucleic acid and lipids. In addition to the cationic lipids, there are typically neutral or anionic helper lipids which are composed of unsaturated fatty acids and are postulated to assist in fusion between the lipoplex and the cellular membrane, and PEGylated lipids, which prevent aggregation during formulation and storage and non-specific interactions in vivo.

Lipids are water insoluble and nucleic acids are organic solvent insoluble. To mix these components in a controlled manner such that formulations are repeatable and relatively homogenous in size, detergents or water-miscible organic solvents such as ethanol are used. After formation of electrostatically-associated complexes, the amphipathic detergent or solvent is then removed by dialysis or solvent exchange. Depending on the components and the mixing procedure is possible to formulate lipoplexes that are well less than 100 nm.

Although the transfection efficiencies of lipoplexes are difficult to predict and optimization is empirical, there are a few design features that have been identified to aid transfection efficiency in vivo: pH-sensitive cationic lipids, the use of unsaturation in the lipid chains and the hydrophobic-hydrophilic balance of PEG-lipids to balance circulation times and transfection efficiencies.

There have been several studies that have shown a correlation between the pKa of the amine groups of the cationic lipid, which is buffer in the range of the endosomal/lysosomal pathway (pH 4-7), and transfection ability. To synthesize lipids with such pKa values, lipids commonly have closely-spaced amines or imidazole groups. The effect of these weakly basic amine groups in the lipoplexes produces several attractive attributes that facilitate in vivo transfection: reduced surface charge at neutral pH thereby decreasing nonspecific interactions in vivo, increased surface charge in acid environment of endosomes and lysosomes thereby increasing electrostatic interactions with the cellular membrane in these compartments and providing buffering groups that can provide endosomolytic activity via the proton sponge mechanism.

Another common motif observed in cationic and helper lipids used in lipoplexes is the presence of unsaturation in their component fatty acids with oleic (18 carbon chain with one double bond) and linoleic (18 carbons with 2 double bonds) being very common. The incorporation of these groups increases fluidity of membranes, aids in the formation of fusogenic lipid structures and facilitates the release of cationic lipids from nucleic acids.

PEG-conjugated lipids are incorporated into lipoplexes to aid in the formation of nonaggregating small complexes and for the prevention of nonspecific interactions in vivo. Due to the hydrophilicity of PEG, their lipid conjugates are not permanently associated with lipoplexes and diffuse from the complexes with dilution and interaction with amphiphilic components in vivo. This loss of PEG shielding from the surface of the lipoplexes aids in transfection efficiency. In general, longer saturated fatty acid chains increase circulation while unsaturation and shorter chains decrease circulation.

For ease of exposition the formulations, compositions and methods in this section are discussed largely with regard to unmodified siRNA compounds. It may be understood, however, that these formulations, compositions and methods can be practiced with other siRNA compounds, e.g., modified siRNAs. An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the siRNA are delivered into the cell where the siRNA can specifically bind to a target RNA and can mediate RNAi. In some cases the liposomes are also specifically targeted, e.g., to direct the siRNA to particular cell types.

A liposome containing a siRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The siRNA preparation is then added to the micelles that include the lipid component. The cationic groups on the lipid interact with the siRNA and condense around the siRNA to form a liposome. After condensation, the detergent is removed, e.g., by dialysis, to yield a liposomal preparation of siRNA.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Feigner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775: 169, 1984; Kim, et al. Biochim. Biophys. Acta 728:339, 1983; and Fukunaga, et al. Endocrinol. 115:757, 1984.

Commonly used techniques for preparing lipid aggregates of appropriate size for use as delivery vehicles include sonication and freeze-thaw plus extrusion (see, e.g., Mayer, et al. Biochim. Biophys. Acta 858: 161, 1986). Microfluidization can be used when consistently small (50 to 200 nm) and relatively uniform aggregates are desired (Mayhew, et al. Biochim. Biophys. Acta 775: 169, 1984). These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274). One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol. Examples of other methods to introduce liposomes into cells in vitro and include U.S. Pat. Nos. 5,283,185; 5,171,678; WO 94/00569; WO 93/24640; WO 91/16024; Feigner, J. Biol. Chem. 269:2550, 1994; Nabel, Proc. Natl. Acad. Sci. 90: 11307, 1993; Nabel, Human Gene Ther. 3:649, 1992; Gershon, Biochem. 32:7143, 1993; and Strauss EMBO J. 11:417, 1992. In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages. Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Feigner, P. L. et al., Proc. Natl. Acad. Sci., USA 8:7413-7417, 1987 and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Choi”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jo 11a, California) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149: 157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with siRNA are useful for treating a dermatological disorder.

Liposomes that include siRNA can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include siRNA can be delivered, for example, subcutaneously by infection in order to deliver siRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

Other formulations are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations.

Surfactants

For ease of exposition the formulations, compositions and methods in this section are discussed largely with regard to unmodified siRNA compounds. It may be understood, however, that these formulations, compositions and methods can be practiced with other siRNA compounds, e.g., modified siRNA compounds, and such practice is within the scope described herein. Surfactants find wide application in formulations such as emulsions (including microemulsions) and liposomes. siRNA (or a precursor, e.g., a larger dsiRNA which can be processed into a siRNA, or a DNA which encodes a siRNA or precursor) compositions can include a surfactant. In some embodiments, the siRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, N.Y., 1988, p. 285).

If the surfactant molecule is not ionized, it is classified as a nonionic surfactant. Nonionic surfactants find wide application in pharmaceutical products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxy ethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfo succinates, and phosphates. The most important members of the anionic surfactant class are the alkyl sulfates and the soaps. If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class. If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in “Pharmaceutical Dosage Forms,” Marcel Dekker, Inc., New York, N.Y., 1988, p. 285).

Micelles and other Membranous Formulations

For ease of exposition the micelles and other formulations, compositions and methods in this section are discussed largely with regard to unmodified siRNA compounds. It may be understood, however, that these micelles and other formulations, compositions and methods can be practiced with other siRNA compounds, e.g., modified siRNA compounds. The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)) composition can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal Cs to C22 alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxy ethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles

For ease of exposition the particles, formulations, compositions and methods in this section are discussed largely with regard to modified siRNA compounds. It may be understood, however, that these particles, formulations, compositions and methods can be practiced with other siRNA compounds, e.g., unmodified siRNA compounds. In some embodiments, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparations may be incorporated into a particle, e.g., a microparticle. Microparticles can be produced by spray-drying, but may also be produced by other methods including lyophilization, evaporation, fluid bed drying, vacuum drying, or a combination of these techniques.

Pharmaceutical Compositions

The iRNA agents described herein may be formulated for pharmaceutical use. Pharmaceutically acceptable compositions comprise a therapeutically-effective amount of one or more of the dsRNA agents in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a compound described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some embodiments, a formulation comprises an excipient selected from the group consisting of cyclodextrins, celluloses, liposomes, micelle forming agents, e.g., bile acids, and polymeric carriers, e.g., polyesters and polyanhydrides; and a compound described herein. In some embodiments, an aforementioned formulation renders orally bio available a compound described herein.

An agent preparation can be formulated in combination with another agent, e.g., another therapeutic agent or an agent that stabilizes a iRNA, e.g., a protein that complexes with iRNA to form an iRNP. Still other agents include chelators, e.g., EDTA (e.g., to remove divalent cations such as Mg2+), salts, RNAse inhibitors (e.g., a broad specificity RNAse inhibitor such as RNAsin) and so forth.

Methods of preparing these formulations or compositions include the step of bringing into association a compound described herein with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The compounds described herein may be formulated for administration in any convenient way for use in human or veterinary medicine, by analogy with other pharmaceuticals.

The term “treatment” is intended to encompass also prophylaxis, therapy and cure. The patient receiving this treatment is any animal in need, including primates, in particular humans, and other mammals such as equines, cattle, swine and sheep; and poultry and pets in general.

Delivery Vectors

Naked oligonucleotides are defined as systems that contain no agents that are associated with the nucleic acid either covalently or non-covalently. The absence of any delivery vehicle may require that the oligonucleotide itself be sufficiently nuclease resistant, sufficiently long circulating and cell targeted. For small, solid-phase synthesized oligonucleotides such as those used in antisense oligonucleotides, RNAi, and innate immune stimulators, the use of nucleotide may provide the required drug-like properties. Double-stranded RNAi agents are produced in a cell in vivo, e.g., from exogenous DNA templates that are delivered into the cell. For example, the DNA templates can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. The DNA templates, for example, can include two transcription units, one that produces a transcript that includes the top strand of a dsRNA agent and one that produces a transcript that includes the bottom strand of a dsRNA agent. When the templates are transcribed, the dsRNA agent is produced, and processed into siRNA agent fragments that mediate gene silencing.

### Therapeutic Regimens

In some embodiments, the pharmaceutical compositions described herein are administered for therapeutic applications.

In some embodiments, a dsRNA described herein targets SEQ ID NO: 5357 and when administered to a patient in an effective amount reduces eosinophil count. In some cases, the patient comprises nasal polyposis, chronic sinusitis, allergic rhinitis, NSAID-exacerbated respiratory disease, or asthma, or a combination thereof. In some cases, the patient has nasal polyposis. In some cases, the patient has chronic sinusitis. In some cases, the patient has allergic rhinitis. In some cases, the patient has an NSAID-exacerbated respiratory disease. In some cases, the systemic or local eosinophil count is reduced by about 10% or more, as compared to the amount prior to administration. In some cases, the dsRNA is present in a pharmaceutical composition. The pharmaceutical composition may comprise a pharmaceutically acceptable carrier. The dsRNA may comprise a nucleoside modification. The dsRNA may comprise a modified internucleoside linkage.

In some embodiments, a dsRNA described herein targets SEQ ID NO: 5357 and when administered in an effective amount to a patient comprising nasal polyps, the nasal polyps are reduced in number and/or size in the patient. In some cases, the nasal polyps are reduced in number and/or size by about 10% or more by CT scan or endoscopic assessment, as compared to the number and/or size prior to administration. In some cases, the dsRNA is present in a pharmaceutical composition. The pharmaceutical composition may comprise a pharmaceutically acceptable carrier. The dsRNA may comprise a nucleoside modification. The dsRNA may comprise a modified internucleoside linkage.

In some embodiments, a dsRNA described herein targets SEQ ID NO: 5357 and when administered to a patient in an effective amount increases nasal inspiratory peak flow in the patient. In some cases, the nasal inspiratory peak flow is increased by about 10% or more as compared to the amount prior to administration. In some cases, the dsRNA is present in a pharmaceutical composition. The pharmaceutical composition may comprise a pharmaceutically acceptable carrier. The dsRNA may comprise a nucleoside modification. The dsRNA may comprise a modified internucleoside linkage.

In some embodiments, a dsRNA described herein targets SEQ ID NO: 5357 and when administered to a patient in an effective amount to reduce airway symptoms in the patient, wherein the patient comprises nasal polyposis, chronic sinusitis, allergic rhinitis, asthma or NSAID-exacerbated respiratory disease, or a combination thereof. In some cases, the patient has nasal polyposis. In some cases, the patient has chronic sinusitis. In some cases, the patient has allergic rhinitis. In some cases, the patient has an NSAID-exacerbated respiratory disease. In some cases, the airway symptoms are reduced by about 10% or more on a patient-reported outcome measure, as compared to prior to administration. In some cases, the dsRNA is present in a pharmaceutical composition. The pharmaceutical composition may comprise a pharmaceutically acceptable carrier. The dsRNA may comprise a nucleoside modification. The dsRNA may comprise a modified internucleoside linkage.

In some embodiments, a dsRNA described herein targets SEQ ID NO: 5357 and when administered to a patient in an effective amount to improve sense of smell in the patient, wherein the patient comprises nasal polyposis, chronic sinusitis, allergic rhinitis, asthma, or NSAID-exacerbated respiratory disease, or a combination thereof. In some cases, the patient has nasal polyposis. In some cases, the patient has chronic sinusitis. In some cases, the patient has allergic rhinitis. In some cases, the patient has an NSAID-exacerbated respiratory disease. In some cases, the sense of smell is improved by about 10% or more on a patient-reported outcome measure, as compared to prior to administration. In some cases, the dsRNA is present in a pharmaceutical composition. The pharmaceutical composition may comprise a pharmaceutically acceptable carrier. The dsRNA may comprise a nucleoside modification. The dsRNA may comprise a modified internucleoside linkage.

In some embodiments, provided is a method of treating one or more disorders of the upper and lower airway in a patient in need thereof, the method comprising administering to the patient an siRNA that targets a portion of mRNA encoding ALOX15, wherein the siRNA comprises a sense strand and an antisense strand, and a double-stranded nucleic acid region, and wherein there is greater than 90% sequence identity or greater than 90% sequence complementarity between the double-stranded nucleic acid region of the siRNA and the portion of mRNA encoding ALOX15 that is targeted by the siRNA; wherein the one or more disorders of the upper and lower airways comprises nasal polyposis, chronic sinusitis, allergic rhinitis, or NSAID-exacerbated respiratory disease, or a combination thereof. In some cases, the portion comprises from about 14 to about 30 contiguous nucleosides of the mRNA encoding ALOX15.

In some embodiments, a method of treating one or more disorders of the upper and lower airway in a patient in need thereof, the method comprising administering to the patient an agent comprising a siRNA directed towards a ALOX15 target sequence, the siRNA comprising: a) a sense sequence comprising a nucleic acid sequence identical to or substantially identical to the ALOX15 target sequence, wherein the sense strand that is substantially identical to the ALOX15 target sequence differs in identity from the ALOX15 target sequence by no more than five nucleosides; and b) an anti-sense sequence comprising a nucleic acid sequence complementary to or substantially complementary to the ALOX15 target sequence, wherein the antisense strand that is substantially complementary to the ALOX15 target sequence differs in complementarity from the ALOX15 target sequence by no more than five nucleosides; wherein the method is effective in treating the one or more disorders, and the one or more disorders comprises nasal polyposis, chronic sinusitis, allergic rhinitis, asthma or NSAID-exacerbated respiratory disease, or a combination thereof. In some cases, the patient has nasal polyposis. In some cases, the patient has chronic sinusitis. In some cases, the patient has allergic rhinitis. In some cases, the patient has an NSAID-exacerbated respiratory disease. In some cases, the ALOX15 target sequence comprises from about 14 to about 30 contiguous nucleosides of SEQ ID NO: 5357. In some cases, the siRNA is administered in an effective amount to reduce eosinophil count in the patient. In some cases, the siRNA is administered in an effective amount to reduce nasal polyps in the patient. In some cases, the siRNA is administered in an effective amount to reduce eosinophil infiltration in the patient. In some cases, the siRNA is administered in an effective amount to reduce airway inflammation in the patient.

In some any of the exemplary methods provided, the dsRNA may comprise the formulas and/or patterns described elsewhere herein. For instance, the dsRNA may comprise Formula IA, IB, IIA, IIB, IIIA, IIIB, IVA, IVB, VA, VB, VIA, VIB, VIIA, VIIB, VIIIA, or VIIIB, or a combination thereof. The dsRNA may comprise pattern 1S, 1AS, 2S, 2AS, 3S, 3AS, 4S, 4AS, or 5S, or a combination thereof.

In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

Routes of Delivery

A composition that includes an iRNA can be delivered to a subject by a variety of routes. Exemplary routes include: intravenous, subcutaneous, topical, rectal, anal, vaginal, nasal, pulmonary, ocular.

The iRNA molecules described herein can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Nasal Delivery Formulations

Nasal drug delivery has been accomplished using several methods. One of the oldest methods of delivering liquids to the nasal cavity is the use of drops. Drops are advantageous as they are low-cost and relatively straightforward to manufacture. A common method for the nasal administration of liquids on the market today is the use of meter-dosed pump sprays. Meter-dosed pump sprays accurately deliver volumes between 25 and 200 μL. The particle size of the drops from pump sprays is a product of the device, patient handling, as well as the formulation, which varies based on the viscosity and surface tension of the product. Nasal formulations can also be delivered as powders. Powder drug delivery provides a high mass of active ingredients for a given volume.

Provided herein are compositions of lipoxygenase inhibitors, methods for making the same and methods for treating conditions mediated by lipoxygenase. Lipoxygenase enzymes play an important role in various diseases such as asthma, rheumatoid arthritis, gout, psoriasis, allergic rhinitis, Crohn's disease, respiratory distress syndrome, chronic obstructive pulmonary disease, acne, atherosclerosis, aortic aneurysm, sickle cell disease, acute lung injury, ischemia/reperfusion injury, nasal polyposis and/or inflammatory bowel disease among others. Accordingly, compounds which inhibit lipoxygenase activity are useful in the treatment and/or prevention of such diseases.

The poor solubility in water of some lipoxygenase inhibitors prevents these beneficial agents from broader use than they would otherwise enjoy if aqueous formulations could be prepared at therapeutically effective concentrations for administration, particularly, formulations for nasal or intratracheal.

Methods for modification of a poorly soluble or insoluble drug itself in an attempt to render it more suitable for administration include altering the morphology or molecular structure of the drug. Other methods include vehicle modification of a poorly soluble or insoluble drug and include the use of salt formation, co-solvent/solubilization, solid carrier systems, micellization, lipid vesicle, oil-water partitioning, and complexation. One approach for delivering a poorly soluble or insoluble agent is to formulate the drug as a solid particle suspension. Drugs that are insoluble in water can provide the significant benefit of stability when formulated as a suspension of particles in an aqueous medium to create a microparticulate or nanoparticulate suspension. Suspensions of solid particles having effective average size of from about 15 to about 1 micron are commonly referred to as nanosuspensions, and are most suitable for administration. These suspensions generally include small particles of insoluble compounds. Forming small particle compositions of lipoxygenase inhibitors can lead to increased therapeutic efficacy and increased therapeutic applications of the drug. In addition, small particle suspensions can be prepared having higher concentrations of the lipoxygenase inhibitor for later dilution prior to injection. Injectable formulations of lipoxygenase inhibitors could permit its use in treating a broad array of conditions mediated by lipoxygenase.

Once small particle suspensions having therapeutically effective concentrations of lipoxygenase inhibitors have been prepared, solid concentrates can also be prepared by known methods, such as lyophilization, spray-drying and/or supercritical fluid extraction. These solid concentrates can then be resuspended at the time of administration. Also, these solid concentrates may also be compounded to produce a single dosage form such as tablets, capsules, lozenges, suppositories, coated tablets, capsules, ampoules, suppositories, delayed release formulations, controlled release formulations, extended release formulations, pulsatile release formulations, immediate release formulations, gastroretentive formulations, effervescent tablets, fast melt tablets, oral liquid and sprinkle formulations. The solid concentrates may also be formulated in a form selected from the group consisting of a patch, a powder preparation for inhalation, a suspension, an ointment and an emulsion.

Small particle compositions lipoxygenase inhibitors can also be formulated in therapeutically effective concentrations for delivery as an aerosol for respiratory delivery to the lungs, as a suspension for topical ophthalmic delivery or as a suspension for intranasal delivery.

Small particles of can be made using any appropriate method including, but not limited to, precipitation methods, mechanical/physical particle size reduction methods such as milling and homogenization, phospholipids coating methods, surfactant coating methods, spray-drying methods, supercritical fluid methods, and hot melt methods. The processes can be separated into four general categories. Each of the categories of processes share the steps of: (1) dissolving lipoxygenase inhibitor in a water miscible organic solvent to create a first solution; (2) mixing the first solution with a second solution that contains water, to precipitate the lipoxygenase inhibitor to create a pre-suspension; and, optionally, (3) adding energy to the pre-suspension in the form of high-shear mixing or heat to provide a stable form of the lipoxygenase inhibitor having the desired size ranges defined above.

Like other routes of administration, cyclodextrins and surfactants can be used to increase the solubility of drugs. The most common cyclodextrins are (α-, β-, and γ-cyclodextrins, consisting of 6, 7 and 8 α-1,4-linked glucose units, respectively. The number of these units determines the size of the cavity. Cyclodextrins are capable of forming inclusion complexes with hydrophobic molecules by taking up a whole molecule, or some part of it, into the cavity. The stability of the complex formed depends on how well the guest molecule fits into the cyclodextrin cavity. A composition comprising a lipoxygenase inhibitor and a cyclodextrin may include inclusion complexes of the lipoxygenase inhibitor and the cyclodextrin as well as lipoxygenase inhibitor and cyclodextrin that are not part of inclusion complexes. α-, β-, and γ-cyclodextrins, have limited aqueous solubility and show some toxicity when given by injection. For example, although P-cyclodextrins form the most stable complex with many drugs, they have the lowest water solubility of the cyclodextrins. Therefore, to overcome these shortfalls, the cyclodextrin structure has been chemically modified to generate a safer cyclodextrin derivative with increased solubility. The modifications are typically made at one or more of the 2, 3, or 6 position hydroxyl groups. As used herein, the term “cyclodextrin” is intended to encompass unmodified cyclodextrins as well as chemically modified derivatives thereof.

Although (α-, β- and γ-cyclodextrins can be used for complex formation with lipoxygenase inhibitors, preferred cyclodextrins are the β- and γ-cyclodextrins and even more preferred are the β-cyclodextrins. Preferred β-cyclodextrins include 2-hydroxypropyl-β-cyclodextrin, methyl-β-cyclodextrin and sulfobutyl derivatized β-cyclodextrin.).

Examples of surfactants include PEG-based nonionic surfactants polysorbate 20, polysorbate 80 and poloxamers. Additionally, cyclodextrins and surfactants can act as permeation enhancers to increase the bioavailability for poorly permeable drugs. Another approach to providing larger doses is using powder delivery formulations. Depending on the bulk density of the powder, quantities up to about 50 mg can be dosed intranasally. The pH of the solution may be modulated to affect the solubility and permeability of the poorly water-soluble drugs. The nasal mucosa can withstand buffers with pH range of 3-10.

To increase the residence time for nasal absorption of drugs after delivery, formulators may add viscosity-increasing and mucoadhesive agents to the formulations. To permit effective dosing of the formulation while maintaining an increased residence time, an in-situ gelling formulation may be used, example of a gel forming agents are gellan and pectin. Another strategy is the use of thixotropic rheological properties that have a low viscosity during actuation. Another method of utilizing mucoadhesive excipients in the formulation intended for nasal delivery is to produce microspheres of drug within the excipient.

Methods Routes for Administration

Provided herein are methods for nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered drop wise to the subject's eye, or a cream formulation can be administered to a wound site.

The compositions described herein can be administered parenterally such as, for example, by intravenous, intramuscular, intrathecal or subcutaneous injection. Parenteral administration can be accomplished by incorporating the compositions described herein into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120 C, dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the disclosure, “pulmonary” is also meant to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

The pharmaceutical compositions described herein may be co-administered with one or more additional agents separately or in the same formulation. Such additional agents include, for example, anti-histamines, beta agonists (e.g., albuterol), antibiotics, anti-inflammatories (e.g. ibuprofen, prednisone (corticosteroid) or pentoxifylline), anti-fungals, (e.g. Amphotericin B, Fluconazole, Ketoconazol, and Itraconazol), steroids, decongestants, bronchodialators, and the like. The formulation may also contain preserving agents, solubilizing agents, chemical buffers, surfactants, emulsifiers, colorants, odorants and sweeteners.

The pharmaceutical composition described herein can be used to treat a patient suffering from a condition mediated by lipoxygenase and/or leukotriene activity. In some embodiments, the condition is mediated by 15-lipoxygenase activity. In some embodiments, the condition is an inflammatory condition.

Conditions mediated by lipoxygenase and/or leukotriene activity include, but are not limited to asthma, rheumatoid arthritis, gout, psoriasis, allergic rhinitis, respiratory distress syndrome, chronic obstructive pulmonary disease, acne, atopic dermatitis, atherosclerosis, aortic aneurysm, sickle cell disease, acute lung injury, ischemia/reperfusion injury, nasal polyposis, inflammatory bowel disease (including, for example, ulcerative colitis and Crohn's disease), irritable bowel syndrome, cancer, tumors, respiratory syncytial virus, sepsis, endotoxin shock and myocardial infarction. In some embodiments, the condition mediated by lipoxygenase and/or leukotriene activity is an inflammatory condition. Inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, or Hodgkins disease.

Nasal Delivery Devices

For most purposes, a broad distribution of the drug on the mucosal surfaces appears desirable for drugs intended for local action or systemic absorption and for vaccines. In chronic sinusitis and nasal polyposis, targeted delivery to the middle and superior meatuses where the sinus openings are, and where the polyps originate, appears desirable.

Nasal drug delivery devices: Liquid nasal formulations are often aqueous solutions, but suspensions and emulsions can also be delivered. Liquid formulations are considered convenient particularly for topical indications where humidification counteracts the dryness and crusting often accompanying chronic nasal diseases. In traditional spray pump systems, preservatives are typically required to maintain microbiological stability in liquid formulations.

Drops delivered with pipette: Drops may be administered by sucking liquid into a glass dropper, inserting the dropper into the nostril with an extended neck before squeezing the rubber top to emit the drops.

Delivery of liquid with rhinyle catheter and squirt tube: A simple way for a physician or trained assistant to deposit drug in the nose is to insert the tip of a fine catheter or micropipette to the desired area under visual control and squirt the liquid into the desired location.

Squeeze bottles are mainly used to deliver some over-the counter (OTC) products like topical decongestants. By squeezing a partly air-filled plastic bottle, the drug is atomized when delivered from a jet outlet. The dose and particle size vary with the force applied.

Metered-dose spray pumps: The pumps typically deliver 100 μl (25-200 μl) per spray, and they offer high reproducibility of the emitted dose and plume geometry in in vitro tests. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination. Pump manufacturers have developed different spray systems that avoid the need for preservatives. These systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a headdown application is recommended. Some systems have a ball valve at the tip to prevent contamination of the liquid inside the applicator tip. Pumps have been designed with side-actuation. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available. Importantly, the in vivo deposition and clinical performance of metered-dose spray pumps can be enhanced for some applications by adapting the pumps to a novel breath powered “Bi-Directional” delivery technology.

Single- and duo-dose spray devices: Metered-dose spray pumps require priming and some degree of overfill to maintain dose conformity for the labeled number of doses. For expensive drugs and vaccines intended for single administration or sporadic use and where tight control of the dose and formulation is of particular importance, single-dose or duo-dose spray devices are preferred. A simple variant of a single-dose spray device (MAD) is a nosepiece with a spray tip is fitted to a standard syringe. The liquid drug to be delivered is first drawn into the syringe and then the spray tip is fitted onto the syringe.

Nasal pressurized metered-dose inhalers (pMDIs): Many drugs intended for local nasal action are delivered by spray pumps, but some have also been delivered as nasal aerosols produced by pMDIs. The particles from a pMDI are released at a high speed and the expansion of a compressed gas. The particles emitted from the traditional pMDIs had a particle velocity much higher than a spray pump (5,200 vs. 1,500 cm/s at a distance 1-2 cm from the actuator tip). The issues related to the high particle speed and “cold Freon effect” have been reduced with hydrofluoroalkane (HFA)-based pMDI for nasal use offering lower particle speeds. Like spray pumps, nasal pMDIs produce a localized deposition on the anterior non-ciliated epithelium of the nasal vestibule and in the anterior parts of the narrow nasal valve, but due to quick evaporation of the spray delivered with a pMDI, noticeable “drip-out” may be less of an issue.

Powered nebulizers and atomizers: Nebulizers use compressed gasses (air, oxygen, and nitrogen) or ultrasonic or mechanical power to break up medical solutions and suspensions into small aerosol droplets that can be directly inhaled into the mouth or nose. The smaller particles and slow speed of the nebulized aerosol are advocated to increase penetration to the target sites in the middle and superior meatuses and the paranasal sinuses.

Powder devices: Powder medication formulations can offer advantages, including greater stability than liquid formulations and potential that preservatives may not be required. Powders tend to stick to the moist surface of the nasal mucosa before being dissolved and cleared. The use of bioadhesive excipients or agents that slow ciliary action may decrease clearance rates and improve absorption. A number of factors like moisture sensitivity, solubility, particle size, particle shape, and flow characteristics will impact deposition and absorption.

The function of nasal powder devices is usually based on one of three principles: 1. Powder sprayers with a compressible compartment to provide a pressure that when released creates a plume of powder particles fairly similar to that of a liquid spray; 2. Breath-actuated inhalers where the subject uses his own breath to inhale the powder into the nostril from a blister or capsule; and 3. Nasal insufflators describe devices consisting of a mouthpiece and a nosepiece that are fluidly connected. Delivery occurs when the subject exhales into the mouthpiece to close the velum, and the airflow carries the powder particles into the nose through the device nosepiece similar to the rhinyle catheter described above. The principle can be applied to different dispersion technologies.

Dosage

Described herein are methods of administering a dsRNA agent, e.g., a siRNA agent, to a subject (e.g., a human subject). The method includes administering a unit dose of the dsRNA agent, e.g., a siRNA agent, e.g., double stranded siRNA agent that (a) the double-stranded part is 14-30 nucleotides (nt) long, for example, 21-23 nt, (b) is complementary to a target RNA (e.g., an endogenous or pathogen target RNA), and, optionally, (c) includes at least one 3′ overhang 1-5 nucleotide long. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight.

The defined amount can be an amount effective to treat or prevent a disease or disorder, e.g., a disease or disorder associated with the target RNA. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In some embodiments, the unit dose is not administered with a frequency (e.g., not a regular frequency). For example, the unit dose may be administered a single time.

In some embodiments, the effective dose is administered with other traditional therapeutic modalities. In some embodiments, the subject has a viral infection and the modality is an antiviral agent other than a dsRNA agent, e.g., other than a siRNA agent. In some embodiments, the subject has atherosclerosis and the effective dose of a dsRNA agent, e.g., a siRNA agent, is administered in combination with, e.g., after surgical intervention, e.g., angioplasty.

In some embodiments, a subject is administered an initial dose and one or more maintenance doses of a dsRNA agent, e.g., a siRNA agent, (e.g., a precursor, e.g., a larger dsRNA agent which can be processed into a siRNA agent, or a DNA which encodes a dsRNA agent, e.g., a siRNA agent, or precursor thereof). The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days.

Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed. The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA agent species. In some embodiments, the dsRNA agent species has sequences that are non-overlapping and non-adjacent to another species with respect to a naturally occurring target sequence. In some embodiments, the plurality of dsRNA agent species is specific for different naturally occurring target genes. In some embodiments, the dsRNA agent is allele specific.

The dsRNA agents described herein can be administered to mammals, particularly large mammals such as nonhuman primates or humans in a number of ways.

In some embodiments, the administration of the dsRNA agent, e.g., a siRNA agent, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Described herein are methods, compositions, and kits, for rectal administration or delivery of dsRNA agents described herein

### Methods of Inhibiting Expression of the Target Gene

Embodiments described herein also relate to methods for inhibiting the expression of a target gene. The method comprises the step of administering the dsRNA agents in any of the preceding embodiments, in an amount sufficient to inhibit expression of the target gene.

Described herein are methods of modulating the expression of a target gene in a cell, comprising providing to said cell a dsRNA agent provided herein. In some embodiments, the target gene is ALOX15.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

This disclosure is further illustrated by the following examples, which should not be construed as further limiting.

### EXAMPLES

### Example 1. Determination of Genomic Loci Associated with Eosinophilic Airway Disease Traits

Approximately 24,000,000 imputed and directly genotyped variants in 4,125 individuals with a diagnosis of nasal polyps and 319,054 controls without a diagnosis of nasal polyps were evaluated. Logistic regression adjusting for age and sex was carried out, and adjusted for population substructure using principal components as covariates. Applicants discovered about 20 genomic loci significantly associated with diagnosis of nasal polyps, which are represented in a Manhattan plot in FIG. 7.

One cluster of association was at chromosome 17p13.2, encompassing the ALOX15 gene. The most significantly associated variant (rs34210653) at this locus was a low frequency missense variant (minor allele frequency ˜1.7%) in exon 13 of the ALOX15 gene which was consistently associated with the evaluated phenotypes (Table 1). This variant was associated with reduced risk of nasal polyps; carriers of the minor allele of this variant had less than half the risk of nasal polyps as non-carriers (p=2×10\hat{ }−15; OR=0.38). This variant was also associated with reduced risk of chronic rhinosinusitis (p=7×10\hat{ }−12; OR=0.65), allergic rhinitis (p=5×10\hat{ }−9; OR=0.80), asthma (p=9×10\hat{ }−6; OR=0.93) and reduced risk of undergoing sinus surgery including nasal polypectomy (p=5×10\hat{ }−11; OR=0.46). This variant was also associated with reduced blood eosinophil counts (p=2×10\hat{ }−65; beta=−0.02).

TABLE 1
Association of ALOX15 Variant rs34210653 with Disease Traits
ALOX15 rs34210653 (AAF = 0.017)

| | Case | Control | | |
|---|---|---|---|---|
| Phenotype | Count | Count | Effect Size | P Value |
| Nasal Polyps | 4,125 | 319,054 | 0.38 (OR) | 1.8E−15 |
| Chronic Rhinosinusitis | 9,535 | 319,054 | 0.65 (OR) | 7.2E−12 |
| Sinus Surgery | 3,592 | 319,054 | 0.46 (OR) | 5.3E−11 |
| Allergic Rhinitis | 22,509 | 259,537 | 0.80 (OR) | 4.5E−09 |
| Asthma | 41,307 | 281,367 | 0.93 (OR) | 9.1E−06 |
| Eosinophil Count | 324,572 | NA | −0.02 (Beta) | 1.5E−65 |

In addition to the protective loss of function variant rs34210653, Applicants discovered that a separate, independent ALOX15 variant is associated with risk of these diseases. This variant, rs2255888, is an ALOX15 regulatory variant that is associated with increased expression of ALOX15 in whole blood (FIG. 3). The data used for the analyses presented in FIG. 3 were obtained from: the GTEx Portal (Genotype-Tissue Expression Project, supported by the Common Fund of the Office of the Director of the National Institutes of Health, and by NCI, NHGRI, NHLBI, NIDA, NIMH, and NINDS) on 02/01/18.

In an analysis conditioning on the loss of function (LOF) variant, the T allele of rs2255888, which is associated with increased expression of ALOX15 in whole blood, is associated with increased risk of nasal polyps (p=7×10\hat{ }−5; OR=1.2) and increased blood eosinophil counts (p=2×10\hat{ }−22; beta=0.004). In combination with the T560M loss of function variant, Applicants have therefore identified an ALOX15 allelic series that modulates risk for nasal polyps and blood eosinophils. This allelic series consist of an ALOX15 loss of function variant (rs34210653) that is associated with decreased risk of nasal polyps, chronic rhinosinusitis, allergic rhinitis, asthma and decreased blood eosinophil counts, and an ALOX15 regulatory variant that increases ALOX15 expression (rs2255888) that is associated with increased risk of nasal polyps and increased blood eosinophil counts, further suggesting therapeutic inhibition of ALOX15 as a novel, genetically-informed method of treatment of nasal polyps and related eosinophilic diseases of the airway.

Applicants also identified three additional rare variants with evidence for being protein truncating or damaging to the protein and analyzed them collectively in a gene burden test (Table 2). rs113604586 is a rare (AAF 0.003) missense variant that is predicted to be damaging to the protein by 2 separate functional prediction algorithms. rs550406686 is a rare (AAF 0.0006) stop gain variant in ALOX15 at amino acid position 134 (of 662 for full length protein). rs144038526 is a rare (AAF 0.0003) missense variant that has been experimentally shown to reduce ALOX15 catalytic activity.

TABLE 2
Damaging and Loss of Function (LofF) Variants in ALOX15

| | | | Functional | Functional |
|---|---|---|---|---|
| Variant | EAF | Coding Consequence | Effect | Evidence |
| rs34210653 | 0.017 | missense (Thr560Met) | LoF | Experi- |
| | | | | mental |
| rs113604586 | 0.003 | missense (Tyr139Cys) | Damaging | Predicted |
| rs550406686 | 0.0006 | stop gain (Glu134Ter) | LoF | Predicted |
| rs144038526 | 0.0003 | missense (Arg402Trp) | Hypomorph | Experi- |
| | | | | mental |

Aggregated variants in the burden test (without T560M) are associated with protection from nasal polyps and lower blood eosinophils, confirming that these variants are deleterious (Table 3). When aggregated with T560M carriers, there is an increase in statistical significance and a consistent effect size compared to T560M carriers alone. These results are strongly supportive of the hypothesis that inactivation of ALOX15 is protective for eosinophilic airway disease and that therapeutic inhibition will be efficacious for the treatment of these diseases.

TABLE 3
Gene Burden Association Results for ALOX15

| | ALOX15 | | ALOX15 Burden | | ALOX15 Burden | |
|---|---|---|---|---|---|---|
| | T560M | | (w/o T560M) | | (w/T560M) | |
| Phenotype | Effect Size | P Value | Effect Size | P Value | Effect Size | P Value |
| Nasal | 0.38 (OR) | 1.8E−15 | 0.53 (OR) | 0.0008 | 0.40 (OR) | 8.4E−17 |
| Polyps | | | | | | |
| Eosinophil | −0.02 (Beta) | 1.5E−65 | −0.016 (Beta) | 1.4E−09 | −0.019 (Beta) | 1.3E−72 |
| Count | | | | | | |

Taking a slightly different approach and as a further demonstration of the power of human genetics to model therapeutic target biology, the ALOX15 associations with eosinophilic airway diseases were placed in the context of approved and investigational therapies targeting these diseases, focusing on the novel Th2 cytokine monoclonal antibodies. Variants in or near ALOX15 (rs34210653), IL13 (rs1881457), IL5RA (rs13090169) and TSLP (rs1898671) were evaluated for association with multiple traits relevant to eosinophilic airways disease. Variants for the Th2 cytokine genes (IL13, IL5RA and TSLP) were selected for inclusion based on being the variants in or near those genes that were most significantly associated with blood eosinophil counts. Linear regression adjusting for age and sex was carried out, adjusting for population substructure using principal components as covariates. Association results with effect sizes and p-values for association testing with nasal polyps, chronic sinusitis, sinus surgery, nasal steroid use, allergic rhinitis, asthma and blood eosinophil counts are shown in Tables 4A and 4B.

TABLE 4A
Association of ALOX15 and IL13 Variants with Disease Traits

| | ALOX15 rs34210653 | | IL13 rs1881457 | |
|---|---|---|---|---|
| | (EAF = 0.017) | | (EAF = 0.77) | |
| Trait | beta | p-value | beta | p-value |
| Nasal polyps | 0.38 (OR) | 1.8E−15 | 0.90 (OR) | 5.9E−04 |
| Chronic | 0.65 (OR) | 7.2E−12 | 0.95 (OR) | 8.8E−03 |
| Rhinosinusitis | | | | |
| Sinus Surgery | 0.46 (OR) | 5.3E−11 | 0.92 (OR) | 0.01 |
| Nasal Steroid | 0.73 (OR) | 3.5E−05 | 0.95 (OR) | 0.04 |
| Use | | | | |
| Allergic | 0.80 (OR) | 4.5E−09 | — | — |
| rhinitis | | | | |
| Asthma | 0.93 (OR) | 9.1E−06 | 0.91 (OR) | 4.7E−21 |
| Eosinophil | −0.02 (beta) | 1.5E−65 | −0.009 (beta) | 2.6E−87 |
| count | | | | |

TABLE 4B
Association of IL5RA and TSLP Variants with Disease Traits

| | IL5RA rs13090169 | | TSLP rs1898671 | |
|---|---|---|---|---|
| | (EAF = 0.89) | | (EAF = 0.68) | |
| Trait | Effect Size | p-value | Effect Size | p-value |
| Nasal polyps | — | — | 0.85 (OR) | 2.4E−12 |
| Chronic | — | — | 0.94 (OR) | 6.5E−05 |
| Rhinosinusitis | | | | |
| Sinus Surgery | — | — | 0.87 (OR) | 3.2E−08 |
| Nasal Steroid | — | — | 0.90 (OR) | 1.3E−07 |
| Use | | | | |
| Allergic | — | — | 0.93 (OR) | 2.5E−14 |
| rhinitis | | | | |
| Asthma | — | — | 0.91 (OR) | 6.3E−31 |
| Eosinophil | −0.004 (beta) | 3.2E−15 | −0.005 (beta) | 2.0E−52 |
| count | | | | |

Only results where the p value was <0.05 are shown. There is noted variation in the effect allele frequencies (EAF) and functional consequences of the selected genetic variant. Nonetheless, when comparing patterns of association within each target gene, the evidence is strongly supportive of the comparative efficacy of ALOX15 inhibition. In this analysis, ALOX15 compares extremely favorably to the genes targeted by the new generation of biologics for the treatment of eosinophilic airway diseases, and in the majority of comparisons appears to be much more consistent in its effects on the upper airway.

### Example 2. RNA Synthesis and Duplex Annealing

1. Oligonucleotide Synthesis: Oligonucleotides were synthesized on an AKTAoligopilot synthesizer or an ABI 394 synthesizer. Commercially available controlled pore glass solid support (dT-CPG, 500A, Prime Synthesis) and RNA phosphoramidites with standard protecting groups, 5′-O-dimethoxytrityl N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2˜isobutryl-2′-t-butyldimethylsilyl-guanosine-3′-O—N,N′-diisopropyl-2-cyanoethylphosphoramidite, and 5′-O-dimethoxytrityl-2′-t-butyldimethylsilyl-uridine-3′-O—N,N′-diisopropyl-2-cyanoethylphosphoramidite (Pierce Nucleic Acids Technologies) were used for the oligonucleotide synthesis unless otherwise specified. The 2′-F phosphoramidites, 5′-O-dimethoxytrityl-N4-acetyl-2′-fluro-cytidine-3′-O—N,N′-diisopropyl-2-cyanoethyl-phosphoramidite and 5′-O-dimethoxytrityl-2′-fluro-uridine-3′-O—N,N′-diisopropyl-2-cyanoethyl-phosphoramidite were purchased from (Promega). All phosphoramidites were used at a concentration of 0.2M in acetonitrile (CH3CN) except for guanosine which was used at 0.2M concentration in 10% THF/ANC (v/v). Coupling/recycling time of 16 minutes was used. The activator was 5-ethyl thiotetrazole (0.75M, American International Chemicals), for the PO-oxidation Iodine/Water/Pyridine was used and the PS-oxidation PADS (2%>) in 2,6-lutidine/ACN (1:1 v/v) was used.

2. Deprotection-1 (Nucleobase Deprotection): After completion of synthesis, the support was transferred to a 100 ml glass bottle (VWR). The oligonucleotide was cleaved from the support with simultaneous deprotection of base and phosphate groups with 80 mL of a mixture of ethanolic ammonia [ammonia:ethanol (3:1)] for 6.5 h at 55° C. The bottle was cooled briefly on ice and then the ethanolic ammonia mixture was filtered into a new 250 ml bottle. The CPG was washed with 2×40 mL portions of ethanol/water (1:1 v/v). The volume of the mixture was then reduced to ˜ 30 ml by roto-vap. The mixture was then frozen on dry ice and dried under vacuum on a speed vac.

3. Deprotection-II (Removal of 2′ TBDMS group): The dried residue was resuspended in 26 ml of triethylamine, triethylamine trihydro fluoride (TEA.3HF) or pyridine-HF and DMSO (3:4:6) and heated at 60° C. for 90 minutes to remove the tert-butyldimethylsilyl (TBDMS) groups at the 2′ position. The reaction was then quenched with 50 ml of 20 mM sodium acetate and pH adjusted to 6.5, and stored in freezer until purification.

4. Analysis: The oligonucleotides were analyzed by high-performance liquid chromatography (HPLC) prior to purification and selection of buffer and column depends on nature of the sequence and/or conjugated ligand.

5. HPLC Purification: The ligand conjugated oligonucleotides were purified by reverse phase preparative HPLC. The unconjugated oligonucleotides were purified by anion-exchange HPLC on a TSK gel column packed in house. The buffers were 20 mM sodium phosphate (pH 8.5) in 10% CH3CN (buffer A) and 20 mM sodium phosphate (pH 8.5) in 10% CH3CN, 1M NaBr (buffer B). Fractions containing full-length oligonucleotides were pooled, desalted, and lyophilized. Approximately 0.15 OD of desalted oligonucleotides were diluted in water to 150 μl and then pipetted in special vials for CGE and LC/MS analysis. Compounds were finally analyzed by LC-ESMS and CGE.

6. siRNA preparation: For the preparation of siRNA, equimolar amounts of sense and antisense strand were heated in 1×PBS at 95° C. for 5 min and slowly cooled to room temperature. Integrity of the duplex was confirmed by HPLC analysis.

### Example 3. siRNA-Mediated Knockdown of ALOX15 in A549 Cells

Two siRNAs targeted to the ALOX15 mRNA were demonstrated to downregulate levels of ALOX15 mRNA and 15(S)-HETE (15-hydroxyeicosatetraenoic acid), an arachidonic acid metabolite produced by the ALOX15 gene product 15-lipoxygenase, when administered to cultured A549 cells (a non-small cell lung cancer line).

A549 cells (Sigma Cat. No. 86012804-1VL, Lot No. 16J012) were thawed and maintained according to the manufacturer's protocol in Ham's F12 medium (Corning Cat. No. 10-080-CV, Lot. No. 19717005) supplemented with 10% Fetal Bovine Serum (Atlanta Biologicals, Cat. No. S11550, Lot. No. D17056). The cells were maintained in a T75 flask and subcultured via trypsinization for one week before seeding for experiments.

On Day 0, the A549 cells were seeded at 150,000 cells/mL into a Falcon 24-well tissue culture plate (Cat. No. 353047) at 0.5 mL per well. On Day 1, cell culture media in appropriate wells was replaced with fresh media containing 20 ng/mL IL-13 (Sino Biologicals, Cat. No. 10369HNAC5, Lot No. LCL11JU0901).

Two siRNAs master mixes were prepared; the ALOX15 siRNA master mix contained 350 μL of Opti-MEM and 3.5 μL of a mixture of the two ALOX15 siRNAs (ThermoFisher Cat. No. 4427037-s1288 Lot No. AS02B02D and s1289 Lot No. AS02B02E, 10 μM stock). The negative control siRNA master mix contained 350 μL of Opti-MEM and 3.5 μL of negative control siRNA (ThermoFisher Cat. No. 4390843, Lot No. AS028XCJ, 10 μM stock). Next, 3 μL of TransIT-X2 (Mirus Cat. No. MIR6000, Lot. No. 71113956) was added to each master mix. These were incubated for 15 minutes to allow transfection complexes to form, then 51 μL of the appropriate master mix+TransIT-X2 was added to duplicate wells of A549 cells yielding a final siRNA concentration of 10 nM. The sequence of the ALOX15 siRNA s1288 is as follows: sense strand, 5′-GGAAAUCAUCUAUCGGUAUtt-3′ (SEQ ID NO: 5972), antisense strand, 5′-AUACCGAUAGAUGAUUUCCtt-3′ (SEQ ID NO: 5973). These sequences correspond to SEQ ID NOS: 2851 and 2852, respectively, wherein the two nucleotides at the 3′ end of each sequence are replaced by tt. The sequence of the ALOX15 siRNA s1289 is as follows: sense strand, 5′-GCACACUGUUCGAAGCUGAtt-3′ (SEQ ID NO: 5974), antisense strand, 5′-UCAGCUUCGAACAGUGUGCtt-3′ (SEQ ID NO: 5975). These correspond to SEQ ID NOS: 1667 and 1668, respectively, wherein the two nucleotides at the 3′ end of each sequence are replaced by tt.

On Day 2, approximately 24 hours post transfection, the wells were supplemented with 10 μM arachidonic acid (Millipore Sigma Cat. No. 10931, Lot No. BCBU4737). On Day 3, approximately 48 hours post transfection, an aliquot of cell supernatant was taken from each well and stored at −80° C. Cells were then lysed using the Cells-to-Ct kit according to the manufacturer's protocol (ThermoFisher Cat. No. 4399002, Lot No. 1707055). In brief, cells were washed with 50 μL using cold 1×PBS and then lysed by adding 49.5 μL of Lysis Solution and 0.5 μL DNase I per well and pipetting up and down 5 times and incubating for 5 minutes at room temperature. The Stop Solution (5 μL/well) was added to each well and mixed by pipetting up and down five times and then incubating at room temperature for 2 minutes. The reverse transcriptase reaction was performed using 22.5 μL of the lysate according to the manufacturer's protocol. Samples were stored at −80° C. until real-time qPCR was performed in triplicate using TaqMan Gene Expression Assays (Applied Biosystems FAMIALOX15 Cat. No. 4331182-Hs00993765_g1, Lot No. P180613-002-C10; VIC/CyclophilinA Cat. No. 4448489-Hs99999904_m1, Lot No. P180613-003-G09) using a BioRad iCycler.

The cell supernatant was collected 48 hours post siRNA transfection of the A549 cells, and the 15(S)-HETE quantified by ELISA (Cayman Chemical, Cat. No. 534721) according to manufacturer's recommended protocol. Comparison was made between the treatment groups.

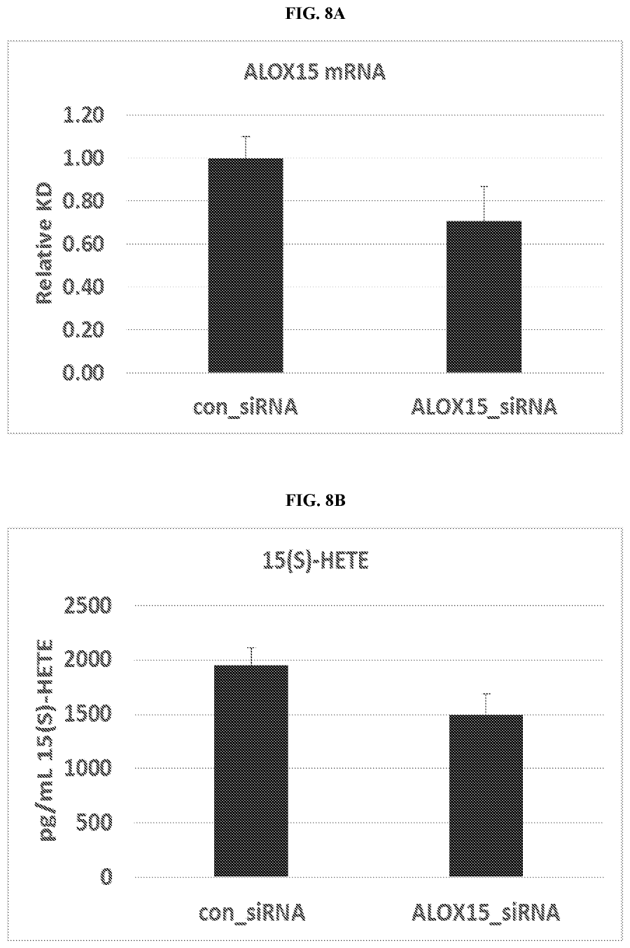

The results are shown in FIGS. 8A and 8B. A decrease in ALOX15 mRNA expression in the A549 cells was observed after transfection with the ALOX15 siRNAs (ALOX15_siRNA) compared to ALOX15 mRNA levels in A549 cells transfected with the non-specific control siRNA (con_siRNA) 48 hours after transfection (p=0.04) (FIG. 8A). A decrease in the amount of 15(S)-HETE in the media from wells containing A549 cells transfected with the ALOX15 siRNAs (ALOX15_siRNA) relative to the amount of 15(S)-HETE in the media from wells containing A549 cells transfected with a non-specific control siRNA (con_siRNA) 48 hours after transfection (p=0.03) was observed (FIG. 8B). These results show that the ALOX15 siRNAs elicited knockdown of ALOX15 mRNA in A549 cells and that the decrease in ALOX15 expression correlated with a decrease in 15(S)-HETE production. These results confirm that siRNA knockdown of ALOX15 mRNA recapitulates the functional effects of the ALOX15 rs34210653 missense variant (T560M) described above. Specifically, both siRNA knockdown of ALOX15 mRNA and a threonine to methionine exchange at position 560 of the ALOX15 protein result in reduced ALOX15-mediated production of 15(S)-HETE.

### Example 4. Selection of Sequences in Order to Identify Therapeutic siRNAs to Downmodulate Expression of the ALOX15 mRNA

Screening sets were defined based by bioinformatic analysis. The therapeutic siRNA molecule has to target human ALOX15 as well as the ALOX15 sequence of at least one toxicology-relevant species, in this case, the non-human primates (NHP) rhesus and cynomolgus monkeys. The key drivers for the design of the screening set were predicted specificity of the siRNAs against the transcriptome of the relevant species as well as cross-reactivity between species. The bioinformatical approach assumes a canonical siRNA structure. Positions 2-18 (5′-3′) of the sense and antisense strand were used for the specificity calculations. The transcriptome database used to for these analyses was NCBI RefSeq DB release 92 (released January 2019). Predicted specificity in human, rhesus monkey, cynomolgus monkey, mouse, rat and dog was determined for sense (S) and antisense (AS) strand. These were assigned a “specificity score” which considers the likelihood of unintended downregulation of any other transcript by full or partial complementarity of an siRNA strand (up to 4 mismatches within positions 2-18) as well as the number and positions of mismatches. Thus, the predicted most likely off-target(s) for antisense and sense strand of each siRNA can be identified. In addition, the number of potential off-targets is used as an additional specificity factor in the specificity score. It is preferable to identify siRNAs with high specificity and a low number of predicted off-targets.

In addition to selecting siRNA sequences with high sequence specificity to ALOX15 mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). In order to circumvent siRNAs to act via functional miRNA binding sites, siRNA strands are avoided that contain natural miRNA seed regions. The miRNA database used for these analyses was miRbase R22 (released March 2018). Seed regions identified in miRNAs from human, mouse, rat, rhesus monkey, dog, rabbit and pig are referred to as “conserved”. Combining the “specificity score” with miRNA seed analysis yields the “specificity category”. This is divided into categories 1-4, with 1 having the highest specificity and 4 having the lowest specificity. Each strand of the siRNA is assigned to a specificity category.

Species cross-reactivity was assessed for human, cynomolgus monkey, rhesus monkey, mouse, rat, dog, and rabbit. The analysis was based on a canonical siRNA design using 19 bases (position 1-19 5′-3′) and/or 17 bases (without considering positions 1 and 19) for cross-reactivity. Full match (FM) as well as single mismatch (SMM) analysis was included.

Analysis of the human Single Nucleotide Polymorphism (SNP) database (NCBI-DB-SNP Build 151, released October 2017) to identify siRNAs targeting regions with known SNPs was also carried out in order to identify siRNAs that may be non-functional in individuals containing the SNP. Positions 1-19 (5′-3′) of the antisense strand was used for cross-reactivity and human SNP analysis. Information regarding the positions of SNPs within the target sequence as well as minor allele frequency (MAF) in case data was obtained in this analysis.

Initial analysis of the relevant ALOX15 mRNA sequence revealed few sequences can be identified that fulfill the specificity requirements and at the same time target ALOX15 mRNA in all relevant species. Therefore, it was decided to design independent screening subsets for the therapeutic siRNAs.

All siRNAs in these subsets recognize the human ALOX15 sequence, as a human cell culture system was selected for determination of in vitro activity. Therefore, all siRNAs in these subsets can be used to target human ALOX15 in a therapeutic setting.

Exemplary 19mer sequences derived from human ALOX15 mRNA (NM_001140.4) include the first 19 nucleobases of SEQ ID NOS: 1-5349, 5366-5971, 6000-6038, 6050-6074. Prioritizing sequences for target specificity, species cross-reactivity, miRNA seed region sequences and SNPs as described above yields siRNA Subset A. The siRNAs in Subset A have the following characteristics:

Cross-reactivity: With 19mer in human ALOX15 mRNA, with 17mer/19mer in NHP ALOX15 mRNA

Specificity category: For human and NHP: - AS2 or better, SS3 or better miRNA seeds: AS+SS strand: - seed region not conserved in human, mouse, and rat and not present in >3 species

Off-target frequency: ≤20 human off-targets matched with 2 mismatches by antisense strand

SNPs: siRNA target sites do not harbor SNPs with a MAF ≥1% (pos. 2-18)

Subset A is composed of 77 siRNAs: Sense Strand SEQ ID NOS: 23, 31, 33, 37, 39, 41, 43, 51, 195, 197, 199, 251, 257, 263, 273, 275, 279, 2389, 2391, 2397, 2399, 2409, 2415, 2419, 2421, 2423, 2425, 2427, 2431, 2437, 2439, 2769, 2771, 2773, 2881, 2899, 2903, 2905, 2909, 2913, 2915, 3013, 3015, 3017, 3019, 3027, 3029, 3037, 3039, 3041, 3047, 3053, 3055, 3063, 3065, 3183, 3231, 3233, 3243, 3439, 3443, 3449, 3457, 3461, 3463, 3573, 3703, 3705, 3875, 3889, 3981, 4089, 4265, 4321, 4339, 4347, 4507; Antisense Strand SEQ ID NOS: 24, 32, 34, 38, 40, 42, 44, 52, 196, 198, 200, 252, 258, 264, 274, 276, 280, 2390, 2392, 2398, 2400, 2410, 2416, 2420, 2422, 2424, 2426, 2428, 2432, 2438, 2440, 2770, 2772, 2774, 2882, 2900, 2904, 2906, 2910, 2914, 2916, 3014, 3016, 3018, 3020, 3028, 3030, 3038, 3040, 3042, 3048, 3054, 3056, 3064, 3066, 3184, 3232, 3234, 3244, 3440, 3444, 3450, 3458, 3462, 3464, 3574, 3704, 3706, 3876, 3890, 3982, 4090, 4266, 4322, 4340, 4348, 4508.

Additional human siRNAs that would cross-react with the mouse ALOX15 mRNA (NM_009660.3) were also identified using bioinformatics. The siRNAs in the human/mouse additional subset (Subset B) has the following characteristics:

Cross-reactivity: With 19mer in human ALOX15 mRNA

Specificity category: For human: AS2 or better, SS3 or better

miRNA seeds: AS+SS strand: seed region not conserved in human, mouse, and rat//not present in >3 species

Off-target frequency: ≤20 human off-targets matched with 2 mismatches by AS strand

SNPs: siRNA target sites do not harbor SNPs with a MAF ≥1% (pos. 2-18)

Cross-reactivity: Mouse: 0 siRNAs (17mer/19mer FM)-11 siRNAs (17mer/19mer SSM)

Specificity category: For mouse: AS2 or better, SS3 or better

This human/mouse additional subset, Subset B, is composed of 5 siRNAs: Sense Strand SEQ ID NOS: 53, 2627, 2629, 3355, 3365; Antisense Strand SEQ ID NOS: 54, 2628, 2630, 3356, 3366.

Additional human siRNAs that would cross-react with the rabbit ALOX15 mRNA (NM_001082282.1) were also identified using bioinformatics. The siRNAs in the human/rabbit additional subset (Subset C) has the following characteristics:

Cross-reactivity: With 19mer in human ALOX15 mRNA

Specificity category: For human: AS2 or better, SS3 or better

miRNA seeds: AS+SS strand: seed region not conserved in human, mouse, and rat//not present in >3 species

Off-target frequency: ≤20 human off-targets matched with 2 mismatches by AS strand

SNPs: siRNA target sites do not harbor SNPs with a MAF ≥1% (pos. 2-18)

Cross-reactivity: Rabbit: 11 siRNAs (17mer/19mer FM)-30 siRNAs (17mer/19mer SMM)

This human/rabbit additional subset, Subset C, is composed of 14 siRNAs: Sense Strand SEQ ID NOS: 87, 2329, 2379, 2381, 2521, 2525, 2607, 3057, 3061, 3067, 3501, 3503, 3873, 4179; Antisense Strand SEQ ID NOS: 88, 2330, 2380, 2382, 2522, 2526, 2608, 3058, 3062, 3068, 3502, 3504, 3874, 4180.

### Example 5. Chemically Modified ALOX15 siRNAs for Screening

The sequences of Screening Subsets A-C were synthesized with chemical modifications, with the sense strand having the pattern 5′ fNsmNsfN-mN-fN-mN-fN-fN-fN-mN-fN-mN-fN-mN-fN-mN-fN-mN-fNsmNsmN 3′ (pattern 1S) and antisense strand having the pattern 5′ mNsfN-mN-fN-mN-fN-mN-fN-mN-fN-mN-mN-mN-fN-mN-fN-mN-fN-mNsmNsmN 3′ (pattern 1AS). “N” can be any nucleoside (for example ribose, deoxyribose, or derivatives thereof), “fN” is a 2′ fluoro-modified nucleoside, “mN” is a 2′ O-methyl modified nucleoside, “-” is a phosphodiester and “s” is a phosphorothioate. Sense and antisense strands were annealed to prepare the following siRNAs: ETD00138, ETD00139, ETD00140, ETD00141, ETD00142, ETD00143, ETD00144, ETD00145, ETD00146, ETD00147, ETD00148, ETD00149, ETD00150, ETD00151, ETD00152, ETD00153, ETD00154, ETD00155, ETD00156, ETD00157, ETD00158, ETD00159, ETD00160, ETD00161, ETD00162, ETD00163, ETD00164, ETD00165, ETD00166, ETD00167, ETD00168, ETD00169, ETD00170, ETD00171, ETD00172, ETD00173, ETD00174, ETD00175, ETD00176, ETD00177, ETD00178, ETD00179, ETD00180, ETD00181, ETD00182, ETD00183, ETD00184, ETD00185, ETD00186, ETD00187, ETD00188, ETD00189, ETD00190, ETD00191, ETD00192, ETD00193, ETD00194, ETD00195, ETD00196, ETD00197, ETD00198, ETD00199, ETD00200, ETD00201, ETD00202, ETD00203, ETD00204, ETD00205, ETD00206, ETD00207, ETD00208, ETD00209, ETD00210, ETD00211, ETD00212, ETD00213, ETD00214, ETD00215, ETD00216, ETD00217, ETD00218, ETD00219, ETD00220, ETD00221, ETD00222, ETD00223, ETD00224, ETD00225, ETD00226, ETD00227, ETD00228, ETD00229, ETD00230, ETD00231, ETD00232 and ETD00233. A subset of sequences were synthesized with chemical modifications with the sense strand having the pattern 5′ mNsmNsmN-mN-fN-mN-fN-fN-fN-mN-mN-mN-mN-mN-mN-mN-mN-mN-mNsmNs mN 3′ (pattern 2S) and antisense strand having the pattern 5′ mNsfNsmN-mN-mN-fN-mN-mN-mN-mN-mN-mN-mN-fN-mN-fN-mN-mN-mNsmNsmN 3′ (pattern 3AS). Sense and antisense strands were annealed to prepare the following siRNAs: ETD00234, ETD00235, ETD00236, ETD00237 and ETD00238.

Table 5A provides a summary of exemplary ALOX15 siRNA nucleic acid sequences. Starting from base sense and antisense strands, such as those provided in SEQ ID NOS: 1-5349, 5366-5971, 6000-6038, 6050-6074, the two 3′ terminal nucleosides were removed from both the sense strand and the antisense strand; and then the terminal 3′ nucleoside of the sense strand was replaced with “A” and the terminal 5′ nucleoside of the antisense strand was replaced with “U.” Table 5A also indicates the species reactivity for each dsRNA, wherein hu is human, NHP is non-human primates, mo is mouse, rab is rabbit, 1 mm is one mismatch.

Table 5B provides the exemplary ALOX15 siRNA nucleic acid sequences with the chemical modifications described in the present example. In each siRNA, the two bases at the 3′ ends of both the sense and antisense strands were uridine. Uridine was used as the base at the 5′ end of all antisense strands and adenine was used as the base at position 19 of all sense strands. Sense and antisense strands were annealed to prepare the siRNA.

TABLE 5A
ALOX15 siRNA

| | | | Base | siRNA sense | siRNA antisense | siRNA sense | siRNA antisense | Position |
|---|---|---|---|---|---|---|---|---|
| | | Base | antisense | strand after | strand after | strand after | strand after | (ALOX15 |
| | Species | sense strand | strand | 3′ deletion | 3′ deletion | “A” sub | “U” sub | mRNA |
| Duplex Name | Activity | (SEQ ID NO) | (SEQ ID NO) | (SEQ ID NO) | (SEQ ID NO) | (SEQ ID NO) | (SEQ ID NO) | NM 001140) |
| ETD00138 | hu, NHP | 23 | 24 | 5568 | 5669 | 5770 | 5871 | 12 |
| ETD00139 | hu, NHP | 31 | 32 | 5569 | 5670 | 5771 | 5872 | 16 |
| ETD00140 | hu, NHP, | 33 | 34 | 5570 | 5671 | 5772 | 5873 | 17 |
| | mo 1 mm | | | | | | | |
| ETD00141 | hu, NHP, | 37 | 38 | 5571 | 5672 | 5773 | 5874 | 19 |
| | mo 1 mm | | | | | | | |
| ETD00142 | hu, NHP, | 39 | 40 | 5572 | 5673 | 5774 | 5875 | 20 |
| | mo 1 mm | | | | | | | |
| ETD00143 | hu, NHP, | 41 | 42 | 5573 | 5674 | 5775 | 5876 | 21 |
| | mo 1 mm | | | | | | | |
| ETD00144 | hu, NHP, | 43 | 44 | 5574 | 5675 | 5776 | 5877 | 22 |
| | mo 1 mm | | | | | | | |
| ETD00145 | hu, NHP, | 51 | 52 | 5575 | 5676 | 5777 | 5878 | 26 |
| | mo 1 mm | | | | | | | |
| ETD00146 | hu, NHP | 195 | 196 | 5576 | 5677 | 5778 | 5879 | 98 |
| ETD00147 | hu, NHP | 197 | 198 | 5577 | 5678 | 5779 | 5880 | 99 |
| ETD00148 | hu, NHP | 199 | 200 | 5578 | 5679 | 5780 | 5881 | 100 |
| ETD00149 | hu, NHP | 251 | 252 | 5579 | 5680 | 5781 | 5882 | 126 |
| ETD00150 | hu, NHP | 257 | 258 | 5580 | 5681 | 5782 | 5883 | 129 |
| ETD00151 | hu, NHP | 263 | 264 | 5581 | 5682 | 5783 | 5884 | 132 |
| ETD00152 | hu, NHP | 273 | 274 | 5582 | 5683 | 5784 | 5885 | 137 |
| ETD00153 | hu, NHP | 275 | 276 | 5583 | 5684 | 5785 | 5886 | 138 |
| ETD00154 | hu, NHP | 279 | 280 | 5584 | 5685 | 5786 | 5887 | 140 |
| ETD00155 | hu, NHP, | 2389 | 2390 | 5585 | 5686 | 5787 | 5888 | 1195 |
| | rab | | | | | | | |
| ETD00156 | hu, NHP, | 2391 | 2392 | 5586 | 5687 | 5788 | 5889 | 1196 |
| | rab | | | | | | | |
| ETD00157 | hu, NHP | 2397 | 2398 | 5587 | 5688 | 5789 | 5890 | 1199 |
| ETD00158 | hu, NHP | 2399 | 2400 | 5588 | 5689 | 5790 | 5891 | 1200 |
| ETD00159 | hu, NHP | 2409 | 2410 | 5589 | 5690 | 5791 | 5892 | 1205 |
| ETD00160 | hu, NHP | 2415 | 2416 | 5590 | 5691 | 5792 | 5893 | 1208 |
| ETD00161 | hu, NHP | 2419 | 2420 | 5591 | 5692 | 5793 | 5894 | 1210 |
| ETD00162 | hu, NHP | 2421 | 2422 | 5592 | 5693 | 5794 | 5895 | 1211 |
| ETD00163 | hu, NHP | 2423 | 2424 | 5593 | 5694 | 5795 | 5896 | 1212 |
| ETD00164 | hu, NHP | 2425 | 2426 | 5594 | 5695 | 5796 | 5897 | 1213 |
| ETD00165 | hu, NHP | 2427 | 2428 | 5595 | 5696 | 5797 | 5898 | 1214 |
| ETD00166 | hu, NHP | 2431 | 2432 | 5596 | 5697 | 5798 | 5899 | 1216 |
| ETD00167 | hu, NHP | 2437 | 2438 | 5597 | 5698 | 5799 | 5900 | 1219 |
| ETD00168 | hu, NHP | 2439 | 2440 | 5598 | 5699 | 5800 | 5901 | 1220 |
| ETD00169 | hu, NHP | 2769 | 2770 | 5599 | 5700 | 5801 | 5902 | 1385 |
| ETD00170 | hu, NHP | 2771 | 2772 | 5600 | 5701 | 5802 | 5903 | 1386 |
| ETD00171 | hu, NHP | 2773 | 2774 | 5601 | 5702 | 5803 | 5904 | 1387 |
| ETD00172 | hu, NHP | 2881 | 2882 | 5602 | 5703 | 5804 | 5905 | 1441 |
| ETD00173 | hu, NHP | 2899 | 2900 | 5603 | 5704 | 5805 | 5906 | 1450 |
| ETD00174 | hu, NHP | 2903 | 2904 | 5604 | 5705 | 5806 | 5907 | 1452 |
| ETD00175 | hu, NHP | 2905 | 2906 | 5605 | 5706 | 5807 | 5908 | 1453 |
| ETD00176 | hu, NHP | 2909 | 2910 | 5606 | 5707 | 5808 | 5909 | 1455 |
| ETD00177 | hu, NHP | 2913 | 2914 | 5607 | 5708 | 5809 | 5910 | 1457 |
| ETD00178 | hu, NHP | 2915 | 2916 | 5608 | 5709 | 5810 | 5911 | 1458 |
| ETD00179 | hu, NHP | 3013 | 3014 | 5609 | 5710 | 5811 | 5912 | 1507 |
| ETD00180 | hu, NHP | 3015 | 3016 | 5610 | 5711 | 5812 | 5913 | 1508 |
| ETD00181 | hu, NHP | 3017 | 3018 | 5611 | 5712 | 5813 | 5914 | 1509 |
| ETD00182 | hu, NHP | 3019 | 3020 | 5612 | 5713 | 5814 | 5915 | 1510 |
| ETD00183 | hu, NHP, | 3027 | 3028 | 5613 | 5714 | 5815 | 5916 | 1514 |
| | rab | | | | | | | |
| ETD00184 | hu, NHP, | 3029 | 3030 | 5614 | 5715 | 5816 | 5917 | 1515 |
| | rab | | | | | | | |
| ETD00185 | hu, NHP, | 3037 | 3038 | 5615 | 5716 | 5817 | 5918 | 1519 |
| | rab | | | | | | | |
| ETD00186 | hu, NHP, | 3039 | 3040 | 5616 | 5717 | 5818 | 5919 | 1520 |
| | rab | | | | | | | |
| ETD00187 | hu, NHP, | 3041 | 3042 | 5617 | 5718 | 5819 | 5920 | 1521 |
| | rab | | | | | | | |
| ETD00188 | hu, NHP, | 3047 | 3048 | 5618 | 5719 | 5820 | 5921 | 1524 |
| | rab | | | | | | | |
| ETD00189 | hu, NHP, | 3053 | 3054 | 5619 | 5720 | 5821 | 5922 | 1527 |
| | rab | | | | | | | |
| ETD00190 | hu, NHP, | 3055 | 3056 | 5620 | 5721 | 5822 | 5923 | 1528 |
| | rab | | | | | | | |
| ETD00191 | hu, NHP, | 3063 | 3064 | 5621 | 5722 | 5823 | 5924 | 1532 |
| | rab | | | | | | | |
| ETD00192 | hu, NHP, | 3065 | 3066 | 5622 | 5723 | 5824 | 5925 | 1533 |
| | rab | | | | | | | |
| ETD00193 | hu, NHP, | 3183 | 3184 | 5623 | 5724 | 5825 | 5926 | 1592 |
| | rab | | | | | | | |
| ETD00194 | hu, NHP | 3231 | 3232 | 5624 | 5725 | 5826 | 5927 | 1616 |
| ETD00195 | hu, NHP | 3233 | 3234 | 5625 | 5726 | 5827 | 5928 | 1617 |
| ETD00196 | hu, NHP | 3243 | 3244 | 5626 | 5727 | 5828 | 5929 | 1622 |
| ETD00197 | hu, NHP | 3439 | 3440 | 5627 | 5728 | 5829 | 5930 | 1720 |
| ETD00198 | hu, NHP | 3443 | 3444 | 5628 | 5729 | 5830 | 5931 | 1722 |
| ETD00199 | hu, NHP | 3449 | 3450 | 5629 | 5730 | 5831 | 5932 | 1725 |
| ETD00200 | hu, NHP | 3457 | 3458 | 5630 | 5731 | 5832 | 5933 | 1729 |
| ETD00201 | hu, NHP | 3461 | 3462 | 5631 | 5732 | 5833 | 5934 | 1731 |
| ETD00202 | hu, NHP | 3463 | 3464 | 5632 | 5733 | 5834 | 5935 | 1732 |
| ETD00203 | hu, NHP | 3573 | 3574 | 5633 | 5734 | 5835 | 5936 | 1787 |
| ETD00204 | hu, NHP | 3703 | 3704 | 5634 | 5735 | 5836 | 5937 | 1852 |
| ETD00205 | hu, NHP | 3705 | 3706 | 5635 | 5736 | 5837 | 5938 | 1853 |
| ETD00206 | hu, NHP, | 3875 | 3876 | 5636 | 5737 | 5838 | 5939 | 1938 |
| | rab | | | | | | | |
| ETD00207 | hu, NHP | 3889 | 3890 | 5637 | 5738 | 5839 | 5940 | 1945 |
| ETD00208 | hu, NHP | 3981 | 3982 | 5638 | 5739 | 5840 | 5941 | 1991 |
| ETD00209 | hu, NHP | 4089 | 4090 | 5639 | 5740 | 5841 | 5942 | 2045 |
| ETD00210 | hu, NHP | 4265 | 4266 | 5640 | 5741 | 5842 | 5943 | 2133 |
| ETD00211 | hu, NHP | 4321 | 4322 | 5641 | 5742 | 5843 | 5944 | 2161 |
| ETD00212 | hu, NHP | 4339 | 4340 | 5642 | 5743 | 5844 | 5945 | 2170 |
| ETD00213 | hu, NHP | 4347 | 4348 | 5643 | 5744 | 5845 | 5946 | 2174 |
| ETD00214 | hu, NHP | 4507 | 4508 | 5644 | 5745 | 5846 | 5947 | 2254 |
| ETD00215 | hu, mo | 53 | 54 | 5645 | 5746 | 5847 | 5948 | 27 |
| | 1 mm | | | | | | | |
| ETD00216 | hu, mo | 2627 | 2628 | 5646 | 5747 | 5848 | 5949 | 1314 |
| | 1 mm, rab | | | | | | | |
| | 1 mm | | | | | | | |
| ETD00217 | hu, mo | 2629 | 2630 | 5647 | 5748 | 5849 | 5950 | 1315 |
| | 1 mm, rab | | | | | | | |
| | 1 mm | | | | | | | |
| ETD00218 | hu, mo | 3355 | 3356 | 5648 | 5749 | 5850 | 5951 | 1678 |
| | 1 mm | | | | | | | |
| ETD00219 | hu, mo | 3365 | 3366 | 5649 | 5750 | 5851 | 5952 | 1683 |
| | 1 mm | | | | | | | |
| ETD00220 | hu, rab | 87 | 88 | 5650 | 5751 | 5852 | 5953 | 44 |
| | 1 mm | | | | | | | |
| ETD00221 | hu, | 2329 | 2330 | 5651 | 5752 | 5853 | 5954 | 1165 |
| | rab, 1 mm | | | | | | | |
| ETD00222 | hu, rab | 2379 | 2380 | 5652 | 5753 | 5854 | 5955 | 1190 |
| | 1 mm | | | | | | | |
| ETD00223 | hu, rab | 2381 | 2382 | 5653 | 5754 | 5855 | 5956 | 1191 |
| | 1 mm | | | | | | | |
| ETD00224 | hu, rab | 2521 | 2522 | 5654 | 5755 | 5856 | 5957 | 1261 |
| | 1 mm | | | | | | | |
| ETD00225 | hu, rab | 2525 | 2526 | 5655 | 5756 | 5857 | 5958 | 1263 |
| | 1 mm | | | | | | | |
| ETD00226 | hu, rab | 2607 | 2608 | 5656 | 5757 | 5858 | 5959 | 1304 |
| | 1 mm | | | | | | | |
| ETD00227 | hu, NHP, | 3057 | 3058 | 5657 | 5758 | 5859 | 5960 | 1529 |
| | rab | | | | | | | |
| ETD00228 | hu, NHP, | 3061 | 3062 | 5658 | 5759 | 5860 | 5961 | 1531 |
| | rab | | | | | | | |
| ETD00229 | hu, rab | 3067 | 3068 | 5659 | 5760 | 5861 | 5962 | 1534 |
| ETD00230 | hu, rab | 3501 | 3502 | 5660 | 5761 | 5862 | 5963 | 1751 |
| | 1 mm | | | | | | | |
| ETD00231 | hu, rab | 3503 | 3504 | 5661 | 5762 | 5863 | 5964 | 1752 |
| | 1 mm | | | | | | | |
| ETD00232 | hu, rab | 3873 | 3874 | 5662 | 5763 | 5864 | 5965 | 1937 |
| | 1 mm | | | | | | | |
| ETD00233 | hu, rab | 4179 | 4180 | 5663 | 5764 | 5865 | 5966 | 2090 |
| | 1 mm | | | | | | | |
| ETD00234 | hu, NHP, | 3027 | 3028 | 5664 | 5765 | 5866 | 5967 | 1514 |
| | rab | | | | | | | |
| ETD00235 | hu, NHP, | 3037 | 3038 | 5665 | 5766 | 5867 | 5968 | 1519 |
| | rab | | | | | | | |
| ETD00236 | hu, NHP, | 3183 | 3184 | 5666 | 5767 | 5868 | 5969 | 1592 |
| | rab | | | | | | | |
| ETD00237 | hu, NHP | 4265 | 4266 | 5667 | 5768 | 5869 | 5970 | 2133 |
| ETD00238 | hu, mo | 2629 | 2630 | 5668 | 5769 | 5870 | 5971 | 1315 |
| | 1 mm, rab | | | | | | | |
| | 1 mm | | | | | | | |

TABLE 5B
Chemically modified ALOX15 siRNAs derived from Screening Subsets A-C

| | Sense | | | |
|---|---|---|---|---|
| | Strand | | Antisense | Antisense |
| siRNA | SEQ | Sense Strand Sequence | SEQ ID | Strand Sequence |
| Name | ID NO | (5′-3′) | NO | Strand (5′-3′) |
| ETD001 | 5366 | fCsmUsfUmUfGmAfGfCfAmAfG | 5367 | mUsfGsmAfCmCfCmAfUmCfUm |
| 38 | | mAfUmGfGmGfUmCfAsmUsmU | | UmGmCfUmCfAmAfAmGsmUs |
| | | | | mU |
| ETD001 | 5368 | fGsmAsfGmCfAmAfGfAfUmGfG | 5369 | mUsfUsmAfGmAfGmAfCmCfC |
| 39 | | mGfUmCfUmCfUmAfAsmUsmU | | mAmUmCfUmUfGmCfUmCsmU |
| | | | | smU |
| ETD001 | 5370 | fAsmGsfCmAfAmGfAfUfGmGfG | 5371 | mUsfGsmUfAmGfAmGfAmCfC |
| 40 | | mUfCmUfCmUfAmCfAsmUsmU | | mCmAmUfCmUfUmGfCmUsmU |
| | | | | smU |
| ETD001 | 5372 | fCsmAsfAmGfAmUfGfGfGmUfC | 5373 | mUsfCsmGfGmUfAmGfAmGfA |
| 41 | | mUfCmUfAmCfCmGfAsmUsmU | | mCmCmCfAmUfCmUfUmGsmU |
| | | | | smU |
| ETD001 | 5374 | fAsmAsfGmAfUmGfGfGfUmCfU | 5375 | mUsfGsmCfGmGfUmAfGmAfG |
| 42 | | mCfUmAfCmCfGmCfAsmUsmU | | mAmCmCfCmAfUmCfUmUsmU |
| | | | | smU |
| ETD001 | 5376 | fAsmGsfAmUfGmGfGfUfCmUfC | 5377 | mUsfUsmGfCmGfGmUfAmGfA |
| 43 | | mUfAmCfCmGfCmAfAsmUsmU | | mGmAmCfCmCfAmUfCmUsmU |
| | | | | smU |
| ETD001 | 5378 | fGsmAsfUmGfGmGfUfCfUmCfU | 5379 | mUsfAsmUfGmCfGmGfUmAfG |
| 44 | | mAfCmCfGmCfAmUfAsmUsmU | | mAmGmAfCmCfCmAfUmCsmU |
| | | | | smU |
| ETD001 | 5380 | fGsmGsfUmCfUmCfUfAfCmCfG | 5381 | mUsfGsmCfGmGfAmUfGmCfG |
| 45 | | mCfAmUfCmCfGmCfAsmUsmU | | mGmUmAfGmAfGmAfCmCsmU |
| | | | | smU |
| ETD001 | 5382 | fCsmUsfGmGfUmCfGfGfCmCfA | 5383 | mUsfCsmCfCmGfUmGfCmUfGm |
| 46 | | mGfCmAfCmGfGmGfAsmUsmU | | GmCmCfGmAfCmCfAmGsmUs |
| | | | | mU |
| ETD001 | 5384 | fUsmGsfGmUfCmGfGfCfCmAfG | 5385 | mUsfCsmCfCmCfGmUfGmCfUm |
| 47 | | mCfAmCfGmGfGmGfAsmUsmU | | GmGmCfCmGfAmCfCmAsmUs |
| | | | | mU |
| ETD001 | 5386 | fGsmGsfUmCfGmGfCfCfAmGfC | 5387 | mUsfUsmCfCmCfCmGfUmGfCm |
| 48 | | mAfCmGfGmGfGmAfAsmUsmU | | UmGmGfCmCfGmAfCmCsmUs |
| | | | | mU |
| ETD001 | 5388 | fUsmCsfGmGfGmAfAfGfCmGfA | 5389 | mUsfGsmCfCmAfCmAfGmUfCm |
| 49 | | mCfUmGfUmGfGmCfAsmUsmU | | GmCmUfUmCfCmCfGmAsmUs |
| | | | | mU |
| ETD001 | 5390 | fGsmGsfAmAfGmCfGfAfCmUfG | 5391 | mUsfCsmGfGmGfCmCfAmCfAm |
| 50 | | mUfGmGfCmCfCmGfAsmUsmU | | GmUmCfGmCfUmUfCmCsmUs |
| | | | | mU |
| ETD001 | 5392 | fAsmGsfCmGfAmCfUfGfUmGfG | 5393 | mUsfGsmUfGmCfGmGfGmCfC |
| 51 | | mCfCmCfGmCfAmCfAsmUsmU | | mAmCmAfGmUfCmGfCmUsmU |
| | | | | smU |
| ETD001 | 5394 | fCsmUsfGmUfGmGfCfCfCmGfC | 5395 | mUsfGsmCfCmCfCmGfUmGfCm |
| 52 | | mAfCmGfGmGfGmCfAsmUsmU | | GmGmGfCmCfAmCfAmGsmUs |
| | | | | mU |
| ETD001 | 5396 | fUsmGsfUmGfGmCfCfCfGmCfA | 5397 | mUsfUsmGfCmCfCmCfGmUfGm |
| 53 | | mCfGmGfGmGfCmAfAsmUsmU | | CmGmGfGmCfCmAfCmAsmUs |
| | | | | mU |
| ETD001 | 5398 | fUsmGsfGmCfCmCfGfCfAmCfG | 5399 | mUsfCsmUfUmGfCmCfCmCfGm |
| 54 | | mGfGmGfCmAfAmGfAsmUsmU | | UmGmCfGmGfGmCfCmAsmUs |
| | | | | mU |
| ETD001 | 5400 | fCsmCsfAmCfCmUfGfCfGmAfU | 5401 | mUsfAsmGfGmGfUmGfUmAfU |
| 55 | | mCfAmCfCmCfUmGfAsmUsmU | | mUmCmGfCmAfGmGfUmGsmU |
| | | | | smU |
| ETD001 | 5402 | fCsmAsfCmCfUmGfCfGfAmUfA | 5403 | mUsfCsmAfGmGfGmUfGmUfA |
| 56 | | mCfAmCfCmCfUmGfAsmUsmU | | mUmCmGfCmAfGmGfUmGsmU |
| | | | | smU |
| ETD001 | 5404 | fCsmUsfGmCfGmAfUfAfCmAfC | 5405 | mUsfUsmUfCmCfAmGfGmGfU |
| 57 | | mCfCmUfGmGfAmAfAsmUsmU | | mGmUmAfUmCfGmCfAmGsmU |
| | | | | smU |
| ETD001 | 5406 | fUsmGsfCmGfAmUfAfCfAmCfC | 5407 | mUsfUsmUfUmCfCmAfGmGfG |
| 58 | | mCfUmGfGmAfAmAfAsmUsmU | | mUmGmUfAmUfCmGfCmAsmU |
| | | | | smU |
| ETD001 | 5408 | fUsmAsfCmAfCmCfCfUfGmGfA | 5409 | mUsfGsmUfUmAfAmUfUmUfC |
| 59 | | mAfAmUfUmAfAmCfAsmUsmU | | mCmAmGfGmGfUmGfUmAsmU |
| | | | | smU |
| ETD001 | 5410 | fAsmCsfCmCfUmGfGfAfAmAfU | 5411 | mUsfGsmAfCmGfUmUfAmAfU |
| 60 | | mUfAmAfCmGfUmCfAsmUsmU | | mUmUmCfCmAfGmGfGmUsmU |
| | | | | smU |
| ETD001 | 5412 | fCsmCsfUmGfGmAfAfAfUmUfA | 5413 | mUsfCsmGfGmAfCmGfUmUfA |
| 61 | | mAfCmGfUmCfCmGfAsmUsmU | | mAmUmUfUmCfCmAfGmGsmU |
| | | | | smU |
| ETD001 | 5414 | fCsmUsfGmGfAmAfAfUfUmAfA | 5415 | mUsfCsmCfGmGfAmCfGmUfU |
| 62 | | mCfGmUfCmCfGmGfAsmUsmU | | mAmAmUfUmUfCmCfAmGsmU |
| | | | | smU |
| ETD001 | 5416 | fUsmGsfGmAfAmAfUfUfAmAfC | 5417 | mUsfCsmCfCmGfGmAfCmGfUm |
| 63 | | mGfUmCfCmGfGmGfAsmUsmU | | UmAmAfUmUfUmCfCmAsmUs |
| | | | | mU |
| ETD001 | 5418 | fGsmGsfAmAfAmUfUfAfAmCfG | 5419 | mUsfGsmCfCmCfGmGfAmCfGm |
| 64 | | mUfCmCfGmGfGmCfAsmUsmU | | UmUmAfAmUfUmUfCmCsmUs |
| | | | | mU |
| ETD001 | 5420 | fGsmAsfAmAfUmUfAfAfCmGfU | 5421 | mUsfGsmGfCmCfCmGfGmAfCm |
| 65 | | mCfCmGfGmGfCmCfAsmUsmU | | GmUmUfAmAfUmUfUmCsmUs |
| | | | | mU |
| ETD001 | 5422 | fAsmAsfUmUfAmAfCfGfUmCfC | 5423 | mUsfCsmUfGmGfCmCfCmGfGm |
| 66 | | mGfGmGfCmCfAmGfAsmUsmU | | AmCmGfUmUfAmAfUmUsmUs |
| | | | | mU |
| ETD001 | 5424 | fUsmAsfAmCfGmUfCfCfGmGfG | 5425 | mUsfGsmUfCmCfUmGfGmCfCm |
| 67 | | mCfCmAfGmGfAmCfAsmUsmU | | CmGmGfAmCfGmUfUmAsmUs |
| | | | | mU |
| ETD001 | 5426 | fAsmAsfCmGfUmCfCfGfGmGfC | 5427 | mUsfAsmGfUmCfCmUfGmGfC |
| 68 | | mCfAmGfGmAfCmUfAsmUsmU | | mCmCmGfGmAfCmGfUmUsmU |
| | | | | smU |
| ETD001 | 5428 | fGsmUsfGmAfAmGfUfCfUmUfC | 5429 | mUsfAsmUfAmGfAmAfGmGfA |
| 69 | | mCfUmUfCmUfAmUfAsmUsmU | | mAmGmAfCmUfUmCfAmCsmU |
| | | | | smU |
| ETD001 | 5430 | fUsmGsfAmAfGmUfCfUfUmCfC | 5431 | mUsfCsmAfUmAfGmAfAmGfG |
| 70 | | mUfUmCfUmAfUmGfAsmUsmU | | mAmAmGfAmCfUmUfCmAsmU |
| | | | | smU |
| ETD001 | 5432 | fGsmAsfAmGfUmCfUfUfCmCfU | 5433 | mUsfGsmCfAmUfAmGfAmAfG |
| 71 | | mUfCmUfAmUfGmCfAsmUsmU | | mGmAmAfGmAfCmUfUmCsmU |
| | | | | smU |
| ETD001 | 5434 | fGsmUsfAmUfGmUfGfGfAmAfG | 5435 | mUsfAsmCfGmAfUmUfCmCfU |
| 72 | | mGfAmAfUmCfGmUfAsmUsmU | | mUmCmCfAmCfAmUfAmCsmU |
| | | | | smU |
| ETD001 | 5436 | fAsmGsfGmAfAmUfCfGfUmGfA | 5437 | mUsfUsmGfGmAfGmAfCmUfC |
| 73 | | mGfUmCfUmCfCmAfAsmUsmU | | mAmCmGfAmUfUmCfCmUsmU |
| | | | | smU |
| ETD001 | 5438 | fGsmAsfAmUfCmGfUfGfAmGfU | 5439 | mUsfAsmGfUmGfGmAfGmAfC |
| 74 | | mCfUmCfCmAfCmUfAsmUsmU | | mUmCmAfCmGfAmUfUmCsmU |
| | | | | smU |
| ETD001 | 5440 | fAsmAsfUmCfGmUfGfAfGmUfC | 5441 | mUsfUsmAfGmUfGmGfAmGfA |
| 75 | | mCfAmCfUmAfUmAfAsmUsmU | | mGmAmCfUmCfAmCfGmAsmU |
| | | | | smU |
| ETD001 | 5442 | fUsmCsfGmUfGmAfGfUfCmUfC | 5443 | mUsfUsmAfUmAfGmUfGmGfA |
| 76 | | mCfAmCfUmAfUmAfAsmUsmU | | mGmAmCfUmCfAmCfGmAsmU |
| | | | | smU |
| ETD001 | 5444 | fGsmUsfGmAfGmUfCfUfCmCfA | 5445 | mUsfCsmUfUmAfUmAfGmUfG |
| 77 | | mCfUmAfUmAfAmGfAsmUsmU | | mGmAmGfAmCfUmCfAmCsmU |
| | | | | smU |
| ETD001 | 5446 | fUsmGsfAmGfUmCfUfCfCmAfC | 5447 | mUsfUsmCfUmUfAmUfAmGfU |
| 78 | | mUfAmUfAmAfGmAfAsmUsmU | | mGmGmAfGmAfCmUfCmAsmU |
| | | | | smU |
| ETD001 | 5448 | fGsmCsfAmGfAmCfCfUfGmGfU | 5449 | mUsfUsmCfUmCfGmAfCmAfCm |
| 79 | | mGfUmCfGmAfGmAfAsmUsmU | | CmAmGfGmUfCmUfGmCsmUs |
| | | | | mU |
| ETD001 | 5450 | fCsmAsfGmAfCmCfUfGfGmUfG | 5451 | mUsfCsmUfCmUfCmGfAmCfAm |
| 80 | | mUfCmGfAmGfAmGfAsmUsmU | | CmCmAfGmGfUmCfUmGsmUs |
| | | | | mU |
| ETD001 | 5452 | fAsmGsfAmCfCmUfGfGfUmGfU | 5453 | mUsfUsmCfUmCfUmCfGmAfCm |
| 81 | | mCfGmAfGmAfGmAfAsmUsmU | | AmCmCfAmGfGmUfCmUsmUs |
| | | | | mU |
| ETD001 | 5454 | fGsmAsfCmCfUmGfGfUfGmUfC | 5455 | mUsfAsmUfCmUfCmUfCmGfA |
| 82 | | mGfAmGfAmGfAmUfAsmUsmU | | mCmAmCfCmAfGmGfUmCsmU |
| | | | | smU |
| ETD001 | 5456 | fUsmGsfGmUfGmUfCfGfAmGfA | 5457 | mUsfAsmGfUmGfAmUfCmUfC |
| 83 | | mGfAmUfCmAfCmUfAsmUsmU | | mUmCmGfAmCfAmCfCmAsmU |
| | | | | smU |
| ETD001 | 5458 | fGsmGsfUmGfUmCfGfAfGmAfG | 5459 | mUsfCsmAfGmUfGmAfUmCfU |
| 84 | | mAfUmCfAmCfUmGfAsmUsmU | | mCmUmCfGmAfCmAfCmCsmU |
| | | | | smU |
| ETD001 | 5460 | fUsmCsfGmAfGmAfGfAfUmCfA | 5461 | mUsfAsmUfUmUfCmAfGmUfG |
| 85 | | mCfUmGfAmAfAmUfAsmUsmU | | mAmUmCfUmCfUmCfGmAsmU |
| | | | | smU |
| ETD001 | 5462 | fCsmGsfAmGfAmGfAfUfCmAfC | 5463 | mUsfGsmAfUmUfUmCfAmGfU |
| 86 | | mUfGmAfAmAfUmCfAsmUsmU | | mGmAmUfCmUfCmUfCmGsmU |
| | | | | smU |
| ETD001 | 5464 | fGsmAsfGmAfGmAfUfCfAmCfU | 5465 | mUsfCsmGfAmUfUmUfCmAfG |
| 87 | | mGfAmAfAmUfCmGfAsmUsmU | | mUmGmAfUmCfUmCfUmCsmU |
| | | | | smU |
| ETD001 | 5466 | fAsmGsfAmUfCmAfCfUfGmAfA | 5467 | mUsfGsmCfCmCfGmAfUmUfU |
| 88 | | mAfUmCfGmGfGmCfAsmUsmU | | mCmAmGfUmGfAmUfCmUsmU |
| | | | | smU |
| ETD001 | 5468 | fUsmCsfAmCfUmGfAfAfAmUfC | 5469 | mUsfGsmCfAmGfCmCfCmGfAm |
| 89 | | mGfGmGfCmUfGmCfAsmUsmU | | UmUmUfCmAfGmUfGmAsmUs |
| | | | | mU |
| ETD001 | 5470 | fCsmAsfCmUfGmAfAfAfUmCfG | 5471 | mUsfUsmGfCmAfGmCfCmCfGm |
| 90 | | mGfGmCfUmGfCmAfAsmUsmU | | AmUmUfUmCfAmGfUmGsmUs |
| | | | | mU |
| ETD001 | 5472 | fGsmAsfAmAfUmCfGfGfGmCfU | 5473 | mUsfCsmCfCmUfUmGfCmAfGm |
| 91 | | mGfCmAfAmGfGmGfAsmUsmU | | CmCmCfGmAfUmUfUmCsmUs |
| | | | | mU |
| ETD001 | 5474 | fAsmAsfAmUfCmGfGfGfCmUfG | 5475 | mUsfCsmCfCmCfUmUfGmCfAm |
| 92 | | mCfAmAfGmGfGmGfAsmUsmU | | GmCmCfCmGfAmUfUmUsmUs |
| | | | | mU |
| ETD001 | 5476 | fCsmAsfGmGfUmUfUfGfCmCfA | 5477 | mUsfGsmAfCmAfAmAfGmUfG |
| 93 | | mCfUmUfUmGfUmCfAsmUsmU | | mGmCmAfAmAfCmCfUmGsmU |
| | | | | smU |
| ETD001 | 5478 | fUsmGsfUmAfUmCfUfUfCmAfC | 5479 | mUsfGsmGfUmGfCmAfGmGfU |
| 94 | | mCfUmGfCmAfCmCfAsmUsmU | | mGmAmAfGmAfUmAfCmAsmU |
| | | | | smU |
| ETD001 | 5480 | fGsmUsfAmUfCmUfUfCfAmCfC | 5481 | mUsfCsmGfGmUfGmCfAmGfG |
| 95 | | mUfGmCfAmCfCmGfAsmUsmU | | mUmGmAfAmGfAmUfAmCsmU |
| | | | | smU |
| ETD001 | 5482 | fUsmUsfCmAfCmCfUfGfCmAfC | 5483 | mUsfUsmUfGmGfCmCfGmGfU |
| 96 | | mCfGmGfCmCfAmAfAsmUsmU | | mGmCmAfGmGfUmGfAmAsmU |
| | | | | smU |
| ETD001 | 5484 | fAsmAsfCmCfAmCfCfAfAmGfG | 5485 | mUsfGsmUfUmGfCmAfUmCfC |
| 97 | | mAfUmGfCmAfAmCfAsmUsmU | | mUmUmGfGmUfGmGfUmUsmU |
| | | | | smU |
| ETD001 | 5486 | fCsmCsfAmCfCmAfAfGfGmAfU | 5487 | mUsfGsmCfGmUfUmGfCmAfU |
| 98 | | mGfCmAfAmCfGmCfAsmUsmU | | mCmCmUfUmGfGmUfGmGsmU |
| | | | | smU |
| ETD001 | 5488 | fCsmCsfAmAfGmGfAfUfGmCfA | 5489 | mUsfCsmCfAmGfCmGfUmUfG |
| 99 | | mAfCmGfCmUfGmGfAsmUsmU | | mCmAmUfCmCfUmUfGmGsmU |
| | | | | smU |
| ETD002 | 5490 | fGsmGsfAmUfGmCfAfAfCmGfC | 5491 | mUsfGsmUfCmUfCmCfAmGfCm |
| 00 | | mUfGmGfAmGfAmCfAsmUsmU | | GmUmUfGmCfAmUfCmCsmUs |
| | | | | mU |
| ETD002 | 5492 | fAsmUsfGmCfAmAfCfGfCmUfG | 5493 | mUsfCsmUfGmUfCmUfCmCfAm |
| 01 | | mGfAmGfAmCfAmGfAsmUsmU | | GmCmGfUmUfGmCfAmUsmUs |
| | | | | mU |
| ETD002 | 5494 | fUsmGsfCmAfAmCfGfCfUmGfG | 5495 | mUsfAsmCfUmGfUmCfUmCfCm |
| 02 | | mAfGmAfCmAfGmUfAsmUsmU | | AmGmCfGmUfUmGfCmAsmUs |
| | | | | mU |
| ETD002 | 5496 | fCsmAsfGmAfUmGfUfCfCmAfU | 5497 | mUsfCsmCfAmAfGmUfGmAfU |
| 03 | | mCfAmCfUmUfGmGfAsmUsmU | | mGmGmAfCmAfUmCfUmGsmU |
| | | | | smU |
| ETD002 | 5498 | fGsmGsfAmGfGmAfGfUfAmUfU | 5499 | mUsfCsmCfCmGfAmAfAmAfA |
| 04 | | mUfUmUfCmGfGmGfAsmUsmU | | mUmAmCfUmCfCmUfCmCsmU |
| | | | | smU |
| ETD002 | 5500 | fGsmAsfGmGfAmGfUfAfUmUfU | 5501 | mUsfGsmCfCmCfGmAfAmAfA |
| 05 | | mUfUmCfGmGfGmCfAsmUsmU | | mAmUmAfCmUfCmCfUmCsmU |
| | | | | smU |
| ETD002 | 5502 | fUsmCsfCmGfGmAfAfUfGmCfA | 5503 | mUsfCsmCfAmGfCmUfUmUfG |
| 06 | | mAfAmGfCmUfGmGfAsmUsmU | | mCmAmUfUmCfCmGfGmAsmU |
| | | | | smU |
| ETD002 | 5504 | fUsmGsfCmAfAmAfGfCfUmGfG | 5505 | mUsfGsmGfCmAfUmGfUmCfC |
| 07 | | mAfCmAfUmGfCmCfAsmUsmU | | mAmGmCfUmUfUmGfCmAsmU |
| | | | | smU |
| ETD002 | 5506 | fGsmAsfAmAfAmCfAfGfUmGfU | 5507 | mUsfGsmAfUmGfGmCfCmAfC |
| 08 | | mGfGmCfCmAfUmCfAsmUsmU | | mAmCmUfGmUfUmUfUmCsmU |
| | | | | smU |
| ETD002 | 5508 | fCsmAsfCmCfCmAfAfGfCmCfA | 5509 | mUsfCsmAfGmCfUmUfGmUfG |
| 09 | | mCfAmAfGmCfUmGfAsmUsmU | | mGmCmUfUmGfGmGfUmGsmU |
| | | | | smU |
| ETD002 | 5510 | fGsmCsfAmCfCmUfUfUfUmCfA | 5511 | mUsfGsmAfGmAfCmCfAmUfG |
| 10 | | mUfGmGfUmCfUmCfAsmUsmU | | mAmAmAfAmGfGmUfGmCsmU |
| | | | | smU |
| ETD002 | 5512 | fGsmAsfAmCfAmCfAfUfUmUfU | 5513 | mUsfCsmUfAmGfAmGfUmAfA |
| 11 | | mAfCmUfCmUfAmGfAsmUsmU | | mAmAmUfGmUfGmUfUmCsmU |
| | | | | smU |
| ETD002 | 5514 | fUsmUsfAmCfUmCfUfAfGmAfG | 5515 | mUsfGsmUfGmAfUmGfCmCfU |
| 12 | | mGfCmAfUmCfAmCfAsmUsmU | | mCmUmAfGmAfGmUfAmAsmU |
| | | | | smU |
| ETD002 | 5516 | fUsmCsfUmAfGmAfGfGfCmAfU | 5517 | mUsfCsmCfAmGfGmUfGmAfU |
| 13 | | mCfAmCfCmUfGmGfAsmUsmU | | mGmCmCfUmCfUmAfGmAsmU |
| | | | | smU |
| ETD002 | 5518 | fUsmCsfUmUfCmAfUfUfCmAfG | 5519 | mUsfUsmAfUmAfGmAfUmCfU |
| 14 | | mAfUmCfUmAfUmAfAsmUsmU | | mGmAmAfUmGfAmAfGmAsmU |
| | | | | smU |
| ETD002 | 5520 | fGsmUsfCmUfCmUfAfCfCmGfC | 5521 | mUsfCsmGfCmGfGmAfUmGfC |
| 15 | | mAfUmCfCmGfCmGfAsmUsmU | | mGmGmUfAmGfAmGfAmCsmU |
| | | | | smU |
| ETD002 | 5522 | fAsmAsfGmCfUmGfGfAfGmCfC | 5523 | mUsfUsmUfAmGfGmAfAmGfG |
| 16 | | mUfUmCfCmUfAmAfAsmUsmU | | mCmUmCfCmAfGmCfUmUsmU |
| | | | | smU |
| ETD002 | 5524 | fAsmGsfCmUfGmGfAfGfCmCfU | 5525 | mUsfGsmUfUmAfGmGfAmAfG |
| 17 | | mUfCmCfUmAfAmCfAsmUsmU | | mGmCmUfCmCfAmGfCmUsmU |
| | | | | smU |
| ETD002 | 5526 | fUsmUsfGmGfGmUfGfCfCmUfA | 5527 | mUsfGsmGfUmGfCmAfUmUfA |
| 18 | | mAfUmGfCmAfCmCfAsmUsmU | | mGmGmCfAmCfCmCfAmAsmU |
| | | | | smU |
| ETD002 | 5528 | fUsmGsfCmCfUmAfAfUfGmCfA | 5529 | mUsfUsmGfCmAfGmGfGmUfG |
| 19 | | mCfCmCfUmGfCmAfAsmUsmU | | mCmAmUfUmAfGmGfCmAsmU |
| | | | | smU |
| ETD002 | 5530 | fGsmUsfGmUfCmCfAfCfUmGfG | 5531 | mUsfCsmGfAmGfGmCfCmCfCm |
| 20 | | mGfGmCfCmUfCmGfAsmUsmU | | AmGmUfGmGfAmCfAmCsmUs |
| | | | | mU |
| ETD002 | 5532 | fGsmAsfUmAfCmAfUfCfCmUfA | 5533 | mUsfUsmUfGmAfAmGfAmUfA |
| 21 | | mUfCmUfUmCfAmAfAsmUsmU | | mGmGmAfUmGfUmAfUmCsmU |
| | | | | smU |
| ETD002 | 5534 | fAsmUsfUmCfCmCfCfAfCmCfU | 5535 | mUsfGsmUfAmUfCmGfCmAfG |
| 22 | | mGfCmGfAmUfAmCfAsmUsmU | | mGmUmGfGmGfGmAfAmUsmU |
| | | | | smU |
| ETD002 | 5536 | fUsmUsfCmCfCmCfAfCfCmUfG | 5537 | mUsfUsmGfUmAfUmCfGmCfA |
| 23 | | mCfGmAfUmAfCmAfAsmUsmU | | mGmGmUfGmGfGmGfAmAsmU |
| | | | | smU |
| ETD002 | 5538 | fUsmUsfUmCfGmAfCfCfAmGfA | 5539 | mUsfCsmUfCmAfUmUfAmUfC |
| 24 | | mUfAmAfUmGfAmGfAsmUsmU | | mUmGmGfUmCfGmAfAmAsmU |
| | | | | smU |
| ETD002 | 5540 | fUsmCsfGmAfCmCfAfGfAmUfA | 5541 | mUsfUsmGfCmUfCmAfUmUfA |
| 25 | | mAfUmGfAmGfCmAfAsmUsmU | | mUmCmUfGmGfUmCfGmAsmU |
| | | | | smU |
| ETD002 | 5542 | fCsmUsfGmCfUmCfAfAfGmCfA | 5543 | mUsfUsmCfCmAfGmCfUmUfG |
| 26 | | mAfGmCfUmGfGmAfAsmUsmU | | mCmUmUfGmAfGmCfAmGsmU |
| | | | | smU |
| ETD002 | 5544 | fAsmCsfUmGfAmAfAfUfCmGfG | 5545 | mUsfUsmUfGmCfAmGfCmCfCm |
| 27 | | mGfCmUfGmCfAmAfAsmUsmU | | GmAmUfUmUfCmAfGmUsmUs |
| | | | | mU |
| ETD002 | 5546 | fUsmGsfAmAfAmUfCfGfGmGfC | 5547 | mUsfCsmCfUmUfGmCfAmGfCm |
| 28 | | mUfGmCfAmAfGmGfAsmUsmU | | CmCmGfAmUfUmUfCmAsmUs |
| | | | | mU |
| ETD002 | 5548 | fAsmAsfUmCfGmGfGfCfUmGfC | 5549 | mUsfGsmCfCmCfCmUfUmGfCm |
| 29 | | mAfAmGfGmGfGmCfAsmUsmU | | AmGmCfCmCfGmAfUmUsmUs |
| | | | | mU |
| ETD002 | 5550 | fAsmUsfGmGfCmGfAfCfAmCfU | 5551 | mUsfGsmUfUmGfGmGfCmAfG |
| 30 | | mGfCmCfCmAfAmCfAsmUsmU | | mUmGmUfCmGfCmCfAmUsmU |
| | | | | smU |
| ETD002 | 5552 | fUsmGsfGmCfGmAfCfAfCmUfG | 5553 | mUsfAsmGfUmUfGmGfGmCfA |
| 31 | | mCfCmCfAmAfCmUfAsmUsmU | | mGmUmGfUmCfGmCfCmAsmU |
| | | | | smU |
| ETD002 | 5554 | fAsmUsfCmCfGmGfAfAfUmGfC | 5555 | mUsfCsmAfGmCfUmUfUmGfC |
| 32 | | mAfAmAfGmCfUmGfAsmUsmU | | mAmUmUfCmCfGmGfAmUsmU |
| | | | | smU |
| ETD002 | 5556 | fCsmCsfCmAfAmGfUfCfCmCfA | 5557 | mUsfAsmAfGmAfGmGfGmUfG |
| 33 | | mCfCmCfUmCfUmUfAsmUsmU | | mGmGmAfCmUfUmGfGmGsmU |
| | | | | smU |
| ETD002 | 5558 | mUsmGsmGmUfGmUfCfGfAmG | 5559 | mUsfAsmGmUmGfAmUmCmUm |
| 34 | | mAmGmAmUmCmAmCmUmAs | | CmUmCmGfAmCfAmCmCmAs |
| | | mUsmU | | mUsmU |
| ETD002 | 5560 | mUsmCsmGmAfGmAfGfAfUmC | 5561 | mUsfAsmUmUmUfCmAmGmUm |
| 35 | | mAmCmUmGmAmAmAmUmAs | | GmAmUmCfUmCfUmCmGmAs |
| | | mUsmU | | mUsmU |
| ETD002 | 5562 | mCsmAsmGmGfUmUfUfGfCmC | 5563 | mUsfGsmAmCmAfAmAmGmUm |
| 36 | | mAmCmUmUmUmGmUmCmAs | | GmGmCmAfAmAfCmCmUmGs |
| | | mUsmU | | mUsmU |
| ETD002 | 5564 | mGsmCsmAmCfCmUfUfUfUmC | 5565 | mUsfGsmAmGmAfCmCmAmUm |
| 37 | | mAmUmGmGmUmCmUmCmAs | | GmAmAmAfAmGfGmUmGmCs |
| | | mUsmU | | mUsmU |
| ETD002 | 5566 | mAsmGsmCmUfGmGfAfGfCmC | 5567 | mUsfGsmUmUmAfGmGmAmA |
| 38 | | mUmUmCmCmUmAmAmCmAs | | mGmGmCmUfCmCfAmGmCmU |
| | | mUsmU | | smUsmU |

### Example 6. Screening ALOX15 siRNAs for Activity in Cells in Culture

The chemically modified ALOX15 siRNAs derived from sequences in Screening Subset A, human/mouse subset (Subset B) and human/rabbit subset (Subsest C) were assayed for ALOX15 mRNA knockdown activity in cells in culture. MCF7 (ATCC® HTB-22™) cells were seeded in 96-well tissue culture plates at a cell density of 10,000 cells per well in DMEM supplemented with 10% fetal bovine serum and incubated overnight in a water-jacketed, humidified incubator at 37° C. in an atmosphere composed of air plus 5% carbon dioxide. The ALOX15 siRNAs were individually transfected into MCF7 cells in duplicate wells at 10 nM final concentration using 0.3 μL Lipofectamine RNAiMax (Fisher) per well. Silencer Select Negative Control #1 (ThermoFisher, Catalog #4390843) and Silencer Select ALOX15 siRNA s1289 (ThermoFisher, Catalog #4392420) were transfected at 10 nM final concentration as controls. After incubation for 48 hours at 37° C., total RNA was harvested from each well and cDNA prepared using TaqMan® Fast Advanced Cells-to-CT™ Kit (ThermoFisher, Catalog #A35374) according to the manufacturer's instructions. The level of ALOX15 mRNA from each well was measured in triplicate by real-time qPCR on an Applied Biosystems 7500 Fast Real-Time PCR machine using TaqMan Gene Expression Assay for human ALOX15 (ThermoFisher, assay #Hs00993765_g1). The level of PPIA mRNA was measured using TaqMan Gene Expression Assay (ThermoFisher, assay #Hs99999904_m1) and used to determine relative ALOX15 mRNA levels in each well using the delta-delta Ct method. All data were normalized to relative ALOX15 mRNA levels in untreated MCF7 cells. Results are shown in Table 6.

TABLE 6
Activity of ALOX15 siRNAs from Screening Set
A, the Human/Mouse Subset and the Human/Rabbit
Subset Transfected in MCF7 Cells at 10 nM

| siRNA (10 nM) | Relative ALOX15 mRNA Level |
|---|---|
| — | 1.000 |
| Negative Control | 0.927 |
| siRNA | |
| siALOX15 | 0.176 |
| ETD00138 | 0.918 |
| ETD00139 | 0.566 |
| ETD00140 | 0.654 |
| ETD00141 | 0.747 |
| ETD00142 | 0.704 |
| ETD00143 | 0.745 |
| ETD00144 | 0.868 |
| ETD00145 | 0.689 |
| ETD00146 | 0.674 |
| ETD00147 | 0.666 |
| ETD00148 | 0.589 |
| ETD00149 | 0.731 |
| ETD00150 | 0.662 |
| ETD00151 | 0.642 |
| ETD00152 | 0.674 |
| ETD00153 | 0.801 |
| ETD00154 | 0.634 |
| ETD00155 | 0.213 |
| ETD00156 | 0.243 |
| ETD00157 | 0.348 |
| ETD00158 | 0.325 |
| ETD00159 | 0.296 |
| ETD00160 | 0.354 |
| ETD00161 | 0.249 |
| ETD00162 | 0.556 |
| ETD00163 | 0.447 |
| ETD00164 | 0.615 |
| ETD00165 | 0.724 |
| ETD00166 | 0.739 |
| ETD00167 | 0.694 |
| ETD00168 | 0.722 |
| ETD00169 | 0.149 |
| ETD00170 | 0.233 |
| ETD00171 | 0.342 |
| ETD00172 | 0.184 |
| ETD00173 | 0.227 |
| ETD00174 | 0.231 |
| ETD00175 | 0.211 |
| ETD00176 | 0.117 |
| ETD00177 | 0.179 |
| ETD00178 | 0.223 |
| ETD00179 | 0.569 |
| ETD00180 | 0.275 |
| ETD00181 | 0.372 |
| ETD00182 | 0.279 |
| ETD00183 | 0.194 |
| ETD00184 | 0.414 |
| ETD00185 | 0.262 |
| ETD00186 | 0.416 |
| ETD00187 | 0.362 |
| ETD00188 | 0.512 |
| ETD00189 | 0.584 |
| ETD00190 | 0.315 |
| ETD00191 | 0.868 |
| ETD00192 | 0.674 |
| ETD00193 | 0.223 |
| ETD00194 | 0.695 |
| ETD00195 | 0.359 |
| ETD00196 | 0.716 |
| ETD00197 | 0.691 |
| ETD00198 | 0.339 |
| ETD00199 | 0.430 |
| ETD00200 | 0.510 |
| ETD00201 | 0.195 |
| ETD00202 | 0.498 |
| ETD00203 | 0.470 |
| ETD00204 | 0.217 |
| ETD00205 | 0.394 |
| ETD00206 | 0.231 |
| ETD00207 | 0.201 |
| ETD00208 | 0.413 |
| ETD00209 | 0.185 |
| ETD00210 | 0.131 |
| ETD00211 | 0.152 |
| ETD00212 | 0.182 |
| ETD00213 | 0.228 |
| ETD00214 | 0.187 |
| ETD00215 | 0.723 |
| ETD00216 | 0.410 |
| ETD00217 | 0.229 |
| ETD00218 | 0.490 |
| ETD00219 | 0.359 |
| ETD00220 | 0.637 |
| ETD00221 | 0.223 |
| ETD00222 | 0.373 |
| ETD00223 | 0.254 |
| ETD00224 | 0.188 |
| ETD00225 | 0.268 |
| ETD00226 | 0.478 |
| ETD00227 | 0.408 |
| ETD00228 | 0.612 |
| ETD00229 | 0.537 |
| ETD00230 | 0.217 |
| ETD00231 | 0.217 |
| ETD00232 | 0.220 |
| ETD00233 | 0.355 |

“—”, untreated MCF7 cells;
Negative Control siRNA, Silencer Select Negative Control #1;
siALOX15, Silencer Select ALOX15 siRNA s1289

The 42 siRNAs showing the greatest degree of knockdown of ALOX15 siRNA at 10 nM were tested in a second screen for activity at 1 nM concentration using MCF7 cells and the transfection procedures as described above. Results are shown in Table 7.

TABLE 7
Activity of 42 ALOX15 siRNAs from Screening Set
A, the Human/Mouse Subset and the Human/Rabbit
Subset Transfected in MCF7 Cells at 1 nM

| siRNA (1 nM) | Relative ALOX15 mRNA Level |
|---|---|
| — | 1.000 |
| Negative Control | 0.980 |
| siRNA | |
| siALOX15 | 0.352 |
| ETD00155 | 0.507 |
| ETD00156 | 0.586 |
| ETD00158 | 0.533 |
| ETD00159 | 0.552 |
| ETD00161 | 0.309 |
| ETD00169 | 0.236 |
| ETD00170 | 0.320 |
| ETD00171 | 0.397 |
| ETD00172 | 0.216 |
| ETD00173 | 0.270 |
| ETD00174 | 0.337 |
| ETD00175 | 0.316 |
| ETD00176 | 0.134 |
| ETD00177 | 0.201 |
| ETD00178 | 0.287 |
| ETD00180 | 0.418 |
| ETD00182 | 0.387 |
| ETD00183 | 0.246 |
| ETD00184 | 0.423 |
| ETD00185 | 0.199 |
| ETD00190 | 0.423 |
| ETD00193 | 0.218 |
| ETD00195 | 0.231 |
| ETD00198 | 0.397 |
| ETD00201 | 0.263 |
| ETD00204 | 0.217 |
| ETD00206 | 0.496 |
| ETD00207 | 0.542 |
| ETD00209 | 0.291 |
| ETD00210 | 0.225 |
| ETD00211 | 0.222 |
| ETD00212 | 0.340 |
| ETD00213 | 0.365 |
| ETD00214 | 0.315 |
| ETD00217 | 0.346 |
| ETD00221 | 0.198 |
| ETD00223 | 0.623 |
| ETD00224 | 0.499 |
| ETD00225 | 0.316 |
| ETD00230 | 0.371 |
| ETD00231 | 0.372 |
| ETD00232 | 0.307 |
| ETD00214 | 0.315 |
| ETD00217 | 0.346 |
| ETD00221 | 0.198 |
| ETD00223 | 0.623 |
| ETD00224 | 0.499 |
| ETD00225 | 0.316 |
| ETD00230 | 0.371 |
| ETD00231 | 0.372 |
| ETD00232 | 0.307 |

“—”, untreated MCF7 cells;
Negative Control siRNA, Silencer Select Negative Control #1;
siALOX15, Silencer Select ALOX15 siRNA s1289

### Example 7. Determining the IC50 of ALOX15 siRNAs

The IC50 values for knockdown of ALOX15 mRNA by select ALOX15 siRNAs were determined in MCF7 cells. The ETD00183, ETD00185, ETD00193, ETD00210 and ETD00217 siRNAs were assayed individually at 3 nM, 1 nM, 0.3 nM, 0.1 nM and 0.03 nM. MCF7 (ATCC® HTB-22™) cells were seeded in 96-well tissue culture plates at a cell density of 10,000 cells per well in DMEM supplemented with 10% fetal bovine serum and incubated overnight in a water-jacketed, humidified incubator at 37° C. in an atmosphere composed of air plus 5% carbon dioxide. The ALOX15 siRNAs were individually transfected into MCF7 cells in triplicate wells using 0.3 μL Lipofectamine RNAiMax (Fisher) per well. Silencer Select Negative Control #1 (ThermoFisher, Catalog #4390843) was transfected at 3 nM final concentration as controls. After incubation for 48 hours at 37° C., total RNA was harvested from each well and cDNA prepared using TaqMan® Fast Advanced Cells-to-CT™ Kit (ThermoFisher, Catalog #A35374) according to the manufacturer's instructions. The level of ALOX15 mRNA from each well was measured in triplicate by real-time qPCR on an Applied Biosystems 7500 Fast Real-Time PCR machine using TaqMan Gene Expression Assay for human ALOX15 (ThermoFisher, assay #Hs00993765_g1). The level of PPIA mRNA was measured using TaqMan Gene Expression Assay (ThermoFisher, assay #Hs99999904_m1) and used to determine relative ALOX15 mRNA levels in each well using the delta-delta Ct method. All data were normalized to relative ALOX15 mRNA levels in untreated MCF7 cells. Curve fit was accomplish using the [inhibitor] vs. response (three parameters) function in GraphPad Prism software. Results are shown in Table 8.

TABLE 8
IC50 Values of ALOX15 siRNAs

| | | | Relative ALOX15 | | R |
|---|---|---|---|---|---|
| siRNA | [siRNA] | | mRNA Levels | IC50 | squared |
| — | — | | 1.000 | ND | ND |
| Negative | 3 | nM | 1.283 | ND | ND |
| Control | | | | | |
| siRNA | | | | | |
| ETD00183 | 3 | nM | 0.214 | 0.044 | 0.958 |
| | 1 | nM | 0.312 | | |
| | 0.3 | nM | 0.293 | | |
| | 0.1 | nM | 0.435 | | |
| | 0.03 | nM | 0.610 | | |
| ETD00185 | 3 | nM | 0.138 | 0.029 | 0.954 |
| | 1 | nM | 0.211 | | |
| | 0.3 | nM | 0.200 | | |
| | 0.1 | nM | 0.276 | | |
| | 0.03 | nM | 0.417 | | |
| ETD00193 | 3 | nM | 0.240 | 0.094 | 0.993 |
| | 1 | nM | 0.265 | | |
| | 0.3 | nM | 0.333 | | |
| | 0.1 | nM | 0.397 | | |
| | 0.03 | nM | 0.510 | | |
| ETD00210 | 3 | nM | 0.189 | 0.082 | 0.995 |
| | 1 | nM | 0.271 | | |
| | 0.3 | nM | 0.394 | | |
| | 0.1 | nM | 0.573 | | |
| | 0.03 | nM | 0.849 | | |
| ETD00217 | 3 | nM | 0.302 | 0.158 | 0.989 |
| | 1 | nM | 0.415 | | |
| | 0.3 | nM | 0.505 | | |
| | 0.1 | nM | 0.742 | | |
| | 0.03 | nM | 0.887 | | |

“—”, untreated MCF7 cells;
Negative Control siRNA, Silencer Select Negative Control #1;
ND, not determined

### Example 8. Determining the IC50 of ALOX15 siRNAs with Alternative 2′ Modification Patterns

The IC50 values for knockdown of ALOX15 mRNA by ALOX15 siRNAs with alternative 2′ modification patterns were determined in MCF7 cells. The ETD00234, ETD00235, ETD00236, ETD00237 and ETD00238 siRNAs were assayed individually at 3 nM, 1 nM, 0.3 nM, 0.1 nM and 0.03 nM. MCF7 (ATCC® HTB-22™) cells were seeded in 96-well tissue culture plates at a cell density of 10,000 cells per well in DMEM supplemented with 10% fetal bovine serum and incubated overnight in a water-jacketed, humidified incubator at 37° C. in an atmosphere composed of air plus 500 carbon dioxide. The ALOX15 siRNAs were individually transfected into MCF7 cells in triplicate wells using 0.3 μL Lipofectamine RNAiMax (Fisher) per well. Silencer Select Negative Control #1 (ThermoFisher, Catalog #4390843) was transfected at 3 nM final concentration as controls. After incubation for 48 hours at 37° C., total RNA was harvested from each well and cDNA prepared using TaqMan® Fast Advanced Cells-to-CT™ Kit (ThermoFisher, Catalog #A35374) according to the manufacturer's instructions. The level of ALOX15 mRNA from each well was measured in triplicate by real-time qPCR on an Applied Biosystems 7500 Fast Real-Time PCR machine using TaqMan Gene Expression Assay for human ALOX15 (ThermoFisher, assay #Hs00993765_g1). The level of PPIA mRNA was measured using TaqMan Gene Expression Assay (ThermoFisher, assay #Hs99999904_m1) and used to determine relative ALOX15 mRNA levels in each well using the delta-delta Ct method. All data were normalized to relative ALOX15 mRNA levels in untreated MCF7 cells. Curve fit was accomplish using the [inhibitor] vs. response (three parameters) function in GraphPad Prism software. Results are shown in Table 9.

TABLE 9
IC50 Values of ALOX15 siRNAs with Alternative
2′ Modification Patterns

| | | | Relative ALOX15 | | R |
|---|---|---|---|---|---|
| siRNA | [siRNA] | | mRNA Levels | IC50 | squared |
| — | — | | 1.000 | ND | ND |
| Negative | 3 | nM | 1.150 | ND | ND |
| Control | | | | | |
| siRNA | | | | | |
| siALOX15 | 3 | nM | 0.461 | ND | ND |
| ETD00234 | 0.03 | nM | 0.753 | 0.653 | 0.954 |
| | 0.1 | nM | 0.714 | | |
| | 0.3 | nM | 0.666 | | |
| | 1 | nM | 0.396 | | |
| | 3 | nM | 0.353 | | |
| ETD00235 | 0.03 | nM | 0.544 | 1.342 | 0.239 |
| | 0.1 | nM | 0.357 | | |
| | 0.3 | nM | 0.522 | | |
| | 1 | nM | 0.400 | | |
| | 3 | nM | 0.383 | | |
| ETD00236 | 0.03 | nM | 0.537 | 0.100 | 0.967 |
| | 0.1 | nM | 0.485 | | |
| | 0.3 | nM | 0.372 | | |
| | 1 | nM | 0.360 | | |
| | 3 | nM | 0.336 | | |
| ETD00238 | 0.03 | nM | 0.958 | 0.447 | 0.959 |
| | 0.1 | nM | 0.796 | | |
| | 0.3 | nM | 0.729 | | |
| | 1 | nM | 0.599 | | |
| | 3 | nM | 0.437 | | |

### Example 9. Assessing the Extent of Nuclease Resistance of ALOX15 siRNAs

Resistance of select ALOX15 siRNAs to nuclease digestion was assessed by incubating the siRNAs in rat liver tritosomes. Each siRNA (7 ng/μL final concentration) was placed into a PCR tube containing a cocktail prepared on ice containing 1× catabolic buffer (Xenotech, Catalog #K5200, Lot #18-1-0698), 0.5× rat tritosomes (Xenotech, Catalog #R0610.LT, Lot #1610405), 0.1 U/μL porcine intestinal heparin (Zageno, Catalog #H3149-10KU). An aliquot was removed, an equal volume of 50 mM EDTA was added, and the sample placed at −80° C. This sample was designated as the 0 hr timepoint. The remainder of the reaction was placed in an Eppendorf Mastercycler Gradient and incubated at 37° C. After incubation for 4 and 24 hours, an aliquot was removed from the reaction and stopped by addition of an equal volume of 50 mM EDTA and placed at −80° C. until analysis by gel electrophoresis. All samples were then thawed on ice and 6×DNA Gel Loading Dye (ThermoFisher Catalog #R0611) was added to 1× final concentration. 20 μL of each sample was loaded onto a 20% polyacrylamide TBE gel (ThermoFisher, Catalog #EC63155BOX). Electrophoresis was carried out at a constant 100V for 75 minutes in an XCell SureLock Mini-Cell Electrophoresis System (ThermoFisher) using 1×TBE (Tris/boric/EDTA) (Fisher, Catalog #FERB52) as the tank buffer. The siRNA was visualized by staining the gel with a 1:10,000 dilution of SYBR Gold (ThermoFisher, Catalog #S-11494) in TBE for 15 minutes at room temperature with rocking. The gel was washed with 1×TBE for 15 minutes and then placed on a FotoPrep1 UV transilluminator (Fotodyne). The gel was imaged using the camera app set on MONO on an iPhone 6s with a yellow gel filter (Neewer) placed over the lens. Band intensity was measured using NIH ImageJ using the “Analyze: Gels” function. The remaining siRNA percent was normalized to the value obtained at the 0 hr timepoint for that siRNA. Results are shown in Table 10. By using this assay, we were able to determine that some siRNAs are more resistant to nuclease digestion with more remaining intact over time compared with other siRNAs with the same modification pattern.

TABLE 10
Resistance of ALOX15 siRNAs to Nucleases
Present in Rat Liver Tritosomes

| siRNA | Timepoint (hr) | % remaining |
|---|---|---|
| ETD00169 | 0 | 100% |
| | 4 | 65% |
| | 24 | 36% |
| ETD00172 | 0 | 100% |
| | 4 | 73% |
| | 24 | 74% |
| ETD00176 | 0 | 100% |
| | 4 | 50% |
| | 24 | 17% |
| ETD00177 | 0 | 100% |
| | 4 | 60% |
| | 24 | 41% |
| ETD00183 | 0 | 100% |
| | 4 | 83% |
| | 24 | 70% |
| ETD00185 | 0 | 100% |
| | 4 | 80% |
| | 24 | 68% |
| ETD00193 | 0 | 100% |
| | 4 | 81% |
| | 24 | 61% |
| ETD00210 | 0 | 100% |
| | 4 | 77% |
| | 24 | 56% |
| ETD00211 | 0 | 100% |
| | 4 | 85% |
| | 24 | 66% |
| ETD00217 | 0 | 100% |
| | 4 | 67% |
| | 24 | 19% |
| ETD00234 | 0 | 1.00 |
| | 4 | 1.18 |
| | 24 | 0.74 |
| ETD00235 | 0 | 1.00 |
| | 4 | 1.10 |
| | 24 | 0.69 |
| ETD00236 | 0 | 1.00 |
| | 4 | 1.26 |
| | 24 | 0.45 |
| ETD00237 | 0 | 1.00 |
| | 4 | 0.79 |
| | 24 | 0.40 |
| ETD00238 | 0 | 1.00 |
| | 4 | 0.81 |
| | 24 | 0.29 |

### Example 10. Inhibition of ALOX15 in a Mouse Model for Nasal Polyps Using Modified ALOX15 siRNAs

In this experiment, a murine model of nasal polyps is used to evaluate the effect of siRNA inhibition of ALOX15. In the murine model, chronic rhinitis is induced with ovalbumin (OVA) treatment, followed by treatment with a combination of ovalbumin and Staphylococcus aureus enterotoxin B (SEB) leading to nasal polyps formation.

Briefly, mice are divided into three groups: Group 1—a nasal polyp group treated with non-targeting control siRNA composed of annealed sense strand SEQ ID NO: 5358 and antisense strands SEQ ID NO: 5359, Group 2—a nasal polyp group treated with ALOX15 siRNA ETD00217 (SEQ ID NOS: 5524 and 5525), Group 3—a nasal polyp group treated with ALOX15 siRNA ETD00238 (SEQ ID NOS: 5566 and 5567) with five mice in each group. 4-week-old female BALB/c mice are sensitized with an intraperitoneal injection of 25 ug of OVA on days 0 and 7. From day 14 to day 20 mice are nasally challenged daily with 6% OVA. From day 20, 6% OVA+Staphylococcus aureus enterotoxin B (10 ng) is instilled into the nasal cavity of the mice three times per week for 8 weeks.

At weeks 0 and 4 of OVA+SEB, siRNA is applied. Anesthesia for administration of siRNA is achieved with intraperitoneal injection of 0.2 ml nembutal (5 mg/ml). Naked siRNA is resuspended in Opti-MEM, and 10 ug siRNA in 5 μL volume is delivered to each nostril with a micropipette. At the end of eight weeks of OVA+SEB and siRNA treatment, mice are sacrificed by cervical dislocation following injection of 0.3 ml nembutal. Nasal cavity samples are prepared using a large scalpel to remove the snout with a transverse cut behind the back molars. The external nares are flushed with PBS to wash out any blood within the nasal cavity. Histologic quantification of the number of nasal polyps and the amount of eosinophil infiltration is performed using hematoxylin and eosin (H&E) staining of the nasal cavity. The number of polyps and amount of eosinophilic infiltration are compared in the non-targeting siRNA Group 1 and the ALOX15 siRNA Groups 2 and 3. Additionally, nasal mucosa is removed using a small curette after bisecting the nasal tissue sagitally along the nasal septum. Cell lysates are prepared using a RIPA buffer, and these lysates are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol. Metabolite levels are compared between non-targeting siRNA Group 1 and the ALOX15 siRNA Groups 2 and 3.

### Example 11. siRNA Inhibition of ALOX15 in a Mouse Model for Nasal Polyps

In this experiment, a murine model of nasal polyps is used to evaluate the effect of siRNA inhibition of ALOX15. In the murine model, chronic rhinitis is induced with ovalbumin (OVA) treatment, followed by treatment with a combination of ovalbumin and Staphylococcus aureus enterotoxin B (SEB) leading to nasal polyps formation.

Briefly, mice are divided into two groups: a nasal polyp+scrambled siRNA group (NP+siRNA scrambled) and a nasal polyp group+ALOX15 siRNA group (NP+siRNA ALOX15), with five mice in each group. 4-week-old female BALB/c mice are sensitized with an intraperitoneal injection of 25 μg of OVA on days 0 and 7. From day 14 to day 20 mice are nasally challenged daily with 6% OVA. From day 20, 6% OVA+Staphylococcus aureus enterotoxin B (10 ng) is instilled into the nasal cavity of the mice three times per week for 8 weeks.

At weeks 0 and 4 of OVA+SEB, siRNA is applied. Anesthesia for administration of siRNA is achieved with intraperitoneal injection of 0.2 ml nembutal (5 mg/ml). The ALOX15 targeting siRNA is cross-reactive with the mouse and human ALOX15 mRNA and consists of a double stranded RNA oligonucleotide with sense and antisense sequences as follows: 5′ mGsmCmUmGfUmGmCmUfGmAmAmGfAmAmGmUfUfCmAsdTsdT 3′ (SEQ ID NO: 5354, or SEQ ID NO: 3765 with 2′ modifications and having the last two residues replaced by two T residues) and 5′ mUsfGmAmAmCfUfUmCmUfUfCfAmGfCmAmCmAfGmCsdTsdT 3′ (SEQ ID NO: 5355, or SEQ ID NO: 3766 with 2′ modifications and having the last two residues replaced by two T residues). This siRNA targets nucleotide positions 1890-1908 of the murine ALOX15 mRNA (GenBank Acc. #NM_009660.3) (full-length murine mRNA set forth as SEQ ID NO: 5356) and positions 1883-2001 of the human ALOX15 mRNA (GenBank Acc. #NM_001140.4) (full-length human mRNA set forth as SEQ ID NO: 5357). The non-targeting control siRNA consists of the following sense and antisense sequences: 5′ mGsmUmCmCfAmUmCmAmfGmCmUmCmfGmGmUmUfAfGmAsdTsdT 3′ (SEQ ID NO: 5358) and 5′ mUsfCmUmAmAfCfCmGmAfGfCfUmGfAmUmGmGfAmCdTdT 3′ (SEQ ID NO: 5359). The letter “m” before a nucleotide indicates a 2′O-methyl substitution, the letter “f” indicates a 2′flouro substitution, the letter “d” indicates a deoxyribonucleotide substitution, and the letter “s” indicates a phosphorothioate linkage.

Naked siRNA is resuspended in Opti-MEM, and 10 ug siRNA in 5 ul volume is delivered to each nostril with a micropipette. At the end of eight weeks of OVA+SEB and siRNA treatment, mice are sacrificed by cervical dislocation following injection of 0.3 ml nembutal. Nasal cavity samples are prepared using a large scalpel to remove the snout with a transverse cut behind the back molars. The external nares are flushed with PBS to wash out any blood within the nasal cavity. Histologic quantification of the number of nasal polyps and the amount of eosinophil infiltration is performed using hematoxylin and eosin (H&E) staining of the nasal cavity. The number of polyps and amount of eosinophilic infiltration are compared in the NP+siRNA non-targeting group and the NP+siRNA ALOX15 group. Additionally, nasal mucosa is removed using a small curette after bisecting the nasal tissue sagitally along the nasal septum. Cell lysates are prepared using a RIPA buffer, and these lysates are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol. Metabolite levels are compared between the NP+siRNA non-targeting group and the NP+siRNA ALOX15 group.

### Example 12. Inhibition of ALOX15 in Human Cells Using Modified ALOX15 siRNAs

In this experiment siRNA inhibition of ALOX15 is performed in primary human nasal epithelial cells (pHNEC) and a human eosinophil cell line (EoL-1), to evaluate the effect on arachidonic acid metabolite production under conditions of no treatment versus IL13 treatment. IL13 treatment serves to induce ALOX15 expression, and is a critical regulator of the Th2 inflammation present in human nasal polyposis patients.

EoL-1 cells are initially cultured in the presence of dbcAMP (100 uM) for 7 days to differentiate to a mature eosinophil cell type. Next, differentiated EoL-1 cells and pHNECs are transfected in a 24-well plate with a non-targeting siRNA composed of annealed sense strand SEQ ID NO: 5358 and antisense strands SEQ ID NO: 5359 or ALOX15 targeting siRNAs ETD00183, ETD00185, ETD00193, ETD00210, ETD00217, ETD00234, ETD00235, ETD00236, ETD00237 and ETD00238 (see Table 5B for siRNA sequences), immediately followed by +/− supplementation with IL13 (20 ng/ml). Additionally, all wells are supplemented with 3 μM arachidonic acid so that substrate is a not a limiting factor in metabolite production.

Briefly, transfections are performed using TransIT TKO (Mirus) following the manufacturer's recommended protocol. For each well, 1.4 μL siRNA (10 uM stock), 2.5 μL TransIT-TKO, and 50 μL OptiMEM are mixed, incubated at room temperature for 30 minutes, and added dropwise to each well. At 72 hrs post-transfection, supernatant is collected, cells are trypsinized, and cell lysates prepared using a RIPA buffer. Supernatants are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol. Cell lysates are used to perform Western blots, using an ALOX15 antibody (Abcam) and a GAPDH antibody (Abcam) as a loading control. Comparison made between the groups treated with ALOX15 targeting siRNAs, untreated cells, and cells treated with a non-targeting control siRNA.

### Example 13. siRNA Inhibition of ALOX15 in Human Cells

In this experiment siRNA inhibition of ALOX15 is performed in primary human nasal epithelial cells (pHNEC) and a human eosinophil cell line (EoL-1), to evaluate the effect on arachidonic acid metabolite production under conditions of no treatment versus IL13 treatment. IL13 treatment serves to induce ALOX15 expression, and is a critical regulator of the Th2 inflammation present in human nasal polyposis patients.

EoL-1 cells are initially cultured in the presence of dbcAMP (100 uM) for 7 days to differentiate to a mature eosinophil cell type. Next, differentiated EoL-1 cells and pHNECs are transfected in a 24-well plate with a non-targeting or ALOX15 targeting siRNA immediately followed by +/− supplementation with IL13 (20 ng/ml). Additionally, all wells are supplemented with 10 uM arachidonic acid so that substrate is a not a limiting factor in metabolite production. The ALOX15 targeting siRNA consisted of a double stranded RNA oligonucleotide with sense and antisense sequences as follows: 5′ GCUGUGCUGAAGAAGUUCAdTdT 3′ (SEQ ID NO: 5360, or SEQ ID NO: 3765 having the last two residues replaced by two T residues) and 5′ UGAACUUCUUCAGCACAGCdTdT 3′ (SEQ ID NO: 5361, or SEQ ID NO: 3766 having the last two residues replaced by two T residues). This siRNA targets nucleotide positions 1883-2001 of the human ALOX15 mRNA (GenBank Acc. #NM_001140.4). The non-targeting control siRNA consists of the following sense and antisense sequences: 5′ GUCCAUCAGCUCGGUUAGAdTdT 3′ (SEQ ID NO: 5362) and 5′ UCUAACCGAGCUGAUGGACdTdT 3′ (SEQ ID NO: 5363). The letter “d” indicates a deoxyribonucleotide substitution. Briefly, transfections are performed using TransIT TKO (Mirus) following the manufacturer's recommended protocol. For each well, 1.4 μL siRNA (10 μM stock), 2.5 μL TransIT-TKO, and 50 μL OptiMEM are mixed, incubated at room temperature for 30 minutes, and added dropwise to each well. At 72 hrs post-transfection, supernatant is collected, cells are trypsinized, and cell lysates prepared using a RIPA buffer. Supernatants are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol. Cell lysates are used to perform Western blots, using an ALOX15 antibody (Abcam) and a GAPDH antibody (Abcam) as a loading control. Comparison made between the groups treated with ALOX15 targeting siRNA, untreated cells, and cells treated with a non-targeting control siRNA.

### Example 14. Inhibition of ALOX15 in a Rabbit Model for Nasal Polyps Using Modified ALOX15 siRNAs

In this experiment, a rabbit model of nasal polyps is used to evaluate the effect of siRNA inhibition of ALOX15. In the rabbit model eosinophilic nasal polyps are induced by eliciting an allergic reaction in animals with ovalbumin (OVA) and poly-L-arginine treatment. This model has characteristics of the human disease.

Male New Zealand white rabbits are obtained from Charles River Laboratories. The animals are kept in environmentally controlled rooms under specific pathogen-free conditions (temperature, 20-26° C.; humidity, 30-70%) with a 12-hour light-dark cycle for 2 weeks before use. Food and water are available ad libitum. All animals are used in accordance with animal care guidelines.

Maxillary sinusitis is induced in age-matched (13-week-old) rabbits divided into four groups. Group A (n=4) is a control group in which rabbits are treated with neither reagent nor surgery. For groups B-D (n=6 for each), rabbits are sensitized by subcutaneous injection with 1 mL of saline containing 2.5% OVA plus 0.4% alum on days 0 and 7. On day 14, under anesthesia with i.v. injection of 25 mg/kg of pentobarbital sodium (Nembutal, Dainippon Sumitomo Pharma, Osaka, Japan), nasal dorsa are incised to expose maxillary sinus cavities and both sides of the ostia were occluded with plugs of sterile cotton wool and butylcyanoacrylate tissue glue (Histoacryl; B. Braun, Melsungen AG, Germany) under a microscope. After 2 weeks of wound closure, OVA is administered into both sides of the maxillary sinuses (0.5 mL/sinus of 2.5% OVA in saline, three times a week for 2 weeks). Thereafter, the animals in groups B-D receive 5 mg/mL poly-L-arginine in saline three times a week for 4 weeks.

After induction of maxillary sinusitis with OVA and poly-L-arginine, the animals receive 0.5 mL/sinus of saline (group B), 2 mg/mL ALOX15 siRNA in saline (group C), or 2 mg/mL control siRNA in saline (group D) three times a week for 4 weeks. One week after the last administration into the maxillary sinus, the rabbits are sacrificed by i.v. injection of pentobarbital sodium and the anterior nasal region with the attached bone, excluding the ocular bulb, is dissected. The mucosal tissues for gene analysis are collected from the right side of the maxillary sinus and stored in RNAlater RNA stabilization reagent (Ambion, Austin, Tex.) at 4° C. until analysis. The left side of the maxillary sinus is used for histopathological analysis. Anesthesia for administration of siRNA is achieved with intraperitoneal injection of 0.2 ml nembutal (5 mg/ml). The ALOX15 targeting siRNA ETD00234 is cross-reactive with the rabbit and human ALOX15 mRNA. The non-targeting control siRNA is composed of annealed sense strand SEQ ID NO: 5358 and antisense strands SEQ ID NO: 5359.

Antibodies to OVA are measured using enzyme-linked immunosorbent assay (ELISA). Blood samples are collected from the pinna vein on day 13 to measure OVA-specific IgG levels. ELISA is performed according to the protocol of a previous study. The titers of samples are calculated by comparison with internal standard serum, prepared from the rabbits immunized with 2.5% OVA plus 0.4% alum eight times. The value of this standard is arbitrarily calculated as 10,000 U/ml.

Histopathological analysis of nasal tissue is carried out on tissue fixed with 10% neutral buffered formalin solution for 1 week, decalcified in 0.5 mol/L of ethylenediaminetetraacetic acid at 37° C., embedded in paraffin, and cut into 2-μm-thick sections. After hematoxylin-eosin staining, histopathological analysis is performed by selecting a representative field of mucosa per sinus where the most prominent change is detected with 200× magnification. The number of eosinophils and the area of mucosa in each field are measured to calculate the density of eosinophils (cells/mm2). The width of the lamina propria (μm) is measured as an indirect indication of mucosal hypertrophy. The degree of polyp formation is graded semiquantitatively according to the following score: 0=little or no polyp formation detected; 1=slight (slight prominence of mucosa); 2=moderate (polyp of a size from one-quarter to one-half of the field); 3=severe (polyp of a size more than one-half of the field).

ALOX15 mRNA knockdown in the mucosal tissues is quantified using real-time polymerase chain reaction. Total RNA is reverse transcribed to cDNA using a Maxima First Strand cDNA Synthesis Kit for RT-qPCR (Invitrogen, Carlsbad, Calif.). Real-time quantitative polymerase chain reaction (PCR) is performed on an Applied Biosystems 7500 Fast Real-Time PCR machine using TaqMan Gene Expression Assay for rabbit ALOX15 (ThermoFisher, assay #Oc03823548_s1). The level of rabbit GAPDH mRNA is measured using TaqMan Gene Expression Assay (ThermoFisher, assay #Oc03823402_g1) and used to determine relative ALOX15 mRNA levels in each rabbit using the delta-delta Ct method.

### Example 15. siRNA Inhibition of ALOX15 in a Rabbit Model for Nasal Polyps

In this experiment, a rabbit model of nasal polyps is used to evaluate the effect of siRNA inhibition of ALOX15. In the rabbit model eosinophilic nasal polyps are induced by eliciting an allergic reaction in animals with ovalbumin (OVA) and poly-L-arginine treatment. This model has characteristics of the human disease.

Male New Zealand white rabbits are obtained from Charles River Laboratories. The animals are kept in environmentally controlled rooms under specific pathogen-free conditions (temperature, 20-26° C.; humidity, 30-70%) with a 12-hour light-dark cycle for 2 weeks before use. Food and water are available ad libitum. All animals are used in accordance with animal care guidelines.

Maxillary sinusitis is induced in age-matched (13-week-old) rabbits divided into four groups. Group A (n=4) is a control group in which rabbits are treated with neither reagent nor surgery. For groups B-D (n=6 for each), rabbits are sensitized by subcutaneous injection with 1 mL of saline containing 2.5% OVA plus 0.4% alum on days 0 and 7. On day 14, under anesthesia with i.v. injection of 25 mg/kg of pentobarbital sodium (Nembutal, Dainippon Sumitomo Pharma, Osaka, Japan), nasal dorsa are incised to expose maxillary sinus cavities and both sides of the ostia were occluded with plugs of sterile cotton wool and butylcyanoacrylate tissue glue (Histoacryl; B. Braun, Melsungen AG, Germany) under a microscope. After 2 weeks of wound closure, OVA is administered into both sides of the maxillary sinuses (0.5 mL/sinus of 2.5% OVA in saline, three times a week for 2 weeks). Thereafter, the animals in groups B-D receive 5 mg/mL poly-L-arginine in saline three times a week for 4 weeks.

After induction of maxillary sinusitis with OVA and poly-L-arginine, the animals receive 0.5 mL/sinus of saline (group B), 2 mg/mL ALOX15 siRNA in saline (group C), or 2 mg/mL control siRNA in saline (group D) three times a week for 4 weeks. One week after the last administration into the maxillary sinus, the rabbits are sacrificed by i.v. injection of pentobarbital sodium and the anterior nasal region with the attached bone, excluding the ocular bulb, is dissected. The mucosal tissues for gene analysis are collected from the right side of the maxillary sinus and stored in RNAlater RNA stabilization reagent (Ambion, Austin, Tex.) at 4° C. until analysis. The left side of the maxillary sinus is used for histopathological analysis. Anesthesia for administration of siRNA is achieved with intraperitoneal injection of 0.2 ml nembutal (5 mg/ml). The ALOX15 targeting siRNA is cross-reactive with the rabbit and human ALOX15 mRNA and consists of a double stranded RNA oligonucleotide with sense and antisense sequences as follows: 5′ mCsmUmGmUfGmGmAmUfGmAmGmCfGmAmUmUfUfCmUsdTsdT 3′ (SEQ ID NO: 5364, or SEQ ID NO: 1007 having the last two residues replaced by two T residues) and 5′ mAsfGmAmAmAfUfCmGmCfUfCfAmUfCmCmAmCfAmGsdTsdT 3′ (SEQ ID NO: 5365, or SEQ ID NO: 1007 having the last two residues replaced by two T residues). This siRNA targets nucleotide positions 512-530 of the rabbit ALOX15 mRNA (GenBank Acc. #NM_009660.3) and positions 504-522 of the human ALOX15 mRNA (GenBank Acc. #NM_001140.4). The non-targeting control siRNA consists of the following sense and antisense sequences: 5′ mGsmUmCmCfAmUmCmAmfGmCmUmCmfGmGmUmUfAfGmAsdTsdT 3′ (SEQ ID NO: 5358) and 5′ mUsfCmUmAmAfCfCmGmAfGfCfUmGfAmUmGmGfAmCdTdT 3′ (SEQ ID NO: 5359). The letter “m” before the nucleotide indicates a 2′O-methyl substitution, the letter “f” indicates a 2′flouro substitution, the letter “d” indicates a deoxyribonucleotide substitution, and the letter “s” indicates a phosphorothioate linkage. Naked siRNA is resuspended in Opti-MEM, and 400 ug siRNA in 200 μL volume is delivered to each nostril with a micropipette.

Antibodies to OVA are measured using enzyme-linked immunosorbent assay (ELISA). Blood samples are collected from the pinna vein on day 13 to measure OVA-specific IgG levels. ELISA is performed according to the protocol of a previous study. The titers of samples are calculated by comparison with internal standard serum, prepared from the rabbits immunized with 2.5% OVA plus 0.4% alum eight times. The value of this standard is arbitrarily calculated as 10,000 U/ml.

Histopathological analysis of nasal tissue is carried out on tissue fixed with 10% neutral buffered formalin solution for 1 week, decalcified in 0.5 mol/L of ethylenediaminetetraacetic acid at 37° C., embedded in paraffin, and cut into 2-μm-thick sections. After hematoxylin-eosin staining, histopathological analysis is performed by selecting a representative field of mucosa per sinus where the most prominent change is detected with 200× magnification. The number of eosinophils and the area of mucosa in each field are measured to calculate the density of eosinophils (cells/mm²). The width of the lamina propria (μm) is measured as an indirect indication of mucosal hypertrophy. The degree of polyp formation is graded semiquantitatively according to the following score: 0=little or no polyp formation detected; 1=slight (slight prominence of mucosa); 2=moderate (polyp of a size from one-quarter to one-half of the field); 3=severe (polyp of a size more than one-half of the field).

ALOX15 mRNA knockdown is quantified using real-time polymerase chain reaction. Total RNA is reverse transcribed to cDNA using a First-Strand III cDNA Synthesis kit (Invitrogen, Carlsbad, Calif.). Real-time quantitative polymerase chain reaction (PCR) is performed using the ABI Prism 7900 Sequence Detection System (Applied Biosystems, Foster City, Calif.). Amplification by PCR is performed according to the manufacturer's protocols (Applied Biosystems). Primers and probes for ALOX15 used in this study and p-actin are designed with the assistance of the computer program Primer Express (Applied Biosystems). Searches using a nucleotide basic local alignment search tool (BLASTN) database are conducted to confirm their specificity and the absence of DNA polymorphisms.

### Example 16. Sequences

SEQ ID NOS: 1-5349, 5366-5971, 6000-6038, 6050-6074 are exemplary ALOX15 siRNA sequences directed against human ALOX15 mRNA. The sequences are listed in the 5′-3′ orientation. Sequence ID numbers are indicated prior to each sequence. In some embodiments, for SEQ ID NOS: 1-5349, odd numbered sequences are the sense, or passenger, strand and even numbered sequences in each strand are the antisense, or guide, strand. Each sense and antisense strand can be annealed to produce the siRNA. In some embodiments, at least the final two 3′ nucleotides in each strand are substituted with different nucleotides. In some embodiments, one or more nucleotides in each sequence is modified. Table 11 outlines some sequences provided in this disclosure.

TABLE 11
Certain Sequences

| SEQ ID | |
|---|---|
| NO | PROTEIN/GENE |
| 1-5349 | ALOX15 siRNA oligonucleotide sequences |
| 5350 | An RFGF containing a hydrophobic membrane |
| | translocation sequence (MTS) peptide |
| 5351 | An RFGF analogue containing a hydrophobic |
| | membrane translocation sequence (MTS) |
| | peptide |
| 5352 | Peptide from HIV Tat protein |
| 5353 | Peptide from Drosophila Antennapedia protein |
| 5354 | ALOX15 targeting siRNA sense |
| 5355 | ALOX15 targeting siRNA antisense |
| 5356 | Full-length ALOX15 murine mRNA |
| | (GenBank Acc. # NM_009660.3) |
| 5357 | Full-length ALOX15 human mRNA |
| | (GenBank Acc. # NM_001140.4) |
| 5358 | Non-targeting control siRNA sense |
| 5359 | Non-targeting control siRNA antisense |
| 5360 | ALOX15 targeting siRNA sense |
| 5361 | ALOX15 targeting siRNA antisense |
| 5362 | Non-targeting control siRNA sense |
| 5363 | Non-targeting control siRNA antisense |
| 5364 | ALOX15 targeting siRNA sense |
| 5365 | ALOX15 targeting siRNA antisense |

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A pharmaceutical composition comprising an siRNA molecule comprising a sense strand and an antisense strand, which targets SEQ ID NO: 5357 and when administered to a patient in an effective amount reduces eosinophil count, wherein (i) the siRNA comprises a modification comprising a modified nucleoside and/or a modified internucleoside linkage, and/or (ii) the pharmaceutical composition comprises a pharmaceutically acceptable carrier; and wherein the patient comprises nasal polyposis, chronic sinusitis, allergic rhinitis, or NSAID-exacerbated respiratory disease, or a combination thereof.

2. The pharmaceutical composition of claim 1, wherein the systemic or local eosinophil count is reduced by about 10% or more as compared to the eosinophil count prior to administration.

3. A pharmaceutical composition comprising an siRNA molecule which targets SEQ ID NO: 5357 and when administered in an effective amount to a patient comprising nasal polyps, the nasal polyps are reduced in number and/or size the patient, wherein (i) the siRNA comprises a modification comprising a modified nucleoside and/or a modified internucleoside linkage, and/or (ii) the pharmaceutical composition comprises a pharmaceutically acceptable carrier.

4. The pharmaceutical composition of claim 3, wherein the nasal polyps are reduced in number and/or size by about 10% or more by CT scan or endoscopic assessment, as compared to the number and/or size prior to administration.

5. A pharmaceutical composition comprising an siRNA molecule which targets SEQ ID NO: 5357 and when administered to a patient in an effective amount increases nasal inspiratory peak flow in the patient, wherein (i) the siRNA comprises a modification comprising a modified nucleoside and/or a modified internucleoside linkage, and/or (ii) the pharmaceutical composition comprises a pharmaceutically acceptable carrier.

6. The pharmaceutical composition of claim 5, wherein the nasal inspiratory peak flow is increased by about 10% or more, as compared to prior to administration.

7. A pharmaceutical composition comprising an siRNA molecule which targets SEQ ID NO: 5357 and when administered to a patient in an effective amount reduces airway symptoms in the patient, wherein the patient comprises nasal polyposis, chronic sinusitis, allergic rhinitis, or NSAID-exacerbated respiratory disease, or a combination thereof, and wherein (i) the siRNA comprises a modification comprising a modified nucleoside and/or a modified internucleoside linkage, and/or (ii) the pharmaceutical composition comprises a pharmaceutically acceptable carrier.

8. The pharmaceutical composition of claim 7, wherein the airway inflammation symptoms are reduced by about 10% or more on a patient-reported outcome measure, as compared to prior to administration.

9. A pharmaceutical composition comprising an siRNA molecule which targets SEQ ID NO: 5357 and when administered to a patient in an effective amount improves sense of smell in the patient, wherein the patient comprises nasal polyposis, chronic sinusitis, allergic rhinitis, or NSAID-exacerbated respiratory disease, or a combination thereof, and wherein (i) the siRNA comprises a modification comprising a modified nucleoside and/or a modified internucleoside linkage, and/or (ii) the pharmaceutical composition comprises a pharmaceutically acceptable carrier.

10. The pharmaceutical composition of claim 9, wherein the sense of smell is improved by about 10% or more on a patient-reported outcome measure, as compared to prior to administration.

11. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6000 (C AGAGCTGCAG ACCTGGTGTC GAGAGATCAC TGAAATCGGG CTGCAAGGGG CCCAGGACCG AGGGTTTCCT GTCTCTTTAC AGGCTCGGGA CCAGGTTTGC CACTTTGTCA CCATGTGTAT CTTCACCTGC ACCGGCCAAC ACGCCTCTGT); wherein (i) the siRNA comprises a modification comprising a modified nucleoside and/or a modified internucleoside linkage, and/or (ii) the pharmaceutical composition comprises a pharmaceutically acceptable carrier.

12. The pharmaceutical composition of claim 11, wherein the sense strand comprises about 14-30 contiguous nucleosides of SEQ ID NO: 6001 (C AGAGCTGCAG ACCTGGTGTC GAGAGATCAC TGAAATCGGG CTGCAAGGGG) or SEQ ID NO: 6002 (CTTTAC AGGCTCGGGA CCAGGTTTGC CACTTTGTCA CCATGTGTAT CTTCA).

13. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6003 (A CCCTCTTCCC ATGTCCCACC CTCCCTAGAG GGGCACCTTT TCATGGTCTC TGCACCCAGT GAACACATTT TACTCTAGAG GCATCACCTG GGACCTTACT); wherein (i) the siRNA comprises a modification comprising a modified nucleoside and/or a modified internucleoside linkage, and/or (ii) the pharmaceutical composition comprises a pharmaceutically acceptable carrier.

14. The pharmaceutical composition of claim 13, wherein the sense strand comprises about 14-30 contiguous nucleosides of SEQ ID NO: 6004 (TAGAG GGGCACCTTT TCATGGTCTC TGCACCCAGT GAACACATTT TACTC).

15. A pharmaceutical composition comprising an siRNA molecule that inhibits the expression of ALOX15, wherein the siRNA comprises a sense strand and an antisense strand, each strand is independently about 14-30 nucleosides in length, and at least one of the sense strand and the antisense strand comprises a nucleoside sequence comprising about 14-30 contiguous nucleosides of SEQ ID NO: 6005 (G ACATGGGAAT TTTCGACCAG ATAATGAGCA CTGGTGGGGG AGGCCACGTG CAGCTGCTCA AGCAAGCTGG AGCCTTCCTA ACCTACAGCT CCTTCTGTCC CCCTGATGAC TTGGCCGACC GGGGGCTCCT GGGAGTGAAG TCTTCCTTCT); wherein (i) the siRNA comprises a modification comprising a modified nucleoside and/or a modified internucleoside linkage, and/or (ii) the pharmaceutical composition comprises a pharmaceutically acceptable carrier.

16. The pharmaceutical composition of claim 15, wherein the sense strand comprises about 14-30 contiguous nucleosides of SEQ ID NO: 6006 (G CAGCTGCTCA AGCAAGCTGG AGCCTTCCTA ACCTACAGCT CCTTCTGTCC).

17. The pharmaceutical composition of any one of claims 1-16, comprising the modified internucleoside linkage.

18. The pharmaceutical composition of claim 17, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

19. The pharmaceutical composition of claim 18, wherein the modified internucleoside linkage comprises one or more phosphorothioate linkages.

20. The pharmaceutical composition of any one of claims 1-19, comprising the modified nucleoside.

21. The pharmaceutical composition of claim 20, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

22. The pharmaceutical composition of claim 20 or claim 21, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

23. The pharmaceutical composition of any one of claims 20-22, wherein the modified nucleoside comprises one or more 2′fluoro modified nucleosides.

24. The pharmaceutical composition of any one of claims 20-23, wherein the modified nucleoside comprises a 2′ O-alkyl modified nucleoside.

25. The pharmaceutical composition of any one of claims 1-24, wherein the siRNA comprises a ribose.

26. The pharmaceutical composition of any one of claims 1-25, further comprising a lipid attached at either 3′ or 5′ terminus of the sense strand and/or antisense strand of the siRNA.

27. The pharmaceutical composition of claim 26, wherein the lipid comprises cholesterol, myristoyl, palmitoyl, stearoyl, lithocholoyl, docosanoyl, docosahexaenoyl, myristyl, palmityl stearyl, or a-tocopherol, or a combination thereof.

28. The pharmaceutical composition of any one of claims 1-27, wherein the sense strand and the antisense strand form a double-stranded RNA duplex.

29. The pharmaceutical composition of claim 28, wherein the double-stranded RNA duplex comprises from about 14 to about 30 nucleosides.

30. The pharmaceutical composition of claim 28 or claim 29, wherein the first base pair of the double-stranded RNA duplex is an AU base pair.

31. The pharmaceutical composition of any one of claims 1-30, wherein the sense strand comprises pattern 1S: 5′ fN s mN s fN-mN-fN-mN-fN-fN-fN-mN-fN-mN-fN-mN-fN-mN-fN-mN-fN s mN s mN 3′, wherein “fN” is a 2′ fluoro-modified nucleoside, “mN” is a 2′ O-methyl modified nucleoside, “-” is a phosphodiester, and “s” is a phosphorothioate.

32. The pharmaceutical composition of any one of claims 1-30, wherein the sense strand comprises pattern 2S: 5′ mN s mN s mN-mN-fN-mN-fN-fN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

33. The pharmaceutical composition of any one of claims 1-30, wherein the sense strand comprises pattern 3S: 5′ mN s mN s mN-mN-fN-mN-fN-mN-fN-mN-mN-mN-mN-mN-mN-mN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

34. The pharmaceutical composition of any one of claims 1-30, wherein the sense strand comprises pattern 4S: 5′ fN s mN s fN-mN-fN-mN-fN-fN-fN-mN-fN-mN-fN-mN-fN-mN-fN-mN-fN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides.

35. The pharmaceutical composition of any one of claims 1-30, wherein the sense strand comprises pattern 5S: 5′ mN s mN s mN-mN-fN-mN-fN-fN-fN-mN-mN-mN-mN-mN-mN-mN-mN-mN-mN s mN s mN-N-Lipid 3′, wherein “fN” is a 2′ fluoro-modified nucleoside, “mN” is a 2′ O-methyl modified nucleoside, “-” is a phosphodiester, “s” is a phosphorothioate, and N comprises one or more nucleosides.

36. The pharmaceutical composition of any one of claims 1-35, wherein the antisense strand comprises pattern 1AS: 5′ mN s fN s mN-fN-mN-fN-mN-fN-mN-fN-mN-mN-mN-fN-mN-fN-mN-fN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

37. The pharmaceutical composition of any one of claims 1-35, wherein the antisense strand comprises pattern 2AS: 5′ mN s fN s mN-mN-mN-fN-mN-fN-fN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

38. The pharmaceutical composition of any one of claims 1-35, wherein the antisense strand comprises pattern 3AS: 5′ mN s fN s mN-mN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

39. The pharmaceutical composition of any one of claims 1-35, wherein the antisense strand comprises pattern 4AS: 5′ mN s fN s mN-fN-mN-fN-mN-mN-mN-mN-mN-mN-mN-fN-mN-fN-mN-mN-mN s mN s mN 3′, wherein “fN” is a 2′ fluoro-modified nucleoside, “mN” is a 2′ O-methyl modified nucleoside, “-” is a phosphodiester, and “s” is a phosphorothioate.

40. The pharmaceutical composition of any one of claims 1-30, wherein the sense strand comprises pattern 1S and the antisense strand comprises pattern 1AS, 2AS, 3AS, or 4AS.

41. The pharmaceutical composition of any one of claims 1-30, wherein the sense strand comprises pattern 2S and the antisense strand comprises pattern 1AS, 2AS, 3AS, or 4AS.

42. The pharmaceutical composition of any one of claims 1-30, wherein the sense strand comprises pattern 3S and the antisense strand comprises pattern 1AS, 2AS, 3AS, or 4AS.

43. The pharmaceutical composition of any one of claims 1-30, wherein the sense strand comprises pattern 4S and the antisense strand comprises pattern 1AS, 2AS, 3AS, or 4AS.

44. The pharmaceutical composition of any one of claims 1-29, wherein
(i) the sense strand comprises SEQ ID NO: 3027 or a nucleic acid sequence thereof having about 1 or 2 nucleoside substitutions, additions, or deletions, and the antisense strand comprises SEQ ID NO: 3028 or a nucleic acid sequence thereof having about 1 or 2 nucleoside substitutions, additions, or deletions;
(ii) the sense strand comprises SEQ ID NO: 5664 or a nucleic acid sequence thereof having about 1 or 2 nucleoside substitutions, additions, or deletions, and the antisense strand comprises SEQ ID NO: 5765 or a nucleic acid sequence thereof having about 1 or 2 nucleoside substitutions, additions, or deletions;
(iii) the sense strand comprises SEQ ID NO: 5866 or a nucleic acid sequence thereof having about 1 or 2 nucleoside substitutions, additions, or deletions, and the antisense strand comprises SEQ ID NO: 5967 or a nucleic acid sequence thereof having about 1 or 2 nucleoside substitutions, additions, or deletions;
(iv) the sense strand comprises SEQ ID NO: 5558 or a nucleic acid sequence thereof having about 1 or 2 nucleoside substitutions, additions, or deletions, and the antisense strand comprises SEQ ID NO: 5559 or a nucleic acid sequence thereof having about 1 or 2 nucleoside substitutions, additions, or deletions; or
(v) the sense strand comprises SEQ ID NO: 5456 or a nucleic acid sequence thereof having about 1 or 2 nucleoside substitutions, additions, or deletions, and the antisense strand comprises SEQ ID NO: 5457 or a nucleic acid sequence thereof having about 1 or 2 nucleoside substitutions, additions, or deletions.

45. The pharmaceutical composition of any one of claims 1-29, wherein
(i) the sense strand comprises SEQ ID NO: 3037 or a nucleic acid sequence thereof having about 1 or 2 nucleoside substitutions, additions, or deletions, and the antisense strand comprises SEQ ID NO: 3038 or a nucleic acid sequence thereof having about 1 or 2 nucleoside substitutions, additions, or deletions;
(ii) the sense strand comprises SEQ ID NO: 5665 or a nucleic acid sequence thereof having about 1 or 2 nucleoside substitutions, additions, or deletions, and the antisense strand comprises SEQ ID NO: 5766 or a nucleic acid sequence thereof having about 1 or 2 nucleoside substitutions, additions, or deletions;
(iii) the sense strand comprises SEQ ID NO: 5867 or a nucleic acid sequence thereof having about 1 or 2 nucleoside substitutions, additions, or deletions, and the antisense strand comprises SEQ ID NO: 5968 or a nucleic acid sequence thereof having about 1 or 2 nucleoside substitutions, additions, or deletions;
(iv) the sense strand comprises SEQ ID NO: 5560 or a nucleic acid sequence thereof having about 1 or 2 nucleoside substitutions, additions, or deletions, and the antisense strand comprises SEQ ID NO: 5561 or a nucleic acid sequence thereof having about 1 or 2 nucleoside substitutions, additions, or deletions; or
(v) the sense strand comprises SEQ ID NO: 5460 or a nucleic acid sequence thereof having about 1 or 2 nucleoside substitutions, additions, or deletions, and the antisense strand comprises SEQ ID NO: 5461 or a nucleic acid sequence thereof having about 1 or 2 nucleoside substitutions, additions, or deletions.

46. The pharmaceutical composition of any one of claims 1-29, wherein
(i) the sense strand comprises SEQ ID NO: 3183 or a nucleic acid sequence thereof having about 1 or 2 nucleoside substitutions, additions, or deletions, and the antisense strand comprises SEQ ID NO: 3184 or a nucleic acid sequence thereof having about 1 or 2 nucleoside substitutions, additions, or deletions;
(ii) the sense strand comprises SEQ ID NO: 5666 or a nucleic acid sequence thereof having about 1 or 2 nucleoside substitutions, additions, or deletions, and the antisense strand comprises SEQ ID NO: 5767 or a nucleic acid sequence thereof having about 1 or 2 nucleoside substitutions, additions, or deletions;
(iii) the sense strand comprises SEQ ID NO: 5868 or a nucleic acid sequence thereof having about 1 or 2 nucleoside substitutions, additions, or deletions, and the antisense strand comprises SEQ ID NO: 5969 or a nucleic acid sequence thereof having about 1 or 2 nucleoside substitutions, additions, or deletions;
(iv) or the sense strand comprises SEQ ID NO: 5562 or a nucleic acid sequence thereof having about 1 or 2 nucleoside substitutions, additions, or deletions, and the antisense strand comprises SEQ ID NO: 5563 or a nucleic acid sequence thereof having about 1 or 2 nucleoside substitutions, additions, or deletions; or
(v) the sense strand comprises SEQ ID NO: 5476 or a nucleic acid sequence thereof having about 1 or 2 nucleoside substitutions, additions, or deletions, and the antisense strand comprises SEQ ID NO: 5477 or a nucleic acid sequence thereof having about 1 or 2 nucleoside substitutions, additions, or deletions.

47. The pharmaceutical composition of any one of claims 1-29, wherein
(i) the sense strand comprises SEQ ID NO: 4265 or a nucleic acid sequence thereof having about 1 or 2 nucleoside substitutions, additions, or deletions, and the antisense strand comprises SEQ ID NO: 4266 or a nucleic acid sequence thereof having about 1 or 2 nucleoside substitutions, additions, or deletions;
(ii) the sense strand comprises SEQ ID NO: 5667 or a nucleic acid sequence thereof having about 1 or 2 nucleoside substitutions, additions, or deletions, and the antisense strand comprises SEQ ID NO: 5768 or a nucleic acid sequence thereof having about 1 or 2 nucleoside substitutions, additions, or deletions;
(iii) the sense strand comprises SEQ ID NO: 5869 or a nucleic acid sequence thereof having about 1 or 2 nucleoside substitutions, additions, or deletions, and the antisense strand comprises SEQ ID NO: 5970 or a nucleic acid sequence thereof having about 1 or 2 nucleoside substitutions, additions, or deletions;
(iv) the sense strand comprises SEQ ID NO: 5564 or a nucleic acid sequence thereof having about 1 or 2 nucleoside substitutions, additions, or deletions, and the antisense strand comprises SEQ ID NO: 5565 or a nucleic acid sequence thereof having about 1 or 2 nucleoside substitutions, additions, or deletions; or
(v) the sense strand comprises SEQ ID NO: 5510 or a nucleic acid sequence thereof having about 1 or 2 nucleoside substitutions, additions, or deletions, and the antisense strand comprises SEQ ID NO: 5511 or a nucleic acid sequence thereof having about 1 or 2 nucleoside substitutions, additions, or deletions.

48. The pharmaceutical composition of any one of claims 1-29, wherein
(i) the sense strand comprises SEQ ID NO: 2629 or a nucleic acid sequence thereof having about 1 or 2 nucleoside substitutions, additions, or deletions, and the antisense strand comprises SEQ ID NO: 2630 or a nucleic acid sequence thereof having about 1 or 2 nucleoside substitutions, additions, or deletions;
(ii) the sense strand comprises SEQ ID NO: 5668 or a nucleic acid sequence thereof having about 1 or 2 nucleoside substitutions, additions, or deletions, and the antisense strand comprises SEQ ID NO: 5769 or a nucleic acid sequence thereof having about 1 or 2 nucleoside substitutions, additions, or deletions;
(iii) the sense strand comprises SEQ ID NO: 5870 or a nucleic acid sequence thereof having about 1 or 2 nucleoside substitutions, additions, or deletions, and the antisense strand comprises SEQ ID NO: 5971 or a nucleic acid sequence thereof having about 1 or 2 nucleoside substitutions, additions, or deletions;
(iv) the sense strand comprises SEQ ID NO: 5566 or a nucleic acid sequence thereof having about 1 or 2 nucleoside substitutions, additions, or deletions, and the antisense strand comprises SEQ ID NO: 5567 or a nucleic acid sequence thereof having about 1 or 2 nucleoside substitutions, additions, or deletions; or
(v) the sense strand comprises SEQ ID NO: 5524 or a nucleic acid sequence thereof having about 1 or 2 nucleoside substitutions, additions, or deletions, and the antisense strand comprises SEQ ID NO: 5525 or a nucleic acid sequence thereof having about 1 or 2 nucleoside substitutions, additions, or deletions.

49. The pharmaceutical composition of any one of claims 1-29, wherein the sense strand comprises mUsmGsmGmUfGmUfCfGfAmGmAmGmAmUmCmAmCmUmAsmUsmU (SEQ ID NO: 5558), mUsmCsmGmAfGmAfGfAfUmCmAmCmUmGmAmAmAmUmAsmUsmU (SEQ ID NO: 5560), mCsmAsmGmGfUmUfUfGfCmCmAmCmUmUmUmGmUmCmAsmUsmU (SEQ ID NO: 5562), mGsmCsmAmCfCmUfUfUfUmCmAmUmGmGmUmCmUmCmAsmUsmU (SEQ ID NO: 5564), mAsmGsmCmUfGmGfAfGfCmCmUmUmCmCmUmAmAmCmAsmUsmU (SEQ ID NO: 5566), fUsmGsfGmUfGmUfCfGfAmGfAmGfAmUfCmAfCmUfAsmUsmU (SEQ ID NO: 5456), fUsmCsfGmAfGmAfGfAfUmCfAmCfUmGfAmAfAmUfAsmUsmU (SEQ ID NO: 5460), fCsmAsfGmGfUmUfUfGfCmCfAmCfUmUfUmGfUmCfAsmUsmU (SEQ ID NO: 5476), fGsmCsfAmCfCmUfUfUfUmCfAmUfGmGfUmCfUmCfAsmUsmU (SEQ ID NO: 5510), fAsmGsfCmUfGmGfAfGfCmCfUmUfCmCfUmAfAmCfAsmUsmU (SEQ ID NO: 5524), mUsmGsmGmUfGmUfCmGfAmGmAmGmAmUmCmAmCmUmAsmUsmU (SEQ ID NO: 6050), mUsmCsmGmAfGmAfGmAfUmCmAmCmUmGmAmAmAmUmAsmUsmU (SEQ ID NO: 6051), mCsmAsmGmGfUmUfUmGfCmCmAmCmUmUmUmGmUmCmAsmUsmU (SEQ ID NO: 6052), mGsmCsmAmCfCmUfUmUfUmCmAmUmGmGmUmCmUmCmAsmUsmU (SEQ ID NO: 6053), mAsmGsmCmUfGmGfAmGfCmCmUmUmCmCmUmAmAmCmAsmUsmU (SEQ ID NO: 6054), fUsmGsfGmUfGmUfCfGfAmGfAmGfAmUfCmAfCmUfAsmUsmUNNN-lipid (SEQ ID NO: 6055), fUsmCsfGmAfGmAfGfAfUmCfAmCfUmGfAmAfAmUfAsmUsmUNNN-lipid (SEQ ID NO: 6056), fCsmAsfGmGfUmUfUfGfCmCfAmCfUmUfUmGfUmCfAsmUsmUNNN-lipid (SEQ ID NO: 6057), fGsmCsfAmCfCmUfUfUfUmCfAmUfGmGfUmCfUmCfAsmUsmUNNN-lipid (SEQ ID NO: 6058), fAsmGsfCmUfGmGfAfGfCmCfUmUfCmCfUmAfAmCfAsmUsmUNNN-lipid (SEQ ID NO: 6059), mUsmGsmGmUfGmUfCfGfAmGmAmGmAmUmCmAmCmUmAsmUsmUNNN-lipid (SEQ ID NO: 6060), mUsmCsmGmAfGmAfGfAfUmCmAmCmUmGmAmAmAmUmAsmUsmUNNN-lipid (SEQ ID NO: 6061), mCsmAsmGmGfUmUfUfGfCmCmAmCmUmUmUmGmUmCmAsmUsmUNNN-lipid (SEQ ID NO: 6062), mGsmCsmAmCfCmUfUfUfUmCmAmUmGmGmUmCmUmCmAsmUsmUNNN-lipid (SEQ ID NO: 6063), or mAsmGsmCmUfGmGfAfGfCmCmUmUmCmCmUmAmAmCmAsmUsmUNNN-lipid (SEQ ID NO: 6064), or any combination thereof.

50. The pharmaceutical composition of any one of claims 1-30, wherein the antisense strand comprises mUsfAsmGmUmGfAmUmCmUmCmUmCmGfAmCfAmCmCmAsmUsmU (SEQ ID NO: 5559), mUsfAsmUmUmUfCmAmGmUmGmAmUmCfUmCfUmCmGmAsmUsmU (SEQ ID NO: 5561), mUsfGsmAmCmAfAmAmGmUmGmGmCmAfAmAfCmCmUmGsmUsmU (SEQ ID NO: 5563), mUsfGsmAmGmAfCmCmAmUmGmAmAmAfAmGfGmUmGmCsmUsmU (SEQ ID NO: 5565), mUsfGsmUmUmAfGmGmAmAmGmGmCmUfCmCfAmGmCmUsmUsmU (SEQ ID NO: 5567), mUsfAsmGfUmGfAmUfCmUfCmUmCmGfAmCfAmCfCmAsmUsmU (SEQ ID NO: 5457), mUsfAsmUfUmUfCmAfGmUfGmAmUmCfUmCfUmCfGmAsmUsmU (SEQ ID NO: 5461), mUsfGsmAfCmAfAmAfGmUfGmGmCmAfAmAfCmCfUmGsmUsmU (SEQ ID NO: 5477), mUsfGsmAfGmAfCmCfAmUfGmAmAmAfAmGfGmUfGmCsmUsmU (SEQ ID NO: 5511), mUsfGsmUfUmAfGmGfAmAfGmGmCmUfCmCfAmGfCmUsmUsmU (SEQ ID NO: 5525), mUsfAsmGmUmGfAmUfCfUmCmUmCmGfAmCfAmCmCmAsmUsmU (SEQ ID NO: 6065), mUsfAsmUmUmUfCmAfGfUmGmAmUmCfUmCfUmCmGmAsmUsmU (SEQ ID NO: 6066), mUsfGsmAmCmAfAmAfGfUmGmGmCmAfAmAfCmCmUmGsmUsmU (SEQ ID NO: 6067), mUsfGsmAmGmAfCmCfAfUmGmAmAmAfAmGfGmUmGmCsmUsmU (SEQ ID NO: 6068), mUsfGsmUmUmAfGmGfAfAmGmGmCmUfCmCfAmGmCmUsmUsmU (SEQ ID NO: 6069), mUsfAsmGfUmGfAmUmCmUmCmUmCmGfAmCfAmCmCmAsmUsmU (SEQ ID NO: 6070), mUsfAsmUfUmUfCmAmGmUmGmAmUmCfUmCfUmCmGmAsmUsmU (SEQ ID NO: 6071), mUsfGsmAfCmAfAmAmGmUmGmGmCmAfAmAfCmCmUmGsmUsmU (SEQ ID NO: 6072), mUsfGsmAfGmAfCmCmAmUmGmAmAmAfAmGfGmUmGmCsmUsmU (SEQ ID NO: 6073), or mUsfGsmUfUmAfGmGmAmAmGmGmCmUfCmCfAmGmCmUsmUsmU (SEQ ID NO: 6074), or any combination thereof.

51. The pharmaceutical composition of any one of claims 1-29, wherein the sense strand or antisense strand comprises one or more sequences selected from SEQ ID NOS: 1-5349, 5366-5971, 6000-6038, 6050-6074, or a nucleic acid sequence thereof having about 1 or 2 nucleoside substitutions, additions, or deletions.

52. The pharmaceutical composition of any one of claims 1-29, wherein the sense strand or antisense strand comprises one or more sequences comprising the first 19 nucleobases of a sequence selected from SEQ ID NOS: 1-5349, 5366-5971, 6000-6038, 6050-6074, or a nucleic acid sequence thereof having about 1 or 2 nucleoside substitutions, additions, or deletions.

53. A pharmaceutical composition comprising a nucleic acid sequence comprising Formula IA: 5′ Z1-U-Z2-G-Z3-U-C-Z4-A-U-U-Z5-L 3′
wherein
Z1 is 0, 3, 5, 11, or 13 nucleosides;
Z2 is 1 nucleoside;
Z3 is 0, 2, 5, 8, or 10 nucleosides;
Z4 is 0, 2, 3, 5, 8, or 11 nucleosides;
Z5 is 0 or 3 nucleosides;
each “-” is independently a phosphodiester or modified internucleoside linkage; and
L is an optional lipid;
wherein the nucleic acid comprises a modified nucleoside and/or modified internucleoside linkage.

54. The pharmaceutical composition of claim 53, wherein Z1 comprises 0 nucleosides or UGGUG, CAGGU, CAGGUUUGCCACU (SEQ ID NO: 6007), GCACCUUUUCA (SEQ ID NO: 6008), or AGC; Z2 comprises C, G, or U; Z3 comprises 0 nucleosides or UG, UGUCGAGAGA (SEQ ID NO: 6009), AGAGA, AGAGA, CCACUUUG, or AGCCU; Z4 comprises 0 nucleosides or GAGAGAUCACU (SEQ ID NO: 6010), ACU, ACUGAAAU, UC, or CUAAC; and Z5 comprises 0 nucleosides or UUU, UUC, UUA, UUG, CUU, CUC, CUA, CUG, AUU, AUC, AUA, AUG, GUU, GUC, GUA, GUG, UCU, UCC, UCA, UCG, CCU, CCC, CCA, CCG, ACU, ACC, ACA, ACG, GCU, GCC, GCA, GCG, UAU, UAC, UAA, UAG, CAU, CAC, CAA, CAG, AAU, AAC, AAA, AAG, GAU, GAC, GAA, GAG, UGU, UGC, UGA, UGG, CGU, CGC, CGA, CGG, AGU, AGC, AGA, AGG, GGU, GGC, GGA, or GGG.

55. A pharmaceutical composition comprising a nucleic acid sequence comprising Formula IB: 5′ U-Z6-G-A-Z7-C-Z8-A-Z9-U-U 3′
wherein
Z6 represents 0, 2, 3, 5, 8, or 11 nucleosides;
Z7 represents 0, 2, 5, 8, or 10 nucleosides;
Z8 represents 1 nucleoside;
Z9 represents 0, 3, 5, 11, or 13 nucleosides; and
each “-” is independently a phosphodiester or modified internucleoside linkage.

56. The pharmaceutical composition of claim 53 or claim 54, wherein the nucleic acid sequence comprising Formula IA is a sense strand, and the pharmaceutical composition further comprises an anti-sense strand comprising Formula IB: 5′ U-Z6-G-A-Z7-C-Z8-A-Z9-U-U 3′
wherein
Z6 represents 0, 2, 3, 5, 8, or 11 nucleosides;
Z7 represents 0, 2, 5, 8, or 10 nucleosides;
Z8 represents 1 nucleoside;
Z9 represents 0, 3, 5, 11, or 13 nucleosides; and
each “-” is independently a phosphodiester or modified internucleoside linkage.

57. The pharmaceutical composition of claim 55 or claim 56, wherein Z6 is 0 nucleosides or AGU, AGUGAUCUCUC (SEQ ID NO: 6016), AUUUCAGU, GA, or GUUAG; Z7 is 0 nucleosides or UCUCU, CA, UCUCU, CAAAGUGG, GA, or AGGCU; Z8 is C, G, or A; and Z9 is CACCA, AGUGGCAAACCUG (SEQ ID NO: 6017), ACCUG, UGAAAAGGUGC (SESQ ID NO: 6018), or GCU.

58. A pharmaceutical composition comprising a sense strand and an antisense strand, wherein
(i) the sense strand comprises SEQ ID NO: 3027 or a nucleic acid sequence thereof having about 1 or 2 nucleoside substitutions, additions, or deletions, and the antisense strand comprises SEQ ID NO: 3028 or a nucleic acid sequence thereof having about 1 or 2 nucleoside substitutions, additions, or deletions;
(ii) the sense strand comprises SEQ ID NO: 5664 or a nucleic acid sequence thereof having about 1 or 2 nucleoside substitutions, additions, or deletions, and the antisense strand comprises SEQ ID NO: 5765 or a nucleic acid sequence thereof having about 1 or 2 nucleoside substitutions, additions, or deletions;
(iii) the sense strand comprises SEQ ID NO: 5866 or a nucleic acid sequence thereof having about 1 or 2 nucleoside substitutions, additions, or deletions, and the antisense strand comprises SEQ ID NO: 5967 or a nucleic acid sequence thereof having about 1 or 2 nucleoside substitutions, additions, or deletions;
(iv) the sense strand comprises SEQ ID NO: 5558 or a nucleic acid sequence thereof having about 1 or 2 nucleoside substitutions, additions, or deletions, and the antisense strand comprises SEQ ID NO: 5559 or a nucleic acid sequence thereof having about 1 or 2 nucleoside substitutions, additions, or deletions; or
(v) the sense strand comprises SEQ ID NO: 5456 or a nucleic acid sequence thereof having about 1 or 2 nucleoside substitutions, additions, or deletions, and the antisense strand comprises SEQ ID NO: 5457 or a nucleic acid sequence thereof having about 1 or 2 nucleoside substitutions, additions, or deletions.

59. A pharmaceutical composition comprising a sense strand and an antisense strand, wherein
(i) the sense strand comprises SEQ ID NO: 3037 or a nucleic acid sequence thereof having about 1 or 2 nucleoside substitutions, additions, or deletions, and the antisense strand comprises SEQ ID NO: 3038 or a nucleic acid sequence thereof having about 1 or 2 nucleoside substitutions, additions, or deletions;
(ii) the sense strand comprises SEQ ID NO: 5665 or a nucleic acid sequence thereof having about 1 or 2 nucleoside substitutions, additions, or deletions, and the antisense strand comprises SEQ ID NO: 5766 or a nucleic acid sequence thereof having about 1 or 2 nucleoside substitutions, additions, or deletions;
(iii) the sense strand comprises SEQ ID NO: 5867 or a nucleic acid sequence thereof having about 1 or 2 nucleoside substitutions, additions, or deletions, and the antisense strand comprises SEQ ID NO: 5968 or a nucleic acid sequence thereof having about 1 or 2 nucleoside substitutions, additions, or deletions;
(iv) the sense strand comprises SEQ ID NO: 5560 or a nucleic acid sequence thereof having about 1 or 2 nucleoside substitutions, additions, or deletions, and the antisense strand comprises SEQ ID NO: 5561 or a nucleic acid sequence thereof having about 1 or 2 nucleoside substitutions, additions, or deletions; or
(v) the sense strand comprises SEQ ID NO: 5460 or a nucleic acid sequence thereof having about 1 or 2 nucleoside substitutions, additions, or deletions, and the antisense strand comprises SEQ ID NO: 5461 or a nucleic acid sequence thereof having about 1 or 2 nucleoside substitutions, additions, or deletions.

60. A pharmaceutical composition comprising a sense strand and an antisense strand, wherein
(i) the sense strand comprises SEQ ID NO: 3183 or a nucleic acid sequence thereof having about 1 or 2 nucleoside substitutions, additions, or deletions, and the antisense strand comprises SEQ ID NO: 3184 or a nucleic acid sequence thereof having about 1 or 2 nucleoside substitutions, additions, or deletions;
(ii) the sense strand comprises SEQ ID NO: 5666 or a nucleic acid sequence thereof having about 1 or 2 nucleoside substitutions, additions, or deletions, and the antisense strand comprises SEQ ID NO: 5767 or a nucleic acid sequence thereof having about 1 or 2 nucleoside substitutions, additions, or deletions;
(iii) the sense strand comprises SEQ ID NO: 5868 or a nucleic acid sequence thereof having about 1 or 2 nucleoside substitutions, additions, or deletions, and the antisense strand comprises SEQ ID NO: 5969 or a nucleic acid sequence thereof having about 1 or 2 nucleoside substitutions, additions, or deletions;
(iv) or the sense strand comprises SEQ ID NO: 5562 or a nucleic acid sequence thereof having about 1 or 2 nucleoside substitutions, additions, or deletions, and the antisense strand comprises SEQ ID NO: 5563 or a nucleic acid sequence thereof having about 1 or 2 nucleoside substitutions, additions, or deletions; or
(v) the sense strand comprises SEQ ID NO: 5476 or a nucleic acid sequence thereof having about 1 or 2 nucleoside substitutions, additions, or deletions, and the antisense strand comprises SEQ ID NO: 5477 or a nucleic acid sequence thereof having about 1 or 2 nucleoside substitutions, additions, or deletions.

61. A pharmaceutical composition comprising a sense strand and an antisense strand, wherein
(i) the sense strand comprises SEQ ID NO: 4265 or a nucleic acid sequence thereof having about 1 or 2 nucleoside substitutions, additions, or deletions, and the antisense strand comprises SEQ ID NO: 4266 or a nucleic acid sequence thereof having about 1 or 2 nucleoside substitutions, additions, or deletions;
(ii) the sense strand comprises SEQ ID NO: 5667 or a nucleic acid sequence thereof having about 1 or 2 nucleoside substitutions, additions, or deletions, and the antisense strand comprises SEQ ID NO: 5768 or a nucleic acid sequence thereof having about 1 or 2 nucleoside substitutions, additions, or deletions;
(iii) the sense strand comprises SEQ ID NO: 5869 or a nucleic acid sequence thereof having about 1 or 2 nucleoside substitutions, additions, or deletions, and the antisense strand comprises SEQ ID NO: 5970 or a nucleic acid sequence thereof having about 1 or 2 nucleoside substitutions, additions, or deletions;
(iv) the sense strand comprises SEQ ID NO: 5564 or a nucleic acid sequence thereof having about 1 or 2 nucleoside substitutions, additions, or deletions, and the antisense strand comprises SEQ ID NO: 5565 or a nucleic acid sequence thereof having about 1 or 2 nucleoside substitutions, additions, or deletions; or
(v) the sense strand comprises SEQ ID NO: 5510 or a nucleic acid sequence thereof having about 1 or 2 nucleoside substitutions, additions, or deletions, and the antisense strand comprises SEQ ID NO: 5511 or a nucleic acid sequence thereof having about 1 or 2 nucleoside substitutions, additions, or deletions.

62. A pharmaceutical composition comprising a sense strand and an antisense strand, wherein
(i) the sense strand comprises SEQ ID NO: 2629 or a nucleic acid sequence thereof having about 1 or 2 nucleoside substitutions, additions, or deletions, and the antisense strand comprises SEQ ID NO: 2630 or a nucleic acid sequence thereof having about 1 or 2 nucleoside substitutions, additions, or deletions;
(ii) the sense strand comprises SEQ ID NO: 5668 or a nucleic acid sequence thereof having about 1 or 2 nucleoside substitutions, additions, or deletions, and the antisense strand comprises SEQ ID NO: 5769 or a nucleic acid sequence thereof having about 1 or 2 nucleoside substitutions, additions, or deletions;
(iii) the sense strand comprises SEQ ID NO: 5870 or a nucleic acid sequence thereof having about 1 or 2 nucleoside substitutions, additions, or deletions, and the antisense strand comprises SEQ ID NO: 5971 or a nucleic acid sequence thereof having about 1 or 2 nucleoside substitutions, additions, or deletions;
(iv) the sense strand comprises SEQ ID NO: 5566 or a nucleic acid sequence thereof having about 1 or 2 nucleoside substitutions, additions, or deletions, and the antisense strand comprises SEQ ID NO: 5567 or a nucleic acid sequence thereof having about 1 or 2 nucleoside substitutions, additions, or deletions; or
(v) the sense strand comprises SEQ ID NO: 5524 or a nucleic acid sequence thereof having about 1 or 2 nucleoside substitutions, additions, or deletions, and the antisense strand comprises SEQ ID NO: 5525 or a nucleic acid sequence thereof having about 1 or 2 nucleoside substitutions, additions, or deletions.

63. A pharmaceutical composition comprising a sense strand and an antisense strand, wherein the sense strand or antisense strand comprises one or more sequences selected from SEQ ID NOS: 1-5349, 5366-5971, 6000-6038, 6050-6074; or a nucleic acid sequence thereof having about 1 or 2 nucleoside substitutions, additions, or deletions.

64. The pharmaceutical composition of claim 63, wherein the sense strand comprises SEQ ID NO: 5866.

65. The pharmaceutical composition of claim 63, wherein the sense strand comprises SEQ ID NO: 5867.

66. The pharmaceutical composition of claim 63, wherein the sense strand comprises SEQ ID NO: 5868.

67. The pharmaceutical composition of claim 63, wherein the sense strand comprises SEQ ID NO: 5869.

68. The pharmaceutical composition of claim 63, wherein the sense strand comprises SEQ ID NO: 5870.

69. The pharmaceutical composition of any one of claims 63-68, wherein the antisense strand comprises SEQ ID NO: 5967.

70. The pharmaceutical composition of any one of claims 63-68, wherein the antisense strand comprises SEQ ID NO: 5968.

71. The pharmaceutical composition of any one of claims 63-68, wherein the antisense strand comprises SEQ ID NO: 5969.

72. The pharmaceutical composition of any one of claims 63-68, wherein the antisense strand comprises SEQ ID NO: 5970.

73. The pharmaceutical composition of any one of claims 63-68, wherein the antisense strand comprises SEQ ID NO: 5971.

74. The pharmaceutical composition of any one of claims 63-73, wherein the sense strand comprises pattern 1S.

75. The pharmaceutical composition of any one of claims 63-73, wherein the sense strand comprises pattern 2S.

76. The pharmaceutical composition of any one of claims 63-73, wherein the sense strand comprises pattern 3S.

77. The pharmaceutical composition of any one of claims 63-73, wherein the sense strand comprises pattern 4S.

78. The pharmaceutical composition of any one of claims 63-73, wherein the sense strand comprises pattern 5S.

79. The pharmaceutical composition of any one of claims 63-78, wherein the antisense strand comprises pattern 1AS.

80. The pharmaceutical composition of any one of claims 63-78, wherein the antisense strand comprises pattern 2AS.

81. The pharmaceutical composition of any one of claims 63-78, wherein the antisense strand comprises pattern 3AS.

82. The pharmaceutical composition of any one of claims 63-78, wherein the antisense strand comprises pattern 4AS.

83. A pharmaceutical composition comprising a sense strand and an antisense strand, wherein the sense strand or antisense strand comprises one or more sequences comprising the first 19 nucleobases of a sequence selected from SEQ ID NOS: 1-5349, 5366-5971, 6000-6038, 6050-6074; or a nucleic acid sequence thereof having about 1 or 2 nucleoside substitutions, additions, or deletions.

84. The pharmaceutical composition of any one of claims 58-83, comprising a pharmaceutically acceptable carrier.

85. The pharmaceutical composition of any one claims 58-84, comprising a modified internucleoside linkage.

86. The pharmaceutical composition of claim 85, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

87. The pharmaceutical composition of claim 86, wherein the modified internucleoside linkage comprises one or more phosphorothioate linkages.

88. The pharmaceutical composition of any one of claims 58-87, comprising a modified nucleoside.

89. The pharmaceutical composition of claim 88, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

90. The pharmaceutical composition of claim 88 or claim 89, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

91. The pharmaceutical composition of any one of claims 88-90, wherein the modified nucleoside comprises one or more 2′fluoro modified nucleosides.

92. The pharmaceutical composition of any one of claims 88-91, wherein the modified nucleoside comprises a 2′ O-alkyl modified nucleoside.

93. The pharmaceutical composition of any one of claims 88-92, comprising a ribose.

94. The pharmaceutical composition of any one of claims 88-93, further comprising a lipid.

95. The pharmaceutical composition of claim 94, wherein the lipid comprises cholesterol, myristoyl, palmitoyl, stearoyl, lithocholoyl, docosanoyl, docosahexaenoyl, myristyl, palmityl stearyl, or a-tocopherol, or a combination thereof.

96. The pharmaceutical composition of any one of claims 58-95, wherein the sense strand and the antisense strand form a double-stranded RNA duplex.

97. The pharmaceutical composition of claim 96, wherein the first base pair of the double-stranded RNA duplex is an AU base pair.

98. A method of treating one or more disorders of the upper and lower airway in a patient in need thereof, the method comprising administering to the patient a pharmaceutical composition of any one of claims 1-97.

99. The method of claim 98, wherein the one or more disorders of the upper and lower airways comprises nasal polyposis, chronic sinusitis, allergic rhinitis, or NSAID-exacerbated respiratory disease, or a combination thereof.

100. The method of claim 98 or claim 99, wherein the siRNA is administered in an effective amount to reduce eosinophil count in the patient.

101. The method of claim 99 or claim 100, wherein the siRNA is administered in an effective amount to reduce number and/or size of nasal polyps in the patient.

102. The method of claim 99 or claim 100, wherein the siRNA is administered in an effective amount to increase nasal inspiratory peak in the patient.

103. The method of claim 99 or claim 100, wherein the siRNA is administered in an effective amount to reduce airway symptoms in the patient.
